SEQUENCE LISTING (92 sequences)

SEQ ID NO: 1-2 - skipped (2 entries)

SEQ ID NO: 3 - DNA, 49 bp, synthetic construct
  source 1..49: mol_type = other DNA
ctctctacggctaacctgaatggactacgacatagtctagtccgccaag

SEQ ID NO: 4 - DNA, 101 bp, synthetic construct
  source 1..101: mol_type = other DNA
actctagctagcagtgttaaatcattcagctacctgagaggggcccctataactctctacggctaacctgaatggactacgacatagtctagtccgccaag

SEQ ID NO: 5 - DNA, 24 bp, synthetic construct
  source 1..24: mol_type = other DNA
caaaggctcttttcagagccacca

SEQ ID NO: 6 - DNA, 430 bp, Homo sapiens
  source 1..430: mol_type = genomic DNA
gcacactgccagtctcactgtgggaaggccagctgccccagccagatggactccagcctgcctggcaggctgtctggccaggcctcttggcagtcacatctcttcctccgaggccagtacctttccatttattctttgatcttcagggaactgcatagattgatcaaagtgtaaacaccatagggacccattctacacagagcaggactgcacagcgtcctgtccacacccagctcagcatttccacaccaagcagcaacagcaaatcacgaccactgatagatgtctattcttgttggagacatgggatgattattttctgttctatttgtgcttagtccaattccttgcacatagtaggtacccaattcaattactattgaatgaattaagaattggttgccataaaaataaatcagttcatttaaaa

SEQ ID NO: 7 - DNA, 1401 bp, Homo sapiens
  source 1..1401: mol_type = genomic DNA
actccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 8 - DNA, 304 bp, Homo sapiens
  source 1..304: mol_type = genomic DNA
gtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 9 - DNA, 209 bp, Homo sapiens
  source 1..209: mol_type = genomic DNA
gtacatggaggccaacaagaacataggcaagatcgtcctggaactgccccagtgaaggaggatggggcaggacaggacgcggccaccccaggcctttccagagcaaacctggagaagattcacaatagacaggccaagaaacccggtgcttcctccagagccgtttaaagctgatatgaggaaataaagagtgaactggaaaaaaaaaa

SEQ ID NO: 10 - DNA, 47 bp, Homo sapiens
  source 1..47: mol_type = genomic DNA
gggagatgccccacagcactgcccttccagaggcaagaccaaccaag

SEQ ID NO: 11 - DNA, 51 bp, Homo sapiens
  source 1..51: mol_type = genomic DNA
gggagatatatataggattgaagatctctcagttaagtctacatgaaaagg

SEQ ID NO: 12 - DNA, 33 bp, Homo sapiens
  source 1..33: mol_type = genomic DNA
gggactgcccttccagaggcaagaccaaccaag

SEQ ID NO: 13 - DNA, 63 bp, Homo sapiens
  source 1..63: mol_type = genomic DNA
gggagtatattagtgctaatttccctccgtttgtcctagcttttctcttctgtcaaccccaca

SEQ ID NO: 14 - DNA, 96 bp, Homo sapiens
  source 1..96: mol_type = genomic DNA
gggagataaaaagccagctccagcaggcgctgctcactcctccccatcctctccctctgtccctctgtccctctgaccctgcactgtcccagcacc

SEQ ID NO: 15 - DNA, 61 bp, Homo sapiens
  source 1..61: mol_type = genomic DNA
gggagcaatcctttctttcagctggagtgctcctcaggagccagccccacccttagaaaag

SEQ ID NO: 16 - DNA, 474 bp, Homo sapiens
  source 1..474: mol_type = genomic DNA
ggggtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagac

SEQ ID NO: 17 - DNA, 85 bp, Homo sapiens
  source 1..85: mol_type = genomic DNA
ggggcttactcggcgcccgcgcctcgggccgtcgggagcggagcctcctcgggaccaggacttcagggccacaggtgctgccaag

SEQ ID NO: 18 - DNA, 82 bp, Homo sapiens
  source 1..82: mol_type = genomic DNA
gggattcatgaaaatccactactccagacagacggctttggaatccaccagctacatccagctccctgaggcagagttgaga

SEQ ID NO: 19 - DNA, 264 bp, Homo sapiens
  source 1..264: mol_type = genomic DNA
gggacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaca

SEQ ID NO: 20 - DNA, 47 bp, synthetic construct
  source 1..47: mol_type = other DNA
gggaaataagagagaaaagaagagtaagaagaaatataagagccacc

SEQ ID NO: 21 - DNA, 47 bp, synthetic construct
  source 1..47: mol_type = other DNA
gggagatcagagagaaaagaagagtaagaagaaatataagagccacc

SEQ ID NO: 22 - DNA, 145 bp, synthetic construct
  source 1..145: mol_type = other DNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcaac

SEQ ID NO: 23 - DNA, 42 bp, synthetic construct
  source 1..42: mol_type = other DNA
gggagacaagcttggcattccggtactgttggtaaagccacc

SEQ ID NO: 24 - DNA, 47 bp, synthetic construct; identical to SEQ ID NO: 21
  source 1..47: mol_type = other DNA

SEQ ID NO: 25 - DNA, 145 bp, synthetic construct; identical to SEQ ID NO: 22
  source 1..145: mol_type = other DNA

SEQ ID NO: 26 - DNA, 42 bp, synthetic construct; identical to SEQ ID NO: 23
  source 1..42: mol_type = other DNA

SEQ ID NO: 27 - DNA, 47 bp, synthetic construct
  source 1..47: mol_type = other DNA
gggaattaacagagaaaagaagagtaagaagaaatataagagccacc

SEQ ID NO: 28 - DNA, 47 bp, synthetic construct
  source 1..47: mol_type = other DNA
gggaaattagacagaaaagaagagtaagaagaaatataagagccacc

SEQ ID NO: 29 - DNA, 47 bp, synthetic construct
  source 1..47: mol_type = other DNA
gggaaataagagagtaaagaacagtaagaagaaatataagagccacc

SEQ ID NO: 30 - DNA, 47 bp, synthetic construct
  source 1..47: mol_type = other DNA
gggaaaaaagagagaaaagaagactaagaagaaatataagagccacc

SEQ ID NO: 31 - DNA, 47 bp, synthetic construct
  source 1..47: mol_type = other DNA
gggaaataagagagaaaagaagagtaagaagatatataagagccacc

SEQ ID NO: 32 - DNA, 47 bp, synthetic construct
  source 1..47: mol_type = other DNA
gggaaataagagacaaaacaagagtaagaagaaatataagagccacc

SEQ ID NO: 33 - DNA, 47 bp, synthetic construct
  source 1..47: mol_type = other DNA
gggaaattagagagtaaagaacagtaagtagaattaaaagagccacc

SEQ ID NO: 34 - DNA, 47 bp, synthetic construct
  source 1..47: mol_type = other DNA
gggaaataagagagaatagaagagtaagaagaaatataagagccacc

SEQ ID NO: 35 - DNA, 47 bp, synthetic construct
  source 1..47: mol_type = other DNA
gggaaataagagagaaaagaagagtaagaagaaaattaagagccacc

SEQ ID NO: 36 - DNA, 47 bp, synthetic construct
  source 1..47: mol_type = other DNA
gggaaataagagagaaaagaagagtaagaagaaatttaagagccacc

SEQ ID NO: 37 - DNA, 47 bp, synthetic construct
  source 1..47: mol_type = other DNA
gggaaataagagagaaaagaagagtaagaagaaatataagagcctcc

SEQ ID NO: 38 - DNA, 41 bp, synthetic construct
  source 1..41: mol_type = other DNA
gggaaataagagagaaaagaagagtaagaagaaatatatga

SEQ ID NO: 39 - DNA, 38 bp, synthetic construct
  source 1..38: mol_type = other DNA
gggaaataagagagaaaagaagagtaagaagaaatata

SEQ ID NO: 40 - DNA, 371 bp, Homo sapiens
  source 1..371: mol_type = genomic DNA
gcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggcctttggtctttgaataaagcctgagtaggaagtctaga

SEQ ID NO: 41 - DNA, 568 bp, Homo sapiens
  source 1..568: mol_type = genomic DNA
gcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaataaaacaacctgcaacatctcatggtctttgaataaagcctgagtaggaagtctaga

SEQ ID NO: 42 - DNA, 289 bp, Homo sapiens
  source 1..289: mol_type = genomic DNA
acacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaatggtctttgaataaagcctgagtaggaagtctaga

SEQ ID NO: 43 - DNA, 379 bp, Homo sapiens
  source 1..379: mol_type = genomic DNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctaga

SEQ ID NO: 44 - DNA, 118 bp, Homo sapiens
  source 1..118: mol_type = genomic DNA
gctgccttctgcggggcttgccttctggccatgcccttcttctctcccttgcacctgtacctcttggtctttgaataaagcctgagtaggaaggcggccgctcgagcatgcatctaga

SEQ ID NO: 45 - DNA, 908 bp, Homo sapiens
  source 1..908: mol_type = genomic DNA
gccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccatggtctttgaataaagcctgagtaggaaggcggccgctcgagcatgcatctaga

SEQ ID NO: 46 - DNA, 835 bp, Homo sapiens
  source 1..835: mol_type = genomic DNA
actcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 47 - DNA, 297 bp, Homo sapiens
  source 1..297: mol_type = genomic DNA
cgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccat

SEQ ID NO: 48 - DNA, 602 bp, Homo sapiens
  source 1..602: mol_type = genomic DNA
ggggctagagccctctccgcacagcgtggagacggggcaaggaggggggttattaggattggtggttttgttttgctttgtttaaagccgtgggaaaatggcacaactttacctctgtgggagatgcaacactgagagccaaggggtgggagttgggataatttttatataaaagaagtttttccactttgaattgctaaaagtggcatttttcctatgtgcagtcactcctctcatttctaaaatagggacgtggccaggcacggtggctcatgcctgtaatcccagcactttgggaggccgaggcaggcggctcacgaggtcaggagatcgagactatcctggctaacacggtaaaaccctgtctctactaaaagtacaaaaaattagctgggcgtggtggtgggcacctgtagtcccagctactcgggaggctgaggcaggagaaaggcatgaatccaagaggcagagcttgcagtgagctgagatcacgccattgcactccagcctgggcaacagtgttaagactctgtctcaaatataaataaataaataaataaataaataaataaataaaaataaagcgagatgttgccctcaaa

SEQ ID NO: 49 - DNA, 785 bp, Homo sapiens
  source 1..785: mol_type = genomic DNA
ggccctgccccgtcggactgcccccagaaagcctcctgccccctgccagtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattcctttacaactaaataacacagatattgttataaataaaattgt

SEQ ID NO: 50 - DNA, 3001 bp, Homo sapiens
  source 1..3001: mol_type = genomic DNA
atattaaggatcaagctgttagctaataatgccacctctgcagttttgggaacaggcaaataaagtatcagtatacatggtgatgtacatctgtagcaaagctcttggagaaaatgaagactgaagaaagcaaagcaaaaactgtatagagagatttttcaaaagcagtaatccctcaattttaaaaaaggattgaaaattctaaatgtctttctgtgcatattttttgtgttaggaatcaaaagtattttataaaaggagaaagaacagcctcattttagatgtagtcctgttggattttttatgcctcctcagtaaccagaaatgttttaaaaaactaagtgtttaggatttcaagacaacattatacatggctctgaaatatctgacacaatgtaaacattgcaggcacctgcattttatgttttttttttcaacaaatgtgactaatttgaaacttttatgaacttctgagctgtccccttgcaattcaaccgcagtttgaattaatcatatcaaatcagttttaattttttaaattgtacttcagagtctatatttcaagggcacattttctcactactattttaatacattaaaggactaaataatctttcagagatgctggaaacaaatcatttgctttatatgtttcattagaataccaatgaaacatacaacttgaaaattagtaatagtatttttgaagatcccatttctaattggagatctctttaatttcgatcaacttataatgtgtagtactatattaagtgcacttgagtggaattcaacatttgactaataaaatgagttcatcatgttggcaagtgatgtggcaattatctctggtgacaaaagagtaaaatcaaatatttctgcctgttacaaatatcaaggaagacctgctactatgaaatagatgacattaatctgtcttcactgtttataatacggatggattttttttcaaatcagtgtgtgttttgaggtcttatgtaattgatgacatttgagagaaatggtggctttttttagctacctctttgttcatttaagcaccagtaaagatcatgtctttttatagaagtgtagattttctttgtgactttgctatcgtgcctaaagctctaaatataggtgaatgtgtgatgaatactcagattatttgtctctctatataattagtttggtactaagtttctcaaaaaattattaacacatgaaagacaatctctaaaccagaaaaagaagtagtacaaattttgttactgtaatgctcgcgtttagtgagtttaaaacacacagtatcttttggttttataatcagtttctattttgctgtgcctgagattaagatctgtgtatgtgtgtgtgtgtgtgtgtgcgtttgtgtgttaaagcagaaaagacttttttaaaagttttaagtgataaatgcaatttgttaattgatcttagatcactagtaaactcagggctgaattataccatgtatattctattagaagaaagtaaacaccatctttattcctgccctttttcttctctcaaagtagttgtagttatatctagaaagaagcaattttgatttcttgaaaaggtagttcctgcactcagtttaaactaaaaataatcatacttggattttatttatttttgtcatagtaaaaattttaatttatatatatttttatttagtattatcttattctttgctatttgccaatcctttgtcatcaattgtgttaaatgaattgaaaattcatgccctgttcattttattttactttattggttaggatatttaaaggatttttgtatatataatttcttaaattaatattccaaaaggttagtggacttagattataaattatggcaaaaatctaaaaacaacaaaaatgatttttatacattctatttcattattcctctttttccaataagtcatacaattggtagatatgacttattttatttttgtattattcactatatctttatgatatttaagtataaataattaaaaaaatttattgtaccttatagtctgtcaccaaaaaaaaaaaattatctgtaggtagtgaaatgctaatgttgatttgtctttaagggcttgttaactatcctttattttctcatttgtcttaaattaggagtttgtgtttaaattactcatctaagcaaaaaatgtatataaatcccattactgggtatatacccaaaggattataaatcatgctgctataaagacacatgcacacgtatgtttattgcagcactattcacaatagcaaagacttggaaccaacccaaatgtccatcaatgatagacttgattaagaaaatgtgcacatatacaccatggaatactatgcagccataaaaaaggatgagttcatgtcctttgtagggacatggataaagctggaaaccatcattctgagcaaactattgcaaggacagaaaaccaaacactgcatgttctcactcataggtgggaattgaacaatgagaacacttggacacaaggtggggaacaccacacaccagggcctgtcatggggtggggggagtggggagggatagcattaggagatatacctaatgtaaatgatgagttaatgggtgcagcacaccaacatggcacatgtatacatatgtagcaaacctgcacgttgtgcacatgtaccctagaacttaaagtataattaaaaaaaaaaagaaaacagaagctatttataaagaagttatttgctgaaataaatgtgatctttcccattaaaaaaataaagaaattttggggtaaaaaaacacaatatattgtattcttgaaaaattctaagagagtggatgtgaagtgttctcaccacaaaagtgataactaattgaggtaatgcacatattaattagaaagattttgtcattccacaatgtatatatacttaaaaatatgttatacacaataaatacatacattaaaaaataagtaaatgta

SEQ ID NO: 51 - DNA, 1037 bp, Homo sapiens
  source 1..1037: mol_type = genomic DNA
cccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctc

SEQ ID NO: 52 - DNA, 577 bp, Homo sapiens
  source 1..577: mol_type = genomic DNA
agaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 53 - DNA, 2212 bp, Homo sapiens
  source 1..2212: mol_type = genomic DNA
ctccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataatccgcagtggcctcctaatttccttctttggagttgggggaggggtagacatggggaaggggctttggggtgatgggcttgccttccattcctgccctttccctccccactattctcttctagatccctccataaccccactcccctttctctcacccttcttataccgcaaacctttctacttcctctttcattttctattcttgcaatttccttgcaccttttccaaatcctcttctcccctgcaataccatacaggcaatccacgtgcacaacacacacacacactcttcacatctggggttgtccaaacctcatacccactccccttcaagcccatccactctccaccccctggatgccctgcacttggtggcggtgggatgctcatggatactgggagggtgaggggagtggaacccgtgaggaggacctgggggcctctccttgaactgacatgaagggtcatctggcctctgctcccttctcacccacgctgacctcctgccgaaggagcaacgcaacaggagaggggtctgctgagcctggcgagggtctgggagggaccaggaggaaggcgtgctccctgctcgctgtcctggccctgggggagtgagggagacagacacctgggagagctgtggggaaggcactcgcaccgtgctcttgggaaggaaggagacctggccctgctcaccacggactgggtgcctcgacctcctgaatccccagaacacaacccccctgggctggggtggtctggggaaccatcgtgcccccgcctcccgcctactcctttttaagctt

SEQ ID NO: 54 - DNA, 729 bp, Homo sapiens
  source 1..729: mol_type = genomic DNA
ttggccaggcctgaccctcttggacctttcttctttgccgacaaccactgcccagcagcctctgggacctcggggtcccagggaacccagtccagcctcctggctgttgacttcccattgctcttggagccaccaatcaaagagattcaaagagattcctgcaggccagaggcggaacacacctttatggctggggctctccgtggtgttctggacccagcccctggagacaccattcacttttactgctttgtagtgactcgtgctctccaacctgtcttcctgaaaaaccaaggcccccttcccccacctcttccatggggtgagacttgagcagaacaggggcttccccaagttgcccagaaagactgtctgggtgagaagccatggccagagcttctcccaggcacaggtgttgcaccagggacttctgcttcaagttttggggtaaagacacctggatcagactccaagggctgccctgagtctgggacttctgcctccatggctggtcatgagagcaaaccgtagtcccctggagacagcgactccagagaacctcttgggagacagaagaggcatctgtgcacagctcgatcttctacttgcctgtggggaggggagtgacaggtccacacaccacactgggtcaccctgtcctggatgcctctgaagagagggacagaccgtcagaaactggagagtttctattaaaggtcatttaaacca

SEQ ID NO: 55 - DNA, 847 bp, Homo sapiens
  source 1..847: mol_type = genomic DNA
tcctccgggaccccagccctcaggattcctgatgctccaaggcgactgatgggcgctggatgaagtggcacagtcagcttccctgggggctggtgtcatgttgggctcctggggcgggggcacggcctggcatttcacgcattgctgccaccccaggtccacctgtctccactttcacagcctccaagtctgtggctcttcccttctgtcctccgaggggcttgccttctctcgtgtccagtgaggtgctcagtgatcggcttaacttagagaagcccgccccctccccttctccgtctgtcccaagagggtctgctctgagcctgcgttcctaggtggctcggcctcagctgcctgggttgtggccgccctagcatcctgtatgcccacagctactggaatccccgctgctgctccgggccaagcttctggttgattaatgagggcatggggtggtccctcaagaccttcccctaccttttgtggaaccagtgatgcctcaaagacagtgtcccctccacagctgggtgccaggggcaggggatcctcagtatagccggtgaaccctgataccaggagcctgggcctccctgaacccctggcttccagccatctcatcgccagcctcctcctggacctcttggcccccagccccttccccacacagccccagaagggtcccagagctgaccccactccaggacctaggcccagcccctcagcctcatctggagcccctgaagaccagtcccacccacctttctggcctcatctgacactgctccgcatcctgctgtgtgtcctgttccatgttccggttccatccaaatacactttctggaacaaa

SEQ ID NO: 56 - DNA, 110 bp, Homo sapiens
  source 1..110: mol_type = genomic DNA
gctggagcctcggtggccatgcttcttgccccttgggcctccccccagcccctcctccccttcctgcacccgtacccccgtggtctttgaataaagtctgagtgggcggc

SEQ ID NO: 57 - DNA, 168 bp, Homo sapiens
  source 1..168: mol_type = genomic DNA
cgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagcc

SEQ ID NO: 58 - DNA, 18 bp, synthetic construct
  source 1..18: mol_type = other DNA
aattctgacatccggcgg

SEQ ID NO: 59 - DNA, 10 bp, synthetic construct
  source 1..10: mol_type = other DNA
gcatatgact

SEQ ID NO: 60 - DNA, 54 bp, Homo sapiens
  source 1..54: mol_type = genomic DNA
gagaataaactagtattcttctggtccccacagactcagagagaacccgccacc

SEQ ID NO: 61 - DNA, 295 bp, synthetic construct
  source 1..295: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 62 - DNA, 57 bp, synthetic construct
  source 1..57: mol_type = other DNA
gggaaataagagagaaaagaagagtaagaagaaatataagaccccggcgccgccacc

SEQ ID NO: 63 - DNA, 110 bp, Homo sapiens
  source 1..110: mol_type = genomic DNA
gctggagcctcggtggcctagcttcttgccccttgggcctccccccagcccctcctccccttcctgcacccgtacccccgtggtctttgaataaagtctgagtgggcggc

SEQ ID NO: 64 - DNA, 1692 bp, synthetic construct
  source 1..1692: mol_type = other DNA
atggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaa

SEQ ID NO: 65 - DNA, 1350 bp, synthetic construct
  source 1..1350: mol_type = other DNA
atgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtag

SEQ ID NO: 66 - DNA, 759 bp, synthetic construct
  source 1..759: mol_type = other DNA
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtga

SEQ ID NO: 67 - DNA, 294 bp, synthetic construct
  source 1..294: mol_type = other DNA
atgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 68 - DNA, 660 bp, synthetic construct
  source 1..660: mol_type = other DNA
atggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatga

SEQ ID NO: 69 - DNA, 1497 bp, synthetic construct
  source 1..1497: mol_type = other DNA
atggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaggagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttga

SEQ ID NO: 70 - DNA, 759 bp, synthetic construct
  source 1..759: mol_type = other DNA
atgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtga

SEQ ID NO: 71 - DNA, 1701 bp, synthetic construct
  source 1..1701: mol_type = other DNA
atgaagactatcattgctttgagctacattctatgtctggttttcactcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtaaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagtttttcttgcataaggggatctagtagtagtttctttagtagattaaattggttgacccatttaaactacacatatccagcactgaacgtgactatgccaaacaaggaacaatttgacaaattgtacatttggggggttcaccacccgggtacggacaaggaccaaatctccctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtgaacaggatcacatacggggcctgtcccagatatgttaagcaaagcactctgaaattggcaacaggaatgcgaaatgtaccagagaaacaaactagaggcatatttggtgcaatagcgggtttcatagaaaatggttgggagggaatggtggatggttggtacggtttcagacatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaacgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatctgcatttga

SEQ ID NO: 72 - DNA, 1410 bp, synthetic construct
  source 1..1410: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccgaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtgacattacaggatttgcacctttctctaaggacaattcgattaggctttccgctggtggggacatctgggtgacaagagaaccttatgtgtcatgcgaccttgacaagtgttatcaatttgcacttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgccacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcagggcttgttggagacacacccagaaaaagcgacagctccagcagtagccattgtttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagtggatagaggtgataggtccggatacagcggtattttctctgttgaaggcaaaagctgcatcaatcggtgcttctatgtggagttgattaggggaagaaaagaggaaactgaagtcttgtggacctcaaacagtattgttgtgttttgtggcacctcaggtacatatggaacaggctcatggcctgatggggcggacctcaatctcatgcatacataa

SEQ ID NO: 73 - DNA, 1755 bp, synthetic construct
  source 1..1755: mol_type = other DNA
atgaaggcaataattgtactactcatggtagtaacatccaatgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggatttttttcaacaatggcttgggctgttccaaaggacaactacaaaaatgcaacgaacccacaaacagtggaagtaccatacatttgtacagaaggggaagaccaaattactgtttgggggttccattcggataacaaaacccaaatgaagtccctctatggagactcaaatcctcaaaagttcacctcatctgctaatggagtgaccacacattatgtttctcaaattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaatattctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatttgtctataa

SEQ ID NO: 74 - DNA, 1401 bp, synthetic construct
  source 1..1401: mol_type = other DNA
atgctaccttcaactatacaaacgttaaccttatttctcacattagggggagtgctattatcactatatgtgtcagcttcattgtcatacttactatattcggatatattgctaaaattttcacgaacagaagtaactgcaccaataatgccattggattgtgcaaacgcatcaaatgtccaggctgtgaatcgttctgcaacaaaaggggtgacacctcttctcccagaaccggagtggacgtaccctcgtttatcttgcccgggctcaacctttcagaaagcactcctaattagcccccatagattcggagaaaccaaaggaaactcagctcccttaataataagggaaccttttattgcttgtggaccaaaggaatgcaaacattttgctctaacccattatgcagctcaaccagggggatactacaatggaacaagagaagacagaaacaagctgaggcatctaatttcagtcaaattgggcaaaatcccaacagtagaaaactctattttccacatggcagcttggagtgggtccgcatgccatgatggtagagaatggacttacatcggagttgatggcccagacagtaatgcattgctcaaaataaaatatggagaagcatatactgacacataccattcctatgcaaaaaacatcctaaggacacaagaaagtgcctgcaattgcatcgggggagattgttatctgatgataactgatggcccagcttcagggattagtgaatgcagattccttaagattcgagagggccgaataataaaagaaatatttccaacaggaagagtaaaacacactgaggaatgcacatgcggatttgccagcaacaaaaccatagaatgtgcctgtagagataacagttacacagcaaaaagaccctttgtcaaattaaatgtggagaccgatacagcggaaataagattgatgtgcacaaagacttatttggacacccccagaccaaatgatggaagcataacagggccttgcgaatctgatggaaacgaagggggtggaggcatcaagggaggatttgttcaccaaagaatggcatccaagattggaaggtggtactctcggacgatgtctaaaactcaaagaatggggatgggactgtatgtaaagtatgatggagacccatggactgacagtgaagcccttgctcttagtggagtaatggtttcaatgggagaacctggttggtattcctttggcttcgaaataaaagataagaaatgtgatgtcccctgtattgggatagaaatggtacatgatggtgggaaaacgacttggcactcagcggcaacagccatttactgtttaatgggctcaggacaactgctgtgggacactgtcacaggtgttaatatgactctgtaa

SEQ ID NO: 75 - DNA, 61 bp, synthetic construct
  source 1..61: mol_type = other DNA
gggcccctataactctctacggctaacctgaatggactacgacatagtctagtccgccaag

SEQ ID NO: 76 - DNA, 44 bp, Venezuelan equine encephalitis virus
  source 1..44: mol_type = other DNA
ataggcggcgcatgagagaagcccagaccaattacctacccaaa

SEQ ID NO: 77 - DNA, 117 bp, Venezuelan equine encephalitis virus
  source 1..117: mol_type = other DNA
atacagcagcaattggcaagctgcttacatagaactcgcggcgattggcatgccgccttaaaatttttattttatttttcttttcttttccgaatcggattttgtttttaatatttc

SEQ ID NO: 78 - DNA, 19 bp, Venezuelan equine encephalitis virus
  source 1..19: mol_type = other DNA
attttgtttttaatatttc

SEQ ID NO: 79 - DNA, 341 bp, Sindbis virus
  source 1..341: mol_type = other DNA
ggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 80 - DNA, 19 bp, Sindbis virus
  source 1..19: mol_type = other DNA
attttgtttttaacatttc

SEQ ID NO: 81 - DNA, 14091 bp, synthetic construct
  source 1..14091: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 82 - DNA, 14103 bp, synthetic construct
  source 1..14103: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtaggggcccctataactctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 83 - DNA, 14103 bp, synthetic construct
  source 1..14103: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaagggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 84 - DNA, 14115 bp, synthetic construct
  source 1..14115: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaagggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtaggggcccctataactctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 85 - skipped

SEQ ID NO: 86 - DNA, 80 bp, synthetic construct
  source 1..80: mol_type = other DNA
tcattcagctacctgagaggggcccctataactctctacggctaacctgaatggactacgacatagtctagtccgccaag

SEQ ID NO: 87 - DNA, 71 bp, synthetic construct
  source 1..71: mol_type = other DNA
gggcccctataactctctacggctaacctgaatggacctgaatggactacgacatagtctagtccgccaag

SEQ ID NO: 88 - DNA, 575 bp, synthetic construct
  source 1..575: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 89 - DNA, 10 bp, synthetic construct
  source 1..10: mol_type = other DNA
ccccggcgcc

SEQ ID NO: 90 - skipped

SEQ ID NO: 91 - DNA, 20 bp, synthetic construct
  source 1..20: mol_type = other DNA
gcgccccgcggcgccccgcg

SEQ ID NO: 92 - DNA, 18 bp, synthetic construct
  source 1..18: mol_type = other DNA
taatacgactcactatag

(19) United States
(12) Patent Application Publication
(10) Pub. No.: US 2026/0034204 A1
(43) Pub. Date: Feb. 5, 2026

(54) RNA ENCODING VIRUS-LIKE PARTICLES AND USES THEREOF

(71) Applicant: SEQIRUS INC., Holly Springs, NC (US)

(72) Inventors: Sukhmani BEDI, Holly Spring, NC (US); Cheng CHANG, Holly Spring, NC (US); Pirada ALLEN, Holly Springs, NC (US)

(21) Appl. No.: 19/101,399

(22) PCT Filed: Aug. 8, 2023
(86) PCT No.: PCT/IB2023/057979

§ 371 Date: Feb. 5, 2025

Related U.S. Application Data
(60) Provisional application No. 63/370,725, filed on Aug. 8, 2022.

(51) Int. Cl. A61K 39/145; A61K 39/00; C07K 14/005; C12N 7/00

(52) U.S. Cl. CPC A61K 39/145; C07K 14/005; C12N 7/00; A61K 2039/5258; A61K 2039/53; A61K 2039/55555; C12N 2760/16123; C12N 2760/16134; C12N 2770/36122; C12N 2830/50; C12N 2840/203

(57) ABSTRACT

The present disclosure relates to a composition comprising one or more ribonucleic acids (RNAs) encoding virus-like particle (VLP) forming elements of an influenza virus. The present disclosure further provides uses of the composition.

### RELATED APPLICATION DATA

The present application claims priority from U.S. Patent Application No. 63/370,725 filed 8 Aug. 2022 entitled “RNA encoding virus-like particles and uses thereof”, the entire contents of which is hereby incorporated by reference.

### SEQUENCE LISTING

The present application is filed together with a Sequence Listing in electronic format. The entire contents of the Sequence Listing are hereby incorporated by reference.

### FIELD

The present disclosure relates to a composition comprising one or more ribonucleic acids (RNAs) encoding virus-like particle (VLP) forming elements of an influenza virus. The present disclosure further provides uses of the composition.

### BACKGROUND

Influenza viral infections are a significant threat to human health and lives. The World Health Organisation (WHO) estimates there are approximately 1 billion cases of influenza each year, 3 to 5 million of those are severe cases, and 290,000-650,000 result in influenza-related respiratory deaths. Currently, infections from influenza are treated with antivirals or other drugs.

Viral vaccines for influenza rely upon the induction of antibodies that protect against infection by neutralizing virions or blocking the virus's entry into cells. Humoral immune responses target viral surface proteins. To enhance the immune response (e.g., antibody response) mounted to the influenza virus surface proteins, various adjuvants and immuno-potentiating agents are included in the vaccine formulation. However, safety and efficacy issues remain.

Currently, egg-based manufacturing processes are the most common way that influenza vaccines are produced. This process requires a significant amount of time to optimize virus growth in the eggs, as well as resources (i.e., eggs) to produce sufficient amounts of vaccine, particularly during a pandemic. Furthermore, given the long development time required, vaccine strain selection is conducted before the vaccine is made available, making it difficult to respond to changes in the virus. Influenza vaccines have also been produced using cell-based manufacturing processes involving cultured mammalian cells (e.g. Madin-Darby Canine Kidney cells) in place of eggs, and viral-based manufacturing processes involving recombinant virus (e.g. baculovirus encoding an antigen of influenza).

There remains a need for the development of specific and efficient influenza vaccines that can be produced more rapidly than current egg-based techniques, for the treatment or prevention of influenza. Nucleic acid-based vaccines offer distinct advantages over the current egg-based, cell-based and viral-based manufacturing platform, although some challenges remain. For example, nucleic acid-based vaccines which produce antigenic portions of a pathogen can often result in poor recognition by the immune system resulting in limited ability of the nucleic acid-based vaccines to produce a strong, durable immune response. Therefore, it will be apparent to the skilled person that there is a need in the art for an influenza virus vaccine which results in improved induction of an immune response in a subject.

### SUMMARY

The present disclosure is based on the inventors' search for a composition that has improved ability to elicit an immune response in a subject against an influenza virus. In particular, the inventors determined that including a sequence encoding structural protein of an influenza virus matrix-1 (M1) along with influenza virus antigens hemagglutinin (HA) protein and neuraminidase (NA) protein into ribonucleic acid(s) (RNA(s)) of the composition permits the expression of virus-like particles (VLPs) of the influenza virus which is predicted to elicit improved recognition of the influenza virus antigens by the immune system compared to free-floating antigens (e.g., HA alone) of the influenza virus. Furthermore, the inventors determined that the stability of VLPs produced from a composition described herein could be increased by increasing M1 protein incorporation in the VLPs. The inventors also determined that efficient release of VLPs from, for example, a cell could be increased by increasing NA protein incorporation in the VLPs.

The findings by the inventors provide the basis for a composition comprising one or more ribonucleic acids (RNAs) encoding VLP forming elements hemagglutinin (HA) protein, neuraminidase (NA) protein, and matrix-1 (M1) protein of an influenza virus. The findings by the inventors also provides the basis for methods of treating, preventing or delaying progression of influenza in a subject using the composition.

Accordingly, the present disclosure provides a composition comprising one or more ribonucleic acids (RNAs), each RNA comprising in 5′ to 3′ order:
a) a first nucleotide sequence comprising a 5′-untranslated region (5′-UTR), a fragment and/or a variant thereof,
b) one or more nucleotide sequence(s) encoding a virus-like particle (VLP) forming element;
c) a second nucleotide sequence comprising a 3′-untranslated region (3′-UTR), a fragment and/or a variant thereof, and
   wherein the VLP forming element is selected from a hemagglutinin (HA) protein, a neuraminidase (NA) protein, and a matrix-1 (M1) protein of an influenza virus, and
   wherein the composition comprises nucleotide sequences encoding each of the HA protein, the NA protein and the M1 protein.

In one example, upon introduction into a cell, the level of expression of the M1 protein is expressed at a higher level than the level of the HA protein and/or the NA protein. As exemplified herein, the inventors have shown that increasing the level of expression of the M1 protein increases the production of intact VLPs and/or stability of the resulting VLPs.

The present disclosure provides a method of increasing production of an intact VLP and/or stability of a VLP, the method comprising introducing a nucleotide sequence encoding a matrix-1 (M1) protein of an influenza virus into a composition, wherein the composition comprises one or more ribonucleic acids (RNAs), each RNA comprising in 5′ to 3′ order:
a) a first nucleotide sequence comprising a 5′-untranslated region (5′-UTR), a fragment and/or a variant thereof,
b) one or more nucleotide sequence(s) encoding a virus-like particle (VLP) forming element;
c) a second nucleotide sequence comprising a 3′-untranslated region (3′-UTR), a fragment and/or a variant thereof,
   wherein the VLP forming element is selected from a hemagglutinin (HA) protein, a neuraminidase (NA) protein, and the M1 protein of an influenza virus,
   wherein the composition comprises nucleotide sequences encoding each of the HA protein, the NA protein and the M1 protein,
   and wherein following introduction of the RNA(s) into a cell, the M1 protein is expressed at a higher level than the HA protein and/or the NA protein.

The present disclosure further provides a method of increasing efficient release of a virus-like particle (VLP), the method comprising introducing a nucleotide sequence encoding a neuraminidase (NA) protein of an influenza virus into a composition, wherein the composition comprises one or more ribonucleic acids (RNAs), each RNA comprising in 5′ to 3′ order:
a) a first nucleotide sequence comprising a 5′-untranslated region (5′-UTR), a fragment and/or a variant thereof,
b) one or more nucleotide sequence(s) encoding a virus-like particle (VLP) forming element;
c) a second nucleotide sequence comprising a 3′-untranslated region (3′-UTR), a fragment and/or a variant thereof,
   wherein the VLP forming element is selected from a hemagglutinin (HA) protein, the NA protein, and a M1 protein of an influenza virus,
   wherein the composition comprises nucleotide sequences encoding each of the HA protein, the NA protein and the M1 protein,
   and wherein following introduction of the RNA(s) into a cell, the NA protein is expressed at a higher level than the M1 protein or the HA protein.

The present disclosure also provides a method of increasing stability and efficient release of a virus-like particle (VLP), the method comprising introducing a nucleotide sequence encoding a matrix-1 (M1) protein and a nucleotide sequence encoding a neuraminidase (NA) protein of an influenza virus into a composition, wherein the composition comprises one or more ribonucleic acids (RNAs), each RNA comprising in 5′ to 3′ order:
a) a first nucleotide sequence comprising a 5′-untranslated region (5′-UTR), a fragment and/or a variant thereof,
b) one or more nucleotide sequence(s) encoding a virus-like particle (VLP) forming element;
c) a second nucleotide sequence comprising a 3′-untranslated region (3′-UTR), a fragment and/or a variant thereof,
   wherein the VLP forming element is selected from a hemagglutinin (HA) protein, the NA protein, and the M1 protein of an influenza virus,
   wherein the composition comprises nucleotide sequences encoding each of the HA protein, the NA protein and the M1 protein,
   and wherein introducing the nucleotide sequence encoding the M1 protein and the NA protein into the composition increases the stability and efficient release of the VLP produced from the composition.

In one example, the composition comprises a RNA comprising a nucleotide sequence encoding the HA protein, a nucleotide sequence encoding the NA protein, and a nucleotide sequence encoding the M1 protein.

In one example, the composition comprises a RNA comprising, in 5′ to 3′ order, a nucleotide sequence encoding the HA protein, a nucleotide sequence encoding the NA protein, and a nucleotide sequence encoding the M1 protein. In one example, the sequence encoding the NA protein is linked to an extended subgenomic promoter and the sequence encoding the M1 protein is linked to an extended subgenomic promoter. In one example, the sequence encoding the NA protein is linked to extended subgenomic promoter v2 and the sequence encoding the M1 protein is linked to subgenomic promoter v1.

In one example, the composition comprises a first and a second RNA, wherein the first RNA comprises a nucleotide sequence encoding the HA protein, the NA protein, or the M1 protein; and a second RNA comprising nucleotide sequences encoding a combination of:
a) the HA protein and the M1 protein;
b) the NA protein and the M1 protein; or
c) the HA protein and the NA protein, and wherein the first RNA and second RNA encode different VLP forming elements.

For example, the first RNA comprises a nucleotide sequence encoding HA protein, and second RNA comprises a nucleotide sequence encoding a M1 protein and a nucleotide sequence encoding NA protein. For example, the first RNA comprises a nucleotide sequence encoding NA protein, and second RNA comprises a nucleotide sequence encoding a M1 protein and a nucleotide sequence encoding HA protein. For example, the first RNA comprises a nucleotide sequence encoding M1 protein, and second RNA comprising a nucleotide sequence encoding a HA protein and a nucleotide sequence encoding NA protein.

In one example, the composition comprises a first RNA comprising a nucleotide sequence encoding the HA protein, a second RNA comprising a nucleotide sequence encoding the NA protein, and a third RNA comprising a nucleotide sequence encoding the M1 protein.

In one example, the disclosure provides a composition comprising a RNA comprising sequences encoding the HA protein, the NA protein, and the M1 protein.

In one example, the order of the coding sequences (5′-3′) is sequence encoding the HA protein, sequence encoding the NA protein, and sequence encoding the M1 protein. In one example, the sequence encoding the NA protein and the sequence encoding the M1 protein are each linked to a subgenomic promoter. For example, the sequence encoding the NA protein is linked to subgenomic promoter v2 and the sequence encoding the M1 protein is linked to the subgenomic promoter v1. For example, the sequence encoding the NA protein is linked to subgenomic promoter v2 and the sequence encoding the M1 protein is linked to the subgenomic promoter v2.

In one example, the disclosure provides a composition comprising a RNA comprising sequences encoding the HA protein, the NA protein, the M1 protein and the M2 protein.

In one example, the order of the coding sequences (5′-3′) is sequence encoding the M1 protein, sequence encoding the M2 protein, sequence encoding the NA protein and sequence encoding the HA protein. In one example, the sequence encoding the M1 protein, the sequence encoding the NA protein and the sequence encoding the M1 protein are each linked to a subgenomic promoter. In one example, the sequence encoding the M2 protein, the sequence encoding the NA protein and the sequence encoding the HA protein are each linked to a subgenomic promoter.

In the foregoing examples, an exemplary HA protein is a H5 protein. An exemplary NA protein is a N1 protein.

In one example, the composition of the present disclosure comprises nucleotide sequences encoding a HA protein, a NA protein and a M1 protein, wherein:
a) the HA protein is a H1, H2, H3, H4, H5, H6, H7, H8, H9, H10, H11, H12, H13, H14, H15, H16, H17, or H18 protein; and
b) the NA protein is a N1, N2, N3, N4, N5, N6, N7, N8, N9, N10, or N11 protein.

For example, the composition comprises nucleotide sequences encoding M1 protein, H1 protein and N1 protein. For example, the composition comprises nucleotide sequences encoding M1 protein, H1 protein, and N2 protein. For example, the composition comprises nucleotide sequences encoding M1 protein, H1 protein, and N3 protein. For example, the composition comprises nucleotide sequences encoding M1 protein, H1 protein, and N4 protein. For example, the composition comprises nucleotide sequences encoding M1 protein, H1 protein, and N5 protein. For example, the composition comprises nucleotide sequences encoding M1, H1, and N6. For example, the composition comprises nucleotide sequences encoding M1 protein, H1 protein, and N7 protein. For example, the composition comprises nucleotide sequences encoding M1 protein, H1 protein, and N8 protein. For example, the composition comprises nucleotide sequences encoding M1 protein, H1 protein, and N9 protein. For example, the composition comprises nucleotide sequences encoding M1 protein, H1 protein, and N10 protein. For example, the composition comprises nucleotide sequences encoding M1 protein, H1 protein, and N11 protein. For example, the composition comprises nucleotide sequences encoding M1 protein, H2 protein and N1 protein. For example, the composition comprises nucleotide sequences encoding M1 protein, H2 protein, and N2 protein. For example, the composition comprises nucleotide sequences encoding M1 protein, H2 protein, and N3 protein. For example, the composition comprises nucleotide sequences encoding M1 protein, H2 protein, and N4 protein. For example, the composition comprises nucleotide sequences encoding M1 protein, H2 protein, and N5 protein. For example, the composition comprises nucleotide sequences encoding M1 protein, H2 protein, and N6 protein. For example, the composition comprises nucleotide sequences encoding M1 protein, H2 protein, and N7 protein. For example, the composition comprises nucleotide sequences encoding M1 protein, H2 protein, and N8 protein. For example, the composition comprises nucleotide sequences encoding M1 protein, H2 protein, and N9 protein. For example, the composition comprises nucleotide sequences encoding M1 protein, H2 protein, and N10 protein. For example, the composition comprises nucleotide sequences encoding M1 protein, H2 protein, and N11 protein. For example, the composition comprises nucleotide sequences encoding M1 protein, H3 protein and N1 protein. For example, the composition comprises nucleotide sequences encoding M1 protein, H3 protein, and N2 protein. For example, the composition comprises nucleotide sequences encoding M1 protein, H3 protein, and N3 protein. For example, the composition comprises nucleotide sequences encoding M1 protein, H3 protein, and N4 protein. For example, the composition comprises nucleotide sequences encoding M1 protein, H3 protein, and N5 protein. For example, the composition comprises nucleotide sequences encoding M1 protein, H3 protein, and N6 protein. For example, the composition comprises nucleotide sequences encoding M1 protein, H3 protein, and N7 protein. For example, the composition comprises nucleotide sequences encoding M1 protein, H3 protein, and N8 protein. For example, the composition comprises nucleotide sequences encoding M1 protein, H3 protein, and N9 protein. For example, the composition comprises nucleotide sequences encoding M1 protein, H3 protein, and N10 protein. For example, the composition comprises nucleotide sequences encoding M1 protein, H3 protein, and N11 protein. For example, the composition comprises nucleotide sequences encoding M1 protein, H4 protein and N1 protein. For example, the composition comprises nucleotide sequences encoding M1 protein, H4 protein, and N2 protein. For example, the composition comprises nucleotide sequences encoding M1 protein, H4 protein, and N3 protein. For example, the composition comprises nucleotide sequences encoding M1 protein, H4 protein, and N4 protein. For example, the composition comprises nucleotide sequences encoding M1 protein, H4 protein, and N5 protein. For example, the composition comprises nucleotide sequences encoding M1 protein, H4 protein, and N6 protein. For example, the composition comprises nucleotide sequences encoding M1 protein, H4 protein, and N7 protein. For example, the composition comprises nucleotide sequences encoding M1 protein, H4 protein, and N8 protein. For example, the composition comprises nucleotide sequences encoding M1 protein, H4 protein, and N9 protein. For example, the composition comprises nucleotide sequences encoding M1 protein, H4 protein, and N10 protein. For example, the composition comprises nucleotide sequences encoding M1 protein, H4 protein, and N11 protein. For example, the composition comprises nucleotide sequences encoding M1 protein, H5 protein and N1 protein. For example, the composition comprises nucleotide sequences encoding M1 protein, H5 protein, and N2 protein. For example, the composition comprises nucleotide sequences encoding M1 protein, H5 protein, and N3 protein. For example, the composition comprises nucleotide sequences encoding M1 protein, H5 protein, and N4 protein. For example, the composition comprises nucleotide sequences encoding M1 protein, H5 protein, and N5 protein. For example, the composition comprises nucleotide sequences encoding M1 protein, H5 protein, and N6 protein. For example, the composition comprises nucleotide sequences encoding M1 protein, H5 protein, and N7 protein. For example, the composition comprises nucleotide sequences encoding M1 protein, H5 protein, and N8 protein. For example, the composition comprises nucleotide sequences encoding M1 protein, H5 protein, and N9 protein. For example, the composition comprises nucleotide sequences encoding M1 protein, H5 protein, and N10 protein. For example, the composition comprises nucleotide sequences encoding M1 protein, H5 protein, and N11 protein. For example, the composition comprises nucleotide sequences encoding M1 protein, H6 protein and N1 protein. For example, the composition comprises nucleotide sequences encoding M1 protein, H6 protein, and N2 protein. For example, the composition comprises nucleotide sequences encoding M1 protein, H6 protein, and N3 protein. For example, the composition comprises nucleotide sequences encoding M1 protein, H6 protein, and N4 protein. For example, the composition comprises nucleotide sequences encoding M1 protein, H6 protein, and N5 protein. For example, the composition comprises nucleotide sequences encoding M1 protein, H6 protein, and N6 protein. For example, the composition comprises nucleotide sequences encoding M1 protein, H6 protein, and N7 protein. For example, the composition comprises nucleotide sequences encoding M1 protein, H6 protein, and N8 protein. For example, the composition comprises nucleotide sequences encoding M1 protein, H6 protein, and N9 protein. For example, the composition comprises nucleotide sequences encoding M1 protein, H6 protein, and N10 protein. For example, the composition comprises nucleotide sequences encoding M1 protein, H6 protein, and N11 protein. For example, the composition comprises nucleotide sequences encoding M1 protein, H7 protein and N1 protein. For example, the composition comprises nucleotide sequences encoding M1 protein, H7 protein, and N2 protein. For example, the composition comprises nucleotide sequences encoding M1 protein, H7 protein, and N3 protein. For example, the composition comprises nucleotide sequences encoding M1 protein, H7 protein, and N4 protein. For example, the composition comprises nucleotide sequences encoding M1 protein, H7 protein, and N5 protein. For example, the composition comprises nucleotide sequences encoding M1 protein, H7 protein, and N6 protein. For example, the composition comprises nucleotide sequences encoding M1 protein, H7 protein, and N7 protein. For example, the composition comprises nucleotide sequences encoding M1 protein, H7 protein, and N8 protein. For example, the composition comprises nucleotide sequences encoding M1 protein, H7 protein, and N9 protein. For example, the composition comprises nucleotide sequences encoding M1 protein, H7 protein, and N10 protein. For example, the composition comprises nucleotide sequences encoding M1 protein, H7 protein, and N11 protein. For example, the composition comprises nucleotide sequences encoding M1 protein, H8 protein and N1 protein. For example, the composition comprises nucleotide sequences encoding M1 protein, H8 protein, and N2 protein. For example, the composition comprises nucleotide sequences encoding M1 protein, H8 protein, and N3 protein. For example, the composition comprises nucleotide sequences encoding M1 protein, H8 protein, and N4 protein. For example, the composition comprises nucleotide sequences encoding M1 protein, H8 protein, and N5 protein. For example, the composition comprises nucleotide sequences encoding M1 protein, H8 protein, and N6 protein. For example, the composition comprises nucleotide sequences encoding M1 protein, H8 protein, and N7 protein. For example, the composition comprises nucleotide sequences encoding M1 protein, H8 protein, and N8 protein. For example, the composition comprises nucleotide sequences encoding M1 protein, H8 protein, and N9 protein. For example, the composition comprises nucleotide sequences encoding M1 protein, H8 protein, and N10 protein. For example, the composition comprises nucleotide sequences encoding M1 protein, H8 protein, and N11 protein. For example, the composition comprises nucleotide sequences encoding M1 protein, H9 protein and N1 protein. For example, the composition comprises nucleotide sequences encoding M1 protein, H9 protein, and N2 protein. For example, the composition comprises nucleotide sequences encoding M1 protein, H9 protein, and N3 protein. For example, the composition comprises nucleotide sequences encoding M1 protein, H9 protein, and N4 protein. For example, the composition comprises nucleotide sequences encoding M1 protein, H9 protein, and N5 protein. For example, the composition comprises nucleotide sequences encoding M1 protein, H9 protein, and N6 protein. For example, the composition comprises nucleotide sequences encoding M1 protein, H9 protein, and N7 protein. For example, the composition comprises nucleotide sequences encoding M1 protein, H9 protein, and N8 protein. For example, the composition comprises nucleotide sequences encoding M1 protein, H9 protein, and N9 protein. For example, the composition comprises nucleotide sequences encoding M1 protein, H9 protein, and N10 protein. For example, the composition comprises nucleotide sequences encoding M1 protein, H9 protein, and N11 protein. For example, the composition comprises nucleotide sequences encoding M1 protein, H10 protein and N1 protein. For example, the composition comprises nucleotide sequences encoding M1 protein, H10 protein, and N2 protein. For example, the composition comprises nucleotide sequences encoding M1 protein, H10 protein, and N3 protein. For example, the composition comprises nucleotide sequences encoding M1 protein, H10 protein, and N4 protein. For example, the composition comprises nucleotide sequences encoding M1 protein, H10 protein, and N5 protein. For example, the composition comprises nucleotide sequences encoding M1 protein, H10 protein, and N6 protein. For example, the composition comprises nucleotide sequences encoding M1 protein, H10 protein, and N7 protein. For example, the composition comprises nucleotide sequences encoding M1 protein, H10 protein, and N8 protein. For example, the composition comprises nucleotide sequences encoding M1 protein, H10 protein, and N9 protein. For example, the composition comprises nucleotide sequences encoding M1 protein, H10 protein, and N10 protein. For example, the composition comprises nucleotide sequences encoding M1 protein, H10 protein, and N11 protein. For example, the composition comprises nucleotide sequences encoding M1 protein, H11 protein and N1 protein. For example, the composition comprises nucleotide sequences encoding M1 protein, H11 protein, and N2 protein. For example, the composition comprises nucleotide sequences encoding M1 protein, H11 protein, and N3 protein. For example, the composition comprises nucleotide sequences encoding M1 protein, H11 protein, and N4 protein. For example, the composition comprises nucleotide sequences encoding M1 protein, H11 protein, and N5 protein. For example, the composition comprises nucleotide sequences encoding M1 protein, H11 protein, and N6 protein. For example, the composition comprises nucleotide sequences encoding M1 protein, H11 protein, and N7 protein. For example, the composition comprises nucleotide sequences encoding M1 protein, H11 protein, and N8 protein. For example, the composition comprises nucleotide sequences encoding M1 protein, H11 protein, and N9 protein. For example, the composition comprises nucleotide sequences encoding M1 protein, H11 protein, and N10 protein. For example, the composition comprises nucleotide sequences encoding M1 protein, H11 protein, and N1 protein. For example, the composition comprises nucleotide sequences encoding M1 protein, H12 protein and N1 protein. For example, the composition comprises nucleotide sequences encoding M1 protein, H12 protein, and N2 protein. For example, the composition comprises nucleotide sequences encoding M1 protein, H12 protein, and N3 protein. For example, the composition comprises nucleotide sequences encoding M1 protein, H12 protein, and N4 protein. For example, the composition comprises nucleotide sequences encoding M1 protein, H12 protein, and N5 protein. For example, the composition comprises nucleotide sequences encoding M1 protein, H12 protein, and N6 protein. For example, the composition comprises nucleotide sequences encoding M1 protein, H12 protein, and N7 protein. For example, the composition comprises nucleotide sequences encoding M1 protein, H12 protein, and N8 protein. For example, the composition comprises nucleotide sequences encoding M1 protein, H12 protein, and N9 protein. For example, the composition comprises nucleotide sequences encoding M1 protein, H12 protein, and N10 protein. For example, the composition comprises nucleotide sequences encoding M1 protein, H12 protein, and N11 protein. For example, the composition comprises nucleotide sequences encoding M1 protein, H13 protein and N1 protein. For example, the composition comprises nucleotide sequences encoding M1 protein, H13 protein, and N2 protein. For example, the composition comprises nucleotide sequences encoding M1 protein, H13 protein, and N3 protein. For example, the composition comprises nucleotide sequences encoding M1 protein, H13 protein, and N4 protein. For example, the composition comprises nucleotide sequences encoding M1 protein, H13 protein, and N5 protein. For example, the composition comprises nucleotide sequences encoding M1 protein, H13 protein, and N6 protein. For example, the composition comprises nucleotide sequences encoding M1 protein, H13 protein, and N7 protein. For example, the composition comprises nucleotide sequences encoding M1 protein, H13 protein, and N8 protein. For example, the composition comprises nucleotide sequences encoding M1 protein, H13 protein, and N9 protein. For example, the composition comprises nucleotide sequences encoding M1 protein, H13 protein, and N10 protein. For example, the composition comprises nucleotide sequences encoding M1 protein, H13 protein, and N11 protein. For example, the composition comprises nucleotide sequences encoding M1 protein, H14 protein and N1 protein. For example, the composition comprises nucleotide sequences encoding M1 protein, H14 protein, and N2 protein. For example, the composition comprises nucleotide sequences encoding M1 protein, H14 protein, and N3 protein. For example, the composition comprises nucleotide sequences encoding M1 protein, H14 protein, and N4 protein. For example, the composition comprises nucleotide sequences encoding M1 protein, H14 protein, and N5 protein. For example, the composition comprises nucleotide sequences encoding M1 protein, H14 protein, and N6 protein. For example, the composition comprises nucleotide sequences encoding M1 protein, H14 protein, and N7 protein. For example, the composition comprises nucleotide sequences encoding M1 protein, H14 protein, and N8 protein. For example, the composition comprises nucleotide sequences encoding M1 protein, H14 protein, and N9 protein. For example, the composition comprises nucleotide sequences encoding M1 protein, H14 protein, and N10 protein. For example, the composition comprises nucleotide sequences encoding M1 protein, H14 protein, and N11 protein. For example, the composition comprises nucleotide sequences encoding M1 protein, H15 protein and N1 protein. For example, the composition comprises nucleotide sequences encoding M1 protein, H15 protein, and N2 protein. For example, the composition comprises nucleotide sequences encoding M1 protein, H15 protein, and N3 protein. For example, the composition comprises nucleotide sequences encoding M1 protein, H15 protein, and N4 protein. For example, the composition comprises nucleotide sequences encoding M1 protein, H15 protein, and N5 protein. For example, the composition comprises nucleotide sequences encoding M1 protein, H15 protein, and N6 protein. For example, the composition comprises nucleotide sequences encoding M1 protein, H15 protein, and N7 protein. For example, the composition comprises nucleotide sequences encoding M1 protein, H15 protein, and N8 protein. For example, the composition comprises nucleotide sequences encoding M1 protein, H15 protein, and N9 protein. For example, the composition comprises nucleotide sequences encoding M1 protein, H15 protein, and N10 protein. For example, the composition comprises nucleotide sequences encoding M1 protein, H15 protein, and N11 protein. For example, the composition comprises nucleotide sequences encoding M1 protein, H16 protein and N1 protein. For example, the composition comprises nucleotide sequences encoding M1 protein, H16 protein, and N2 protein. For example, the composition comprises nucleotide sequences encoding M1 protein, H16 protein, and N3 protein. For example, the composition comprises nucleotide sequences encoding M1 protein, H16 protein, and N4 protein. For example, the composition comprises nucleotide sequences encoding M1 protein, H16 protein, and N5 protein. For example, the composition comprises nucleotide sequences encoding M1 protein, H16 protein, and N6 protein. For example, the composition comprises nucleotide sequences encoding M1 protein, H16 protein, and N7 protein. For example, the composition comprises nucleotide sequences encoding M1 protein, H16 protein, and N8 protein. For example, the composition comprises nucleotide sequences encoding M1 protein, H16 protein, and N9 protein. For example, the composition comprises nucleotide sequences encoding M1 protein, H16 protein, and N10 protein. For example, the composition comprises nucleotide sequences encoding M1 protein, H16 protein, and N11 protein. For example, the composition comprises nucleotide sequences encoding M1 protein, H17 protein and N1 protein. For example, the composition comprises nucleotide sequences encoding M1 protein, H17 protein, and N2 protein. For example, the composition comprises nucleotide sequences encoding M1 protein, H17 protein, and N3 protein. For example, the composition comprises nucleotide sequences encoding M1 protein, H17 protein, and N4 protein. For example, the composition comprises nucleotide sequences encoding M1 protein, H17 protein, and N5 protein. For example, the composition comprises nucleotide sequences encoding M1 protein, H17 protein, and N6 protein. For example, the composition comprises nucleotide sequences encoding M1 protein, H17 protein, and N7 protein. For example, the composition comprises nucleotide sequences encoding M1 protein, H17 protein, and N8 protein. For example, the composition comprises nucleotide sequences encoding M1 protein, H17 protein, and N9 protein. For example, the composition comprises nucleotide sequences encoding M1 protein, H17 protein, and N10 protein. For example, the composition comprises nucleotide sequences encoding M1 protein, H17 protein, and N11 protein. For example, the composition comprises nucleotide sequences encoding M1 protein, H18 protein and N1 protein. For example, the composition comprises nucleotide sequences encoding M1 protein, H18 protein, and N2 protein. For example, the composition comprises nucleotide sequences encoding M1 protein, H18 protein, and N3 protein. For example, the composition comprises nucleotide sequences encoding M1 protein, H18 protein, and N4 protein. For example, the composition comprises nucleotide sequences encoding M1 protein, H18 protein, and N5 protein. For example, the composition comprises nucleotide sequences encoding M1 protein, H18 protein, and N6 protein. For example, the composition comprises nucleotide sequences encoding M1 protein, H18 protein, and N7 protein. For example, the composition comprises nucleotide sequences encoding M1 protein, H18 protein, and N8 protein. For example, the composition comprises nucleotide sequences encoding M1 protein, H18 protein, and N9 protein. For example, the composition comprises nucleotide sequences encoding M1 protein, H18 protein, and N10 protein. For example, the composition comprises nucleotide sequences encoding M1 protein, H18 protein, and N11 protein.

In one example, the nucleotide sequence encoding:
a) the HA protein is at least 90% identical to a nucleotide sequence set forth in SEQ ID Nos: 64 or 71;
b) the NA protein is at least 90% identical to a nucleotide sequence set forth in SEQ ID Nos: 65 or 72; and
c) the M1 protein is at least 90% identical to a nucleotide sequence set forth in SEQ ID Nos: 66 or 70.

For example, the nucleotide sequence encoding:
a) the HA protein is at least 90% identical to a nucleotide sequence set forth in SEQ ID NO: 64;
b) the NA protein is at least 90% identical to a nucleotide sequence set forth in SEQ ID NO: 65; and
c) the M1 protein is at least 90% identical to a nucleotide sequence set forth in SEQ ID NO: 66.

For example, the nucleotide sequence encoding:
a) the HA protein is at least 90% identical to a nucleotide sequence set forth in SEQ ID NO: 64;
b) the NA protein is at least 90% identical to a nucleotide sequence set forth in SEQ ID NO: 65; and
c) the M1 protein is at least 90% identical to a nucleotide sequence set forth in SEQ ID NO: 70.

For example, the nucleotide sequence encoding:
a) the HA protein is at least 90% identical to a nucleotide sequence set forth in SEQ ID NO: 64;
b) the NA protein is at least 90% identical to a nucleotide sequence set forth in SEQ ID NO: 72; and
c) the M1 protein is at least 90% identical to a nucleotide sequence set forth in SEQ ID NO: 66.

For example, the nucleotide sequence encoding:
a) the HA protein is at least 90% identical to a nucleotide sequence set forth in SEQ ID NO: 64;
b) the NA protein is at least 90% identical to a nucleotide sequence set forth in SEQ ID NO: 72; and
c) the M1 protein is at least 90% identical to a nucleotide sequence set forth in SEQ ID NO: 70.

For example, the nucleotide sequence encoding:
a) the HA protein is at least 90% identical to a nucleotide sequence set forth in SEQ ID NO: 71;
b) the NA protein is at least 90% identical to a nucleotide sequence set forth in SEQ ID NO: 65; and
c) the M1 protein is at least 90% identical to a nucleotide sequence set forth in SEQ ID NO: 66.

For example, the nucleotide sequence encoding:
a) the HA protein is at least 90% identical to a nucleotide sequence set forth in SEQ ID NO: 71;
b) the NA protein is at least 90% identical to a nucleotide sequence set forth in SEQ ID NO: 65; and
c) the M1 protein is at least 90% identical to a nucleotide sequence set forth in SEQ ID NO: 70.

For example, the nucleotide sequence encoding:
a) the HA protein is at least 90% identical to a nucleotide sequence set forth in SEQ ID NO: 71;
b) the NA protein is at least 90% identical to a nucleotide sequence set forth in SEQ ID NO: 72; and
c) the M1 protein is at least 90% identical to a nucleotide sequence set forth in SEQ ID NO: 66.

For example, the nucleotide sequence encoding:
a) the HA protein is at least 90% identical to a nucleotide sequence set forth in SEQ ID NO: 71;
b) the NA protein is at least 90% identical to a nucleotide sequence set forth in SEQ ID NO: 72; and
c) the M1 protein is at least 90% identical to a nucleotide sequence set forth in SEQ ID NO: 70.

In one example, the nucleotide sequence encoding:
a) the HA protein is at least 91% identical to a nucleotide sequence set forth in SEQ ID Nos: 64 or 71;
b) the NA protein is at least 91% identical to a nucleotide sequence set forth in SEQ ID Nos: 65 or 72; and
c) the M1 protein is at least 91% identical to a nucleotide sequence set forth in SEQ ID Nos: 66 or 70.

For example, the nucleotide sequence encoding:
a) the HA protein is at least 91% identical to a nucleotide sequence set forth in SEQ ID NO: 64;
b) the NA protein is at least 91% identical to a nucleotide sequence set forth in SEQ ID NO: 65; and
c) the M1 protein is at least 91% identical to a nucleotide sequence set forth in SEQ ID NO: 66.

For example, the nucleotide sequence encoding:
a) the HA protein is at least 91% identical to a nucleotide sequence set forth in SEQ ID NO: 64;
b) the NA protein is at least 91% identical to a nucleotide sequence set forth in SEQ ID NO: 65; and
c) the M1 protein is at least 91% identical to a nucleotide sequence set forth in SEQ ID NO: 70.

For example, the nucleotide sequence encoding:
a) the HA protein is at least 91% identical to a nucleotide sequence set forth in SEQ ID NO: 64;
b) the NA protein is at least 91% identical to a nucleotide sequence set forth in SEQ ID NO: 72; and
c) the M1 protein is at least 91% identical to a nucleotide sequence set forth in SEQ ID NO: 66.

For example, the nucleotide sequence encoding:
a) the HA protein is at least 91% identical to a nucleotide sequence set forth in SEQ ID NO: 64;
b) the NA protein is at least 91% identical to a nucleotide sequence set forth in SEQ ID NO: 72; and
c) the M1 protein is at least 91% identical to a nucleotide sequence set forth in SEQ ID NO: 70.

For example, the nucleotide sequence encoding:
a) the HA protein is at least 91% identical to a nucleotide sequence set forth in SEQ ID NO: 71;
b) the NA protein is at least 91% identical to a nucleotide sequence set forth in SEQ ID NO: 65; and
c) the M1 protein is at least 91% identical to a nucleotide sequence set forth in SEQ ID NO: 66.

For example, the nucleotide sequence encoding:
a) the HA protein is at least 91% identical to a nucleotide sequence set forth in SEQ ID NO: 71;
b) the NA protein is at least 91% identical to a nucleotide sequence set forth in SEQ ID NO: 65; and
c) the M1 protein is at least 91% identical to a nucleotide sequence set forth in SEQ ID NO: 70.

For example, the nucleotide sequence encoding:
a) the HA protein is at least 91% identical to a nucleotide sequence set forth in SEQ ID NO: 71;
b) the NA protein is at least 91% identical to a nucleotide sequence set forth in SEQ ID NO: 72; and
c) the M1 protein is at least 91% identical to a nucleotide sequence set forth in SEQ ID NO: 66.

For example, the nucleotide sequence encoding:
a) the HA protein is at least 91% identical to a nucleotide sequence set forth in SEQ ID NO: 71;
b) the NA protein is at least 91% identical to a nucleotide sequence set forth in SEQ ID NO: 72; and
c) the M1 protein is at least 91% identical to a nucleotide sequence set forth in SEQ ID NO: 70.

In one example, the nucleotide sequence encoding:
a) the HA protein is at least 92% identical to a nucleotide sequence set forth in SEQ ID Nos: 64 or 71;
b) the NA protein is at least 92% identical to a nucleotide sequence set forth in SEQ ID Nos: 65 or 72; and
c) the M1 protein is at least 92% identical to a nucleotide sequence set forth in SEQ ID Nos: 66 or 70.

For example, the nucleotide sequence encoding:
a) the HA protein is at least 92% identical to a nucleotide sequence set forth in SEQ ID NO: 64;
b) the NA protein is at least 92% identical to a nucleotide sequence set forth in SEQ ID NO: 65; and
c) the M1 protein is at least 92% identical to a nucleotide sequence set forth in SEQ ID NO: 66.

For example, the nucleotide sequence encoding:
a) the HA protein is at least 92% identical to a nucleotide sequence set forth in SEQ ID NO: 64;
b) the NA protein is at least 92% identical to a nucleotide sequence set forth in SEQ ID NO: 65; and
c) the M1 protein is at least 92% identical to a nucleotide sequence set forth in SEQ ID NO: 70.

For example, the nucleotide sequence encoding:
a) the HA protein is at least 92% identical to a nucleotide sequence set forth in SEQ ID NO: 64;
b) the NA protein is at least 92% identical to a nucleotide sequence set forth in SEQ ID NO: 72; and
c) the M1 protein is at least 92% identical to a nucleotide sequence set forth in SEQ ID NO: 66.

For example, the nucleotide sequence encoding:
a) the HA protein is at least 92% identical to a nucleotide sequence set forth in SEQ ID NO: 64;
b) the NA protein is at least 92% identical to a nucleotide sequence set forth in SEQ ID NO: 72; and
c) the M1 protein is at least 92% identical to a nucleotide sequence set forth in SEQ ID NO: 70.

For example, the nucleotide sequence encoding:
a) the HA protein is at least 92% identical to a nucleotide sequence set forth in SEQ ID NO: 71;
b) the NA protein is at least 92% identical to a nucleotide sequence set forth in SEQ ID NO: 65; and
c) the M1 protein is at least 92% identical to a nucleotide sequence set forth in SEQ ID NO: 66.

For example, the nucleotide sequence encoding:
a) the HA protein is at least 92% identical to a nucleotide sequence set forth in SEQ ID NO: 71;
b) the NA protein is at least 92% identical to a nucleotide sequence set forth in SEQ ID NO: 65; and
c) the M1 protein is at least 92% identical to a nucleotide sequence set forth in SEQ ID NO: 70.

For example, the nucleotide sequence encoding:
a) the HA protein is at least 92% identical to a nucleotide sequence set forth in SEQ ID NO: 71;
b) the NA protein is at least 92% identical to a nucleotide sequence set forth in SEQ ID NO: 72; and
c) the M1 protein is at least 92% identical to a nucleotide sequence set forth in SEQ ID NO: 66.

For example, the nucleotide sequence encoding:
a) the HA protein is at least 92% identical to a nucleotide sequence set forth in SEQ ID NO: 71;
b) the NA protein is at least 92% identical to a nucleotide sequence set forth in SEQ ID NO: 72; and
c) the M1 protein is at least 92% identical to a nucleotide sequence set forth in SEQ ID NO: 70.

In one example, the nucleotide sequence encoding:
a) the HA protein is at least 93% identical to a nucleotide sequence set forth in SEQ ID Nos: 64 or 71;
b) the NA protein is at least 93% identical to a nucleotide sequence set forth in SEQ ID Nos: 65 or 72; and
c) the M1 protein is at least 93% identical to a nucleotide sequence set forth in SEQ ID Nos: 66 or 70.

For example, the nucleotide sequence encoding:
a) the HA protein is at least 93% identical to a nucleotide sequence set forth in SEQ ID NO: 64;
b) the NA protein is at least 93% identical to a nucleotide sequence set forth in SEQ ID NO: 65; and
c) the M1 protein is at least 93% identical to a nucleotide sequence set forth in SEQ ID NO: 66.

For example, the nucleotide sequence encoding:
a) the HA protein is at least 93% identical to a nucleotide sequence set forth in SEQ ID NO: 64;
b) the NA protein is at least 93% identical to a nucleotide sequence set forth in SEQ ID NO: 65; and
c) the M1 protein is at least 93% identical to a nucleotide sequence set forth in SEQ ID NO: 70.

For example, the nucleotide sequence encoding:
a) the HA protein is at least 93% identical to a nucleotide sequence set forth in SEQ ID NO: 64;
b) the NA protein is at least 93% identical to a nucleotide sequence set forth in SEQ ID NO: 72; and
c) the M1 protein is at least 93% identical to a nucleotide sequence set forth in SEQ ID NO: 66.

For example, the nucleotide sequence encoding:
a) the HA protein is at least 93% identical to a nucleotide sequence set forth in SEQ ID NO: 64;
b) the NA protein is at least 93% identical to a nucleotide sequence set forth in SEQ ID NO: 72; and
c) the M1 protein is at least 93% identical to a nucleotide sequence set forth in SEQ ID NO: 70.

For example, the nucleotide sequence encoding:
a) the HA protein is at least 93% identical to a nucleotide sequence set forth in SEQ ID NO: 71;
b) the NA protein is at least 93% identical to a nucleotide sequence set forth in SEQ ID NO: 65; and
c) the M1 protein is at least 93% identical to a nucleotide sequence set forth in SEQ ID NO: 66.

For example, the nucleotide sequence encoding:
a) the HA protein is at least 93% identical to a nucleotide sequence set forth in SEQ ID NO: 71;
b) the NA protein is at least 93% identical to a nucleotide sequence set forth in SEQ ID NO: 65; and
c) the M1 protein is at least 93% identical to a nucleotide sequence set forth in SEQ ID NO: 70.

For example, the nucleotide sequence encoding:
a) the HA protein is at least 93% identical to a nucleotide sequence set forth in SEQ ID NO: 71;
b) the NA protein is at least 93% identical to a nucleotide sequence set forth in SEQ ID NO: 72; and
c) the M1 protein is at least 93% identical to a nucleotide sequence set forth in SEQ ID NO: 66.

For example, the nucleotide sequence encoding:
a) the HA protein is at least 93% identical to a nucleotide sequence set forth in SEQ ID NO: 71;
b) the NA protein is at least 93% identical to a nucleotide sequence set forth in SEQ ID NO: 72; and
c) the M1 protein is at least 93% identical to a nucleotide sequence set forth in SEQ ID NO: 70.

In one example, the nucleotide sequence encoding:
a) the HA protein is at least 94% identical to a nucleotide sequence set forth in SEQ ID Nos: 64 or 71;
b) the NA protein is at least 94% identical to a nucleotide sequence set forth in SEQ ID Nos: 65 or 72; and
c) the M1 protein is at least 94% identical to a nucleotide sequence set forth in SEQ ID Nos: 66 or 70.

For example, the nucleotide sequence encoding:
a) the HA protein is at least 94% identical to a nucleotide sequence set forth in SEQ ID NO: 64;
b) the NA protein is at least 94% identical to a nucleotide sequence set forth in SEQ ID NO: 65; and
c) the M1 protein is at least 94% identical to a nucleotide sequence set forth in SEQ ID NO: 66.

For example, the nucleotide sequence encoding:
a) the HA protein is at least 94% identical to a nucleotide sequence set forth in SEQ ID NO: 64;
b) the NA protein is at least 94% identical to a nucleotide sequence set forth in SEQ ID NO: 65; and
c) the M1 protein is at least 94% identical to a nucleotide sequence set forth in SEQ ID NO: 70.

For example, the nucleotide sequence encoding:
a) the HA protein is at least 94% identical to a nucleotide sequence set forth in SEQ ID NO: 64;
b) the NA protein is at least 94% identical to a nucleotide sequence set forth in SEQ ID NO: 72; and
c) the M1 protein is at least 94% identical to a nucleotide sequence set forth in SEQ ID NO: 66.

For example, the nucleotide sequence encoding:
a) the HA protein is at least 94% identical to a nucleotide sequence set forth in SEQ ID NO: 64;
b) the NA protein is at least 94% identical to a nucleotide sequence set forth in SEQ ID NO: 72; and
c) the M1 protein is at least 94% identical to a nucleotide sequence set forth in SEQ ID NO: 70.

For example, the nucleotide sequence encoding:
a) the HA protein is at least 94% identical to a nucleotide sequence set forth in SEQ ID NO: 71;
b) the NA protein is at least 94% identical to a nucleotide sequence set forth in SEQ ID NO: 65; and
c) the M1 protein is at least 94% identical to a nucleotide sequence set forth in SEQ ID NO: 66.

For example, the nucleotide sequence encoding:
a) the HA protein is at least 94% identical to a nucleotide sequence set forth in SEQ ID NO: 71;
b) the NA protein is at least 94% identical to a nucleotide sequence set forth in SEQ ID NO: 65; and
c) the M1 protein is at least 94% identical to a nucleotide sequence set forth in SEQ ID NO: 70.

For example, the nucleotide sequence encoding:
a) the HA protein is at least 94% identical to a nucleotide sequence set forth in SEQ ID NO: 71;
b) the NA protein is at least 94% identical to a nucleotide sequence set forth in SEQ ID NO: 72; and
c) the M1 protein is at least 94% identical to a nucleotide sequence set forth in SEQ ID NO: 66.

For example, the nucleotide sequence encoding:
a) the HA protein is at least 94% identical to a nucleotide sequence set forth in SEQ ID NO: 71;
b) the NA protein is at least 94% identical to a nucleotide sequence set forth in SEQ ID NO: 72; and
c) the M1 protein is at least 94% identical to a nucleotide sequence set forth in SEQ ID NO: 70.

In one example, the nucleotide sequence encoding:
a) the HA protein is at least 95% identical to a nucleotide sequence set forth in SEQ ID Nos: 64 or 71;
b) the NA protein is at least 95% identical to a nucleotide sequence set forth in SEQ ID Nos: 65 or 72; and
c) the M1 protein is at least 95% identical to a nucleotide sequence set forth in SEQ ID Nos: 66 or 70.

For example, the nucleotide sequence encoding:
a) the HA protein is at least 95% identical to a nucleotide sequence set forth in SEQ ID NO: 64;
b) the NA protein is at least 95% identical to a nucleotide sequence set forth in SEQ ID NO: 65; and
c) the M1 protein is at least 95% identical to a nucleotide sequence set forth in SEQ ID NO: 66.

For example, the nucleotide sequence encoding:
a) the HA protein is at least 95% identical to a nucleotide sequence set forth in SEQ ID NO: 64;
b) the NA protein is at least 95% identical to a nucleotide sequence set forth in SEQ ID NO: 65; and
c) the M1 protein is at least 95% identical to a nucleotide sequence set forth in SEQ ID NO: 70.

For example, the nucleotide sequence encoding:
a) the HA protein is at least 95% identical to a nucleotide sequence set forth in SEQ ID NO: 64;
b) the NA protein is at least 95% identical to a nucleotide sequence set forth in SEQ ID NO: 72; and
c) the M1 protein is at least 95% identical to a nucleotide sequence set forth in SEQ ID NO: 66.

For example, the nucleotide sequence encoding:
a) the HA protein is at least 95% identical to a nucleotide sequence set forth in SEQ ID NO: 64;
b) the NA protein is at least 95% identical to a nucleotide sequence set forth in SEQ ID NO: 72; and
c) the M1 protein is at least 95% identical to a nucleotide sequence set forth in SEQ ID NO: 70.

For example, the nucleotide sequence encoding:
a) the HA protein is at least 95% identical to a nucleotide sequence set forth in SEQ ID NO: 71;
b) the NA protein is at least 95% identical to a nucleotide sequence set forth in SEQ ID NO: 65; and
c) the M1 protein is at least 95% identical to a nucleotide sequence set forth in SEQ ID NO: 66.

For example, the nucleotide sequence encoding:
a) the HA protein is at least 95% identical to a nucleotide sequence set forth in SEQ ID NO: 71;
b) the NA protein is at least 95% identical to a nucleotide sequence set forth in SEQ ID NO: 65; and
c) the M1 protein is at least 95% identical to a nucleotide sequence set forth in SEQ ID NO: 70.

For example, the nucleotide sequence encoding:
a) the HA protein is at least 95% identical to a nucleotide sequence set forth in SEQ ID NO: 71;
b) the NA protein is at least 95% identical to a nucleotide sequence set forth in SEQ ID NO: 72; and
c) the M1 protein is at least 95% identical to a nucleotide sequence set forth in SEQ ID NO: 66.

For example, the nucleotide sequence encoding:
a) the HA protein is at least 95% identical to a nucleotide sequence set forth in SEQ ID NO: 71;
b) the NA protein is at least 95% identical to a nucleotide sequence set forth in SEQ ID NO: 72; and
c) the M1 protein is at least 95% identical to a nucleotide sequence set forth in SEQ ID NO: 70.

In one example, the nucleotide sequence encoding:
a) the HA protein is at least 96% identical to a nucleotide sequence set forth in SEQ ID Nos: 64 or 71;
b) the NA protein is at least 96% identical to a nucleotide sequence set forth in SEQ ID Nos: 65 or 72; and
c) the M1 protein is at least 96% identical to a nucleotide sequence set forth in SEQ ID Nos: 66 or 70.

For example, the nucleotide sequence encoding:
a) the HA protein is at least 96% identical to a nucleotide sequence set forth in SEQ ID NO: 64;
b) the NA protein is at least 96% identical to a nucleotide sequence set forth in SEQ ID NO: 65; and
c) the M1 protein is at least 96% identical to a nucleotide sequence set forth in SEQ ID NO: 66.

For example, the nucleotide sequence encoding:
a) the HA protein is at least 96% identical to a nucleotide sequence set forth in SEQ ID NO: 64;
b) the NA protein is at least 96% identical to a nucleotide sequence set forth in SEQ ID NO: 65; and
c) the M1 protein is at least 96% identical to a nucleotide sequence set forth in SEQ ID NO: 70.

For example, the nucleotide sequence encoding:
a) the HA protein is at least 96% identical to a nucleotide sequence set forth in SEQ ID NO: 64;
b) the NA protein is at least 96% identical to a nucleotide sequence set forth in SEQ ID NO: 72; and
c) the M1 protein is at least 96% identical to a nucleotide sequence set forth in SEQ ID NO: 66.

For example, the nucleotide sequence encoding:
a) the HA protein is at least 96% identical to a nucleotide sequence set forth in SEQ ID NO: 64;
b) the NA protein is at least 96% identical to a nucleotide sequence set forth in SEQ ID NO: 72; and
c) the M1 protein is at least 96% identical to a nucleotide sequence set forth in SEQ ID NO: 70.

For example, the nucleotide sequence encoding:
a) the HA protein is at least 96% identical to a nucleotide sequence set forth in SEQ ID NO: 71;
b) the NA protein is at least 96% identical to a nucleotide sequence set forth in SEQ ID NO: 65; and
c) the M1 protein is at least 96% identical to a nucleotide sequence set forth in SEQ ID NO: 66.

For example, the nucleotide sequence encoding:
a) the HA protein is at least 96% identical to a nucleotide sequence set forth in SEQ ID NO: 71;
b) the NA protein is at least 96% identical to a nucleotide sequence set forth in SEQ ID NO: 65; and
c) the M1 protein is at least 96% identical to a nucleotide sequence set forth in SEQ ID NO: 70.

For example, the nucleotide sequence encoding:
a) the HA protein is at least 96% identical to a nucleotide sequence set forth in SEQ ID NO: 71;
b) the NA protein is at least 96% identical to a nucleotide sequence set forth in SEQ ID NO: 72; and
c) the M1 protein is at least 96% identical to a nucleotide sequence set forth in SEQ ID NO: 66.

For example, the nucleotide sequence encoding:
a) the HA protein is at least 96% identical to a nucleotide sequence set forth in SEQ ID NO: 71;
b) the NA protein is at least 96% identical to a nucleotide sequence set forth in SEQ ID NO: 72; and
c) the M1 protein is at least 96% identical to a nucleotide sequence set forth in SEQ ID NO: 70.

In one example, the nucleotide sequence encoding:
a) the HA protein is at least 97% identical to a nucleotide sequence set forth in SEQ ID Nos: 64 or 71;
b) the NA protein is at least 97% identical to a nucleotide sequence set forth in SEQ ID Nos: 65 or 72; and
c) the M1 protein is at least 97% identical to a nucleotide sequence set forth in SEQ ID Nos: 66 or 70.

For example, the nucleotide sequence encoding:
a) the HA protein is at least 97% identical to a nucleotide sequence set forth in SEQ ID NO: 64;
b) the NA protein is at least 97% identical to a nucleotide sequence set forth in SEQ ID NO: 65; and
c) the M1 protein is at least 97% identical to a nucleotide sequence set forth in SEQ ID NO: 66.

For example, the nucleotide sequence encoding:
a) the HA protein is at least 97% identical to a nucleotide sequence set forth in SEQ ID NO: 64;
b) the NA protein is at least 97% identical to a nucleotide sequence set forth in SEQ ID NO: 65; and
c) the M1 protein is at least 97% identical to a nucleotide sequence set forth in SEQ ID NO: 70.

For example, the nucleotide sequence encoding:
a) the HA protein is at least 97% identical to a nucleotide sequence set forth in SEQ ID NO: 64;
b) the NA protein is at least 97% identical to a nucleotide sequence set forth in SEQ ID NO: 72; and
c) the M1 protein is at least 97% identical to a nucleotide sequence set forth in SEQ ID NO: 66.

For example, the nucleotide sequence encoding:
a) the HA protein is at least 97% identical to a nucleotide sequence set forth in SEQ ID NO: 64;
b) the NA protein is at least 97% identical to a nucleotide sequence set forth in SEQ ID NO: 72; and
c) the M1 protein is at least 97% identical to a nucleotide sequence set forth in SEQ ID NO: 70.

For example, the nucleotide sequence encoding:
a) the HA protein is at least 97% identical to a nucleotide sequence set forth in SEQ ID NO: 71;
b) the NA protein is at least 97% identical to a nucleotide sequence set forth in SEQ ID NO: 65; and
c) the M1 protein is at least 97% identical to a nucleotide sequence set forth in SEQ ID NO: 66.

For example, the nucleotide sequence encoding:
a) the HA protein is at least 97% identical to a nucleotide sequence set forth in SEQ ID NO: 71;
b) the NA protein is at least 97% identical to a nucleotide sequence set forth in SEQ ID NO: 65; and
c) the M1 protein is at least 97% identical to a nucleotide sequence set forth in SEQ ID NO: 70.

For example, the nucleotide sequence encoding:
a) the HA protein is at least 97% identical to a nucleotide sequence set forth in SEQ ID NO: 71;
b) the NA protein is at least 97% identical to a nucleotide sequence set forth in SEQ ID NO: 72; and
c) the M1 protein is at least 97% identical to a nucleotide sequence set forth in SEQ ID NO: 66.

For example, the nucleotide sequence encoding:
a) the HA protein is at least 97% identical to a nucleotide sequence set forth in SEQ ID NO: 71;
b) the NA protein is at least 97% identical to a nucleotide sequence set forth in SEQ ID NO: 72; and
c) the M1 protein is at least 97% identical to a nucleotide sequence set forth in SEQ ID NO: 70.

In one example, the nucleotide sequence encoding:
a) the HA protein is at least 98% identical to a nucleotide sequence set forth in SEQ ID Nos: 64 or 71;
b) the NA protein is at least 98% identical to a nucleotide sequence set forth in SEQ ID Nos: 65 or 72; and
c) the M1 protein is at least 98% identical to a nucleotide sequence set forth in SEQ ID Nos: 66 or 70.

For example, the nucleotide sequence encoding:
a) the HA protein is at least 98% identical to a nucleotide sequence set forth in SEQ ID NO: 64;
b) the NA protein is at least 98% identical to a nucleotide sequence set forth in SEQ ID NO: 65; and
c) the M1 protein is at least 98% identical to a nucleotide sequence set forth in SEQ ID NO: 66.

For example, the nucleotide sequence encoding:
a) the HA protein is at least 98% identical to a nucleotide sequence set forth in SEQ ID NO: 64;
b) the NA protein is at least 98% identical to a nucleotide sequence set forth in SEQ ID NO: 65; and
c) the M1 protein is at least 98% identical to a nucleotide sequence set forth in SEQ ID NO: 70.

For example, the nucleotide sequence encoding:
a) the HA protein is at least 98% identical to a nucleotide sequence set forth in SEQ ID NO: 64;
b) the NA protein is at least 98% identical to a nucleotide sequence set forth in SEQ ID NO: 72; and
c) the M1 protein is at least 98% identical to a nucleotide sequence set forth in SEQ ID NO: 66.

For example, the nucleotide sequence encoding:
a) the HA protein is at least 98% identical to a nucleotide sequence set forth in SEQ ID NO: 64;
b) the NA protein is at least 98% identical to a nucleotide sequence set forth in SEQ ID NO: 72; and
c) the M1 protein is at least 98% identical to a nucleotide sequence set forth in SEQ ID NO: 70.

For example, the nucleotide sequence encoding:
a) the HA protein is at least 98% identical to a nucleotide sequence set forth in SEQ ID NO: 71;
b) the NA protein is at least 98% identical to a nucleotide sequence set forth in SEQ ID NO: 65; and
c) the M1 protein is at least 98% identical to a nucleotide sequence set forth in SEQ ID NO: 66.

For example, the nucleotide sequence encoding:
a) the HA protein is at least 98% identical to a nucleotide sequence set forth in SEQ ID NO: 71;
b) the NA protein is at least 98% identical to a nucleotide sequence set forth in SEQ ID NO: 65; and
c) the M1 protein is at least 98% identical to a nucleotide sequence set forth in SEQ ID NO: 70.

For example, the nucleotide sequence encoding:
a) the HA protein is at least 98% identical to a nucleotide sequence set forth in SEQ ID NO: 71;
b) the NA protein is at least 98% identical to a nucleotide sequence set forth in SEQ ID NO: 72; and
c) the M1 protein is at least 98% identical to a nucleotide sequence set forth in SEQ ID NO: 66.

For example, the nucleotide sequence encoding:
a) the HA protein is at least 98% identical to a nucleotide sequence set forth in SEQ ID NO: 71;
b) the NA protein is at least 98% identical to a nucleotide sequence set forth in SEQ ID NO: 72; and
c) the M1 protein is at least 98% identical to a nucleotide sequence set forth in SEQ ID NO: 70.

In one example, the nucleotide sequence encoding:
a) the HA protein is at least 99% identical to a nucleotide sequence set forth in SEQ ID Nos: 64 or 71;
b) the NA protein is at least 99% identical to a nucleotide sequence set forth in SEQ ID Nos: 65 or 72; and
c) the M1 protein is at least 99% identical to a nucleotide sequence set forth in SEQ ID Nos: 66 or 70.

For example, the nucleotide sequence encoding:
a) the HA protein is at least 99% identical to a nucleotide sequence set forth in SEQ ID NO: 64;
b) the NA protein is at least 99% identical to a nucleotide sequence set forth in SEQ ID NO: 65; and
c) the M1 protein is at least 99% identical to a nucleotide sequence set forth in SEQ ID NO: 66.

For example, the nucleotide sequence encoding:
a) the HA protein is at least 99% identical to a nucleotide sequence set forth in SEQ ID NO: 64;
b) the NA protein is at least 99% identical to a nucleotide sequence set forth in SEQ ID NO: 65; and
c) the M1 protein is at least 99% identical to a nucleotide sequence set forth in SEQ ID NO: 70.

For example, the nucleotide sequence encoding:
a) the HA protein is at least 99% identical to a nucleotide sequence set forth in SEQ ID NO: 64;
b) the NA protein is at least 99% identical to a nucleotide sequence set forth in SEQ ID NO: 72; and
c) the M1 protein is at least 99% identical to a nucleotide sequence set forth in SEQ ID NO: 66.

For example, the nucleotide sequence encoding:
a) the HA protein is at least 99% identical to a nucleotide sequence set forth in SEQ ID NO: 64;
b) the NA protein is at least 99% identical to a nucleotide sequence set forth in SEQ ID NO: 72; and
c) the M1 protein is at least 99% identical to a nucleotide sequence set forth in SEQ ID NO: 70.

For example, the nucleotide sequence encoding:
a) the HA protein is at least 99% identical to a nucleotide sequence set forth in SEQ ID NO: 71;
b) the NA protein is at least 99% identical to a nucleotide sequence set forth in SEQ ID NO: 65; and
c) the M1 protein is at least 99% identical to a nucleotide sequence set forth in SEQ ID NO: 66.

For example, the nucleotide sequence encoding:
a) the HA protein is at least 99% identical to a nucleotide sequence set forth in SEQ ID NO: 71;
b) the NA protein is at least 99% identical to a nucleotide sequence set forth in SEQ ID NO: 65; and
c) the M1 protein is at least 99% identical to a nucleotide sequence set forth in SEQ ID NO: 70.

For example, the nucleotide sequence encoding:
a) the HA protein is at least 99% identical to a nucleotide sequence set forth in SEQ ID NO: 71;
b) the NA protein is at least 99% identical to a nucleotide sequence set forth in SEQ ID NO: 72; and
c) the M1 protein is at least 99% identical to a nucleotide sequence set forth in SEQ ID NO: 66.

For example, the nucleotide sequence encoding:
a) the HA protein is at least 99% identical to a nucleotide sequence set forth in SEQ ID NO: 71;
b) the NA protein is at least 99% identical to a nucleotide sequence set forth in SEQ ID NO: 72; and
c) the M1 protein is at least 99% identical to a nucleotide sequence set forth in SEQ ID NO: 70.

In one example, the nucleotide sequence encoding:
a) the HA protein is selected from a nucleotide sequence set forth in SEQ ID Nos: 64 or 71;
b) the NA protein is selected from a nucleotide sequence set forth in SEQ ID Nos: 65 or 72; and
c) the M1 protein is selected from a nucleotide sequence set forth in SEQ ID Nos: 66 or 70.

For example, the nucleotide sequence encoding:
a) the HA protein comprises a nucleotide sequence set forth in SEQ ID No: 64;
b) the NA protein comprises a nucleotide sequence set forth in SEQ ID NO: 65; and
c) the M1 protein comprises a nucleotide sequence set forth in SEQ ID NO: 66.

For example, the nucleotide sequence encoding:
a) the HA protein comprises a nucleotide sequence set forth in SEQ ID NO: 64;
b) the NA protein comprises a nucleotide sequence set forth in SEQ ID NO: 65; and
c) the M1 protein comprises a nucleotide sequence set forth in SEQ ID NO: 70.

For example, the nucleotide sequence encoding:
a) the HA protein comprises a nucleotide sequence set forth in SEQ ID NO: 64;
b) the NA protein comprises a nucleotide sequence set forth in SEQ ID NO: 72; and
c) the M1 protein comprises a nucleotide sequence set forth in SEQ ID NO: 66.

For example, the nucleotide sequence encoding:
a) the HA protein comprises a nucleotide sequence set forth in SEQ ID NO: 64;
b) the NA protein comprises a nucleotide sequence set forth in SEQ ID NO: 72; and
c) the M1 protein comprises a nucleotide sequence set forth in SEQ ID NO: 70.

For example, the nucleotide sequence encoding:
a) the HA protein comprises a nucleotide sequence set forth in SEQ ID NO: 71;
b) the NA protein comprises a nucleotide sequence set forth in SEQ ID NO: 65; and
c) the M1 protein comprises a nucleotide sequence set forth in SEQ ID NO: 66.

For example, the nucleotide sequence encoding:
a) the HA protein comprises a nucleotide sequence set forth in SEQ ID NO: 71;
b) the NA protein comprises a nucleotide sequence set forth in SEQ ID NO: 65; and
c) the M1 protein comprises a nucleotide sequence set forth in SEQ ID NO: 70.

For example, the nucleotide sequence encoding:
a) the HA protein comprises a nucleotide sequence set forth in SEQ ID NO: 71;
b) the NA protein comprises a nucleotide sequence set forth in SEQ ID NO: 72; and
c) the M1 protein comprises a nucleotide sequence set forth in SEQ ID NO: 66.

For example, the nucleotide sequence encoding:
a) the HA protein comprises a nucleotide sequence set forth in SEQ ID NO: 71;
b) the NA protein comprises a nucleotide sequence set forth in SEQ ID NO: 72; and
c) the M1 protein comprises a nucleotide sequence set forth in SEQ ID NO: 70.

In one example, the VLP forming elements are from the same influenza virus.

In one example, two or more of the VLP forming elements are from different influenza viruses. For example, two of the VLP forming elements are from different influenza viruses. For example, three of the VLP forming elements are from different influenza viruses.

In one example, the one or more RNA(s) comprises one or more additional nucleotide sequence encoding a matrix-2 (M2), nucleoprotein (NP) and/or a non-structural (NS) protein of an influenza virus, wherein the one or more additional nucleotide sequence is located 3′ or 5′ of the one or more nucleotide sequence(s) encoding the VLP forming element. For example, the one or more RNA(s) comprises an additional nucleotide sequence encoding a matrix-2 (M2). For example, the one or more RNA(s) comprises an additional nucleotide sequence encoding a nucleoprotein (NP) protein. For example, the one or more RNA(s) comprises an additional nucleotide sequence encoding a non-structural (NS) protein. For example, the NS protein is a non-structural 1 (NS1) protein. For example, the NS proteins is a non-structural 2 (NS2) protein. For example, the one or more RNA(s) comprises an additional nucleotide sequence encoding a M2 protein and an additional nucleotide sequence encoding a NP protein. For example, the one or more RNA(s) comprises an additional nucleotide sequence encoding a M2 protein and an additional sequence encoding a NS protein. For example, the one or more RNA(s) comprises an additional nucleotide sequence encoding a NP protein and an additional sequence encoding a NS protein. For example, the one or more RNA(s) comprises an additional nucleotide sequence encoding a M2 protein, an additional sequence encoding a NP protein, and an additional sequence encoding a NS protein.

In one example, the nucleotide sequence encoding:
a) the NP protein is at least 90% identical to a nucleotide sequence set forth in SEQ ID NO: 69; and
b) the NS protein is at least 90% identical to a nucleotide sequence set forth in SEQ ID NO: 68.

In one example, the nucleotide sequence encoding:
a) the NP protein is at least 91% identical to a nucleotide sequence set forth in SEQ ID NO: 69; and
b) the NS protein is at least 91% identical to a nucleotide sequence set forth in SEQ ID NO: 68.

In one example, the nucleotide sequence encoding:
a) the NP protein is at least 92% identical to a nucleotide sequence set forth in SEQ ID NO: 69; and
b) the NS protein is at least 92% identical to a nucleotide sequence set forth in SEQ ID NO: 68.

In one example, the nucleotide sequence encoding:
a) the NP protein is at least 93% identical to a nucleotide sequence set forth in SEQ ID NO: 69; and
b) the NS protein is at least 93% identical to a nucleotide sequence set forth in SEQ ID NO: 68.

In one example, the nucleotide sequence encoding:
a) the NP protein is at least 94% identical to a nucleotide sequence set forth in SEQ ID NO: 69; and
b) the NS protein is at least 94% identical to a nucleotide sequence set forth in SEQ ID NO: 68.

In one example, the nucleotide sequence encoding:
a) the NP protein is at least 95% identical to a nucleotide sequence set forth in SEQ ID NO: 69; and
b) the NS protein is at least 95% identical to a nucleotide sequence set forth in SEQ ID NO: 68.

In one example, the nucleotide sequence encoding:
a) the NP protein is at least 96% identical to a nucleotide sequence set forth in SEQ ID NO: 69; and
b) the NS protein is at least 96% identical to a nucleotide sequence set forth in SEQ ID NO: 68.

In one example, the nucleotide sequence encoding:
a) the NP protein is at least 97% identical to a nucleotide sequence set forth in SEQ ID NO: 69; and
b) the NS protein is at least 97% identical to a nucleotide sequence set forth in SEQ ID NO: 68.

In one example, the nucleotide sequence encoding:
a) the NP protein is at least 98% identical to a nucleotide sequence set forth in SEQ ID NO: 69; and
b) the NS protein is at least 98% identical to a nucleotide sequence set forth in SEQ ID NO: 68.

In one example, the nucleotide sequence encoding:
a) the NP protein is at least 99% identical to a nucleotide sequence set forth in SEQ ID NO: 69; and
b) the NS protein is at least 99% identical to a nucleotide sequence set forth in SEQ ID NO: 68.

In one example, the nucleotide sequence encoding:
a) the NP protein comprises a nucleotide sequence set forth in SEQ ID NO: 69; and
b) the NS protein comprises a nucleotide sequence set forth in SEQ ID NO:68.

In one example, the nucleoprotein (NP) and/or the non-structural (NS) are from the same influenza virus.

In one example, the nucleoprotein (NP) and/or the non-structural (NS) are from different influenza viruses.

In one example, the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 1 collagen (Col1A), alpha 6 collagen (Col6A), alpha-1-antitrypsin (SERPINA1), alpha-1-antichymotrypsin (SERPINA3), arachidonate 5-lipoxygenase (ALOX5), tyrosine hydroxylase (TH gene), tumor protein P53 inducible protein 3 (TP5313) a fragment and/or a variant thereof. In one example, the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 1 collagen (Col1A), alpha 6 collagen (Col6A), alpha-1-antitrypsin (SERPINA1), alpha-1-antichymotrypsin (SERPINA3), arachidonate 5-lipoxygenase (ALOX5), tyrosine hydroxylase (TH gene), tumor protein P53 inducible protein 3 (TP5313), an alphavirus, a fragment and/or a variant thereof. For example, the first nucleotide sequence comprises the 5′-UTR of haptoglobin (HP). For example, the first nucleotide sequence comprises the 5′-UTR of fibrinogen beta chain (FGB). For example, the first nucleotide sequence comprises the 5′-UTR of haptoglobin-related protein (HPR). For example, the first nucleotide sequence comprises the 5′-UTR of albumin (ALB). For example, the first nucleotide sequence comprises the 5′-UTR of complement component 3 (C3). For example, the first nucleotide sequence comprises the 5′-UTR of fibrinogen alpha chain (FGA). For example, the first nucleotide sequence comprises the 5′-UTR of alpha 1 collagen (Col1A). For example, the first nucleotide sequence comprises the 5′-UTR of alpha 6 collagen (Col6A). For example, the first nucleotide sequence comprises the 5′-UTR of alpha-1-antitrypsin (SERPINA1). For example, the first nucleotide sequence comprises the 5′-UTR of alpha-1-antichymotrypsin (SERPINA3). For example, the first nucleotide sequence comprises the 5′-UTR of arachidonate 5-lipoxygenase (ALOX5). For example, the first nucleotide sequence comprises the 5′-UTR of tyrosine hydroxylase (TH gene). For example, the first nucleotide sequence comprises the 5′-UTR of tumor protein P53 inducible protein 3 (TP5313). For example, the first nucleotide sequence comprises the 5′-UTR of an alphavirus. For example, the first nucleotide sequence comprises the 5′-UTR of Venezuelan equine encephalitis virus.

In one example, the 5′-UTR, the fragment and/or the variant thereof is between 40 and 2000 nucleotides in length. For example, the 5′-UTR, the fragment and/or the variant thereof is between 40 and 100 nucleotides in length. For example, the 5′-UTR, the fragment and/or the variant thereof is between 100 and 250 nucleotides in length. For example, the 5′-UTR, the fragment and/or the variant thereof is between 250 and 500 nucleotides in length. For example, the 5′-UTR, the fragment and/or the variant thereof is between 500 and 750 nucleotides in length. For example, the 5′-UTR, the fragment and/or the variant thereof is between 750 and 1000 nucleotides in length. For example, the 5′-UTR, the fragment and/or the variant thereof is between 1000 and 1250 nucleotides in length. For example, the 5′-UTR, the fragment and/or the variant thereof is between 1250 and 1500 nucleotides in length. For example, the 5′-UTR, the fragment and/or the variant thereof is between 1500 and 1750 nucleotides in length. For example, the 5′-UTR, the fragment and/or the variant thereof is between 1750 and 2000 nucleotides in length.

In one example, the 5′-UTR, the fragment and/or the variant thereof comprises a nucleotide sequence at least 90% identical to a nucleotide sequence set forth in any one of SEQ ID NOs: 6 to 19, 60 and 76. For example, the 5′-UTR, the fragment and/or the variant thereof comprises a nucleotide sequence 90%, or 91%, or 92%, or 93%, or 94%, or 95%, or 96%, or 97%, or 98%, or 99% identical to a nucleotide sequence set forth in any one of SEQ ID NOs: 6 to 19, 60 and 76. For example, the 5′-UTR, the fragment and/or the variant thereof comprises a nucleotide sequence at least 90% identical to a nucleotide sequence set forth in SEQ ID NO: 6. For example, the 5′-UTR, the fragment and/or the variant thereof comprises a nucleotide sequence at least 90% identical to a nucleotide sequence set forth in SEQ ID NO: 7. For example, the 5′-UTR, the fragment and/or the variant thereof comprises a nucleotide sequence at least 90% identical to a nucleotide sequence set forth in SEQ ID NO: 8. For example, the 5′-UTR, the fragment and/or the variant thereof comprises a nucleotide sequence at least 90% identical to a nucleotide sequence set forth in SEQ ID NO: 9. For example, the 5′-UTR, the fragment and/or the variant thereof comprises a nucleotide sequence at least 90% identical to a nucleotide sequence set forth in SEQ ID NO: 10. For example, the 5′-UTR, the fragment and/or the variant thereof comprises a nucleotide sequence at least 90% identical to a nucleotide sequence set forth in SEQ ID NO: 11. For example, the 5′-UTR, the fragment and/or the variant thereof comprises a nucleotide sequence at least 90% identical to a nucleotide sequence set forth in SEQ ID NO: 12. For example, the 5′-UTR, the fragment and/or the variant thereof comprises a nucleotide sequence at least 90% identical to a nucleotide sequence set forth in SEQ ID NO: 13. For example, the 5′-UTR, the fragment and/or the variant thereof comprises a nucleotide sequence at least 90% identical to a nucleotide sequence set forth in SEQ ID NO: 14. For example, the 5′-UTR, the fragment and/or the variant thereof comprises a nucleotide sequence at least 90% identical to a nucleotide sequence set forth in SEQ ID NO: 15. For example, the 5′-UTR, the fragment and/or the variant thereof comprises a nucleotide sequence at least 90% identical to a nucleotide sequence set forth in SEQ ID NO: 16. For example, the 5′-UTR, the fragment and/or the variant thereof comprises a nucleotide sequence at least 90% identical to a nucleotide sequence set forth in SEQ ID NO: 17. For example, the 5′-UTR, the fragment and/or the variant thereof comprises a nucleotide sequence at least 90% identical to a nucleotide sequence set forth in SEQ ID NO: 18. For example, the 5′-UTR, the fragment and/or the variant thereof comprises a nucleotide sequence at least 90% identical to a nucleotide sequence set forth in SEQ ID NO: 19. For example, the 5′-UTR, the fragment and/or the variant thereof comprises a nucleotide sequence at least 90% identical to a nucleotide sequence set forth in SEQ ID NO: 60. For example, the 5′-UTR, the fragment and/or the variant thereof comprises a nucleotide sequence at least 90% identical to a nucleotide sequence set forth in SEQ ID NO: 76.

In one example, the 5′-UTR, the fragment and/or the variant thereof comprises a nucleotide sequence at least 95% identical to a nucleotide sequence set forth in any one of SEQ ID NOs: 6 to 19, 60 and 76. For example, the 5′-UTR, the fragment and/or the variant thereof comprises a nucleotide sequence at least 95% identical to a nucleotide sequence set forth in SEQ ID NO: 6. For example, the 5′-UTR, the fragment and/or the variant thereof comprises a nucleotide sequence at least 95% identical to a nucleotide sequence set forth in SEQ ID NO: 7. For example, the 5′-UTR, the fragment and/or the variant thereof comprises a nucleotide sequence at least 95% identical to a nucleotide sequence set forth in SEQ ID NO: 8. For example, the 5′-UTR, the fragment and/or the variant thereof comprises a nucleotide sequence at least 95% identical to a nucleotide sequence set forth in SEQ ID NO: 9. For example, the 5′-UTR, the fragment and/or the variant thereof comprises a nucleotide sequence at least 95% identical to a nucleotide sequence set forth in SEQ ID NO: 10. For example, the 5′-UTR, the fragment and/or the variant thereof comprises a nucleotide sequence at least 95% identical to a nucleotide sequence set forth in SEQ ID NO: 11. For example, the 5′-UTR, the fragment and/or the variant thereof comprises a nucleotide sequence at least 95% identical to a nucleotide sequence set forth in SEQ ID NO: 12. For example, the 5′-UTR, the fragment and/or the variant thereof comprises a nucleotide sequence at least 95% identical to a nucleotide sequence set forth in SEQ ID NO: 13. For example, the 5′-UTR, the fragment and/or the variant thereof comprises a nucleotide sequence at least 95% identical to a nucleotide sequence set forth in SEQ ID NO: 14. For example, the 5′-UTR, the fragment and/or the variant thereof comprises a nucleotide sequence at least 95% identical to a nucleotide sequence set forth in SEQ ID NO: 15. For example, the 5′-UTR, the fragment and/or the variant thereof comprises a nucleotide sequence at least 95% identical to a nucleotide sequence set forth in SEQ ID NO: 16. For example, the 5′-UTR, the fragment and/or the variant thereof comprises a nucleotide sequence at least 95% identical to a nucleotide sequence set forth in SEQ ID NO: 17. For example, the 5′-UTR, the fragment and/or the variant thereof comprises a nucleotide sequence at least 95% identical to a nucleotide sequence set forth in SEQ ID NO: 18. For example, the 5′-UTR, the fragment and/or the variant thereof comprises a nucleotide sequence at least 95% identical to a nucleotide sequence set forth in SEQ ID NO: 19. For example, the 5′-UTR, the fragment and/or the variant thereof comprises a nucleotide sequence at least 95% identical to a nucleotide sequence set forth in SEQ ID NO: 60. For example, the 5′-UTR, the fragment and/or the variant thereof comprises a nucleotide sequence at least 95% identical to a nucleotide sequence set forth in SEQ ID NO: 76.

In one example, the 5′-UTR, the fragment and/or the variant thereof comprises a nucleotide sequence at least 99% identical to a nucleotide sequence set forth in any one of SEQ ID NOs: 6 to 19, 60 and 76. For example, the 5′-UTR, the fragment and/or the variant thereof comprises a nucleotide sequence at least 99% identical to a nucleotide sequence set forth in SEQ ID NO: 6. For example, the 5′-UTR, the fragment and/or the variant thereof comprises a nucleotide sequence at least 99% identical to a nucleotide sequence set forth in SEQ ID NO: 7. For example, the 5′-UTR, the fragment and/or the variant thereof comprises a nucleotide sequence at least 99% identical to a nucleotide sequence set forth in SEQ ID NO: 8. For example, the 5′-UTR, the fragment and/or the variant thereof comprises a nucleotide sequence at least 99% identical to a nucleotide sequence set forth in SEQ ID NO: 9. For example, the 5′-UTR, the fragment and/or the variant thereof comprises a nucleotide sequence at least 99% identical to a nucleotide sequence set forth in SEQ ID NO: 10. For example, the 5′-UTR, the fragment and/or the variant thereof comprises a nucleotide sequence at least 99% identical to a nucleotide sequence set forth in SEQ ID NO: 11. For example, the 5′-UTR, the fragment and/or the variant thereof comprises a nucleotide sequence at least 99% identical to a nucleotide sequence set forth in SEQ ID NO: 12. For example, the 5′-UTR, the fragment and/or the variant thereof comprises a nucleotide sequence at least 99% identical to a nucleotide sequence set forth in SEQ ID NO: 13. For example, the 5′-UTR, the fragment and/or the variant thereof comprises a nucleotide sequence at least 99% identical to a nucleotide sequence set forth in SEQ ID NO: 14. For example, the 5′-UTR, the fragment and/or the variant thereof comprises a nucleotide sequence at least 99% identical to a nucleotide sequence set forth in SEQ ID NO: 15. For example, the 5′-UTR, the fragment and/or the variant thereof comprises a nucleotide sequence at least 99% identical to a nucleotide sequence set forth in SEQ ID NO: 16. For example, the 5′-UTR, the fragment and/or the variant thereof comprises a nucleotide sequence at least 99% identical to a nucleotide sequence set forth in SEQ ID NO: 17. For example, the 5′-UTR, the fragment and/or the variant thereof comprises a nucleotide sequence at least 99% identical to a nucleotide sequence set forth in SEQ ID NO: 18. For example, the 5′-UTR, the fragment and/or the variant thereof comprises a nucleotide sequence at least 99% identical to a nucleotide sequence set forth in SEQ ID NO: 19. For example, the 5′-UTR, the fragment and/or the variant thereof comprises a nucleotide sequence at least 99% identical to a nucleotide sequence set forth in SEQ ID NO: 60. For example, the 5′-UTR, the fragment and/or the variant thereof comprises a nucleotide sequence at least 99% identical to a nucleotide sequence set forth in SEQ ID NO: 76.

In one example, the 5′-UTR, the fragment and/or the variant thereof comprises a nucleotide sequence set forth in any one of SEQ ID NOs: 6 to 19 or 60. For example, the 5′-UTR, the fragment and/or the variant thereof comprises a nucleotide sequence set forth in SEQ ID NO: 6. For example, the 5′-UTR, the fragment and/or the variant thereof comprises a nucleotide sequence set forth in SEQ ID NO: 7. For example, the 5′-UTR, the fragment and/or the variant thereof comprises a nucleotide sequence set forth in SEQ ID NO: 8. For example, the 5′-UTR, the fragment and/or the variant thereof comprises a nucleotide sequence set forth in SEQ ID NO: 9. For example, the 5′-UTR, the fragment and/or the variant thereof comprises a nucleotide sequence set forth in SEQ ID NO: 10. For example, the 5′-UTR, the fragment and/or the variant thereof comprises a nucleotide sequence set forth in SEQ ID NO: 11. For example, the 5′-UTR, the fragment and/or the variant thereof comprises a nucleotide sequence set forth in SEQ ID NO: 12. For example, the 5′-UTR, the fragment and/or the variant thereof comprises a nucleotide sequence set forth in SEQ ID NO: 13. For example, the 5′-UTR, the fragment and/or the variant thereof comprises a nucleotide sequence set forth in SEQ ID NO: 14. For example, the 5′-UTR, the fragment and/or the variant thereof comprises a nucleotide sequence set forth in SEQ ID NO: 15. For example, the 5′-UTR, the fragment and/or the variant thereof comprises a nucleotide sequence set forth in SEQ ID NO: 16. For example, the 5′-UTR, the fragment and/or the variant thereof comprises a nucleotide sequence set forth in SEQ ID NO: 17. For example, the 5′-UTR, the fragment and/or the variant thereof comprises a nucleotide sequence set forth in SEQ ID NO: 18. For example, the 5′-UTR, the fragment and/or the variant thereof comprises a nucleotide sequence set forth in SEQ ID NO: 19. For example, the 5′-UTR, the fragment and/or the variant thereof comprises a nucleotide sequence set forth in SEQ ID NO: 60. For example, the 5′-UTR, the fragment and/or the variant thereof comprises a nucleotide sequence set forth in SEQ ID NO: 76.

In one example, the 5′-UTR is a synthetic 5′-UTR.

In one example, the 5′-UTR is a synthetic 5′-UTR comprising a nucleotide sequence at least 90% identical to a nucleotide sequence set forth in any one of SEQ ID NOs: 20 to 39 or 62. For example, the synthetic 5′-UTR comprises a nucleotide sequence 90%, or 91%, or 92%, or 93%, or 94%, or 95%, or 96%, or 97%, or 98%, or 99% identical to a nucleotide sequence set forth in any one of SEQ ID NOs: 20 to 39 or 62. For example, the synthetic 5′-UTR comprising a nucleotide sequence at least 90% identical to a nucleotide sequence set forth in SEQ ID NO: 20. For example, the synthetic 5′-UTR comprising a nucleotide sequence at least 90% identical to a nucleotide sequence set forth in SEQ ID NO: 21. For example, the synthetic 5′-UTR comprising a nucleotide sequence at least 90% identical to a nucleotide sequence set forth in SEQ ID NO: 22. For example, the synthetic 5′-UTR comprising a nucleotide sequence at least 90% identical to a nucleotide sequence set forth in SEQ ID NO: 23. For example, the synthetic 5′-UTR comprising a nucleotide sequence at least 90% identical to a nucleotide sequence set forth in SEQ ID NO: 24. For example, the synthetic 5′-UTR comprising a nucleotide sequence at least 90% identical to a nucleotide sequence set forth in SEQ ID NO: 25. For example, the synthetic 5′-UTR comprising a nucleotide sequence at least 90% identical to a nucleotide sequence set forth in SEQ ID NO: 26. For example, the synthetic 5′-UTR comprising a nucleotide sequence at least 90% identical to a nucleotide sequence set forth in SEQ ID NO: 27. For example, the synthetic 5′-UTR comprising a nucleotide sequence at least 90% identical to a nucleotide sequence set forth in SEQ ID NO: 28. For example, the synthetic 5′-UTR comprising a nucleotide sequence at least 90% identical to a nucleotide sequence set forth in SEQ ID NO: 29. For example, the synthetic 5′-UTR comprising a nucleotide sequence at least 90% identical to a nucleotide sequence set forth in SEQ ID NO: 30. For example, the synthetic 5′-UTR comprising a nucleotide sequence at least 90% identical to a nucleotide sequence set forth in SEQ ID NO: 31. For example, the synthetic 5′-UTR comprising a nucleotide sequence at least 90% identical to a nucleotide sequence set forth in SEQ ID NO: 32. For example, the synthetic 5′-UTR comprising a nucleotide sequence at least 90% identical to a nucleotide sequence set forth in SEQ ID NO: 33. For example, the synthetic 5′-UTR comprising a nucleotide sequence at least 90% identical to a nucleotide sequence set forth in SEQ ID NO: 34. For example, the synthetic 5′-UTR comprising a nucleotide sequence at least 90% identical to a nucleotide sequence set forth in SEQ ID NO: 35. For example, the synthetic 5′-UTR comprising a nucleotide sequence at least 90% identical to a nucleotide sequence set forth in SEQ ID NO: 36. For example, the synthetic 5′-UTR comprising a nucleotide sequence at least 90% identical to a nucleotide sequence set forth in SEQ ID NO: 37. For example, the synthetic 5′-UTR comprising a nucleotide sequence at least 90% identical to a nucleotide sequence set forth in SEQ ID NO: 38. For example, the synthetic 5′-UTR comprising a nucleotide sequence at least 90% identical to a nucleotide sequence set forth in SEQ ID NO: 39. For example, the synthetic 5′-UTR comprising a nucleotide sequence at least 90% identical to a nucleotide sequence set forth in SEQ ID NO: 62.

In one example, the 5′-UTR is a synthetic 5′-UTR comprising a nucleotide sequence at least 95% identical to a nucleotide sequence set forth in any one of SEQ ID NOs: 20 to 39 or 62. For example, the synthetic 5′-UTR comprising a nucleotide sequence at least 95% identical to a nucleotide sequence set forth in SEQ ID NO: 20. For example, the synthetic 5′-UTR comprising a nucleotide sequence at least 95% identical to a nucleotide sequence set forth in SEQ ID NO: 21. For example, the synthetic 5′-UTR comprising a nucleotide sequence at least 95% identical to a nucleotide sequence set forth in SEQ ID NO: 22. For example, the synthetic 5′-UTR comprising a nucleotide sequence at least 95% identical to a nucleotide sequence set forth in SEQ ID NO: 23. For example, the synthetic 5′-UTR comprising a nucleotide sequence at least 95% identical to a nucleotide sequence set forth in SEQ ID NO: 24. For example, the synthetic 5′-UTR comprising a nucleotide sequence at least 95% identical to a nucleotide sequence set forth in SEQ ID NO: 25. For example, the synthetic 5′-UTR comprising a nucleotide sequence at least 95% identical to a nucleotide sequence set forth in SEQ ID NO: 26. For example, the synthetic 5′-UTR comprising a nucleotide sequence at least 95% identical to a nucleotide sequence set forth in SEQ ID NO: 27. For example, the synthetic 5′-UTR comprising a nucleotide sequence at least 95% identical to a nucleotide sequence set forth in SEQ ID NO: 28. For example, the synthetic 5′-UTR comprising a nucleotide sequence at least 95% identical to a nucleotide sequence set forth in SEQ ID NO: 29. For example, the synthetic 5′-UTR comprising a nucleotide sequence at least 95% identical to a nucleotide sequence set forth in SEQ ID NO: 30. For example, the synthetic 5′-UTR comprising a nucleotide sequence at least 95% identical to a nucleotide sequence set forth in SEQ ID NO: 31. For example, the synthetic 5′-UTR comprising a nucleotide sequence at least 95% identical to a nucleotide sequence set forth in SEQ ID NO: 32. For example, the synthetic 5′-UTR comprising a nucleotide sequence at least 95% identical to a nucleotide sequence set forth in SEQ ID NO: 33. For example, the synthetic 5′-UTR comprising a nucleotide sequence at least 95% identical to a nucleotide sequence set forth in SEQ ID NO: 34. For example, the synthetic 5′-UTR comprising a nucleotide sequence at least 95% identical to a nucleotide sequence set forth in SEQ ID NO: 35. For example, the synthetic 5′-UTR comprising a nucleotide sequence at least 95% identical to a nucleotide sequence set forth in SEQ ID NO: 36. For example, the synthetic 5′-UTR comprising a nucleotide sequence at least 95% identical to a nucleotide sequence set forth in SEQ ID NO: 37. For example, the synthetic 5′-UTR comprising a nucleotide sequence at least 95% identical to a nucleotide sequence set forth in SEQ ID NO: 38. For example, the synthetic 5′-UTR comprising a nucleotide sequence at least 95% identical to a nucleotide sequence set forth in SEQ ID NO: 39. For example, the synthetic 5′-UTR comprising a nucleotide sequence at least 95% identical to a nucleotide sequence set forth in SEQ ID NO: 62.

In one example, the 5′-UTR is a synthetic 5′-UTR comprising a nucleotide sequence at least 99% identical to a nucleotide sequence set forth in any one of SEQ ID NOs: 20 to 39 or 62. For example, the synthetic 5′-UTR comprising a nucleotide sequence at least 99% identical to a nucleotide sequence set forth in SEQ ID NO: 20. For example, the synthetic 5′-UTR comprising a nucleotide sequence at least 99% identical to a nucleotide sequence set forth in SEQ ID NO: 21. For example, the synthetic 5′-UTR comprising a nucleotide sequence at least 99% identical to a nucleotide sequence set forth in SEQ ID NO: 22. For example, the synthetic 5′-UTR comprising a nucleotide sequence at least 99% identical to a nucleotide sequence set forth in SEQ ID NO: 23. For example, the synthetic 5′-UTR comprising a nucleotide sequence at least 99% identical to a nucleotide sequence set forth in SEQ ID NO: 24. For example, the synthetic 5′-UTR comprising a nucleotide sequence at least 99% identical to a nucleotide sequence set forth in SEQ ID NO: 25. For example, the synthetic 5′-UTR comprising a nucleotide sequence at least 99% identical to a nucleotide sequence set forth in SEQ ID NO: 26. For example, the synthetic 5′-UTR comprising a nucleotide sequence at least 99% identical to a nucleotide sequence set forth in SEQ ID NO: 27. For example, the synthetic 5′-UTR comprising a nucleotide sequence at least 99% identical to a nucleotide sequence set forth in SEQ ID NO: 28. For example, the synthetic 5′-UTR comprising a nucleotide sequence at least 99% identical to a nucleotide sequence set forth in SEQ ID NO: 29. For example, the synthetic 5′-UTR comprising a nucleotide sequence at least 99% identical to a nucleotide sequence set forth in SEQ ID NO: 30. For example, the synthetic 5′-UTR comprising a nucleotide sequence at least 99% identical to a nucleotide sequence set forth in SEQ ID NO: 31. For example, the synthetic 5′-UTR comprising a nucleotide sequence at least 99% identical to a nucleotide sequence set forth in SEQ ID NO: 32. For example, the synthetic 5′-UTR comprising a nucleotide sequence at least 99% identical to a nucleotide sequence set forth in SEQ ID NO: 33. For example, the synthetic 5′-UTR comprising a nucleotide sequence at least 99% identical to a nucleotide sequence set forth in SEQ ID NO: 34. For example, the synthetic 5′-UTR comprising a nucleotide sequence at least 99% identical to a nucleotide sequence set forth in SEQ ID NO: 35. For example, the synthetic 5′-UTR comprising a nucleotide sequence at least 99% identical to a nucleotide sequence set forth in SEQ ID NO: 36. For example, the synthetic 5′-UTR comprising a nucleotide sequence at least 99% identical to a nucleotide sequence set forth in SEQ ID NO: 37. For example, the synthetic 5′-UTR comprising a nucleotide sequence at least 99% identical to a nucleotide sequence set forth in SEQ ID NO: 38. For example, the synthetic 5′-UTR comprising a nucleotide sequence at least 99% identical to a nucleotide sequence set forth in SEQ ID NO: 39. For example, the synthetic 5′-UTR comprising a nucleotide sequence at least 99% identical to a nucleotide sequence set forth in SEQ ID NO: 62.

In one example, the 5′-UTR is a synthetic 5′-UTR comprising or consisting of a nucleotide sequence set forth in any one of SEQ ID NOs: 20 to 39 or 62. For example, the synthetic 5′-UTR comprises a nucleotide sequence set forth in any one of SEQ ID NOs: 20 to 39 or 62. For example, the synthetic 5′-UTR comprises a nucleotide sequence set forth in SEQ ID NO: 20. For example, the synthetic 5′-UTR comprises a nucleotide sequence set forth in SEQ ID NO: 21. For example, the synthetic 5′-UTR comprises a nucleotide sequence set forth in SEQ ID NO: 22. For example, the synthetic 5′-UTR comprises a nucleotide sequence set forth in SEQ ID NO: 23. For example, the synthetic 5′-UTR comprises a nucleotide sequence set forth in SEQ ID NO: 24. For example, the synthetic 5′-UTR comprises a nucleotide sequence set forth in SEQ ID NO: 25. For example, the synthetic 5′-UTR comprises a nucleotide sequence set forth in SEQ ID NO: 26. For example, the synthetic 5′-UTR comprises a nucleotide sequence set forth in SEQ ID NO: 27. For example, the synthetic 5′-UTR comprises a nucleotide sequence set forth in SEQ ID NO: 28. For example, the synthetic 5′-UTR comprises a nucleotide sequence set forth in SEQ ID NO: 29. For example, the synthetic 5′-UTR comprises a nucleotide sequence set forth in SEQ ID NO: 30. For example, the synthetic 5′-UTR comprises a nucleotide sequence set forth in SEQ ID NO: 31. For example, the synthetic 5′-UTR comprises a nucleotide sequence set forth in SEQ ID NO: 32. For example, the synthetic 5′-UTR comprises a nucleotide sequence set forth in SEQ ID NO: 33. For example, the synthetic 5′-UTR comprises a nucleotide sequence set forth in SEQ ID NO: 34. For example, the synthetic 5′-UTR comprises a nucleotide sequence set forth in SEQ ID NO: 35. For example, the synthetic 5′-UTR comprises a nucleotide sequence set forth in SEQ ID NO: 36. For example, the synthetic 5′-UTR comprises a nucleotide sequence set forth in SEQ ID NO: 37. For example, the synthetic 5′-UTR comprises a nucleotide sequence set forth in SEQ ID NO: 38. For example, the synthetic 5′-UTR comprises a nucleotide sequence set forth in SEQ ID NO: 39. For example, the synthetic 5′-UTR comprises a nucleotide sequence set forth in SEQ ID NO: 62.

For example, the synthetic 5′-UTR consists of a nucleotide sequence set forth in any one of SEQ ID NOs: 20 to 39 or 62. For example, the synthetic 5′-UTR consists of a nucleotide sequence set forth in SEQ ID NO: 20. For example, the synthetic 5′-UTR consists of a nucleotide sequence set forth in SEQ ID NO: 21. For example, the synthetic 5′-UTR consists of a nucleotide sequence set forth in SEQ ID NO: 22. For example, the synthetic 5′-UTR consists of a nucleotide sequence set forth in SEQ ID NO: 23. For example, the synthetic 5′-UTR consists of a nucleotide sequence set forth in SEQ ID NO: 24. For example, the synthetic 5′-UTR consists of a nucleotide sequence set forth in SEQ ID NO: 25. For example, the synthetic 5′-UTR consists of a nucleotide sequence set forth in SEQ ID NO: 26. For example, the synthetic 5′-UTR consists of a nucleotide sequence set forth in SEQ ID NO: 27. For example, the synthetic 5′-UTR consists of a nucleotide sequence set forth in SEQ ID NO: 28. For example, the synthetic 5′-UTR consists of a nucleotide sequence set forth in SEQ ID NO: 29. For example, the synthetic 5′-UTR consists of a nucleotide sequence set forth in SEQ ID NO: 30. For example, the synthetic 5′-UTR consists of a nucleotide sequence set forth in SEQ ID NO: 31. For example, the synthetic 5′-UTR consists of a nucleotide sequence set forth in SEQ ID NO: 32. For example, the synthetic 5′-UTR consists of a nucleotide sequence set forth in SEQ ID NO: 33. For example, the synthetic 5′-UTR consists of a nucleotide sequence set forth in SEQ ID NO: 34. For example, the synthetic 5′-UTR consists of a nucleotide sequence set forth in SEQ ID NO: 35. For example, the synthetic 5′-UTR consists of a nucleotide sequence set forth in SEQ ID NO: 36. For example, the synthetic 5′-UTR consists of a nucleotide sequence set forth in SEQ ID NO: 37. For example, the synthetic 5′-UTR consists of a nucleotide sequence set forth in SEQ ID NO: 38. For example, the synthetic 5′-UTR consists of a nucleotide sequence set forth in SEQ ID NO: 39. For example, the synthetic 5′-UTR consists of a nucleotide sequence set forth in SEQ ID NO: 62.

In one example, the first nucleotide sequence comprises a combination of two or more 5′-UTRs, fragments and/or variants thereof. In one example, two or more 5′-UTRs are the same. In one example, two or more 5′-UTRs are different.

In one example, the first nucleotide sequence comprises at least one microRNA binding site, an AU rich element (ARE), a GC-rich element, a stem loop, and combinations thereof. For example, the first nucleotide sequence comprises a microRNA binding site. For example, the first nucleotide sequence comprises an AU rich element (ARE). For example, the first nucleotide comprises a GC-rich element. For example, the first nucleotide sequence comprises a stem loop.

In one example, a translation initiation sequence selected from the group consisting of a Kozak consensus sequence, an internal ribosome entry site (IRES), a subgenomic (SG) promoter and combinations thereof is operably linked to the 5′ end of the one or more nucleotide sequence(s) encoding the VLP forming element and/or the one or more additional nucleotide sequence encoding a nucleoprotein (NP) and/or a non-structural (NS) protein. For example, a Kozak consensus sequence is operably linked to the 5′ end of the one or more nucleotide sequence(s) encoding the VLP forming element and/or the one or more additional nucleotide sequence encoding a nucleoprotein (NP) and/or a non-structural (NS) protein. For example, a IRES is operably linked to the 5′ end of the one or more nucleotide sequence(s) encoding the VLP forming element and/or the one or more additional nucleotide sequence encoding a nucleoprotein (NP) and/or a non-structural (NS) protein. For example, a SG promoter is operably linked to the 5′ end of the one or more nucleotide sequence(s) encoding the VLP forming element and/or the one or more additional nucleotide sequence encoding a nucleoprotein (NP) and/or a non-structural (NS) protein.

In one example, the Kozak consensus sequence comprises or consists of a sequence set forth in SEQ ID NO: 1 or SEQ ID NO: 2. In one example, the Kozak consensus sequence consists of a sequence set forth in SEQ ID NO: 1. In one example, the Kozak consensus sequence comprises a sequence set forth in SEQ ID NO: 1. In one example, the Kozak consensus sequence consists of a sequence set forth in SEQ ID NO: 2. In one example, the Kozak consensus sequence comprises a sequence set forth in SEQ ID NO: 2.

In one example, wherein the IRES is an IRES from poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof. For example, the IRES is an IRES from poliovirus (PV). For example, the IRES is an IRES from human enterovirus. For example, the IRES is an IRES from foot-and-mouth disease virus (FMDV). For example, For example, the IRES is an IRES from hepatitis C virus (HCV). For example, the IRES is an IRES from classical swine fever virus (CSFV). For example, the IRES is an IRES from murine leukemia virus (MLV). For example, the IRES is an IRES from simian immunodeficiency virus (SIV). For example, the IRES is an IRES from Eukaryotic translation initiation factor 4G (elF4G). For example, the IRES is an IRES from Death-associated protein 5 (DAP5). For example, the IRES is an IRES from cellular Myc (c-Myc). For example, the IRES is an IRES from NF-κB-repressing factor (NRF). For example, the IRES is an IRES from vascular endothelial growth factor (VEGF). For example, the IRES is an IRES from fibroblast growth factor (FGF-2). For example, the IRES is an IRES from platelet-derived growth factor B (PDGF B). For example, the IRES is an IRES from Antennapedia. For example, the IRES is an IRES from X-linked inhibitor of apoptosis (XIAP or Apaf-1). For example, the IRES is an IRES from immunoglobulin heavy-chain binding protein BiP. For example, the IRES is an IRES from fibroblast growth factor 1a (FGF1A). For example, the IRES is an IRES from GTX.

In one example, the IRES comprises a nucleotide sequence at least 90% identical to a nucleotide sequence set forth in SEQ ID NO: 57 or 58. For example, the IRES comprises a nucleotide sequence 90%, or 91%, or 92%, or 93%, or 94%, or 95%, or 96%, or 97%, or 98%, or 99% identical to a nucleotide sequence set forth in SEQ ID NO: 57 or 58. For example, the IRES comprises a nucleotide sequence at least 90% identical to a nucleotide sequence set forth in SEQ ID NO: 57. For example, the IRES comprises a nucleotide sequence at least 90% identical to a nucleotide sequence set forth in SEQ ID NO: 58.

In one example, the IRES comprises a nucleotide sequence at least 95% identical to a nucleotide sequence set forth in SEQ ID NO: 57 or 58. For example, the IRES comprises a nucleotide sequence at least 95% identical to a nucleotide sequence set forth in SEQ ID NO: 57. For example, the IRES comprises a nucleotide sequence at least 95% identical to a nucleotide sequence set forth in SEQ ID NO: 58.

In one example, the IRES comprises a nucleotide sequence at least 99% identical to a nucleotide sequence set forth in SEQ ID NO: 57 or 58. For example, the IRES comprises a nucleotide sequence at least 99% identical to a nucleotide sequence set forth in SEQ ID NO: 57. For example, the IRES comprises a nucleotide sequence at least 99% identical to a nucleotide sequence set forth in SEQ ID NO: 58.

In one example, the IRES comprises a nucleotide sequence set forth in SEQ ID NO: 57 or 58. For example, the IRES comprises a nucleotide sequence set forth in SEQ ID NO: 57. For example, the IRES comprises a nucleotide sequence set forth in SEQ ID NO: 58.

In one example, the SG promoter is a minimal SG promoter or an extended SG promoter.

In one example, the SG promoter is a minimal SG promoter. For example, the minimal SG promoter is a native SG promoter. For example, the minimal SG promoter is the minimal sequence required for initiation of transcription. In one example, the minimal native SG promoter is 49 nucleotides in length. In another example, the extended SG promoter comprises or consists of SG promoter v1. In one example, the minimal native SG promoter comprises or consists of the sequence set forth in SEQ ID NO: 3. In one example, the SG promoter consists of the sequence set forth in SEQ ID NO: 3.

In one example, the SG promoter is an extended SG promoter. For example, the extended SG promoter is extended at the 5′ end with nucleotides occurring in a sequence encoding a non-structural protein of the RNA virus. In one example, the extended SG promoter is extended at the 5′ end with nucleotides occurring in a sequence encoding an alphavirus NSP4. In one example, the extended SG promoter comprises the minimal SG promoter extended at the 5′ end with nucleotides occurring in a sequence encoding a non-structural protein of an RNA virus. In one example, the extended SG promoter comprises the sequence set forth in SEQ ID NO: 3 extended at the 5′ end with nucleotides occurring in a sequence encoding a non-structural protein of an RNA virus.

In one example, the SG promoter is extended at the 5′ end by 51 or fewer nucleotides occurring in a sequence encoding a non-structural protein. In one example, the extended SG promoter is a minimal SG promoter extended at the 5′ end by no more than 51 nucleotides occurring in a sequence encoding a non-structural protein.

In one example, the extended SG promoter comprises or consists of the sequence set forth in SEQ ID NO: 3 extended at the 5′ end by no more than 51 nucleotides occurring in a sequence encoding a non-structural protein. For example, the extended SG promoter is no more than 100 nucleotides in length. In one example, the extended SG promoter comprises or consists of nucleotides 2 to 101 of SEQ ID NO: 4.

In one example, the extended SG promoter comprises or consists of the sequence set forth in SEQ ID NO: 4. For example, the extended SG promoter comprises the sequence set forth in SEQ ID NO: 4. For example, the extended SG promoter consists of the sequence set forth in SEQ ID NO: 4.

In another example, the extended SG promoter comprises or consists of SG promoter v2. In another example, the extended SG promoter comprises or consists of the sequence set forth in SEQ ID NO: 75. For example, the extended SG promoter comprises the sequence set forth in SEQ ID NO: 75. For example, the extended SG promoter consists of the sequence set forth in SEQ ID NO: 75.

In another example, the extended SG promoter comprises or consists of the sequence set forth in SEQ ID NO: 86. For example, the extended SG promoter comprises the sequence set forth in SEQ ID NO: 86. For example, the extended SG promoter consists of the sequence set forth in SEQ ID NO: 86.

In another example, the extended SG promoter comprises or consists of the sequence set forth in SEQ ID NO: 87. For example, the extended SG promoter comprises the sequence set forth in SEQ ID NO: 87. For example, the extended SG promoter consists of the sequence set forth in SEQ ID NO: 87.

In one example, the extended SG promoter comprises a sequence set forth in SEQ ID NO: 75 or SEQ ID NO: 86 or SEQ ID NO: 87.

The present disclosure further provides a composition comprising a RNA comprising, in 5′ to 3′ order:
a) a nucleotide sequence encoding a hemagglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding a neuraminidase (NA) protein of an influenza virus operably linked to a second subgenomic (SG) promoter; and
c) a nucleotide sequence encoding a matrix-1 (M1) protein of an influenza virus operably linked to a third subgenomic (SG) promoter.

In one example, the is provided a composition comprising a RNA comprising, in 5′ to 3′ order:
a) a nucleotide sequence encoding a hemaglutinin (HA) protein of an influenza virus operably linked to a first subgenomic (SG) promoter;
b) a nucleotide sequence encoding a neuraminidase (NA) protein of an influenza virus operably linked to a second subgenomic (SG) promoter; and
c) a nucleotide sequence encoding a matrix-1 (M1) protein of an influenza virus operably linked to a third subgenomic (SG) promoter.

In one example, the RNA is a self-replicating RNA or a conventional mRNA.

In one example, the first subgenomic (SG) promoter comprises a native subgenomic (SG) promoter.

In one example, the second subgenomic (SG) promoter comprises the sequence set forth in SEQ ID NO: 3 or SEQ ID NO: 75. In one example, the second subgenomic (SG) promoter comprises the sequence set forth in SEQ ID NO: 3. In one example, the second subgenomic (SG) promoter comprises the sequence set forth in SEQ ID NO: 75.

In one example, the third subgenomic (SG) promoter comprises the sequence set forth in SEQ ID NO: 3 or SEQ ID NO: 75. In one example, the third subgenomic (SG) promoter comprises the sequence set forth in SEQ ID NO: 3. In one example, the third subgenomic (SG) promoter comprises the sequence set forth in SEQ ID NO: 75.

In one example, the second subgenomic (SG) promoter comprises the sequence set forth in SEQ ID NO: 75 and the second subgenomic (SG) promoter comprises the sequence set forth in SEQ ID NO: 3. In one example, the second subgenomic (SG) promoter comprises the sequence set forth in SEQ ID NO: 3 and the second subgenomic (SG) promoter comprises the sequence set forth in SEQ ID NO: 75.

In one example, the RNA comprises a 5′UTR, for example as defined herein. In one example, the RNA comprises a 3′UTR, for examples as defined herein. In one example, the RNA comprises a tailing sequence, for example as defined herein. In one example, the RNA comprises a 5′cap, for example as defined herein.

The present disclosure also provides a method of increasing stability of a virus-like particle (VLP), the method comprising introducing a nucleotide sequence encoding a matrix-1 (M1) protein of an influenza virus into a composition, wherein the composition comprises a RNA comprising, in 5′ to 3′ order:
a) a nucleotide sequence encoding a hemagglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding a neuraminidase (NA) protein of an influenza virus operably linked to a second subgenomic (SG) promoter;
c) a nucleotide sequence encoding the M1 protein of an influenza virus operably linked to a third subgenomic (SG) promoter; and
wherein introducing the nucleotide sequence encoding the M1 protein into the composition increases the stability of the VLP produced from the composition.

The present disclosure further provides a method of increasing efficient release of a virus-like particle (VLP), the method comprising introducing a nucleotide sequence encoding a neuraminidase (NA) protein of an influenza virus into a composition, wherein the composition comprises a RNA comprising, in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding the NA protein of an influenza virus operably linked to a second subgenomic (SG) promoter;
c) a nucleotide sequence encoding a matrix-1 (M1) protein of an influenza virus operably linked to a third subgenomic (SG) promoter; and
wherein introducing the nucleotide sequence encoding the NA protein into the composition increases efficient release of the VLP produced from the composition.

The present disclosure also provides a method of increasing stability and efficient release of a virus-like particle (VLP), the method comprising introducing a nucleotide sequence encoding a matrix-1 (M1) protein and a nucleotide sequence encoding a neuraminidase (NA) protein of an influenza virus into a composition, wherein the composition comprises a RNA comprising, in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding the NA protein of an influenza virus operably linked to a second subgenomic (SG) promoter;
c) a nucleotide sequence encoding the M1 protein of an influenza virus operably linked to a third subgenomic (SG) promoter; and
wherein introducing the nucleotide sequence encoding the M1 protein and the NA protein into the composition increases the stability and efficient release of the VLP produced from the composition.

In one example, the nucleotide sequence encoding a hemaglutinin (HA) protein of an influenza virus is operably linked to a nucleotide sequence comprising a 5′-untranslated region (5′-UTR), a fragment and/or a variant thereof.

In one example, the nucleotide sequence encoding a hemaglutinin (HA) protein of an influenza virus is operably linked to a first SG promoter.

In one example, the first SG promoter and second SG promoter are the same.

In one example, the first SG promoter and second SG promoter are different.

In one example, the second SG promoter and third SG promoter are the same.

In one example, the second SG promoter and third SG promoter are different.

In one example, the first SG promoter and third SG promoter are the same.

In one example, the first SG promoter and third SG promoter are different.

In one example, the first SG promoter, the second SG promoter and the third SG promoter are the same.

In one example, the first SG promoter, the second SG promoter and the third SG promoter are different.

In one example, the first subgenomic promoter comprises a minimal native SG promoter. In one example, the first subgenomic promoter comprises or consists of the sequence set forth in SEQ ID NO: 3. In one example, the first subgenomic promoter consists of the sequence set forth in SEQ ID NO: 3.

In one example, the second subgenomic promoter comprises a native SG promoter. In one example, the second subgenomic promoter comprises or consists of the sequence set forth in SEQ ID NO: 3. In one example, the second subgenomic promoter consists of the sequence set forth in SEQ ID NO: 3. In one example, the second subgenomic promoter comprises an extended SG promoter. In one example, the second subgenomic promoter comprises or consists of the sequence set forth in SEQ ID NO: 75. In one example, the second subgenomic promoter consists of the sequence set forth in SEQ ID NO: 75.

In one example, the third subgenomic promoter comprises a native SG promoter. In one example, the third subgenomic promoter comprises or consists of the sequence set forth in SEQ ID NO: 3. In one example, the third subgenomic promoter consists of the sequence set forth in SEQ ID NO: 3. In one example, the third subgenomic promoter comprises an extended SG promoter. In one example, the third subgenomic promoter comprises or consists of the sequence set forth in SEQ ID NO: 75. In one example, the third subgenomic promoter consists of the sequence set forth in SEQ ID NO: 75.

In one example, the first subgenomic promoter consists of the sequence set forth in SEQ ID NO: 3, the second subgenomic promoter consists of the sequence set forth in SEQ ID NO: 3, and the third subgenomic promoter consists of the sequence set forth in SEQ ID NO: 3.

In one example, the first subgenomic promoter consists of the sequence set forth in SEQ ID NO: 3, the second subgenomic promoter consists of the sequence set forth in SEQ ID NO: 75, and the third subgenomic promoter consists of the sequence set forth in SEQ ID NO: 3. In one example, the first subgenomic promoter consists of the sequence set forth in SEQ ID NO: 3, the second subgenomic promoter consists of the sequence set forth in SEQ ID NO: 75, and the third subgenomic promoter consists of the sequence set forth in SEQ ID NO: 75. In one example, the first subgenomic promoter consists of the sequence set forth in SEQ ID NO: 3, the second subgenomic promoter consists of the sequence set forth in SEQ ID NO: 3, and the third subgenomic promoter consists of the sequence set forth in SEQ ID NO: 75.

In one example, the second nucleotide sequence comprises the 3′-UTR of creatine kinase, globin, α-actin, albumin, granulocyte colony stimulating factor (G-CSF), collagen, ribophorin I (RPNI), low density lipoprotein receptor-related protein 1 (LRP1), cardiotrophin-like cytokine factor 1 (CLCF1), calreticulin (Calr), procollagen-lysine 2-oxoglutarate5-dioxygenase 1 (Plodl), nucleobindin1 (Nucbl), amino-terminal enhancer of split (AES), human mitochondrial 12S rRNA (mtRNR1), a fragment and/or a variant thereof. For example, the second nucleotide sequence comprises the 3′-UTR of creatine kinase, a fragment and/or a variant thereof. In one example, the second nucleotide sequence comprises the 3′-UTR of creatine kinase, globin, α-actin, albumin, granulocyte colony stimulating factor (G-CSF), collagen, ribophorin I (RPNI), low density lipoprotein receptor-related protein 1 (LRP1), cardiotrophin-like cytokine factor 1 (CLCF1), calreticulin (Calr), procollagen-lysine 2-oxoglutarate5-dioxygenase 1 (Plodl), nucleobindin1 (Nucbl), amino-terminal enhancer of split (AES), human mitochondrial 12S rRNA (mtRNR1), an alphavirus, a fragment and/or a variant thereof. For example, the second nucleotide sequence comprises the 3′-UTR of globin, a fragment and/or a variant thereof. For example, the second nucleotide sequence comprises the 3′-UTR of α-actin, a fragment and/or a variant thereof. For example, the second nucleotide sequence comprises the 3′-UTR of albumin, a fragment and/or a variant thereof. For example, the second nucleotide sequence comprises the 3′-UTR of granulocyte colony stimulating factor (G-CSF), a fragment and/or a variant thereof. For example, the second nucleotide sequence comprises the 3′-UTR of collagen, a fragment and/or a variant thereof. For example, the second nucleotide sequence comprises ribophorin I (RPNI), a fragment and/or a variant thereof. For example, the second nucleotide sequence comprises the 3′-UTR of low density lipoprotein receptor-related protein 1 (LRP1), a fragment and/or a variant thereof. For example, the second nucleotide sequence comprises the 3′-UTR of cardiotrophin-like cytokine factor 1 (CLCF1), a fragment and/or a variant thereof. For example, the second nucleotide sequence comprises the 3′-UTR of calreticulin (Calr), a fragment and/or a variant thereof. For example, the second nucleotide sequence comprises the 3′-UTR of procollagen-lysine 2-oxoglutarate5-dioxygenase 1 (Plodl), a fragment and/or a variant thereof. For example, the second nucleotide sequence comprises the 3′-UTR of nucleobindin1 (Nucbl), a fragment and/or a variant thereof. For example, the second nucleotide sequence comprises the 3′-UTR of amino-terminal enhancer of split (AES), a fragment and/or a variant thereof. For example, the second nucleotide sequence comprises the 3′-UTR of human mitochondrial 12S rRNA (mtRNR1), a fragment and/or a variant thereof. For example, the second nucleotide sequence comprises the 3′-UTR of an alphavirus. For example, the second nucleotide sequence comprises the 3′-UTR of Venezuelan equine encephalitis virus. For example, the second nucleotide sequence comprises the 3′-UTR of Sindbis virus.

In one example, the 3′-UTR is between 40 and 400 nucleotides in length. For example, the 3′-UTR is between 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300, or 300 and 325, or 325 and 350, or 350 and 375, or 375 and 400 nucleotides in length. For example, the 3′-UTR is between 40 and 50 nucleotides in length. For example, the 3′-UTR is between 50 and 60 nucleotides in length. For example, the 3′-UTR is between 60 and 70 nucleotides in length. For example, the 3′-UTR is between 70 and 80 nucleotides in length. For example, the 3′-UTR is between 80 and 90 nucleotides in length. For example, the 3′-UTR is between 90 and 100 nucleotides in length. For example, the 3′-UTR is between 100 and 125 nucleotides in length. For example, the 3′-UTR is between 125 and 150 nucleotides in length. For example, the 3′-UTR is between 150 and 175 nucleotides in length. For example, the 3′-UTR is between 175 and 200 nucleotides in length. For example, the 3′-UTR is between 200 and 225 nucleotides in length. For example, the 3′-UTR is between 225 and 250 nucleotides in length. For example, the 3′-UTR is between 250 and 275 nucleotides in length. For example, the 3′-UTR is between 275 and 300 nucleotides in length. For example, the 3′-UTR is between 300 and 325 nucleotides in length. For example, the 3′-UTR is between 325 and 350 nucleotides in length. For example, the 3′-UTR is between 350 and 375 nucleotides in length. For example, the 3′-UTR is between 375 and 400 nucleotides in length.

In one example, the 3′-UTR is at least 40 nucleotides in length. For example, the 3′-UTR is at least 45, or 50, or 55, or 60, or 65, or 70, or 75, or 80, or 85, or 90, or 95, or 100, or 110, or 120, or 130, or 140, or 150, or 160, or 170, or 180, or 190, or 200, or 210, or 220, or 230, or 240, or 250, or 260, or 270, or 280, or 290, or 300, or 310, or 320, or 330, or 340, or 350, or 360, or 370, or 380, or 390 nucleotides in length. For example, the 3′-UTR is at least 45 nucleotides in length. For example, the 3′-UTR is at least 50 nucleotides in length. For example, the 3′-UTR is at least 55 nucleotides in length. For example, the 3′-UTR is at least 60 nucleotides in length. For example, the 3′-UTR is at least 65 nucleotides in length. For example, the 3′-UTR is at least 70 nucleotides in length. For example, the 3′-UTR is at least 75 nucleotides in length. For example, the 3′-UTR is at least 80 nucleotides in length. For example, the 3′-UTR is at least 85 nucleotides in length. For example, the 3′-UTR is at least 90 nucleotides in length. For example, the 3′-UTR is at least 95 nucleotides in length. For example, the 3′-UTR is at least 100 nucleotides in length. For example, the 3′-UTR is at least 110 nucleotides in length. For example, the 3′-UTR is at least 120 nucleotides in length. For example, the 3′-UTR is at least 130 nucleotides in length. For example, the 3′-UTR is at least 140 nucleotides in length. For example, the 3′-UTR is at least 150 nucleotides in length. For example, the 3′-UTR is at least 160 nucleotides in length. For example, the 3′-UTR is at least 170 nucleotides in length. For example, the 3′-UTR is at least 180 nucleotides in length. For example, the 3′-UTR is at least 190 nucleotides in length. For example, the 3′-UTR is at least 200 nucleotides in length. For example, the 3′-UTR is at least 210 nucleotides in length. For example, the 3′-UTR is at least 220 nucleotides in length. For example, the 3′-UTR is at least 230 nucleotides in length. For example, the 3′-UTR is at least 240 nucleotides in length. For example, the 3′-UTR is at least 250 nucleotides in length. For example, the 3′-UTR is at least 260 nucleotides in length. For example, the 3′-UTR is at least 270 nucleotides in length. For example, the 3′-UTR is at least 280 nucleotides in length. For example, the 3′-UTR is at least 290 nucleotides in length. For example, the 3′-UTR is at least 300 nucleotides in length. For example, the 3′-UTR is at least 310 nucleotides in length. For example, the 3′-UTR is at least 320 nucleotides in length. For example, the 3′-UTR is at least 330 nucleotides in length. For example, the 3′-UTR is at least 340 nucleotides in length. For example, the 3′-UTR is at least 350 nucleotides in length. For example, the 3′-UTR is at least 360 nucleotides in length. For example, the 3′-UTR is at least 370 nucleotides in length. For example, the 3′-UTR is at least 380 nucleotides in length. For example, the 3′-UTR is at least 390 nucleotides in length.

In one example, the 3′-UTR is 40, or 45, or 50, or 55, or 60, or 65, or 70, or 75, or 80, or 85, or 90, or 95, or 100, or 110, or 120, or 130, or 140, or 150, or 160, or 170, or 180, or 190, or 200, or 210, or 220, or 230, or 240, or 250, or 260, or 270, or 280, or 290, or 300, or 310, or 320, or 330, or 340, or 350, or 360, or 370, or 380, or 390, or 400 nucleotides in length. For example, the 3′-UTR is 40 nucleotides in length. For example, the 3′-UTR is 45 nucleotides in length. For example, the 3′-UTR is 50 nucleotides in length. For example, the 3′-UTR is 55 nucleotides in length. For example, the 3′-UTR is 60 nucleotides in length. For example, the 3′-UTR is 65 nucleotides in length. For example, the 3′-UTR is 70 nucleotides in length. For example, the 3′-UTR is 75 nucleotides in length. For example, the 3′-UTR is 80 nucleotides in length. For example, the 3′-UTR is 85 nucleotides in length. For example, the 3′-UTR is 90 nucleotides in length. For example, the 3′-UTR is 95 nucleotides in length. For example, the 3′-UTR is 100 nucleotides in length. For example, the 3′-UTR is 110 nucleotides in length. For example, the 3′-UTR is 120 nucleotides in length. For example, the 3′-UTR is 130 nucleotides in length. For example, the 3′-UTR is 140 nucleotides in length. For example, the 3′-UTR is 150 nucleotides in length. For example, the 3′-UTR is 160 nucleotides in length. For example, the 3′-UTR is 170 nucleotides in length. For example, the 3′-UTR is 180 nucleotides in length. For example, the 3′-UTR is 190 nucleotides in length. For example, the 3′-UTR is 200 nucleotides in length. For example, the 3′-UTR is 210 nucleotides in length. For example, the 3′-UTR is 220 nucleotides in length. For example, the 3′-UTR is 230 nucleotides in length. For example, the 3′-UTR is 240 nucleotides in length. For example, the 3′-UTR is 250 nucleotides in length. For example, the 3′-UTR is 260 nucleotides in length. For example, the 3′-UTR is 270 nucleotides in length. For example, the 3′-UTR is 280 nucleotides in length. For example, the 3′-UTR is 290 nucleotides in length. For example, the 3′-UTR is 300 nucleotides in length. For example, the 3′-UTR is 310 nucleotides in length. For example, the 3′-UTR is 320 nucleotides in length. For example, the 3′-UTR is 330 nucleotides in length. For example, the 3′-UTR is 340 nucleotides in length. For example, the 3′-UTR is 350 nucleotides in length. For example, the 3′-UTR is 360 nucleotides in length. For example, the 3′-UTR is 370 nucleotides in length. For example, the 3′-UTR is 380 nucleotides in length. For example, the 3′-UTR is 390 nucleotides in length. For example, the 3′-UTR is 400 nucleotides in length.

In one example, the 3′-UTR comprises a nucleotide sequence at least 90% identical to a nucleotide sequence set forth in any one of SEQ ID NO: 40 to 56, 63, 77 and 79. For example, the 3′-UTR, the fragment and/or the variant thereof comprises a nucleotide sequence 90%, or 91%, or 92%, or 93%, or 94%, or 95%, or 96%, or 97%, or 98%, or 99% identical to a nucleotide sequence set forth in any one of SEQ ID NO: 40 to 56, 63, 77 and 79. For example, the 3′-UTR comprises a nucleotide sequence at least 90% identical to a nucleotide sequence set forth in SEQ ID NO: 40. For example, the 3′-UTR comprises a nucleotide sequence at least 90% identical to a nucleotide sequence set forth in SEQ ID NO: 41. For example, the 3′-UTR comprises a nucleotide sequence at least 90% identical to a nucleotide sequence set forth in SEQ ID NO: 42. For example, the 3′-UTR comprises a nucleotide sequence at least 90% identical to a nucleotide sequence set forth in SEQ ID NO: 43. For example, the 3′-UTR comprises a nucleotide sequence at least 90% identical to a nucleotide sequence set forth in SEQ ID NO: 44. For example, the 3′-UTR comprises a nucleotide sequence at least 90% identical to a nucleotide sequence set forth in SEQ ID NO: 45. For example, the 3′-UTR comprises a nucleotide sequence at least 90% identical to a nucleotide sequence set forth in SEQ ID NO: 46. For example, the 3′-UTR comprises a nucleotide sequence at least 90% identical to a nucleotide sequence set forth in SEQ ID NO: 47. For example, the 3′-UTR comprises a nucleotide sequence at least 90% identical to a nucleotide sequence set forth in SEQ ID NO: 48. For example, the 3′-UTR comprises a nucleotide sequence at least 90% identical to a nucleotide sequence set forth in SEQ ID NO: 49. For example, the 3′-UTR comprises a nucleotide sequence at least 90% identical to a nucleotide sequence set forth in SEQ ID NO: 50. For example, the 3′-UTR comprises a nucleotide sequence at least 90% identical to a nucleotide sequence set forth in SEQ ID NO: 51. For example, the 3′-UTR comprises a nucleotide sequence at least 90% identical to a nucleotide sequence set forth in SEQ ID NO: 52. For example, the 3′-UTR comprises a nucleotide sequence at least 90% identical to a nucleotide sequence set forth in SEQ ID NO: 53. For example, the 3′-UTR comprises a nucleotide sequence at least 90% identical to a nucleotide sequence set forth in SEQ ID NO: 54. For example, the 3′-UTR comprises a nucleotide sequence at least 90% identical to a nucleotide sequence set forth in SEQ ID NO: 55. For example, the 3′-UTR comprises a nucleotide sequence at least 90% identical to a nucleotide sequence set forth in SEQ ID NO: 56. For example, the 3′-UTR comprises a nucleotide sequence at least 90% identical to a nucleotide sequence set forth in SEQ ID NO: 63. For example, the 3′-UTR comprises a nucleotide sequence at least 90% identical to a nucleotide sequence set forth in SEQ ID NO: 77. For example, the 3′-UTR comprises a nucleotide sequence at least 90% identical to a nucleotide sequence set forth in SEQ ID NO: 79.

In one example, the 3′-UTR comprises a nucleotide sequence at least 95% identical to a nucleotide sequence set forth in any one of SEQ ID NO: 40 to 56, 63, 77 and 79. For example, the 3′-UTR comprises a nucleotide sequence at least 95% identical to a nucleotide sequence set forth in SEQ ID NO: 40. For example, the 3′-UTR comprises a nucleotide sequence at least 95% identical to a nucleotide sequence set forth in SEQ ID NO: 41. For example, the 3′-UTR comprises a nucleotide sequence at least 95% identical to a nucleotide sequence set forth in SEQ ID NO: 42. For example, the 3′-UTR comprises a nucleotide sequence at least 95% identical to a nucleotide sequence set forth in SEQ ID NO: 43. For example, the 3′-UTR comprises a nucleotide sequence at least 95% identical to a nucleotide sequence set forth in SEQ ID NO: 44. For example, the 3′-UTR comprises a nucleotide sequence at least 95% identical to a nucleotide sequence set forth in SEQ ID NO: 45. For example, the 3′-UTR comprises a nucleotide sequence at least 95% identical to a nucleotide sequence set forth in SEQ ID NO: 46. For example, the 3′-UTR comprises a nucleotide sequence at least 95% identical to a nucleotide sequence set forth in SEQ ID NO: 47. For example, the 3′-UTR comprises a nucleotide sequence at least 95% identical to a nucleotide sequence set forth in SEQ ID NO: 48. For example, the 3′-UTR comprises a nucleotide sequence at least 95% identical to a nucleotide sequence set forth in SEQ ID NO: 49. For example, the 3′-UTR comprises a nucleotide sequence at least 95% identical to a nucleotide sequence set forth in SEQ ID NO: 50. For example, the 3′-UTR comprises a nucleotide sequence at least 95% identical to a nucleotide sequence set forth in SEQ ID NO: 51. For example, the 3′-UTR comprises a nucleotide sequence at least 95% identical to a nucleotide sequence set forth in SEQ ID NO: 52. For example, the 3′-UTR comprises a nucleotide sequence at least 95% identical to a nucleotide sequence set forth in SEQ ID NO: 53. For example, the 3′-UTR comprises a nucleotide sequence at least 95% identical to a nucleotide sequence set forth in SEQ ID NO: 54. For example, the 3′-UTR comprises a nucleotide sequence at least 95% identical to a nucleotide sequence set forth in SEQ ID NO: 55. For example, the 3′-UTR comprises a nucleotide sequence at least 95% identical to a nucleotide sequence set forth in SEQ ID NO: 56. For example, the 3′-UTR comprises a nucleotide sequence at least 95% identical to a nucleotide sequence set forth in SEQ ID NO: 63. For example, the 3′-UTR comprises a nucleotide sequence at least 95% identical to a nucleotide sequence set forth in SEQ ID NO: 77. For example, the 3′-UTR comprises a nucleotide sequence at least 95% identical to a nucleotide sequence set forth in SEQ ID NO: 79.

In one example, the 3′-UTR comprises a nucleotide sequence at least 99% identical to a nucleotide sequence set forth in any one of SEQ ID NO: 40 to 56, 63, 77 and 79. For example, the 3′-UTR comprises a nucleotide sequence at least 99% identical to a nucleotide sequence set forth in SEQ ID NO: 40. For example, the 3′-UTR comprises a nucleotide sequence at least 99% identical to a nucleotide sequence set forth in SEQ ID NO: 41. For example, the 3′-UTR comprises a nucleotide sequence at least 99% identical to a nucleotide sequence set forth in SEQ ID NO: 42. For example, the 3′-UTR comprises a nucleotide sequence at least 99% identical to a nucleotide sequence set forth in SEQ ID NO: 43. For example, the 3′-UTR comprises a nucleotide sequence at least 99% identical to a nucleotide sequence set forth in SEQ ID NO: 44. For example, the 3′-UTR comprises a nucleotide sequence at least 99% identical to a nucleotide sequence set forth in SEQ ID NO: 45. For example, the 3′-UTR comprises a nucleotide sequence at least 99% identical to a nucleotide sequence set forth in SEQ ID NO: 46. For example, the 3′-UTR comprises a nucleotide sequence at least 99% identical to a nucleotide sequence set forth in SEQ ID NO: 47. For example, the 3′-UTR comprises a nucleotide sequence at least 99% identical to a nucleotide sequence set forth in SEQ ID NO: 48. For example, the 3′-UTR comprises a nucleotide sequence at least 99% identical to a nucleotide sequence set forth in SEQ ID NO: 49. For example, the 3′-UTR comprises a nucleotide sequence at least 99% identical to a nucleotide sequence set forth in SEQ ID NO: 50. For example, the 3′-UTR comprises a nucleotide sequence at least 99% identical to a nucleotide sequence set forth in SEQ ID NO: 51. For example, the 3′-UTR comprises a nucleotide sequence at least 99% identical to a nucleotide sequence set forth in SEQ ID NO: 52. For example, the 3′-UTR comprises a nucleotide sequence at least 99% identical to a nucleotide sequence set forth in SEQ ID NO: 53. For example, the 3′-UTR comprises a nucleotide sequence at least 99% identical to a nucleotide sequence set forth in SEQ ID NO: 54. For example, the 3′-UTR comprises a nucleotide sequence at least 99% identical to a nucleotide sequence set forth in SEQ ID NO: 55. For example, the 3′-UTR comprises a nucleotide sequence at least 99% identical to a nucleotide sequence set forth in SEQ ID NO: 56. For example, the 3′-UTR comprises a nucleotide sequence at least 99% identical to a nucleotide sequence set forth in SEQ ID NO: 63. For example, the 3′-UTR comprises a nucleotide sequence at least 99% identical to a nucleotide sequence set forth in SEQ ID NO: 77. For example, the 3′-UTR comprises a nucleotide sequence at least 99% identical to a nucleotide sequence set forth in SEQ ID NO: 79.

In one example, the 3′-UTR comprises a nucleotide sequence set forth in any one of SEQ ID NO: 40 to 56, 63, 77 and 79. For example, the 3′-UTR comprises a nucleotide sequence set forth SEQ ID NO: 40. For example, the 3′-UTR comprises a nucleotide sequence set forth SEQ ID NO: 41. For example, the 3′-UTR comprises a nucleotide sequence set forth SEQ ID NO: 42. For example, the 3′-UTR comprises a nucleotide sequence set forth SEQ ID NO: 43. For example, the 3′-UTR comprises a nucleotide sequence set forth SEQ ID NO: 44. For example, the 3′-UTR comprises a nucleotide sequence set forth SEQ ID NO: 45. For example, the 3′-UTR comprises a nucleotide sequence set forth SEQ ID NO: 46. For example, the 3′-UTR comprises a nucleotide sequence set forth SEQ ID NO: 47. For example, the 3′-UTR comprises a nucleotide sequence set forth SEQ ID NO: 48. For example, the 3′-UTR comprises a nucleotide sequence set forth SEQ ID NO:49. For example, the 3′-UTR comprises a nucleotide sequence set forth SEQ ID NO: 50. For example, the 3′-UTR comprises a nucleotide sequence set forth SEQ ID NO: 51. For example, the 3′-UTR comprises a nucleotide sequence set forth SEQ ID NO: 52. For example, the 3′-UTR comprises a nucleotide sequence set forth SEQ ID NO: 53. For example, the 3′-UTR comprises a nucleotide sequence set forth SEQ ID NO: 54. For example, the 3′-UTR comprises a nucleotide sequence set forth SEQ ID NO: 55. For example, the 3′-UTR comprises a nucleotide sequence set forth SEQ ID NO: 56. For example, the 3′-UTR comprises a nucleotide sequence set forth SEQ ID NO: 63. For example, the 3′-UTR comprises a nucleotide sequence set forth SEQ ID NO: 77. For example, the 3′-UTR comprises a nucleotide sequence set forth SEQ ID NO: 79.

In one example, the second nucleotide sequence comprises the 3′-CSE of a Venezuelan equine encephalitis virus (VEEV) or a Sindbis virus (SIN).

In one example, the second nucleotide sequence comprises a combination of two or more 3′-UTRs. In one example, two or more 3′-UTRs are the same. In one example, two or more 3′-UTRs are different.

In one example, the second nucleotide sequence comprises at least one microRNA binding site, an AU rich element (ARE), a GC-rich element, a triple helix, a stem loop, one or more stop codons, a 3′CSE of an alphavirus and combinations thereof. In one example, the second nucleotide sequence comprises at least one microRNA binding site, an AU rich element (ARE), a GC-rich element, a triple helix, a stem loop, one or more stop codons and combinations thereof. For example, the second nucleotide sequence comprises a microRNA binding site. For example, the second nucleotide sequence comprises an AU rich element (ARE). For example, the second nucleotide sequence comprises a GC-rich element. For example, the second nucleotide sequence comprises a triple helix. For example, the second nucleotide sequence comprises a stem loop. For example, the second nucleotide sequence comprises one or more stop codons. For example, the second nucleotide sequence comprises one or more stop codons located at the 5′end of the 3′-UTR. For example, the second nucleotide sequence comprises a 3′CSE from an alphavirus. For example, the second nucleotide sequence comprises the 3′-CSE of a Venezuelan equine encephalitis virus (VEEV) or a Sindbis virus (SIN). For example, the second nucleotide sequence comprises the 3′-CSE of a Venezuelan equine encephalitis virus (VEEV). For example, the second nucleotide sequence comprises the 3′-CSE of a Sindbis virus (SIN).

In one example, the one or more RNAs comprise a third nucleotide sequence comprising one or more 3′ tailing sequences located at the 3′end of the second nucleotide sequence.

In one example, the one or more 3′ tailing sequences are selected from the group consisting of a poly-A sequence, polyadenylation signal, a G-quadruplex, a poly-C sequence, a stem loop and combinations thereof. For example, the third nucleotide sequence comprises a poly-A sequence. For example, the third nucleotide sequence comprises a polyadenylation signal. For example, the third nucleotide sequence comprises a G-quadruplex. For example, the third nucleotide sequence comprises a poly-C sequence. For example, the third nucleotide sequence comprises a stem loop. For example, the third nucleotide sequence comprises a poly-A sequence and a G-quadruplex.

In one example, the one or more 3′ tailing sequences comprises one or more poly-A sequences each comprising between 10 and 300 consecutive adenosine nucleotides. For example, the one or more poly-A sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive adenosine nucleotides. For example, the one or more poly-A sequences each comprises between 10 and 20 consecutive adenosine nucleotides. For example, the one or more poly-A sequences each comprises between 20 and 30 consecutive adenosine nucleotides. For example, the one or more poly-A sequences each comprises between 30 and 40 consecutive adenosine nucleotides. For example, the one or more poly-A sequences each comprises between 40 and 50 consecutive adenosine nucleotides. For example, the one or more poly-A sequences each comprises between 50 and 60 consecutive adenosine nucleotides. For example, the one or more poly-A sequences each comprises between 60 and 70 consecutive adenosine nucleotides. For example, the one or more poly-A sequences each comprises between 70 and 80 consecutive adenosine nucleotides. For example, the one or more poly-A sequences each comprises between 80 and 90 consecutive adenosine nucleotides. For example, the one or more poly-A sequences each comprises between 90 and 100 consecutive adenosine nucleotides. For example, the one or more poly-A sequences each comprises between 100 and 125 consecutive adenosine nucleotides. For example, the one or more poly-A sequences each comprises between 125 and 150 consecutive adenosine nucleotides. For example, the one or more poly-A sequences each comprises between 150 and 175 consecutive adenosine nucleotides. For example, the one or more poly-A sequences each comprises between 175 and 200 consecutive adenosine nucleotides. For example, the one or more poly-A sequences each comprises between 200 and 225 consecutive adenosine nucleotides. For example, the one or more poly-A sequences each comprises between 225 and 250 consecutive adenosine nucleotides. For example, the one or more poly-A sequences each comprises between 250 and 275 consecutive adenosine nucleotides. For example, the one or more poly-A sequences each comprises between 275 and 300 consecutive adenosine nucleotides.

In one example, the one or more poly-A sequence each comprises 10, or 20, or 30, or 40, or 50, or 60, or 70, or 80, or 90, or 100, or 125, or 150, or 175, or 200, or 225, or 250, or 275, or 300 consecutive adenosine nucleotides. For example, the one or more poly-A sequence each comprises 10 consecutive adenosine nucleotides. For example, the one or more poly-A sequence each comprises 20 consecutive adenosine nucleotides. For example, the one or more poly-A sequence each comprises 30 consecutive adenosine nucleotides. For example, the one or more poly-A sequence each comprises 40 consecutive adenosine nucleotides. For example, the one or more poly-A sequence each comprises 50 consecutive adenosine nucleotides. For example, the one or more poly-A sequence each comprises 60 consecutive adenosine nucleotides. For example, the one or more poly-A sequence each comprises 70 consecutive adenosine nucleotides. For example, the one or more poly-A sequence each comprises 80 consecutive adenosine nucleotides. For example, the one or more poly-A sequence each comprises 90 consecutive adenosine nucleotides. For example, the one or more poly-A sequence each comprises 100 consecutive adenosine nucleotides. For example, the one or more poly-A sequence each comprises 125 consecutive adenosine nucleotides. For example, the one or more poly-A sequence each comprises 150 consecutive adenosine nucleotides. For example, the one or more poly-A sequence each comprises 175 consecutive adenosine nucleotides. For example, the poly-A sequence each comprises 200 consecutive adenosine nucleotides. For example, the one or more poly-A sequence each comprises 225 consecutive adenosine nucleotides. For example, the one or more poly-A sequence each comprises 250 consecutive adenosine nucleotides. For example, the one or more poly-A sequence each comprises 275 consecutive adenosine nucleotides. For example, the one or more poly-A sequence each comprises 300 consecutive adenosine nucleotides.

In one example, the one or more poly-A sequences is separated by an interrupting linker. For example, the third nucleotide sequence comprising the one or more 3′tailing sequences comprises, in order of 5′ to 3′: consecutive adenosine nucleotides, an interrupting linker, and further consecutive adenosine nucleotides.

In one example, the interrupting linker is from 10 to 50, or 50 to 100, or 100 to 150 nucleotides in length. For example, the interrupting linker is from 10 to 50 nucleotides in length. For example, the interrupting linker is from 50 to 100 nucleotides in length. For example, the interrupting linker is from 100 to 150 nucleotides in length.

In one example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length. For example, the interrupting linker is 1 nucleotide in length. For example, the interrupting linker is 2 nucleotides in length. For example, the interrupting linker is 3 nucleotides in length. For example, the interrupting linker is 4 nucleotides in length. For example, the interrupting linker is 5 nucleotides in length. For example, the interrupting linker is 6 nucleotides in length. For example, the interrupting linker is 7 nucleotides in length. For example, the interrupting linker is 8 nucleotides in length. For example, the interrupting linker is 9 nucleotides in length. For example, the interrupting linker is 10 nucleotides in length. For example, the interrupting linker is 11 nucleotides in length. For example, the interrupting linker is 12 nucleotides in length. For example, the interrupting linker is 13 nucleotides in length. For example, the interrupting linker is 14 nucleotides in length. For example, the interrupting linker is 15 nucleotides in length. For example, the interrupting linker is 16 nucleotides in length. For example, the interrupting linker is 17 nucleotides in length. For example, the interrupting linker is 18 nucleotides in length. For example, the interrupting linker is 19 nucleotides in length. For example, the interrupting linker is 20 nucleotides in length. For example, the interrupting linker is 25 nucleotides in length. For example, the interrupting linker is 30 nucleotides in length. For example, the interrupting linker is 35 nucleotides in length. For example, the interrupting linker is 40 nucleotides in length. For example, the interrupting linker is 45 nucleotides in length. For example, the interrupting linker is 50 nucleotides in length. For example, the interrupting linker is 55 nucleotides in length. For example, the interrupting linker is 60 nucleotides in length. For example, the interrupting linker is 65 nucleotides in length. For example, the interrupting linker is 70 nucleotides in length. For example, the interrupting linker is 75 nucleotides in length. For example, the interrupting linker is 80 nucleotides in length. For example, the interrupting linker is 85 nucleotides in length. For example, the interrupting linker is 90 nucleotides in length. For example, the interrupting linker is 95 nucleotides in length. For example, the interrupting linker is 100 nucleotides in length. For example, the interrupting linker is 110 nucleotides in length. For example, the interrupting linker is 120 nucleotides in length. For example, the interrupting linker is 130 nucleotides in length. For example, the interrupting linker is 140 nucleotides in length. For example, the interrupting linker is 150 nucleotides in length.

In one example, the interrupting linker comprises or consists of the nucleotide sequence set forth in SEQ ID NO: 59. For example, the interrupting linker comprises the nucleotide sequence set forth in SEQ ID NO: 59. For example, the interrupting linker consists of the nucleotide sequence set forth in SEQ ID NO: 59.

In one example, the one or more RNAs comprising a third nucleotide sequence comprises, in the order from 5′ to 3′: consecutive adenosine nucleotides, an interrupting linker, and a further consecutive adenosine nucleotides.

In one example, the one or more RNAs comprising a third nucleotide sequence comprises, in the order from 5′ to 3′: consecutive adenosine nucleotides, an interrupting linker comprising or consisting of a nucleotide sequence set forth in SEQ ID NO: 59, and a further consecutive adenosine nucleotides.

In one example, the one or more RNAs comprising a third nucleotide sequence comprises, in the order from 5′ to 3′: 30 consecutive adenosine nucleotides, an interrupting linker comprising or consisting of a nucleotide sequence set for in SEQ ID NO: 59, and 70 consecutive adenosine nucleotides.

In one example, the one or more RNAs comprises at least one chemically modified nucleotide.

In one example, the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), 1-methylpseudouridine (m1ψ), and combinations thereof. For example, the chemically modified nucleotide is N6,2′-O-dimethyl-adenosine (m6Am). For example, the chemically modified nucleotide is 5-methyluridine (m5U). For example, the chemically modified nucleotide is N4-acetylcytidine (ac4C). For example, the chemically modified nucleotide is 2-thiocytidine (s2C). For example, the chemically modified nucleotide is 2-thiouridine (s2U). For example, the chemically modified nucleotide is 5-methylcytidine (m5C). For example, the chemically modified nucleotide is N6-methyladenosine (m6a). For example, the chemically modified nucleotide is pseudouridine (ψ). For example, the chemically modified nucleotide is 1-methylpseudouridine (m1ψ).

In one example, the one or more RNAs is conventional mRNA (cRNA) or self-amplifying mRNA (sa-mRNA). For example, the mRNA is cRNA. For example, the mRNA is sa-mRNA.

In one example, the sa-mRNA comprises one or more nucleotide sequence from an alphavirus selected from the group consisting of Semliki Forest virus (SFV), Sindbis virus (SIN), and Venezuelan equine encephalitis virus (VEEV) and combinations thereof. For example, the sa-mRNA comprises one or more nucleotide sequence from an alphavirus of Semliki Forest virus (SFV). For example, the sa-mRNA comprises one or more nucleotide sequence from an alphavirus of Sindbis virus (SIN). For example, the sa-mRNA comprises one or more nucleotide sequence from an alphavirus of Venezuelan equine encephalitis virus (VEEV).

In one example, the sa-mRNA comprises one or more nucleotide sequence encoding non-structural proteins (NSPs) from an alphavirus.

In one example, the sa-mRNA comprises one or more nucleotide sequence comprising or consisting a SG promoter of the alphavirus.

In one example, the sa-mRNA comprises one or more nucleotide sequence from an alphavirus sequence encoding non-structural proteins (NSPs) and one or more nucleotide sequence comprising or consisting a SG promoter of the alphavirus.

In one example, the RNA further comprises a 5′ terminal cap structure.

In one example, the 5′ terminal cap structure is an endogenous cap or analogue thereof. For example, the 5′terminal cap structure is an endogenous cap. For example, the 5′terminal cap structure is an analogue of an endogenous cap.

In one example, the 5′ terminal cap structure comprise a guanine or guanine analogue thereof. For example, the 5′ terminal cap structure comprise a guanine. For example, the 5′ terminal cap structure comprise a guanine analogue of a guanine.

In one example, the 5′ terminal cap structure is selected from a group consisting of anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2. For example, the 5′ terminal cap structure is anti-reverse cap analogue (ARCA). For example, the 5′ terminal cap structure is N7,2′-0-dimethyl-guanosine (mCAP). For example, the 5′ terminal cap structure is inosine. For example, the 5′ terminal cap structure is N1-methyl-guanosine. For example, the 5′ terminal cap structure is 2′fluoro-guanosine. For example, the 5′ terminal cap structure is 7-deaza-guanosine. For example, the 5′ terminal cap structure is 8-oxo-guanosine. For example, the 5′ terminal cap structure is 2-amino-guanosine. For example, the 5′ terminal cap structure is LNA-guanosine. For example, the 5′ terminal cap structure is 2-azido-guanosine. For example, the 5′ terminal cap structure is N6,2′-O-dimethyladenosine. For example, the 5′ terminal cap structure is 7-methylguanosine (m7G). For example, the 5′ terminal cap structure is Cap1. For example, the 5′ terminal cap structure is Cap2.

In one example, the 5′terminal cap structure is linked to the 5′ end of the RNA by a 5′-5′-triphosphate linkage or a 5′-5′ phosphorothioate linkage. For example, the 5′terminal cap structure is linked to the 5′ end of the RNA by a 5′-5′-triphosphate linkage. For example, the 5′terminal cap structure is linked to the 5′ end of the RNA by a 5′-5′ phosphorothioate linkage.

In one example, the one or more RNAs are formulated in a lipid nanoparticle (LNP). For example, the RNA is encapsulated in a LNP. In another example, the RNA is bound to the LNP. For example, the RNA is absorbed on the LNP.

In one example, the LNP further comprises a PEG-lipid, a structural lipid and/or a neutral lipid. For example, the LNP further comprises a PEG-lipid. For example, the LNP further comprises a structural lipid. For example, the LNP further comprises a neutral lipid.

In one example, the LNP comprises an ionisable lipid. For example, the ionisable lipid is a cationic lipid. For example, the ionisable lipid is a zwitterionic lipid.

In one example, the LNP does not comprise an ionisable lipid.

In one example, each RNA is formulated together in the LNP. For example, a composition comprising a first, second and/or third RNA are formulated together in the LNP. For example, a composition comprising a first and second RNA, wherein the first and second RNAs are formulated together in the LNP. For example, a composition comprising a first, second and third RNA, wherein the first, second and third RNAs are formulated together in the LNP.

In one example, each RNA is formulated separately in the LNP. For example, a composition comprising a first, second and/or third RNAs are formulated separately in the LNP. For example, the composition comprising the first and second RNA, wherein the first and second RNA are formulated in separate LNPs. For example, a composition comprising a first, second and third RNA, wherein the first, second and third RNA are each formulated in separate LNPs. For example, a composition comprising a first, second and third RNA, wherein the first and second RNA are formulated together in a LNP and the third RNA is formulated in a separate LNP. For example, a composition comprising a first, second and third RNA, wherein the first and third RNA are formulated together in a LNP and the second RNA is formulated in a separate LNP. For example, a composition comprising a first, second and third RNA, wherein the third and second RNA are formulated together in a LNP and the first RNA is formulated in a separate LNP.

In one example, the composition is an immunogenic composition.

In one example, the composition is a pharmaceutical composition.

The present disclosure further provides a pharmaceutical composition comprising an immunogenic composition of the present disclosure and a pharmaceutically acceptable carrier.

In one example, the present disclosure provides the immunogenic composition or the pharmaceutical composition of the present disclosure for use as a vaccine.

In one example, the immunogenic composition or the pharmaceutical composition of the disclosure is for use in the treatment or prevention or delaying progression of influenza and/or an influenza virus infection.

In one example, the present disclosure provides the immunogenic composition or the pharmaceutical composition of the disclosure for use in the treatment or prevention or delaying progression of influenza and/or an influenza virus infection. For example, the present disclosure provides the immunogenic composition or the pharmaceutical composition of the disclosure for use in the treatment of influenza and/or an influenza virus infection. For example, the present disclosure provides the immunogenic composition or the pharmaceutical composition of the disclosure for use in the prevention of influenza and/or an influenza virus infection. For example, the present disclosure provides the immunogenic composition or the pharmaceutical composition of the disclosure for use in delaying the progression of influenza and/or an influenza virus infection.

In one example, the present disclosure provides the immunogenic composition or the pharmaceutical composition of the disclosure for use in the treatment or prevention or delaying progression of an influenza and/or an influenza virus infection. For example, the present disclosure provides the immunogenic composition or the pharmaceutical composition of the disclosure for use in the treatment of an influenza and/or an influenza virus infection. For example, the present disclosure provides the immunogenic composition or the pharmaceutical composition of the disclosure for use in the prevention of an influenza and/or an influenza virus infection. For example, the present disclosure provides the immunogenic composition or the pharmaceutical composition of the disclosure for use in delaying the progression of an influenza and/or an influenza virus infection.

The present disclosure also provides use of the composition, the immunogenic composition, or the pharmaceutical composition of the present disclosure in the manufacture of a medicament for treating or preventing or delaying progression of influenza and/or an influenza virus infection in a subject in need thereof. In one example, the present disclosure provides use of the composition, the immunogenic composition, or the pharmaceutical composition of the present disclosure in the manufacture of a medicament for treating influenza and/or an influenza virus infection in a subject in need thereof. In one example, the present disclosure provides use of the composition, the immunogenic composition, or the pharmaceutical composition of the present disclosure in the manufacture of a medicament for preventing influenza and/or an influenza virus infection in a subject in need thereof. In one example, the present disclosure provides use of the composition, the immunogenic composition, or the pharmaceutical composition of the present disclosure in the manufacture of a medicament for delaying progression of influenza and/or an influenza virus infection and/or influenza and/or an influenza virus infection virus infection in a subject in need thereof.

The present disclosure also provides a method of treating or preventing or delaying progression of influenza and/or an influenza virus infection in a subject, the method comprising administering the immunogenic composition or the pharmaceutical composition of the present disclosure to a subject in need thereof. For example, the present disclosure provides a method of treating influenza and/or an influenza virus infection in a subject, the method comprising administering the immunogenic composition or the pharmaceutical composition of the present disclosure to a subject in need thereof. For example, the present disclosure provides a method of preventing influenza and/or an influenza virus infection in a subject, the method comprising administering the immunogenic composition or the pharmaceutical composition of the present disclosure to a subject in need thereof. The present disclosure also provides a method of delaying progression of influenza and/or an influenza virus infection in a subject, the method comprising administering the immunogenic composition or the pharmaceutical composition of the present disclosure to a subject in need thereof.

In one example of any method described herein, the composition, the immunogenic composition, or the pharmaceutical composition of the present disclosure is administered before or after the development of influenza and/or an influenza virus infection in a subject. In one example of any method described herein, the composition, the immunogenic composition, or the pharmaceutical composition of the present disclosure is administered before the development of influenza and/or an influenza virus infection. In one example of any method described herein, the composition, the immunogenic composition, or the pharmaceutical composition of the present disclosure is administered after the development of influenza and/or an influenza virus infection in a subject.

The present disclosure provides a method of inducing an immune response in a subject to influenza and/or an influenza virus infection virus, comprising administering the composition, the immunogenic composition or the pharmaceutical composition of the present disclosure to a subject in need thereof.

The present disclosure also provides use of the composition, the immunogenic composition or the pharmaceutical composition of the present disclosure in the manufacture of a medicament for inducing an immune response in a subject in need thereof.

In one example, the composition, the immunogenic composition or the pharmaceutical composition of the present disclosure induces a humoral and/or a cell-mediated immune response. In one example, the composition induces a humoral immune response in the subject. For example, the humoral immune response is an antibody-mediated immune response. For example, production of neutralizing antibodies. In another example, the composition induces a cell-mediated immune response. For example, the cell-mediated immune response includes activation of antigen-specific cytotoxic T cells. For example, the T cells are CD4 T cells and/or CD8 T cells. In one example, the T cells are CD4 T cells. In another example the T cells are CD8 T cells. In a further example, the T cells are CD4 and CD8 T cells.

In one example, administration of the composition, the immunogenic composition or the pharmaceutical composition of the present disclosure induces a CD4 T cell mediated immune response.

In one example, administration of the composition, the immunogenic composition or the pharmaceutical composition of the present disclosure induces a CD8 T cell mediated immune response.

In one example, administration of the composition, the immunogenic composition or the pharmaceutical composition of the present disclosure induces a CD4 and a CD8 T cell mediated immune response.

The present disclosure also provides a method of expressing a virus-like particle (VLP) in a subject comprising administering the composition, the immunogenic composition, or the pharmaceutical composition of the present disclosure to the subject.

The present disclosure further provides use of the composition, or the immunogenic composition, or the pharmaceutical composition of the present disclosure in the manufacture of a medicament for expressing a VLP in a subject in need thereof.

The present disclosure also provides the immunogenic composition, or the pharmaceutical composition of the present disclosure for use in a method of expressing a VLP in a subject in need thereof.

The present disclosure also provides a kit comprising at least one composition of the disclosure.

In one example, the kit comprises a composition of the present disclosure, optionally in a delivery system and/or a pharmaceutically acceptable carrier or diluent, packaged with instructions for use in treating or preventing or delaying influenza and/or an influenza virus infection in a subject in need thereof. For example, the RNA is a mRNA. For example, the mRNA is a cRNA or sa-mRNA. In one example, the kit comprises at least one RNA of the present disclosure, optionally in a delivery system and/or a pharmaceutically acceptable carrier or diluent, packaged with instructions to administer the mRNA to a subject who is suffering from or at risk of suffering from influenza and/or an influenza virus infection.

In one example, the composition, the immunogenic composition or the pharmaceutical composition of the disclosure is supplied in a vial. In another example, the immunogenic composition or the pharmaceutical composition of the disclosure is supplied in a syringe.

Any discussion of a translation initiation sequence (e.g. a Kozak consensus sequence, an IRES, or a SG promoter), a 5′-UTR, a VLP, a M1, a HA, a NA and/or a 3′-UTR herein will be understood to include a fragment and/or a variant of the translation initiation sequence (e.g. the Kozak consensus sequence, the IRES, or the SG promoter), the 5′-UTR, the VLP, the M1, the HA, the NA, and/or the 3′-UTR.

### BRIEF DESCRIPTION OF THE DRAWINGS

FIG. 1 is a Western Blot image of (A) BHK cell culture lysate and (B) BHK cell culture supernatant used to produce VLPs from four different VLP mRNA constructs probed for HA, NA, M1 and GAPDH proteins.

FIG. 2 is a graphical representation showing the expression of HA, NA and M1 protein in (A) BHK cell culture lysate and (B) BHK cell culture supernatant, and the expression of M1 protein in (C) BHK cell culture lysate and (D) BHK cell culture supernatant used to produce VLPs from four different VLP mRNA constructs, as quantified from the Western Blot image of FIG. 1.

### KEY TO SEQUENCE LISTING

SEQ ID NO: 1 is a nucleotide sequence of a Kozak consensus sequence [accatgg]
SEQ ID NO: 2 is a nucleotide sequence of a Kozak consensus sequence [accatg]
SEQ ID NO: 3 is a nucleotide sequence of minimal subgenomic promoter (v1)
SEQ ID NO: 4 is a nucleotide sequence of extended subgenomic promoter (v4)
SEQ ID NO: 5 is a nucleotide sequence of a histone stem loop
SEQ ID NO: 6 is a nucleotide sequence of a 5′-UTR of arachidonate 5-lipoxygenase (ALOX5)
SEQ ID NO: 7 is a nucleotide sequence of a 5′-UTR of alpha 1 collagen (Col1A)
SEQ ID NO: 8 is a nucleotide sequence of a 5′-UTR of tyrosine hydroxylase (TH gene)
SEQ ID NO: 9 is a nucleotide sequence of a 5′-UTR of tumor protein P53 inducible protein 3 (TP5313)
SEQ ID NO: 10 is a nucleotide sequence of a 5′-UTR of haptoglobin (HP)
SEQ ID NO: 11 is a nucleotide sequence of a 5′-UTR of fibrinogen beta chain (FGB)
SEQ ID NO: 12 is a nucleotide sequence of a 5′-UTR of haptoglobin-related protein (HPR)
SEQ ID NO: 13 is a nucleotide sequence of a 5′-UTR of albumin (ALB)
SEQ ID NO: 14 is a nucleotide sequence of a 5′-UTR of complement component 3 (C3)
SEQ ID NO: 15 is a nucleotide sequence of a 5′-UTR of fibrinogen alpha chain (FGA)
SEQ ID NO: 16 is a nucleotide sequence of a 5′-UTR of alpha 1 collagen (Col1A)
SEQ ID NO: 17 is a nucleotide sequence of a 5′-UTR of alpha 6 collagen (Col6A)
SEQ ID NO: 18 is a nucleotide sequence of a 5′-UTR of alpha-1-antitrypsin (SERPINA1)
SEQ ID NO: 19 is a nucleotide sequence of a 5′-UTR of alpha-1-antichymotrypsin (SERPINA3)
SEQ ID NO: 20 is a nucleotide sequence of a synthetic 5′-UTR
SEQ ID NO: 21 is a nucleotide sequence of a synthetic 5′-UTR
SEQ ID NO: 22 is a nucleotide sequence of a synthetic 5′-UTR
SEQ ID NO: 23 is a nucleotide sequence of a synthetic 5′-UTR
SEQ ID NO: 24 is a nucleotide sequence of a synthetic 5′-UTR
SEQ ID NO: 25 is a nucleotide sequence of a synthetic 5′-UTR
SEQ ID NO: 26 is a nucleotide sequence of a synthetic 5′-UTR
SEQ ID NO: 27 is a nucleotide sequence of a synthetic 5′-UTR
SEQ ID NO: 28 is a nucleotide sequence of a synthetic 5′-UTR
SEQ ID NO: 29 is a nucleotide sequence of a synthetic 5′-UTR
SEQ ID NO: 30 is a nucleotide sequence of a synthetic 5′-UTR
SEQ ID NO: 31 is a nucleotide sequence of a synthetic 5′-UTR
SEQ ID NO: 32 is a nucleotide sequence of a synthetic 5′-UTR
SEQ ID NO: 33 is a nucleotide sequence of a synthetic 5′-UTR
SEQ ID NO: 34 is a nucleotide sequence of a synthetic 5′-UTR
SEQ ID NO: 35 is a nucleotide sequence of a synthetic 5′-UTR
SEQ ID NO: 36 is a nucleotide sequence of a synthetic 5′-UTR
SEQ ID NO: 37 is a nucleotide sequence of a synthetic 5′-UTR
SEQ ID NO: 38 is a nucleotide sequence of a synthetic 5′-UTR
SEQ ID NO: 39 is a nucleotide sequence of a synthetic 5′-UTR
SEQ ID NO: 40 is a nucleotide sequence of a 3′-UTR of human creatine kinase
SEQ ID NO: 41 is a nucleotide sequence of a 3′-UTR of human myoglobin
SEQ ID NO: 42 is a nucleotide sequence of a 3′-UTR of human α-actin
SEQ ID NO: 43 is a nucleotide sequence of a 3′-UTR of human albumin
SEQ ID NO: 44 is a nucleotide sequence of a 3′-UTR of human a-globin
SEQ ID NO: 45 is a nucleotide sequence of a 3′-UTR of human G-CSF
SEQ ID NO: 46 is a nucleotide sequence of a 3′-UTR of human colla2 (collagen typeI alpha2)
SEQ ID NO: 47 is a nucleotide sequence of a 3′-UTR of human col6a2 (collagen typeVI alpha2)
SEQ ID NO: 48 is a nucleotide sequence of a 3′-UTR of human RPNI (ribophorinI)
SEQ ID NO: 49 is a nucleotide sequence of a 3′-UTR of human LRP1 (low densitylipoproteinreceptor-related protein 1)
SEQ ID NO: 50 is a nucleotide sequence of a 3′-UTR of human Nntl (cardiotrophin-like cytokine factor 1)
SEQ ID NO: 51 is a nucleotide sequence of a 3′-UTR of human col6al(collagen type VI alpha 1)
SEQ ID NO: 52 is a nucleotide sequence of a 3′-UTR of human Calr (calreticulin)
SEQ ID NO: 53 is a nucleotide sequence of a 3′-UTR of human Colla1 (collagen type I alpha 1)
SEQ ID NO: 54 is a nucleotide sequence of a 3′-UTR of human Plodl (procollagen-lysine 2-oxoglutarate5-dioxygenase 1)
SEQ ID NO: 55 is a nucleotide sequence of a 3′-UTR of human Nucbl (nucleobindin1)
SEQ ID NO: 56 is a nucleotide sequence of a 3′-UTR of human a-globin
SEQ ID NO: 57 is a nucleotide sequence of IRES from human fibroblast growth factor 1a (FGF1A)
SEQ ID NO: 58 is a nucleotide sequence of a fragment of IRES from GTX
SEQ ID NO: 59 is a nucleotide sequence of an interrupting linker
SEQ ID NO: 60 is a nucleotide sequence of a modified 5′-UTR of human a-globin
SEQ ID NO: 61 is a nucleotide sequence of a chimeric 3′-UTR of AES and mtRNR1
SEQ ID NO: 62 is a nucleotide sequence of a synthetic 5′-UTR
SEQ ID NO: 63 is a nucleotide sequence of a 3′-UTR of human globin
SEQ ID NO: 64 Nucleotide sequence of influenza A virus H5 hemagglutinin subtype (A/turkey/Turkey/1/2005)
SEQ ID NO: 65 Nucleotide sequence of influenza A virus N1 neuraminidase subtype (A/turkey/Turkey/1/2005)
SEQ ID NO: 66 Nucleotide sequence of influenza A virus M1 matrix protein (PR8-X)
SEQ ID NO: 67 Nucleotide sequence of influenza A virus M2 matrix protein
SEQ ID NO: 68 Nucleotide sequence of influenza A virus NS1 non-structural protein (A/California/09)
SEQ ID NO: 69 Nucleotide sequence of influenza virus nucleoprotein (A/California/09)
SEQ ID NO: 70 Nucleotide sequence of influenza A virus M1 matrix protein (A/California/09)
SEQ ID NO: 71 Nucleotide sequence of influenza A virus H3 protein (A/Delaware/39/2019)
SEQ ID NO: 72 Nucleotide sequence of influenza A virus N2 protein (A/Delaware/39/2019)
SEQ ID NO: 73 Nucleotide sequence of influenza B virus Hyam (B/Singapore/INFTT 16 0610/16 (By))
SEQ ID NO: 74 Nucleotide sequence of influenza B virus Nyam (B/Singapore/INFTT 16 0610/16 (By))
SEQ ID NO: 75 is a nucleotide sequence of extended subgenomic promoter (v2)
SEQ ID NO: 76 Nucleotide sequence of 5′UTR of VEEV
SEQ ID NO: 77 Nucleotide sequence of 3′UTR of VEEV
SEQ ID NO: 78 Nucleotide sequence of 3′CSE of VEEV
SEQ ID NO: 79 Nucleotide sequence of 3′UTR of SINV
SEQ ID NO: 80 Nucleotide sequence of 3′CSE of SINV
SEQ ID NO: 81 Nucleotide sequence of construct F554
SEQ ID NO: 82 Nucleotide sequence of construct F624
SEQ ID NO: 83 Nucleotide sequence of construct F625
SEQ ID NO: 84 Nucleotide sequence of construct F626
SEQ ID NO: 85 is a nucleotide sequence of a Kozak consensus sequence [accatgg]
SEQ ID NO: 86 is a nucleotide sequence of extended subgenomic promoter (v3)
SEQ ID NO: 87 is a nucleotide sequence of extended subgenomic promoter
SEQ ID NO: 88 is a nucleotide sequence of wild-type EMCV IRES
SEQ ID NO: 89 is a nucleotide sequence of GC-rich element
SEQ ID NO: 90 is a nucleotide sequence of GC-rich element
SEQ ID NO: 91 is a nucleotide sequence of GC-rich element
SEQ ID NO: 92 is a nucleotide sequence of T7 promoter

While the sequence listing refers to the DNA sequence, it is also understood that disclosure of the present application includes the RNA equivalent thereof as well the complements thereof, unless the context clearly dictates otherwise.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure can be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer can be obtained from a particular source albeit not necessarily directly from that source. Similarly, the term “based on” shall be taken to indicate that a specified integer can be developed or used from a particular source albeit not necessarily directly from that source.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### Selected Definitions

As used herein, the term or “ribonucleic acid” or “RNA” refers a molecular chain of nucleotides chemically bonded by a series of ester linkages between the phosphoryl group of one nucleotide and the hydroxyl group of the sugar in an adjacent nucleotide. In one example, the RNA is an mRNA. For example, the mRNA is a conventional mRNA (cRNA) or a self-amplifying RNA (sa-mRNA).

As used herein, the term “conventional mRNA” or “cRNA” or “non-amplifying RNA” refers to a construct that allows expression of heterologous RNA and proteins but the RNA that cannot amplify in host cells.

As used herein, the term “self-replicating RNA” refers to a construct based on an RNA virus that has been engineered to allow expression of heterologous mRNA and proteins. Self-replicating RNA (e.g., in the form of naked RNA) can amplify in host cells leading to expression of the desired gene product in the host cell.

As used herein, the term “multicistronic” (also known as “polycistronic”) in reference to the polynucleotide, RNA, cRNA and/or self-replicating RNA, refers to a RNA that encodes two or more polypeptides. The term encompasses “bicistronic” (or “dicistronic”; i.e., encoding two polypeptides) and “tricistronic” (i.e., encoding three polypeptides) molecules. By “bicistronic” is meant a single nucleic acid that is capable of encoding two distinct polypeptides from different regions of the nucleic acid.

The term “naked” as used herein refers to nucleic acids that are substantially free of other macromolecules, such as lipids, polymers and proteins. A “naked” nucleic acid, such as a self-amplifying RNA, is not formulated with other macromolecules to improve cellular uptake. Accordingly, a naked nucleic acid is not encapsulated in, absorbed on, or bound to a lipid nanoparticle (LNP), a liposome, a polymeric microparticle or an oil-in-water emulsion.

As used herein, the term “fragment” refers to a portion of a nucleotide sequence or polypeptide of a reference nucleotide sequence or polypeptide disclosed herein which maintains a defined activity of the full length nucleotide sequence or polypeptide. In one example, the defined activity is inducing an immune response in a subject administered with a composition of the present disclosure.

As used herein, the term “variant” refers to a nucleotide sequence (e.g. VLP forming element) or polypeptide (e.g. antigenic polypeptide) with difference(s) in one or more nucleotide sequence(s) or amino acid sequence(s) to a reference nucleotide sequence of polypeptide disclosed herein which maintains a defined activity of the nucleotide sequence or polypeptide. The difference(s) in one or more nucleotide sequence(s) or amino acid sequence(s) results from one or modification(s) made to the nucleotide sequence or polypeptide of the present disclosure. In one example, the modification is a chemical modification of one or more nucleotide(s) of the nucleotide sequence. For example, at least one naturally occurring nucleotide of the RNA is replaced with a chemically modified nucleotide (e.g. pseudouridine (ψ), and 1-methylpseudouridine (m1ψ)). In one example, the modification comprises increasing the G/C content of the nucleotide sequence. In one example, the modification comprises codon optimization of the nucleotide sequence. In one example, the defined activity is inducing an immune response in a subject administered with a composition of the present disclosure. For example, the variant is of a VLP forming element. For example, the variant is of a H5 protein and/or a N1 protein of influenza virus.

In one example, the variant has at least 70%, or 75%, or 80%, or 85%, or 90%, or 91%, or 92%, or 93%, or 94%, or 95%, or 96%, or 97%, or 98%, or 99% of sequence identity with the VLP forming element and/or the fragment thereof. In one example, the variant has at least 70%, or 75%, or 80%, or 85%, or 90%, or 91%, or 92%, or 93%, or 94%, or 95%, or 96%, or 97%, or 98%, or 99% of sequence identity with the VLP forming element and/or the fragment thereof. The nucleotide sequence or polypeptide variant disclosed herein can have one or more nucleotide(s) or amino acid(s) deleted or substituted by different nucleotide(s) or amino acid(s). In one example, the substitution is a conservative substitution. A skilled person will appreciate that a conservative substitution with reference to a polypeptide involves replacement of an amino acid in the polypeptide with a different amino acid with similar biochemical properties (e.g. charge, hydrophobicity and size). In one example, the substitution is a non-conservative substitution.

As used herein, the term “encode”, “encodes” or “encoding” refers to a region of a RNA capable of undergoing translation into a polypeptide.

As used herein, the term “antigen” refers to a molecule or structure containing one or more epitopes that induce, elicit, augment or boost a cellular and/or humoral immune response. Antigens can include, for example, proteins and peptides from a pathogen such as a virus, bacteria, fungus, protozoan, plant or from a tumour. For example, an antigen is derived from a gene of interest.

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified.

As used herein, the term “operably linked to” means positioning a translation initiation sequence (e.g. a Kozak consensus sequence, an internal ribosome entry site (IRES), a subgenomic (SG) promoter) or a stability element (e.g., an 5′-UTR) relative to a nucleic acid such that expression of the nucleic acid is controlled or regulated by the sequence or element. For example, a translation initiation sequence can be operably linked to the 5′ end of the one or more nucleotide sequence(s) encoding the VLP forming element.

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

The term “recombinant” shall be understood to mean the product of artificial genetic recombination.

As used herein, the term “lipid nanoparticle” or “LNP” shall be understood to refer to lipid-based particles having at least one dimension on the order of nanometers (e.g., 1-1,000 nm) and which comprises a compound of any formulae described herein. In embodiments, LNPs are formulated in a composition for delivery of a polynucleotide to a desired target such as a cell, tissue, organ, tumor, and the like. For example, the lipid nanoparticle or LNP any lipid composition, including, may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers. As would be understood by the person skilled in the art, LNP can be formed by mixing nucleic acid and lipids in an appropriate solvent system. For example, the LNPs may be formed using techniques known to the person skilled in the art, such as microfluidic mixing using e.g. a herringbone mixer, rapid mixing, T-junction mixing and the like,

As used herein, the term “stability” in the context of a VLP shall be understood to refer to the ability of the VLP to maintain its structure and/or function. The inventors determined using compositions of the present disclosure that when M1 proteins were produced and incorporated in the VLP at increased levels (compared to a VLP with lower levels of M1 protein incorporation), the ability of the VLP to remain intact also increased, and therefore these VLP are more stable.

As used herein, the term “release” in the context of a VLP shall be understood to refer to the release of the VLP from a sialic acid receptor to which the VLP is bound. HA proteins are know to bind to one or more sialic acid receptors (e.g. by those expressed from a cell). The inventors determined using compositions of the present disclosure that when NA proteins were produced and incorporated in the VLP at increased levels (compared to a VLP with lower levels of NA protein incorporation), the ability of the VLP to be released from the cell also increased.

As used herein, the terms “disease”, “disorder” or “condition” refers to a disruption of or interference with normal function.

As used herein, a subject “at risk” of having or developing an influenza virus infection and/or influenza can or can not have detectable disease or symptoms of influenza virus infection and/or influenza, and can or can not have displayed detectable disease or symptoms of influenza virus infection and/or influenza prior to the treatment according to the present disclosure. “At risk” denotes that a subject has one or more risk factors, which are measurable parameters that correlate with development of the influenza virus infection and/or influenza, as known in the art and/or described herein.

As used herein, the terms “treating”, “treat” or “treatment” include administering a RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual can be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

An “effective amount” refers to at least an amount effective, at dosages and for periods of time necessary, to achieve the desired result. For example, the desired result can be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change associated with a disease or condition as hereinbefore described. The effective amount can vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of RNA. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein can vary according to factors such as the disease state, age, sex, and weight of the patient, and the ability of the RNA of the present disclosure to elicit a desired response in the individual. A therapeutically effective amount is also one in which any toxic or detrimental effects of the RNA are outweighed by the therapeutically beneficial effects.

As used herein, the term “prophylactically effective amount” shall be taken to mean a sufficient quantity of the RNA of the disclosure to prevent or inhibit or delay the onset of one or more detectable symptoms of a disease or disorder.

As used herein, the term “subject” shall be taken to mean any animal including humans, for example a mammal. Exemplary subjects include but are not limited to humans and non-human primates. For example, the subject is a human.

### Composition

As used herein, the term “virus-like particle”, “VLP”, “virus-like particles” or “VLPs” shall be taken to mean a multi-subunit protein- and lipid-based structure, made up of virus-like particle (VLP) forming elements, which resembles the form and/or size of a virus particle but does not contain the genetic material of the virus. The VLP or VLPs display antigens which present conformational epitopes that elicit T cell and/or B cell immune responses but are unable to replicate and/or infect a host cell.

As used herein, the term “virus-like particle forming elements” or “VLP forming elements” shall be taken to mean structural and antigenic proteins of a virus which have the ability to self-assemble to form VLP or VLPs. For example, the VLP forming elements are selected from a hemaglutinin (HA) protein, a neuraminidase (NA) protein, and a matrix-1 (M1) protein of an influenza virus.

The composition of the present application comprises one or more ribonucleic acids (RNAs) encoding virus-like particle (VLP) forming elements of an influenza virus. The VLP forming elements are selected from structural polypeptide M1 protein and antigenic polypeptides NA protein and HA proteins. The structural protein (i.e. M1 protein) of the influenza virus produced from the one or more RNAs self-assemble with the antigenic proteins (i.e. NA and HA proteins) of the influenza virus resulting in virus-like particles (VLPs).

### Influenza Virus

VLP forming elements, the fragments and/or the variants thereof of the present disclosure are of an influenza virus.

Influenza viruses are members of the family Orthomyxoviriadae and represents enveloped viruses containing segmented negative-sense single-stranded RNA. It will be apparent to the skilled person that there are currently four influenza viruses—A, B, C and D. Influenza A virus is the most common strain of influenza virus infecting humans, animals, and birds, whilst influenza B virus infection mostly occurs in humans. Infection of influenza C virus does not cause any severe symptom in human or mammals and influenza D virus, to date, has only infected pigs and cattle.

Exemplary influenza A virus subtypes will be apparent to the skilled person. In one example, the influenza A virus is an influenza A(H1N1) or an influenza A(H3N2) subtype. For example, the influenza A virus is an influenza A(H1N1) subtype. For example, the influenza A virus is an influenza A(H3N2) subtype. For example, the influenza A virus is an A/Guangdong-Maonan/SWL1536/2019 (H1N1)pdm09 virus. For example, the influenza A virus is an A/Hawaii/70/2019 (H1N1)pdm09 virus. For example, the influenza A virus is an A/Brisbane/02/2018 (H1N1)pdm09 virus. For example, the influenza A virus is an A/Hong Kong/2671/2019 (H3N2)-like virus. For example, the influenza A virus is an A/Kansas/14/2017 (H3N2) virus. For example, the influenza A virus is an A/Victoria/2570/2019 (H1N1) virus. For example, the influenza A virus is an A/Wisconsin/588/2019 (H1N1) virus. For example, the influenza A virus is an A/Cambodia/e0826360/2020 (H3N2) virus. For example, the influenza A virus is an A/South Australia/34/2019 (H3N2) virus. For example, the influenza A virus is an A/Brisbane/02/2018 (H1N1) virus.

Exemplary influenza B virus lineages will be apparent to the skilled person. In one example, the influenza B virus is of B/Yamagata or B/Victoria lineage. For example, the influenza B virus is of B/Yamagata lineage. For example, the influenza B virus is of B/Victoria lineage. For example, the influenza B virus is a B/Washington/02/2019 (B/Victoria lineage) virus. For example, the influenza B virus is a B/Colorado/06/2017 (B/Victoria lineage) virus. For example, the influenza B virus is a B/Phuket/3073/2013 (Yamagata lineage) virus.

Influenza A virus and influenza B virus comprise a viral envelope made up of glycoproteins hemaglutinin (HA) proteins and neuraminidase (NA) proteins in a lipid bilayer membrane. The HA and NA proteins form spike-like structures on the surface of the viral envelope. HA proteins function to initiate infection of a host cell by binding to sialic acid-containing receptors on the surface of host cells. NA proteins function to enable the influenza virus to be released from infected host cell. Beneath the lipid bilayer membrane of the influenza A virus and influenza B virus is a scaffold formed by matrix-1 (M1) proteins which provide strength and rigidity to the viral envelope. Other components of influenza A virus and influenza B virus include nucleoprotein (NP) and non-structural (NS) proteins.

The viral envelope of influenza A virus further comprises ion channel matrix-2 (M2) proteins.

The viral envelope of influenza B virus further comprises ion channel influenza B matix protein 2 (BM2) and NB protein.

### Ribonucleic Acid (RNA)

The RNA of the present disclosure encompasses a non-self amplifying mRNA (also referred to as conventional mRNA (cRNA)) and a self-amplifying RNA (sa-mRNA). Typically, the cRNA comprises, in order from 5′ to 3′: a 5′cap structure, a 5′-UTR, a nucleotide sequence encoding a polypeptide of interest, a 3′-UTR, a fragment and/or a variant thereof and a tailing sequence (e.g. a polyadenylation signal or poly-A tail). In the present disclosure, the polypeptide of interest is a VLP forming element selected from a hemaglutinin (HA) protein, a neuraminidase (NA) protein, and a matrix-1 (M1) protein of an influenza virus. The cRNA of the present disclosure may further comprise a translation internal ribosome entry site (e.g. Kozak consensus sequence or IRES).

The sa-mRNA of the present disclosure comprises one or more features of a cRNA, however, sa-mRNA further comprises nucleotide sequences encoding non-structural proteins (NSPs) which enables the sa-mRNA to direct its self-amplification. For example, the sa-mRNA comprises NSPs derived from (or based on) an RNA virus (e.g. an alphavirus).

The sa-mRNA typically also includes a subgenomic (SG) promoter and when linked to a nucleotide sequence encoding NSPs and/or an polypeptide of interest, drives the expression of the NSPs and/or polypeptide of interest. The sa-mRNA is positive (+)-stranded so that it can be directly translated after delivery to a cell without the need for intervening replication steps (e.g., reverse transcription). Once introduced into a cell, the NSPs of the sa-mRNA are expressed and combine to form a replicase complex (i.e., an RNA-dependent RNA polymerase). The replicase complex is the component of the sa-mRNA which amplifies the original RNA producing both antisense and sense transcripts, resulting in production of multiple daughter RNAs, and subsequently the encoded polypeptide of interest.

The skilled person will understand that the sa-mRNA is an mRNA based on the genomic RNA of RNA viruses (e.g. an alphavirus). Exemplary alphaviruses include, but are not limited to, Venezuelan equine encephalitis virus (VEEV; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus.

In one example, the self-replicating RNA of the present disclosure comprises the non-structural proteins of an RNA virus, the 5′ and 3′ untranslated regions (UTRs) and the native subgenomic promoter.

In one example, the self-replicating RNA comprises one or more non-structural proteins of the RNA virus. For example, the RNA comprises at least one or more genes selected from the group consisting of a viral replicase (or viral polymerase), a viral protease, a viral helicase and other non-structural viral proteins. For example, the self-replicating RNA comprises a viral replicase (or viral polymerase).

In another example, the self-replicating RNA comprises a 5′- and a 3′-end UTR of the RNA virus. In one example, the self-replicating RNA comprises a 5′-CSE. In one example, the 3′-CSE forms part of the 3′ end UTR. In one example, the self-replicating RNA comprises a 5′-CSE. In one example, the 5′-CSE is a 51 nucleotide conserved sequence element (CSE). IN one example, the 5′CSE is located within the nsp1 coding sequence.

The self-replicating RNA of the present disclosure cannot induce production of infectious viral particles. For example, the self-replicating RNA of the present disclosure does not comprise viral genes encoding structural proteins necessary for production of viral particles.

In one example, the self-replicating RNA is derived from or based on an alphavirus. Suitable alphaviruses will be apparent to the skilled person and/or described herein.

In another example, the self-replicating RNA is derived from or based on a virus other than an alphavirus, for example, a positive-stranded RNA virus. Suitable positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

### Alphavirus

In one example, the self-replicating RNA of the present disclosure is derived from (or based on) an alphavirus.

Alphaviruses are the sole genus in the Togaviridae family and are an enveloped virus with a positive-sense, single-stranded RNA genome. The skilled person will understand that the alphavirus genome comprises two open reading frames (ORFs), non-structural and structural. The first ORF encodes four non-structural proteins (NSP1, NSP2, NSP3 and NSP4) necessary for transcription and replication of viral RNA. The second encodes three structural proteins: the core nucleocapsid protein C, and the envelope proteins P62 and E1, which associate as a heterodimer. The viral membrane-anchored surface glycoproteins are responsible for receptor recognition and entry into target cells through membrane fusion.

In one example, the self-replicating RNA of the present disclosure comprises a viral replicase (or viral polymerase). For example, the viral replicase is an alphavirus replicase, such as an alphavirus protein NSP4. In one example, the self-replicating RNA of the present disclosure comprises NSP1, NSP2, NSP3 and NSP4.

In one example, the self-replicating RNA of the present disclosure does not encode one or more alphavirus structural proteins (e.g., capsid and/or envelope glycoproteins). For example, the self-replicating RNA is unable to produce RNA-containing alphavirus virions (i.e., infectious viral particles).

In one example, the self-replicating RNA comprises a native alphavirus SG promoter (also referred to as “SGP”). For example, the native alphavirus SG promoter is a minimal SG promoter (i.e., the minimal sequence required for initiation of transcription) and comprises a sequence set forth in SEQ ID NO: 3.

The skilled person will be aware of alphaviruses suitable for use in the present disclosure. Exemplary alphaviruses include, but are not limited to, Venezuelan equine encephalitis virus (VEE; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus. In one example, the alphaviruses comprises VEE (e.g., Trinidad donkey, TC83CR). In one example, the alphavirus comprises TC-83 (e.g. as represented by Genbank accession number L01443.1). In one example, the alphavirus comprises Sindbis virus.

### 5′Untranslated Region (5′-UTR)

The present disclosure provides a RNA comprising a first nucleotide sequence comprising a 5′-untranslated region (5′-UTR).

As used herein, the term “5′-untranslated region” or “5′-UTR” refers to a non-coding region of an mRNA located at the 5′end of the translation initiation codon (e.g. AUG).

Exemplary 5′-UTR include, for example, 5′-UTR comprising or consisting of a nucleotide sequence derived from a 5′-UTR of a gene selected from the group consisting of haptoglobin (HP), fibrinogen beta chain (FGB), haptoglobin-related protein (HPR), albumin (ALB), complement component 3 (C3), fibrinogen alpha chain (FGA), alpha 1 collagen (Col1A), alpha 6 collagen (Col6A), alpha-1-antitrypsin (SERPINA1), alpha-1-antichymotrypsin (SERPINA3), arachidonate 5-lipoxygenase (ALOX5), tyrosine hydroxylase (TH gene), tumor protein P53 inducible protein 3 (TP5313) a fragment and/or a variant thereof.

In one example, the 5′UTR comprises or consists of a nucleotide sequence derived from a 5′-UTR of an alphavirus. For example, an alphavirus as defined herein. In one example, the 5′UTR comprises or consists of a nucleotide sequence derived from a 5′-UTR of an Venezuelan equine encephalitis virus (VEEV) or modified forms thereof (e.g. Trinidad donkey, TC-83CR or TC-83). For example, the 5′UTR comprises or consists of a sequence set forth in SEQ ID NO: 76.

In one example, the first nucleotide sequence comprises at least one microRNA binding site, an AU rich element (ARE), a GC-rich element, a stem loop, and combinations thereof.

microRNA Binding Site

As used herein, the term “microRNA binding site” refers to a sequence within a RNA that has sufficient complementarity to all or one region of a miRNA to interact, associate or bind to the microRNA (miRNA).

As used herein, the term “microRNA” or “miRNA” refers to 19-25 nucleotide long non-coding RNAs that bind to the 5′-UTR of RNAs and down-regulate gene expression (e.g. by inhibiting translation). The presence of microRNA binding site(s) in the first nucleotide of the RNA of the present disclosure can function to inhibit translation of the 5′-UTR.

Suitable miRNA binding site for use in the present disclosure will be apparent to the skilled person and/or described herein.

In one example, the miRNA binding site comprises a binding site for tissue specific microRNA or those regulating biological processes. For example, miRNA of the liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-id, miR-149), kidney (miR-192, miR-194, miR-204), and lung epithelial cells (let-7, miR-133, miR-126). For example, microRNA that regulate biological processes such as angiogenesis (miR-132). Further exemplifying miRNA and miRNA binding sites are disclosed in US patent application U.S. Ser. No. 14/043,927.

### AU Rich Element (ARE)

As used herein, the term “AU rich element (ARE)” or “AU rich elements (AREs)” refers to a region of a nucleotide sequence comprising stretches of Adeonisine (A) and Uridine (U). Exemplary ARE include, for example, ARE from cytoplasmic myc (c-myc), myoblast determinatioNA protein 1 (myoD), c-Jun, myogenin, granulocyte-macrophage colony-stimulating factor (GM-CSF) and tumour necrosis factor alpha (TNF-α), or a combination thereof.

In one example, ARE comprises a HuR (also known as Elavl1) specific binding site. HuR is known to bind ARE increasing the stability of the mRNA.

### GC-Rich Element

As used herein, the term “GC-rich element” refers to a nucleotide sequence with a high amount of Guanine (G) and/or Cytosine (C) compared to Adenine (A) and Thymine(T)/Uracil(U). The presence of GC-rich elements in a RNA (e.g. mRNA) can stabilise the mRNA.

In one example, the GC-rich element comprises a sequence of 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 nucleotides in length.

In one example, the GC-rich element comprises between 30% and 40%, or 40% and 50%, or 50% and 60%, or 60% and 70% cytosine. For example, the GC-rich element comprises between 30% and 40% cytosine. For example, the GC-rich element comprises between 40% and 50% cytosine. For example, the GC-rich element comprises between 50% and 60% cytosine. For example, the GC-rich element comprises between 60% and 70% cytosine.

In one example, the GC-rich element comprises 30%, or 40%, or 50%, or 60%, or 70% cytosine. For example, the GC-rich element comprise 30% cytosine. For example, the GC-rich element comprises 40% cytosine. For example, the GC-rich element comprises 50% cytosine. For example, the GC-rich element comprises 60% cytosine. For example, the GC-rich element comprises 60% cytosine. For example, the GC-rich element comprises 70% cytosine.

In one example, the GC-rich element is at least 50% cytosine.

In one example, the GC-rich element is at least 60% cytosine.

In one example, the GC-rich element is at least 70% cytosine.

In one example, the GC-rich element comprises a nucleotide sequence CCCCGGCGCC.

In another example, the GC-rich element comprises a nucleotide sequence CCCCGGC. In a further example, the GC-rich element comprises a nucleotide sequence GCGCCCCGCGGCGCCCCGCG.

In one example, the GC-rich element comprises a nucleotide sequence set forth in SEQ ID NO: 89 to 91. In one example, the GC-rich element comprises a nucleotide sequence set forth in SEQ ID NO: 89 (CCCCGGCGCC). In another example, the GC-rich element comprises a nucleotide sequence set forth in SEQ ID NO: 90 (CCCCGGC). In a further example, the GC-rich element comprises a nucleotide sequence set forth in SEQ ID NO: 91 (GCGCCCCGCGGCGCCCCGCG).

### Stem Loop

As used herein, the term “stem loop” refers to a nucleotide sequence comprising an intramolecular base pairing of two neighboured entirely or partially reverse complementary sequences to form a stem-loop. A stem-loop can occur in single-stranded DNA or, more commonly, in RNA. The stem loop can also be referred to as a hairpin or hairpin loop which usually consists of a stem and a terminal loop within a consecutive sequence, wherein the stem is formed by two neighboured entirely or partially reverse complementary sequences separated by a short sequence which builds the loop into a stem-loop structure.

The stability of the paired stem loop is determined by the length, the number of mismatched or bulges it contains, and the nucleotide composition of the paired region.

In one example, a loop of the stem loop is between 3 and 10 nucleotides in length. For example, the loop of the stem loop is between 3 and 8, or 3 and 7, or 3 and 6, or 4 and 5 nucleotides in length.

In one example, the loop of the stem loop is 4 nucleotides in length.

In one example, the stem loop is a histone stem loop. For example, the histone stem loop comprises or consist of a nucleotide sequence set for in SEQ ID NO: 5.

### Translation Initiation Sequence(s)

In one example, the RNA further comprises a translation initiation sequence. In one example, the translation initiation sequence is linked to the 3′ end of the 5′-UTR. In one example, the translation initiation sequence is operably linked to one or more coding sequence. For example, the translation initiation sequence is operably linked to one or more nucleotide sequence(s) encoding a virus-like particle (VLP) forming element. As used herein, the term “translation initiation sequence” refers to a nucleic acid sequence that initiates translation of an encoded polypeptide (e.g. VLP forming element) in the polynucleotide (e.g. RNA).

Suitable translation initiation sequence(s) will be apparent to a skilled person and/or described herein. For example, the translation initiation sequence is selected from the group consisting of a Kozak consensus sequence, an internal ribosome entry site (IRES), a subgenomic (SG) promoter and combinations thereof.

When the RNA(s) of the present disclosure comprises a translation initiation sequence operably linked to the 5′end of the one or more nucleotide sequence(s) encoding the VLP forming element and/or the one or more additional nucleotide sequence encoding a nucleoprotein (NP) and/or non-structural (NS) protein, each translation initiation sequence can be the same or different. In one example, the translation initiation sequence are two or more SG promoters. In one example, the two or more SG promoters are derived from the same alphavirus. In another example, the two or more SG promoters are derived from different alphavirus. In one example, the translation initiation sequence is one SG promoter and two or more IRES. In one example, the two or more IRES are the same. In one example, the two or more IRES are different. In one example, the translation initiation sequence is one IRES and two or more SG promoter.

In one example, the sa-mRNA comprises, in the order of 5′ to 3′: (i) nucleotide sequence(s) which encodes for the non-structural proteins of an alpha virus selected from NSP1, NSP2, NSP3, NSP4, (ii) a first translation initiation sequence operably linked to 5′ end of a nucleotide sequence encoding a first virus-like particle (VLP) forming element, (iii) a second translation initiation sequence operably linked to 5′ end of a nucleotide sequence encoding a second virus-like particle (VLP) forming element, and (iv) a third translation initiation sequence operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

In one example, the sa-mRNA comprises, in the order of 5′ to 3′: (i) nucleotide sequence(s) which encodes for the non-structural proteins of an alpha virus selected from NSP1, NSP2, NSP3, NSP4, (ii) a first subgenomic (SG) promoter or first IRES operably linked to 5′ end of a nucleotide sequence encoding a first virus-like particle (VLP) forming element, (iii) a second SG promoter or second IRES operably linked to 5′ end of a nucleotide sequence encoding a second virus-like particle (VLP) forming element, and (iv) a third SG promoter or third IRES operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

For example, the sa-mRNA comprises, in the order of 5′ to 3′: (i) nucleotide sequence(s) which encodes for the non-structural proteins of an alphavirus selected from NSP1, NSP2, NSP3, NSP4, (ii) a first subgenomic (SG) promoter operably linked to 5′ end of a nucleotide sequence encoding a first virus-like particle (VLP) forming element, (iii) a second IRES operably linked to 5′ end of a nucleotide sequence encoding a second virus-like particle (VLP) forming element, and (iv) a third IRES operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

For example, the sa-mRNA comprises, in the order of 5′ to 3′: (i) nucleotide sequence(s) which encodes for the non-structural proteins of an alpha virus selected from NSP1, NSP2, NSP3, NSP4, (ii) a first SG promoter operably linked to 5′ end of a nucleotide sequence encoding a first virus-like particle (VLP) forming element, (iii) a second SG promoter operably linked to 5′ end of a nucleotide sequence encoding a second virus-like particle (VLP) forming element, and (iv) a third SG promoter operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

In one example, the first SGP comprises or consists of a native SGP. In one example, the second SGP comprises or consists of the sequence set forth in SEQ ID NO. 3 and the third SGP comprises or consists of the sequence set forth in SEQ ID NO. 3. In one example, the second SGP comprises or consists of the sequence set forth in SEQ ID NO. 3 and the third SGP comprises or consists of the sequence set forth in SEQ ID NO. 75. In one example, the second SGP comprises or consists of the sequence set forth in SEQ ID NO. 75 and the third SGP comprises or consists of the sequence set forth in SEQ ID NO. 4.

For example, the sa-mRNA comprises, in the order of 5′ to 3′: (i) nucleotide sequence(s) which encodes for the non-structural proteins of an alphavirus selected from NSP1, NSP2, NSP3, NSP4, (ii) a first SG promoter operably linked to 5′ end of a nucleotide sequence encoding a first virus-like particle (VLP) forming element, (iii) a second IRES operably linked to 5′ end of a nucleotide sequence encoding a second virus-like particle (VLP) forming element, and (iv) a third SG promoter operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

For example, the sa-mRNA comprises, in the order of 5′ to 3′: (i) nucleotide sequence(s) which encodes for the non-structural proteins of an alpha virus selected from NSP1, NSP2, NSP3, NSP4, (ii) a first SG promoter operably linked to 5′ end of a nucleotide sequence encoding a first virus-like particle (VLP) forming element, (iii) a second SG promoter operably linked to 5′ end of a nucleotide sequence encoding a second virus-like particle (VLP) forming element, and (iv) a third IRES operably linked to 5′ end of a nucleotide sequence encoding a third virus-like particle (VLP) forming element,
wherein the first, second and third VLP forming element is selected from a hemaglutinin (HA) protein, a neuraminidase (NA) protein, and a matrix-1 (M1) protein of an influenza virus, and wherein the sa-mRNA comprises nucleotide sequences encoding each of the HA protein, the NA protein and the M1 protein.

In one example, the first VLP forming element is a hemaglutinin (HA) protein of an influenza virus, the second VLP forming element is a neuraminidase (NA) protein of an influenza virus, and the third VLP forming element is a matrix-1 (M1) protein of an influenza virus.

In one example, the first SGP is a native SGP and the first VLP forming element is a hemaglutinin (HA) protein of an influenza virus, the second SGP comprises or consists of the sequence set forth in SEQ ID NO: 3 and the second VLP forming element is a neuraminidase (NA) protein of an influenza virus, and the third SGP comprises or consists of the sequence set forth in SEQ ID NO: 3 and third VLP forming element is a matrix-1 (M1) protein of an influenza virus. In one example, the first SGP is a native SGP and the first VLP forming element is a hemaglutinin (HA) protein of an influenza virus, the second SGP comprises or consists of the sequence set forth in SEQ ID NO: 3 and the second VLP forming element is a neuraminidase (NA) protein of an influenza virus, and the third SGP comprises or consists of the sequence set forth in SEQ ID NO: 75 and third VLP forming element is a matrix-1 (M1) protein of an influenza virus. In one example, the first SGP is a native SGP and the first VLP forming element is a hemaglutinin (HA) protein of an influenza virus, the second SGP comprises or consists of the sequence set forth in SEQ ID NO: 75 and the second VLP forming element is a neuraminidase (NA) protein of an influenza virus, and the third SGP comprises or consists of the sequence set forth in SEQ ID NO: 3 and third VLP forming element is a matrix-1 (M1) protein of an influenza virus.

In one example, the sa-mRNA comprises a 5′ UTR prior to the nucleotide sequence(s) which encodes for the non-structural proteins of an alpha virus selected from NSP1, NSP2, NSP3, NSP4. In one example, the sa-mRNA comprises a 3′UTR. In one example, the sa-mRNA comprises a polyA tail.

### Kozak Consensus Sequence

As used herein, the term “Kozak consensus sequence” refers to a nucleotide sequence identified in eukaryotic genes that facilitates the translation of the gene by containing a start codon (also referred to as a translation initiation codon) which is recognised by a ribosome.

Exemplary Kozak consensus sequence are known in the art and/or described herein. In one example, the Kozak consensus sequence comprises or consists of a nucleotide sequence set forth in SEQ ID NO: 1. In one example, the Kozak consensus sequence comprises or consists of a nucleotide sequence set forth in SEQ ID NO: 2. In one example, the Kozak consensus sequence comprises or consists of a nucleotide sequence set forth in SEQ ID NO: 85. For example, the Kozak sequence is ACCATG. For example, the Kozak sequence is ACCAUGG. For example, the Kozak sequence is ACCATGG.

### Internal Ribosome Entry Site (IRES)

As used herein, the term “internal ribosome entry site” or “IRES” refers to a sequence of nucleotides within a RNA to which a ribosome or a component thereof, e.g., a 40S subunit of a ribosome, is capable of binding. An IRES need not necessarily comprise nucleic acid that induces translation of a RNA (e.g., a start codon; AUG).

Exemplary IRES include, for example, an IRES from poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

In one example, the IRES is derived from encephalomyocarditis virus (EMCV). For example, the IRES is a wild-type IRES from EMCV. For example, the wild-type EMCV IRES comprises a sequence set forth in SEQ ID NO: 88.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97(4): 1536-41).

In one example, the mRNA comprises, in the order of 5′ to 3′: (i) a first nucleotide sequence comprising a 5′-untranslated region (5′-UTR), a fragment and/or a variant thereof (ii) a nucleotide sequence encoding a first virus-like particle (VLP) forming element, (iii) a nucleotide sequence encoding a second virus-like particle (VLP) forming element (iv) a nucleotide sequence encoding a third virus-like particle (VLP) forming element, and (v) a second nucleotide sequence comprising a 3′-untranslated region (3′-UTR), a fragment and/or a variant thereof,
wherein the first, second and third VLP forming element is selected from a hemaglutinin (HA) protein, a neuraminidase (NA) protein, and a matrix-1 (M1) protein of an influenza virus,
wherein the RNA comprises nucleotide sequences encoding each of the HA protein, the NA protein and the M1 protein, and
wherein an internal ribosome entry site (IRES) is positioned between:
(a) the first nucleotide sequence and the nucleotide sequence encoding the first VLP forming element; and/or
(b) the nucleotide sequences encoding the first VLP forming element and the second VLP forming element; and/or
(c) the nucleotide sequence encoding the second VLP forming element and the nucleotide sequence encoding the third VLP forming element.

For example, the IRES is positioned between the first nucleotide sequence and the nucleotide sequence encoding the first VLP forming element. For example, the IRES is positioned between the nucleotide sequences encoding the first VLP forming element and the second VLP forming element. For example, the IRES is positioned between the nucleotide sequence encoding the second VLP forming element and the nucleotide sequence encoding the third VLP forming element.

For example, the IRES is positioned between:
(a) the first nucleotide sequence and the nucleotide sequence encoding the first VLP forming element;
(b) the nucleotide sequences encoding the first VLP forming element and the second VLP forming element; and
(c) the nucleotide sequence encoding the second VLP forming element and the nucleotide sequence encoding the third VLP forming element.

For example, the IRES is positioned between:
(a) the first nucleotide sequence and the nucleotide sequence encoding the first VLP forming element; and
(b) the nucleotide sequences encoding the first VLP forming element and the second VLP forming element.

For example, the IRES is positioned between:
(a) the nucleotide sequences encoding the first VLP forming element and the second VLP forming element; and
(b) the nucleotide sequence encoding the second VLP forming element and the nucleotide sequence encoding the third VLP forming element.

For example, the IRES is positioned between:
(a) the first nucleotide sequence and the nucleotide sequence encoding the first VLP forming element; and
(b) the nucleotide sequence encoding the second VLP forming element and the nucleotide sequence encoding the third VLP forming element.

### Subgenomic (SG) Promoter

As used herein, the term “subgenomic promoter” (also known as ‘junction region’ promoter) refers to a promoter that directs the expression of a heterologous nucleotide sequence, regulating protein expression.

SG promoters suitable for use in the present disclosure will be apparent to the skilled person and/or are described herein.

In one example, the polynucleotide of the disclosure comprises a SG promoter from any alphavirus. For example, the RNA of the disclosure (e.g., cRNA or selfreplicating RNA) comprises a SG promoter from any alphavirus. In one example, the self-replicating RNA comprises a SG promoter from any alphavirus.

In one example, the SG promoter is derived from or based on an alphavirus SG promoter. For example, the SG promoter is a native alphavirus SG promoter. For example, a native SG promoter is a promoter that is native to the RNA virus from which it is derived and/or based on (e.g., an alphavirus).

In one example, the native SG promoter is a minimal SG promoter. For example, the minimal SG promoter is the minimal sequence required for initiation of transcription. In one example, the minimal SG promoter comprises a sequence set forth in SEQ ID NO: 3.

In one example, the native SG promoter is an extended SG promoter. For example, the extended SG promoter is a minimal SG promoter extended at the 5′ end with nucleotides occurring in a sequence encoding a non-structural protein (e.g., NSP4) of the RNA virus (e.g., an alphavirus). In one example, the extended SG promoter is a minimal SG promoter extended at the 5′ end with nucleotides occurring in a sequence encoding an alphavirus NSP4. In one example, the extended SG promoter is a minimal SG promoter extended at the 5′ end by no more that 51 nucleotides occurring in a sequence encoding an alphavirus NSP4. In one example, the extended SG promoter comprises or consists of a sequence set forth in SEQ ID NO: 3 extended at the 5′ end by no more than 51 nucleotides occurring in a sequence encoding a non-structural protein (e.g., an alphavirus NSP4). For example, the extended SG promoter is no more than 100 nucleotides in length. In one example, the extended SG promoter comprises or consists of nucleotides 2 to 101 of SEQ ID NO: 4.

In one example, the SG promoter is extended at the 5′ end by about 5 nucleotides to about 20 nucleotides, for example by about 5 nucleotides, or about 10 nucleotides, or about 12, or about 15 nucleotides, or about 20 nucleotides, occurring in a sequence encoding a non-structural protein (e.g., an alphavirus NSP4). In another example, the SG promoter is extended at the 5′ end by about 20 to about 35 nucleotides, for example, by about 25 nucleotides or about 27 nucleotides, or about 30 nucleotides, or about 35 nucleotides, occurring in a sequence encoding a non-structural protein (e.g., an alphavirus NSP4).

In one example, the SG promoter is extended at the 5′ end by about 12 nucleotides occurring in a sequence encoding a non-structural protein (e.g., an alphavirus NSP4). In one example, the extended SG promoter comprises the sequence set forth in SEQ ID NO: 3 extended at the 5′ end by 12 nucleotides occurring in a sequence encoding a non-structural protein (e.g., an alphavirus NSP4). For example, the extended SG promoter is no more than 61 nucleotides in length. In one example, the extended SG promoter comprises or consists of nucleotides 41 to 101 of SEQ ID NO: 4. In another example, the extended SG promoter comprises or consists of a sequence set forth in SEQ ID NO: 75.

In one example, the SG promoter is extended at the 5′ end by about 31 nucleotides occurring in a sequence encoding a non-structural protein (e.g., an alphavirus NSP4). In one example, the extended SG promoter comprises a sequence set forth in SEQ ID NO: 3 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter comprises or consists of nucleotides 22 to 101 of SEQ ID NO: 4. In another example, the extended SG promoter comprises or consists of a sequence set forth in SEQ ID NO: 86.

In one example, the SG promoter comprises or consists of the sequence set forth in SEQ ID NO: 3. In one example, the SG promoter comprises or consists of the sequence set forth in SEQ ID NO: 75.

### 3′Untranslated Region (3′-UTR)

The present disclosure provides a RNA comprising a second nucleotide sequence comprising a 3′-untranslated region (3′-UTR), a fragment and/or a variant thereof.

As used herein, the term “3′-UTR” refers to a region of an mRNA located at the 3′end of the translation termination codon (i.e. stop codon). In one example, the 3′UTR is located at the 3′ end of the translation termination codon of the nucleotide sequence encoding the last VLP forming element in the mRNA, for example, he nucleotide sequence encoding the third VLP forming element if the mRNA encodes three VLP forming elements.

Exemplary 3′-UTRs include, for example, a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is derived from or based on an alphavirus 3′UTR. In one example, the 3′UTR is a 3′UTR of an alphavirus, for example, an alphavirus as defined herein. In one example, the 3′UTR is a native alphavirus 3′UTR. For example, a native 3′UTR is a 3′UTR that is native to the RNA virus from which it is derived and/or based on (e.g., an alphavirus). For example, a native 3′UTR is a 3′UTR that is native to the RNA virus from the NSP are derived. In one example, the 3′UTR is a heterologous alphavirus 3′UTR.

In one example, the 3′UTR is a 3′UTR of a Venezuelan equine encephalitis virus (VEEV) or modified forms thereof. For example, the 3′UTR comprises a sequence set forth in SEQ ID NO: 77. In one example, the 3′UTR is a 3′UTR of a Sindbis virus (SINV) or modified forms thereof. For example, the 3′UTR comprises a sequence set forth in SEQ ID NO: 79.

In one example, the 3′-UTR comprises or consists of a nucleotide sequence derived from a 3′-UTR of an albumin gene. In one example, the 3′-UTR comprises or consists of a nucleotide sequence derived from a 3′-UTR of a vertebrate a-globin gene. For example, the 3′-UTR comprises or consists of a nucleotide sequence derived from a 3′-UTR of a mammalian a-globin gene. For example, the 3′-UTR comprises or consists of a nucleotide sequence derived from a 3′-UTR of a human a-globin gene.

In one example, the 3′-UTR of the present disclosure further comprises at least one microRNA binding site, an AU rich element (ARE), a GC-rich element, a triple helix, a stem loop, one or more stop codons or a combination thereof.

In one example, the 3′UTR comprises or consists of a conserved sequence element (CSE). In one example, the 3′UTR comprises or consists of a 3′CSE. In one example, the 3′CSE is derived from or based on an alphavirus 3′CSE. In one example, the 3′CSE is a 3′CSE of an alphavirus, for example, an alphavirus as defined herein. In one example, the 3′CSE is a 3′CSE of a Venezuelan equine encephalitis virus (VEEV) or modified forms thereof. For example, the 3′CSE comprises a sequence set forth in SEQ ID NO: 78. In one example, the 3′CSE is a 3′CSE of a Sindbis virus (SINV) or modified forms thereof. For example, the 3′CSE comprises a sequence set forth in SEQ ID NO: 80.

### Stop Codon

As used herein, the term “stop codon” refers to a trinucleotide sequence within a mRNA that signals the stop of protein synthesis by a ribosome.

In one example, the second nucleotide sequence comprises one or more stop codons linked to the 5′ end of the 3′-UTR, the fragment and/or the variant thereof. For example, the stop codon is selected from UAG, UAA, and UGA.

In one example, the RNA comprises two consecutive stop codons comprising a sequence UGAUGA.

In one example, the RNA comprises two consecutive stop codons comprising a sequence UAAUAG.

### 3′ Tailing Sequence

The RNA of the present disclosure comprises a third nucleotide sequence comprising one or more 3′ tailing sequences located at the 3′end of the third nucleotide sequence.

As described herein, the term “3′ tailing sequence” or “3′ tailing sequences” refers to a nucleotide sequence (e.g. polyadenylation signal) which induces the addition of non-encoded nucleotides to the 3′end of a mRNA or a nucleotide sequence (e.g. poly-A sequence) located at the 3′ end of a mRNA. A skilled person will appreciate that the 3′ tailing sequence and/or products of the 3′ tailing sequence in a mRNA functions to stabilise the mRNA and/or prevent the mRNA from degradation.

As used herein, the term “interrupting linker” in reference to a poly-A or poly-C sequence of the present disclosure refers to a single nucleotide or nucleotide sequence which are linked to, and interrupt, a stretch of consecutive adenosine or cytosine nucleotides in the poly-A or poly-C sequence. For example, the interrupting linker in a poly-A sequence is a single nucleotide or a nucleotide sequence consisting or comprising a nucleotide other than an adenosine nucleotide. For example, the interrupting linker in a poly-C sequence is a single nucleotide or a nucleotide sequence consisting or comprising a nucleotide other than an cytosine nucleotide.

In one example, the 3′ tailing sequences are selected from the group consisting of a poly-A sequence, polyadenylation signal, a G-quadruplex, a poly-C sequence, a stem loop and combinations thereof.

### Poly-A Sequence

As used herein, the term “poly-A sequence” refers to a nucleotide sequence of Adenine (A) located at the 3′end of a mRNA. In the context of the present disclosure, the poly-A sequence may be located within the mRNA or DNA (e.g. a DNA plasmid serving as a template for generating the mRNA by transcription of the vector).

Suitable poly-A sequence for use in the present disclosure will be apparent to the skilled person and/or are described herein. In one example, the poly-A sequence comprises consecutive (i.e. one after the other) adenosine nucleotides of any length (e.g. to 10 to 300). In one example, the poly-A sequence comprises consecutive adenosine nucleotides separated by one or more interrupting linker. In one example, the poly-A sequence comprises consecutive adenosine nucleotides without an interrupting linker.

### Polyadenylation Signal

As used herein, the term “polyadenylation signal” refers to a nucleotide sequence which induces polyadenylation. Polyadenylation is typically understood to be the addition of a poly-A sequence to a RNA (e.g. to a premature mRNA to generate a mature mRNA). The polyadenylation signal may be located within a nucleotide sequence at the 3′-end of the RNA (e.g. mRNA) to be polyadenylated.

Suitable polyadenylation signal for use in the present disclosure will be apparent to the skilled person and/or described herein.

In one example, the polyadenylation signal comprises a hexamer consisting of Adenine and Uracil/Thymidine nucleotides. In one example, the hexamer sequence comprises or consists of AAUAAA.

In one example, the third nucleotide sequence comprising the 3′ tailing sequence comprises a polyadenylation signal but does not comprise a poly-A sequence.

### G-Quadruplex

As used herein, the term “G-quadruplex” or “G4” refers to a nucleotide sequence rich in guanine residues which forms a four stranded secondary structure. For example, the G-quadruplex is a cyclic hydrogen bonded array of four guanine nucleotides formed by G-rich sequences in both DNA and RNA.

In one example, the third nucleotide sequence comprises a poly-A sequence and a G-quadruplex. For example, the third nucleotide sequence comprises a poly-A sequence linked to a G-quadruplex to produce a polyA-G quartet.

### Poly-C Sequence

As used herein, the term “poly-C sequence” refers to a nucleotide sequence of Cytosine (C) located at the 3′end of a mRNA. In the context of the present disclosure, the poly-C sequence may be located within the mRNA or DNA (e.g. a DNA plasmid serving as a template for generating the mRNA by transcription of the vector).

Suitable poly-C sequence for use in the present disclosure will be apparent to the skilled person and/or are described herein.

In one example, the one or more 3′ tailing sequences comprises one or more poly-C sequences each comprising between 10 and 300 consecutive cytosine nucleotides. For example, the poly-C sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive cytosine nucleotides. For example, the poly-C sequence each comprises 10, or 20, or 30, or 40, or 50, or 60, or 70, or 80, or 90, or 100, or 125, or 150, or 175, or 200, or 225, or 250, or 275, or 300 consecutive cytosine nucleotides.

In one example, the one or more poly-C sequences is separated by an interrupting linker. For example, the third nucleotide sequence comprising the one or more 3′tailing sequences comprises, in order of 5′ to 3′: consecutive cytosine nucleotides, an interrupting linker, and further consecutive cytosine nucleotides.

In one example, the interrupting linker is from 10 to 50, or 50 to 100, or 100 to 150 nucleotides in length. For example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length.

### 5′Cap Structure

In one example, the present disclosure provides a mRNA comprising a 5′terminal cap structure.

As used herein, the term “5′cap structure” refers to a structure at the 5′ terminal end of a mRNA involved in nuclear export and binds a mRNA Cap Binding Protein (CBP). The 5′cap structure is known to stabilise mRNA through association of CBP with poly(A) binding protein to form a mature mRNA. Accordingly, the presence of a 5′cap structure in the mRNA of the present disclosure can further increase the stability of the mRNA compared to a mRNA without the 5′cap.

Exemplary 5′cap structure includes, for example, anti-reverse cap analogue (ARCA), N7,2′-O-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2.

Typically, an endogenous mRNA is 5′capped with a guanosine through a (5)′-ppp-(5)′-triphosphate linkage attached to the 5′terminal nucleotide of the mRNA. The guanosine cap can then be methylated to a 7-methylguanosine (m7G) generating a 7mG(5′)ppp(5′)N,pN2p (Cap0 structure), where N represents the first and second 5′terminal nucleotide of the mRNA. The cap0 structure can be further 2′-O-methylated to produce 7mG(5′)ppp(5′)NlmpNp (Cap1), and/or 7mG(5′)-ppp(5′)NlmpN2mp (Cap2).

In one example, the RNA of the present disclosure comprises an endogenous cap.

As used herein, the term “endogenous cap” refers to a 5′cap synthesised in a cell. For example, endogenous cap is a natural 5′cap or a wild-type 5′cap. For example, the endogenous cap is a Cap0, Cap1, or Cap2 structure.

In one example, the RNA of the present disclosure comprises an analog of an endogenous cap (also referred to as cap analog).

As used herein, the term “analogue thereof” in the context of an endogenous cap or “cap analog” refers to a synthetic 5′cap. The cap analog can be used to produce 5′capped mRNA in in vitro transcription reactions. Cap analogs may be chemically (i.e. non-ezymatically) or enzymatically synthesized and/or linked to a nucleotide (e.g. 5′terminal nucleotide of an mRNA). Exemplary cap analogs are commercially available and include, for example, 3′-O-Me-m7G(5′)ppp(5′)G, G(5′)ppp(5′)A, G(5′)ppp(5′)G, m7G(5′)ppp(5′)A, m7G(5′)ppp(5′)G (New England BioLabs). In one example, the cap analog is N7,3′-O-dimethyl-guanosine-5′-triphosphate-5′-guanosine (i.e. anti-reverse cap analogue (ARCA)).

In one example, the 5′cap structure is a non-hydrolyzable cap structure. The non-hydrolyzable cap structure can prevent decapping of the mRNA and increase the half-life of the mRNA.

In one example, the non-hydrolyzable cap structure comprises a modified nucleotide selected from a group consisting or a a-thio-guanosine nucleotide, a-methyl-phosphonate, seleno-phosphate, and a combination thereof. In one example, the modified nucleotide is linked to the 5′end of the mRNA through an a-phosphorothiate linkage. Methods of linking the modified nucleotide to the 5′end of the mRNA will be apparent to the skilled person. For example, using a Vaccinia Capping Enzyme (New England Biolabs).

### Modifications

In one example, the RNA of the present disclosure comprises one or more modification(s). Typically, modifications are introduced into a RNA (e.g. mRNA) to increase the translation efficiency and/or stability of the RNA. Suitable modifications to the RNA will be apparent to the skilled person and/or described herein.

In one example, the first nucleotide sequence comprising the 5′-UTR and/or the fragment thereof is modified. Modification of the first nucleotide sequences comprising the 5′-UTR and/or the fragment thereof results in a variant of the 5′-UTR and/or the fragment thereof.

In one example, the second nucleotide sequence comprising the 3′-UTR and/or the fragment thereof is modified. Modification of the second nucleotide sequences comprising the 3′-UTR and/or the fragment thereof results in a variant of the 3′-UTR and/or the fragment thereof.

In one example, the nucleotide sequence encoding the VLP forming element and/or the fragment thereof is modified. Modification of the nucleotide sequence encoding the VLP forming element and/or the fragment thereof results in a variant of VLP forming element and/or the fragment thereof.

In one example, one or more nucleotide sequence(s) of the RNA are codon optimized. Method of codon optimization will be apparent to the skilled person and/or described herein. For example, tools for codon optimization of RNA include, for example, GeneArt GeneOptimizer (Thermofisher®) or GenSmart® (GeneScript®).

In one example, the RNA is modified to increase the amount of Guanine (G) and/or Cytosine (C) in the RNA. The amount of G/C in the RNA (i.e. G/C content) can influence the stability of the RNA. Accordingly, RNA comprising an increased amount of G/C nucleotides can be functionally more stable than RNAs containing a large amount of Adenine (A) and Thymine (T) or Uracil (U) nucleotides. The G/C content is increased by substituting A or T nucleotides with G or C nucleotides.

In one example, the G/C content is increased in the nucleotide sequence encoding the VLP forming element. The modification(s) in the nucleotide sequence encoding the VLP forming element takes advantage of the ability of substituting codons that contain less favourable combinations of nucleotides (in terms of mRNA stability) with alternative codons encoding the same amino acid, or encoding amino acid(s) of similar chemistry (e.g. conserved amino acid substitution). For example, the G/C content is increased by substituting codons containing A or T nucleotides with codons containing G or C nucleotides that encode for the same amino acid. For example, the G/C content is increased by substituting codons containing A or T nucleotides with codons containing G or C nucleotides that encode for an amino acid of similar chemistry.

In one example, the G/C content is increased in one or more nucleotide sequence of the RNA which do not encode the VLP forming element. For example, the G/C content is increased in the second nucleotide comprising the 3′-UTR and/or the fragment thereof. For example, the G/C content is increased in the first nucleotide sequence comprising a 5′-UTR.

In one example, the RNA comprises at least one chemically modified nucleotide.

As used herein, the term “chemical modification” or “chemically modified” in the context of a nucleotide refers to naturally occurring nucleotides (i.e. A, T, C, G, U) which are modified by replacement, insertion or removal of individual or several atoms or atomic groups compared to the naturally occurring nucleotides. In one example, at least one naturally occurring nucleotide of the RNA is replaced with a chemically modified nucleotide. In one example, at least 10%, or 20%, or 30%, or 40%, or 50%, or 60%, or 70%, or 80%, or 90%, or 100% of naturally occurring nucleotides of the RNA is replaced with a chemically modified nucleotide. Suitable chemically modified nucleotides for use in the present disclosure will be apparent to the skilled person and/or described herein. Exemplary chemically modified nucleotides include, for example, N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), and 1-methylpseudouridine (m1ψ).

### Methods of Production

Suitable methods for the production of a RNA of the present disclosure will be apparent to the skilled person and/or described herein.

The plasmid DNA is produced by inserting the nucleotide sequence comprising the 5′-UTR, optionally the nucleotide sequence encoding the NSPs (e.g. NSP1, NSP2, NSP3 and NSP 4), the nucleotide sequence(s) encoding the VLP forming element(s), and the nucleotide sequence comprising the 3′UTR into a DNA vector. Suitable DNA vector for use will be apparent to the skilled person, and the nucleotide sequences of the present disclosure can be purchased from any commercial supplier. Insertion of the nucleotide sequence(s) into the DNA vector may be performed using standard methods in the art.

In one example, the mRNA is produced using a plasmid DNA. The skilled person will understand that plasmid DNA is relatively stable. Briefly, competent bacterial cells (e.g., Escherichia coli) cells are transformed with a DNA plasmid encoding the mRNA of the present disclosure. Individual bacterial colonies are isolated and the resultant plasmid DNA amplified in E. coli cultures.

In one example, the plasmid DNA is isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In one example, the mRNA is made by in vitro transcription from a linearized DNA template using an RNA polymerase (e.g., T7 RNA polymerase). In some embodiments, the RNA described herein further comprises one or more additional 5′ nucleotides (e.g. an additional 5′G) as an artefact of the promoter use (e.g. T7 promoter (TAATACGACTCACTATAG, SEQ ID NO: 92)). Following in vitro transcription, the DNA template is removed by DNase digestion. The skilled person will understand that synthetic RNA capping is performed to correct mRNA processing and contribute to stabilization of the mRNA. In one example, the RNA is enzymatically 5′-capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the RNA via a 5′-5′ triphosphate bridge. In one example, the 5′ cap is a cap1 structure, for example, the 5′-cap (i.e., cap1) consists of the cap0 with an additional methylation of the 2′O position of the initiating nucleotide. The skilled person will also understand that polyadenylation of the mRNA may be performed in a mRNA comprising a polyadenylation sequence.

In one example, the mRNA is purified. Various methods for purifying mRNA will be apparent to the skilled person. For example, the mRNA is purified using lithium chloride (LiCl) precipitation. In another example, the mRNA is purified using tangential flow filtration (TFF). In one example, the mRNA is purified using an anion exchange chromatography. For example, anion exchange chromatography is performed using an anion exchange resin (e.g. MustangQ® membrane (Pall®)). Following purification, the mRNA is resuspended in e.g., nuclease-free water.

### Compositions

The present disclosure provides an immunogenic composition comprising a mRNA of the present disclosure. For example, the immunogenic composition is a vaccine.

The present disclosure also provides a pharmaceutical composition comprising an immunogenic composition of the present disclosure and a pharmaceutically acceptable carrier.

It will be apparent to the skilled person and/or described herein, that the mRNA of the present disclosure may be present as naked mRNA or in combination with lipids, polymers or other delivery system that facilitates entry into the cells.

### Delivery Systems

In one example, the pharmaceutical composition of the present disclosure further comprises a lipid nanoparticle (LNP), a polymeric microparticle and/or an oil-in-water emulsion. For example, the mRNA is encapsulated in, bound to or adsorbed on a LNP, a polymeric microparticle and/or an oil-in-water emulsion.

### Lipid Nanoparticles

In one example, the pharmaceutical composition of the present disclosure further comprises a LNP.

It will be apparent that the term “lipid nanoparticle” or “LNP” shall be understood to refer to any lipid composition, including, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers. Methods of preparing a LNP are known to the skilled person and/or described herein. In one example, LNP are prepared using a staggered herribone mixer. For example, as described in US patent application 2012/0276209. In another example, liposomes are prepared using a microfluidic device. For example, as described in WO2018220553. In one example, LNP are prepared using a T-junction mixer.

Lipid nanoparticles suitable for use in the present disclosure will be apparent to the skilled person and/or are described herein. For example, the LNP comprises an ionisable lipid.

As used herein, the term “ionisable lipid” or “ionisable lipids” shall refer to a lipid having at least one protonatable or deprotonatable group. For example, the lipid is positively charged at a pH at or below physiological pH (e.g. pH 7.4), and neutral at a second pH (e.g. at or above physiological pH). For example, the lipid is a cationic lipid.

Suitable ionisable lipids can have an anionic, cationic or zwitterionic hydrophilic head group. Exemplary phospholipids (anionic or zwitterionic) for use in the present disclosure include, for example, phosphatidylethanolamines, phosphatidylcholines, phosphatidylserines, and phosphatidylglycerols. In one example, the lipid is a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 2,5-bis((9z,12z)-octadeca-9,12,dien-1-yloxyl)benzyl-4-(dimethylamino)butanoate (LKY750). In one example, the phospholipid is 2,5-bis((9z,12z)-octadeca-9,12,dien-1-yloxyl)benzyl-4-(dimethylamino)butanoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

Lipid moieties suitable for use in the LNP will be apparent to the skilled person and include, for example, a fatty acid, an isoprenoid and combinations thereof. In one example, the lipid moiety is selected from the group consisting of an isoprenoid, a triglyceride, a phospholipid, a cholesteryl ester and combinations thereof.

In one example, the lipid nanoparticle additionally comprises a PEG-lipid, a sterol structural lipid and/or a neutral lipid. In one example, the lipid nanoparticle does not comprise a cationic lipid.

### PEG-Lipids

In one example, the present disclosure provides a LNP comprising a PEGylated lipid.

It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid includes PEG-c-DOMG, PEG-DMG, PEG-DLPE, PEG-DMPE, PEG-DPPC, a PEG-DSPE lipid and combinations thereof.

### Neutral Lipids

In one example, the present disclosure provides a LNP comprising a neutral lipid.

Suitable neutral or zwitterionic lipids for use in the present disclosure will be apparent to the skilled person and include, for example, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

### Structural Lipids

In one example, the present disclosure provides a LNP comprising a structural lipid.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol.

In one example, the structural lipid is a sterol. For example, the structural lipid is cholesterol. In another example, the structural lipid is campesterol.

### Polymeric Microparticles

In one example, the pharmaceutical composition of the present disclosure further comprises a polymeric microparticle.

The skilled person will be aware that various polymers can form microparticles to encapsulate or adsorb the RNA of the present disclosure. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(X-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

### Oil-In-Water Emulsion

In one example, the pharmaceutical composition of the present disclosure further comprises an oil-in-water emulsion e.g. an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1, 2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl [C 16:0] trimethyl ammonium propane (DPTAP) and distearoyltrimethylammonium propane (DSTAP).

In some examples, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

### Pharmaceutically Acceptable Carrier

Suitably, in compositions or methods for administration of the mRNA of the disclosure to a subject, the mRNA is combined with a pharmaceutically acceptable carrier as is understood in the art. Accordingly, one example of the present disclosure provides a composition (e.g., a pharmaceutical composition) comprising the mRNA of the disclosure (and any delivery system e.g. LNP) combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A mRNA of the present disclosure is useful for parenteral, topical, oral, or local administration, intramuscular administration, aerosol administration, or transdermal administration, for prophylactic or for therapeutic treatment. In one example, the mRNA is administered parenterally, such as intramuscularly, subcutaneously or intravenously. For example, the RNA is administered intramuscularly.

Formulation of a mRNA of the present disclosure to be administered will vary according to the route of administration and formulation (e.g., solution, emulsion, capsule) selected. An appropriate pharmaceutical composition comprising a mRNA to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The mRNA can be stored in the liquid stage or can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the molecule of the disclosure are those large enough to produce the desired effect. For example, the composition comprises an effective amount of the mRNA. In one example, the composition comprises a therapeutically effective amount of the mRNA. In another example, the composition comprises a prophylactically effective amount of the mRNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the mRNA is administered at an initial (or loading) dose which is higher than subsequent (maintenance doses). For example, the mRNA is administered at an initial dose of between about 10 mg/kg to about 30 mg/kg. The mRNA is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which the mRNA is initially administered at a lower dose than used in subsequent doses. This dosage regime is useful in the case of subject's initially suffering adverse events.

In the case of a subject that is not adequately responding to treatment, multiple doses in a week may be administered. Alternatively, or in addition, increasing doses may be administered.

A subject may be retreated with the mRNA, by being given more than one exposure or set of doses, such as at least about two exposures of the mRNA, for example, from about 2 to 60 exposures, and more particularly about 2 to 40 exposures, most particularly, about 2 to 20 exposures.

In one example, any retreatment may be given when signs or symptoms of disease return.

In one example, any retreatment may be given when there are no signs or symptoms of disease return.

In another example, any retreatment may be given at defined intervals. For example, subsequent exposures may be administered at various intervals, such as, for example, about 3-4 weeks, or 4-12 weeks, or 24-28 weeks, or 48-56 weeks or longer. For example, such exposures are administered at intervals each of about 3-4 weeks, or 4-8 week, or 4-12 weeks, or 24-26 weeks or about 38-42 weeks, or about 50-54 weeks.

In another example, for subjects experiencing an adverse reaction, the initial (or loading) dose may be split over numerous days in one week or over numerous consecutive days.

Administration of the mRNA according to the methods of the present disclosure can be continuous or intermittent, depending, for example, on the recipient's physiological condition, whether the purpose of the administration is therapeutic or prophylactic, and other factors known to skilled practitioners. The administration of the mRNA may be essentially continuous over a preselected period of time or may be in a series of spaced doses, e.g., either during or after development of a condition.

### Screening Assays

### Virus-Like Particle (VLP) Forming Element Expression

In one example, the composition comprising the mRNA(s) is assessed for expression of the VLP forming elements. For example, antigen expression is detected using antibodies against the VLP forming elements. In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of mRNA is calculated.

### Quantification of Virus-Like Particle (VLP) Release

In one example, the composition comprising of mRNA(s) is assessed for formation and release of VLPs from cells expressing VLP forming elements. For example, VLPs release from cells is analysed using antibodies against VLP forming elements. In a further example, association between VLP forming elements is determined using antibody-mediated co-immunoprecipitation and/or detection of the VLP forming elements (e.g. HA, NA and M1) in the co-immunoprecipitation sample by, for example, Western blot analysis.

### Microneutralization Assay

In one example, the composition comprising the mRNA(s) (naked and/or formulated) is assessed for antibody responses. For example, the composition comprising the mRNA(s) is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

### Hemagglutination Inhibition (HAI) Assay

In one example, the composition comprising the mRNA(s) (naked and/or formulated) is assessed for antibody responses. For example, the composition comprising the mRNA(s) is assessed using a hemagglutination inhibition (HAI) assay. Methods of performing a HAI assay will be apparent to the skilled person and/or described, for example, in WHO (2011) Manual for the laboratory diagnosis and virological surveillance of influenza: WHO Press, World Health Organization.

### Neuraminidase Inhibition Assay (NAI) Assay

In one example, the composition comprising the mRNA(s) (naked and/or formulated) is assessed for antibody responses. For example, the composition comprising the mRNA(s) is assessed using a neuraminidase inhibition (NAI) assay. Methods of performing a NAI assay will be apparent to the skilled person and/or described, for example, in WHO (2011) Manual for the laboratory diagnosis and virological surveillance of influenza: WHO Press, World Health Organization.

Antigen Specific T cell Responses

In one example, the composition comprising the mRNA(s) (naked and/or formulated) is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Methods of Producing Virus-Like Particles

The compositions and RNA described herein can be used to produce virus-like particles. The inventors have found that the stability of VLPs produced from a composition described herein could be increased by increasing M1 protein incorporation in the VLPs. The inventors have also found that efficient release of VLPs from, for example, a cell could be increased by increasing NA protein incorporation in the VLPs. Accordingly, the present disclosure also provides a method of expressing a virus-like particle (VLP) in a subject comprising administering the RNA, the composition, the immunogenic composition, or the pharmaceutical composition of the present disclosure to the subject.

The present disclosure further provides use of the RNA, the composition, or the immunogenic composition, or the pharmaceutical composition of the present disclosure in the manufacture of a medicament for expressing a VLP in a subject in need thereof.

The present disclosure also provides the RNA, the immunogenic composition, or the pharmaceutical composition of the present disclosure for use in a method of expressing a VLP in a subject in need thereof.

The present disclosure also provides a method of increasing stability of a virus-like particle (VLP), the method comprising introducing a nucleotide sequence encoding a matrix-1 (M1) protein of an influenza virus into a composition, wherein the composition comprises a RNA comprising, in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding a neuraminidase (NA) protein of an influenza virus operably linked to a second subgenomic (SG) promoter;
c) a nucleotide sequence encoding the M1 protein of an influenza virus operably linked to a third subgenomic (SG) promoter; and
wherein introducing the nucleotide sequence encoding the M1 protein into the composition increases the stability of the VLP produced from the composition.

The present disclosure further provides a method of increasing efficient release of a virus-like particle (VLP), the method comprising introducing a nucleotide sequence encoding a neuraminidase (NA) protein of an influenza virus into a composition, wherein the composition comprises a RNA comprising, in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding the NA protein of an influenza virus operably linked to a second subgenomic (SG) promoter;
c) a nucleotide sequence encoding a matrix-1 (M1) protein of an influenza virus operably linked to a third subgenomic (SG) promoter; and
wherein introducing the nucleotide sequence encoding the NA protein into the composition increases efficient release of the VLP produced from the composition.

The present disclosure also provides a method of increasing stability and efficient release of a virus-like particle (VLP), the method comprising introducing a nucleotide sequence encoding a matrix-1 (M1) protein and a nucleotide sequence encoding a neuraminidase (NA) protein of an influenza virus into a composition, wherein the composition comprises a RNA comprising, in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding the NA protein of an influenza virus operably linked to a second subgenomic (SG) promoter;
c) a nucleotide sequence encoding the M1 protein of an influenza virus operably linked to a third subgenomic (SG) promoter; and
wherein introducing the nucleotide sequence encoding the M1 protein and the NA protein into the composition increases the stability and efficient release of the VLP produced from the composition.

In one example, the nucleotide sequence encoding a hemaglutinin (HA) protein of an influenza virus is operably linked to a nucleotide sequence comprising a 5′-untranslated region (5′-UTR), a fragment and/or a variant thereof. In one example, the 5′UTR comprises or consists of the sequence set forth in SEQ ID NO: 76.

In one example, the nucleotide sequence encoding a hemaglutinin (HA) protein of an influenza virus is operably linked to a first SG promoter. In one example, the first SG promoter is a native subgenomic promoter. In one example, the first SG promoter is derived from an alphavirus. In one example, the first SG promoter is derived from VEEV.

In one example, the second subgenomic promoter is derived from an alphavirus. In one example, the second subgenomic promoter is derived from VEEV. In one example, the second subgenomic promoter comprises or consists of the sequence set forth in SEQ ID NO: 3 or SEQ ID NO:75. In one example, the second subgenomic promoter consists of the sequence set forth in SEQ ID NO: 3. In one example, the second subgenomic promoter consists of the sequence set forth in SEQ ID NO: 75.

In one example, the third subgenomic promoter is derived from an alphavirus. In one example, the third subgenomic promoter is derived from VEEV. In one example, the third subgenomic promoter comprises or consists of the sequence set forth in SEQ ID NO: 3 or SEQ ID NO:75. In one example, the second subgenomic promoter consists of the sequence set forth in SEQ ID NO: 3. In one example, the second subgenomic promoter consists of the sequence set forth in SEQ ID NO: 75.

In one example, the second subgenomic promoter consists of the sequence set forth in SEQ ID NO: 75 and the third subgenomic promoter consists of the sequence set forth in SEQ ID NO: 3. Without wishing to be bound by theory, the present inventors have found that the combination of a second subgenomic promoter consisting of the sequence set forth in SEQ ID NO: 75 and a third subgenomic promoter consisting of the sequence set forth in SEQ ID NO: 3 increases the amount of NA and M1 protein incorporated in the VLPs.

### Methods of Treatment or Prevention

The present disclosure provides methods of treating or preventing or delaying progression of a influenza and/or influenza virus infection.

### Influenza

The present disclosure provides methods of treating, preventing or delaying the progression of influenza in a subject.

Influenza, also known as “the flu”, is an infectious disease caused by an influenza virus. Symptoms can be mild to severe and the most common symptoms include high fever, runny nose, sore throat, muscle and joint pain, headache, coughing, and feeling tired. Symptoms typically begin two days after exposure to the virus and most last less than a week. Complications of influenza may include viral pneumonia, secondary bacterial pneumonia, sinus infections, and worsening of previous health problems such as asthma or heart failure. Viral pneumonia may also lead to acute respiratory distress syndrome (ARDS).

Thus, in some examples of the present disclosure, the subject has an influenza virus infection. In one example, the subject has influenza. In particular, the influenza is associated with ARDS. In one example, the methods of the present disclosure can be used to treat or prevent ARDS in a subject suffering from an influenza virus infection. In one example, the methods of the present disclosure can be used to treat or prevent ARDS in a subject suffering from influenza.

In one example, the subject is at risk of having an influenza virus infection. In one example, the subject is at risk of having influenza. In particular, the influenza is associated with ARDS. In one example, the methods of the present disclosure can be used to prevent ARDS in a subject suffering from an influenza virus infection. In one example, the methods of the present disclosure can be used to prevent ARDS in a subject suffering from influenza virus infection. In one example, the methods of the present disclosure can be used to delay progression of ARDS in a subject suffering from influenza virus infection.

### Acute Respiratory Distress Syndrome (ARDS)

The present disclosure provides methods of treating, preventing or delaying the progression of ARDS in a subject.

ARDS is a life-threatening condition characterized by bilateral pulmonary infiltrates, severe hypoxemia, and disruption of the alveolar-capillary membrane barrier (i.e., pulmonary vascular leak), leading to non-cardiogenic pulmonary edema. There is currently no effective pharmacological therapy.

Infectious etiologies, including influenza, are leading causes of ARDS. Accordingly, in one example of the present disclosure, the ARDS is associated with an influenza infection. For example, the ARDS is associated with influenza.

ARDS is classified according to the Berlin Definition, which includes:
(1) presentation within 1 week of clinical insult or onset of respiratory symptoms;
(2) acute hypoxemic respiratory failure, as determined by a PaO2/FiO2 ratio of 300 mmHg or less on at least 5 cm of continuous positive airway pressure (CPAP) or positive end expiratory pressure (PEEP), where PaO2 is the partial pressure of oxygen in arterial blood and the FiO2 is the fraction of inspired oxygen;
(3) bilateral opacities on lung radiographs not fully explained by effusions, consolidation, or atelectasis; and
(4) edema/respiratory failure not fully explained by cardiac failure or fluid overload.

In one example, the subject has or suffers from ARDS (i.e., the subject satisfies the Berlin definition of ARDS). For example, the subject is in need of treatment (i.e., in need thereof).

In one example, the subject has or suffers from a symptom associated with ARDS. Symptoms associated with ARDS and methods of identifying subjects at risk of developing ARDS will be apparent to the skilled person and/or are described herein. For example, the subject has one or more or all of the following symptoms:
a) a respiratory frequency of greater than 30 breaths per minute;
b) an oxygen saturation (SpO₂) of 93% or less on room air;
c) a ratio of arterial partial pressure of oxygen to fraction of inspired oxygen (PaO2/FiO2) of less than 300 mmHg;
d) a SpO₂/FiO₂ ratio of less than 218; and
e) radiographic lung infiltrates in an amount of greater than 50%.

Currently, ARDS is classified as mild, moderate or severe with an associated increased mortality. The severity of ARDS can be categorized according to the Berlin definition as follows:
(i) Mild ARDS: PaO₂/FiO₂ of 200-300 mmHg on at least 5 cm CPAP or PEEP;
(ii) Moderate ARDS: PaO₂/FiO₂ of 100-200 mmHg on at least 5 cm PEEP; and
(iii) Severe ARDS: PaO₂/FiO₂ of less than or equal to 100 mmHg on at least 5 cm PEEP.

In one example, the ARDS is mild ARDS. In another example, the ARDS is moderate ARDS. In a further example, the ARDS is severe ARDS.

The methods of the present disclosure can, in addition to treatment of existing ARDS, be used to prevent or delay the onset of ARDS. Thus, in one example, the subject does not have ARDS.

In one example, the subject is at risk of developing one or more symptom(s) associated with ARDS.

### Kits

In one example, the present disclosure provides kits containing a DNA useful for producing the mRNA of the present disclosure. For example, the kit comprises the DNA plasmid and components for generating the mRNA. For example, components for generating the mRNA include sequencing and/or tail PCR primers, transcription reagents (e.g. T7 RNA polymerase), packaged with instructions to produce the mRNA of the present disclosure from the DNA.

In one example, the disclosure provides kits containing a mRNA of the present disclosure useful for the treatment or prevention of an influenza virus infection and/or influenza.

In one example, the kit comprises (a) a container comprising a RNA optionally in a delivery system and/or a pharmaceutically acceptable carrier or diluent; and (b) a package insert with instructions for treating or preventing influenza and/or an influenza virus infection in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the RNA. The label or package insert indicates that the composition is used for treating a subject eligible for treatment, e.g., one having or predisposed to developing influenza, an influenza virus infection, and/or ARDS, with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Generation of the RNA

DNA templates of sa-mRNA comprising a nucleotide sequence of a 5′-UTR set forth in SEQ ID NO: 76 and a nucleotide sequence of the 3′-UTR of SEQ ID NO: 80 were prepared using H5 and N1 subtypes from A/turkey/Turkey/1/2005 and M1 matrix protein from PR8X as the VLP forming elements. Nucleotide sequences encoding the N1 subtypes from A/turkey/Turkey/1/2005 and M1 matrix protein from PR8X were operably linked to a nucleotide sequence of extended subgenomic promoter (v2) set forth in SEQ ID NO: 75 or a nucleotide sequence of alphavirus native subgenomic promoter (v1) set forth in SEQ ID NO: 3. Nucleotide sequences encoding the H5 subtype from A/turkey/Turkey/1/2005 and N1 subtype from A/turkey/Turkey/1/2005 were operably linked to a nucleotide sequence of extended subgenomic promoter (v2) set forth in SEQ ID NO: 75 or a nucleotide sequence of alphavirus native subgenomic promoter (v1) set forth in SEQ ID NO: 3. The following constructs were prepared:
NSP1-4.nativeSGP.H5.SGPv1.N1.SGPv1.M1 (F554, SEQ ID NO: 81);
NSP1-4. nativeSGP.H5.SGPv1.N1.SGPv2.M1 (F624, SEQ ID NO: 82);
NSP1-4. nativeSGP.H5.SGPv2.N1.SGPv1.M1 (F625, SEQ ID NO: 83); and
NSP1-4. nativeSGP.H5.SGPv2.N1.SGPv2.M1 (F626, SEQ ID NO: 84).

sa-mRNAs used herein are made using standard laboratory methods and materials. The DNA templates encoding the sa-mRNA were produced in competent Escherichia coli cells that were transformed with a DNA plasmid. Individual bacterial colonies were isolated and the resultant plasmid DNA were amplified in E. coli cultures. Following fermentation, the plasmid DNA was isolated using Maxiprep DNA kit and linearized by restriction digest. Restriction enzymes were then removed using phenol/chloroform extraction and ethanol precipitation.

sa-mRNA was made by in vitro transcription from the linearized DNA template using a T7 RNA polymerase. Subsequently, the DNA template was removed by DNase digestion. The enzymatic capping was performed with Cap0 to provide functional mRNA. The resultant sa-mRNA was purified and resuspended in nuclease-free water.

The mRNA produced was formulated in a LNP to produce LNP-formulated sa-mRNA constructs.

### Example 2: In Vitro Characterisation of the Self-Replicating RNA and Virus-Like Particles (VLPs)

The composition comprising one or more RNAs comprising nucleotide sequences encoding the VLP forming elements produced in Example 1 was assessed for levels of expression. LNP-formulated mRNA constructs (50 μg) were incubated with a BHK cell line for 48 hrs.

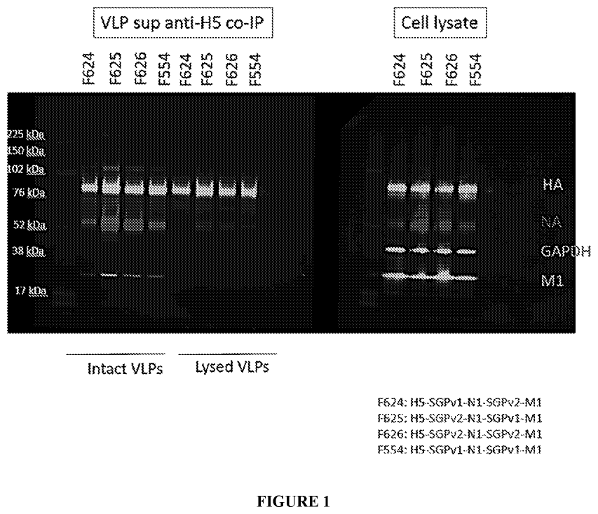

For in vitro quantification and characterization of VLPs, cell culture supernatant and cells from BHK cells incubated with LNP-formulated sa-mRNA constructs were collected. Cell culture supernatant was separated from BHK cells by centrifugation at 1000g for 5 minutes. VLPs present in cell culture supernatant were immunoprecipitated using anti-HA antibody (monoclonal antibody 15A6) conjugated to protein A beads. The incorporation of the three VLP forming elements (HA, NA and M1) into VLPs was assessed using western blot analysis. Western blot analysis was performed on BHK cell lysates and immunoprecipitated cell culture supernatant using anti-HA antibody (avian influenza A virus H5N3 HA antibody clone AT2B7), anti-NA antibody (influenza A H1N1 NA polyclonal), anti-M1 antibody (influenza A M1 monoclonal clone GA2B), and anti-GAPDH (control) (FIG. 1). FIG. 1 shows that intact VLPs were produced for all of the sa-mRNA constructs produced in Example 1, and that the VLPs were released into the supernatant from the BHK cells used to produce the VLPs.

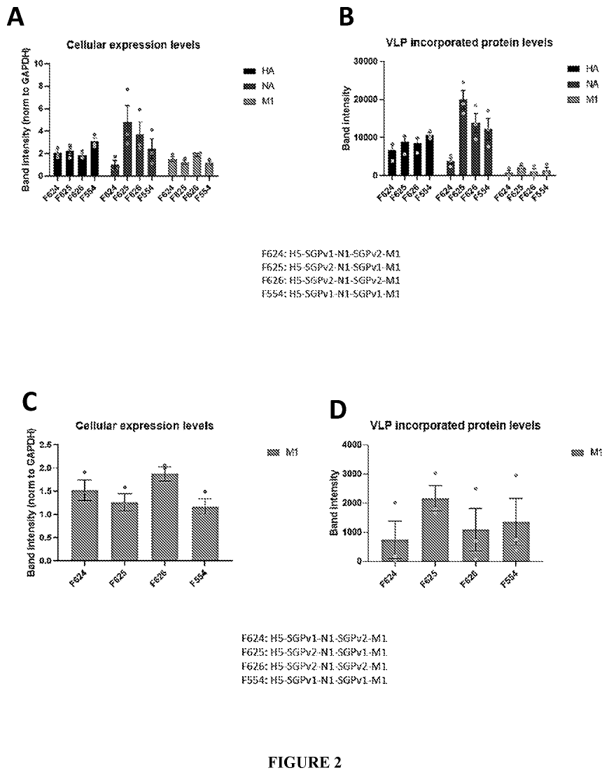

The levels of all three VLP forming elements (HA, NA and M1) in cell culture supernatants were quantified and compared between different sa-mRNA constructs using densitometry of the Western blots (FIG. 2). For further confirmation of VLP production, electron microscopy is used to visualize VLPs released from cells expressing VLP forming elements. The results of the densitometry analysis of the Western blots of FIG. 1 are summarized in Tables 1 and 2 below:

TABLE 1
Comparison of expression of HA, NA and M1 in BHK cells

| Construct design | H5 | N1 | M1 |
|---|---|---|---|
| F624: H5-SGPv1-N1-SGPv2-M1 | +++ | ++ | +++ |
| F625: H5-SGPv2-N1-SGPv1-M1 | +++ | +++++ | ++ |
| F626: H5-SGPv2-N1-SGPv2-M1 | +++ | +++++ | +++ |
| F554: H5-SGPv1-N1-SGPv1-M1 | +++ | +++ | ++ |

TABLE 2
Comparison of expression of HA, NA and M1 incorporation
in VLP of BHK cell culture supernatant

| Construct design | H5 | N1 | M1 |
|---|---|---|---|
| F624: H5-SGPv1-N1-SGPv2-M1 | +++ | ++ | ++ |
| F625: H5-SGPv2-N1-SGPv1-M1 | +++ | +++++ | +++ |
| F626: H5-SGPv2-N1-SGPv2-M1 | +++ | ++++ | ++ |
| F554: H5-SGPv1-N1-SGPv1-M1 | +++ | +++ | ++ |

sa-mRNA construct F626 produced the greatest amount of M1 protein in BHK cells but construct F625 had the highest level of M1 protein incorporated in the VLPs (FIGS. 2C and 2D). sa-mRNA construct F625 had the highest level of incorporation of HA, NA and M1 protein in the VLPs (FIG. 2B and Table 2).

### Example 3: Further Characterisation of the Self-Replicating RNA and Virus-Like Particles (VLPs)

### Antibody Responses

To evaluate the antibody immune response in a preclinical animal model, mice can be immunized with LNP-formulated sa-mRNA constructs at Day 0 with a suitable dose (e.g. 0.1 pg or 0.001 pg), with a second dose at Day 21. To assess antibody responses, serum is collected at the end of study (i.e., 42 days after first or 21 days after the second vaccine dose) and is tested by microneutralization assays and hemagglutination inhibition assay.

For all serological assays sera are treated in the same way, with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) is used as positive control sera three assays.

### Microneutralization Assays

Microneutralization assays, short and long form, are performed in a qualified mammalian cell line (proprietary 33016-PF Madin-Darby Canine Kidney (MDCK)).

### Microneutralization Assay Short Form (MN Assay SF)

Virus fluorescent focus-based microneutralization (FFA MN) assay is performed using an in-house developed protocol. RDE treated test mouse samples and positive control sera are heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 μg/mL streptomycin (GIBCO)). A/turkey/Turkey/1/2005 (H5N1) virus is diluted to ˜1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells are inoculated with this mixture and incubated overnight for 16-18 h at 37° C. with 5% CO₂. MDCK 33016-PF cells are seeded at 3.0E4/well (3.0E6/plate) at 6-8h earlier in the cell growth medium (comprised of D-MEM, supplemented with 10% HyClone fetal bovine serum—FBS (Gibco), 100 U/mL penicillin and 100 μg/mL streptomycin). Following the overnight incubation and prior to immunostaining, cells are fixed with cold mixture of acetone and methanol.

The virus is visualized using separate 1 h incubations at room temperature of monoclonal antibodies specific to the nucleoproteins (NP) of the influenza A viruses (clones A1, A3 Blend, Millipore cat. no. MAB8251) and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). NP viral protein is quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci is enumerated by use of software Immunospot 7.0.12.1 professional analyzer DC, using a custom analysis module. The data is successively logged by this software into an Excel data analysis spreadsheet, then 60% focus reduction endpoint is calculated from the average foci count of virus control wells (for each plate), and 60% focus reduction neutralization titer is calculated by linear interpolation between wells immediately above and below the 60% endpoint (for each sample).

### Microneutralization Assay Long Form (MN Assay LF)

MN assay LF is performed using an in-house developed protocol. RDE treated test mouse samples and positive control sera are heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 μg/mL streptomycin (GIBCO), and 0.33 ug/mL TPCK-trypsin (TPCK treated, Tosyl phenylalanyl chloromethyl ketone, Sigma). A/turkey/Turkey/1/2005 (H5N1) virus is diluted to 100TCID (tissue culture infectious dose) per well in neutralization medium and added in a 1:1 ratio to diluted serum. Serially pre-diluted serum samples are incubated with the virus and allowed to react for 1 h at 37° C., 5% CO₂. In the inoculation step, plates (Cell Culture 96-well plate, Costar) containing MDCK 33016-PF cells (which had been seeded as 3.0E4/well (3.0E6/plate) at day before in the antibiotic free cell growth medium (Irvine Scientific)) are washed with sterile PBS, then infected with this mixture and incubated for 1 h at 37° C. with 5% CO₂. Infection is stopped by aspiration of antibody/virus mixture and washed cells with sterile PBS are inoculated with neutralizing media (100 ul/well) containing twofold serially diluted antibodies, then incubated for 5 days at 37° C. with 5% CO₂. In the final “read-out” step, detection of virus is performed by HA quantification of the virus using 0.5% turkey red blood cells (Lampire Biological Laboratories). The absence of infectivity constitutes a positive neutralization reaction and indicates the presence of virus-specific antibodies in the serum sample.

### Hemagglutination Inhibition (HAI) Assay

HAI assay is performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera are heat inactivated, diluted to a starting dilution of 1:10 with PBS, and twofold serial diluted samples (25 μl) are incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) are added and incubated at RT for 30 minutes. The HAI titer is expressed as the reciprocal of the highest dilution of the samples inhibiting hemagglutination.

### Neuraminidase Inhibition (NAI) Assay

NAI assay is performed as previously described (WHO (2011) Manual for the laboratory diagnosis and virological surveillance of influenza: WHO Press, World Health Organization, 20 Avenue Appia, 1211 Geneva 27, Switzerland). Briefly, virus standards are diluted to a starting dilution of 1:10 in PBS, and fourfold serial diluted samples (50 μl) are incubated with equal volumes of heat inactivated RDE treated test mouse samples and positive control sera at 37° C. for 30 minutes. Mixture of PBS and fetuin (mixture 1:1) are added (50 μl) to the incubated samples and further incubated at 37° C. for 18 hours. The samples are cooled, periodate reagent is added to each sample (50 μl) and incubated at room temperature (RT) for 20 minutes. Arsenite reagent (0.25 ml) is added to the samples followed by 2-TBA reagent (0.5 ml) and boiled for 15 minutes. Samples are cooled to RT and Warrenoff reagent (1 ml) added to produce an organic (butanol) phase which is collected for colorimeteric analaysis at an optical density (OD) reading of 549 nm. The NAI titre is defined as the dilution giving 50% inhibition of NA activity (NAI₅₀).

### Example 4: RNA Induces Cell-Mediated Immune Responses

The composition comprising one or more RNAs comprising nucleotide sequences encoding the VLP forming elements are assessed for their ability to induce antigen specific T cell responses.

Antigen-specific T cell detection is performed on splenic cultures. Briefly, splenocytes are dissociated in dissociation solution (MACS BSA stock 1:20 with autoMACS rinsing solution) and concentrated at 4E7 cells/ml. Briefly, splenocyte cultures are established in 96 well plates in T cell medium containing RPMI, NEAA, pen/strep and PME) and cultured at 37° C./5% CO₂. Anti-CD28 (clone 37.51; BD Biosciences #553294) and anti-CD107a (clone #1D4B; Biolegend #121618) are added to each well. Cell cultures are either stimulated or unstimulated. To stimulate cultures NA pep mix (JPT Peptide Technologies GmbH; PM-INFA-NATur), HA pep mix (JPT Peptide Technologies GmbH; PM-INFA-HAIndo) are added. Following 2 hours of stimulation, Golgi Plug (with brefeldin A; BD Biosciences #555029) are added to each well. Cells are incubated at 37° C. for a total of 6 hours after which the cells are transferred to 4° C. and stored overnight.

Antigen-specific T cell responses are determined using flow cytometry. Briefly, Fc block mixture (clone 2.4G2; BD Biosciences #553142) is added to each well, followed by extracellular stain (comprising Brilliant stain buffer plus (BD Biosciences #566385), ICOS BV711 (clone C398.4A; Biolegend #313548), CD44 BUV395 (clone IM7; BD Biosciences #740215), CD3 BV786 (clone 145-2C11; BD Biosciences #564379), CD4 APC-H7 (clone GK1.5, BD Biosciences #560181), CD8 AF700 (clone 53-6.7, BD Biosciences #557959) and staining buffer). Cells are stained with UltraComp eBeads (eBiosciences #01-222-42) according to the manufacturer's protocol and incubated at 4° C. for 30 mins, protected from the light. Cells are washed with staining buffer, centrifuged, resuspended in staining buffer and data is acquired using a flow cytometer.

SEQUENCES

| SEQ | |
|---|---|
| ID | |
| NO: | Sequence (5′ to 3′) |
| 1 | ACCATGG |
| 2 | ACCATG |
| 3 | CTCTCTACGGCTAACCTGAATGGACTACGACATAGTCTAGTCCGCCAAG |
| 4 | ACTCTAGCTAGCAGTGTTAAATCATTCAGCTACCTGAGAGGGGCCCCTATAACTCTCTACGGCTAACCTGAA |
| | TGGACTACGACATAGTCTAGTCCGCCAAG |
| 5 | CAAAGGCTCTTTTCAGAGCCACCA |
| 6 | GCACACTGCCAGTCTCACTGTGGGAAGGCCAGCTGCCCCAGCCAGATGGACTCCAGCCTGCCTGGCAGGCTG |
| | TCTGGCCAGGCCTCTTGGCAGTCACATCTCTTCCTCCGAGGCCAGTACCTTTCCATTTATTCTTTGATCTTC |
| | AGGGAACTGCATAGATTGATCAAAGTGTAAACACCATAGGGACCCATTCTACACAGAGCAGGACTGCACAGC |
| | GTCCTGTCCACACCCAGCTCAGCATTTCCACACCAAGCAGCAACAGCAAATCACGACCACTGATAGATGTCT |
| | ATTCTTGTTGGAGACATGGGATGATTATTTTCTGTTCTATTTGTGCTTAGTCCAATTCCTTGCACATAGTAG |
| | GTACCCAATTCAATTACTATTGAATGAATTAAGAATTGGTTGCCATAAAAATAAATCAGTTCATTTAAAA |
| 7 | ACTCCCTCCATCCCAACCTGGCTCCCTCCCACCCAACCAACTTTCCCCCCAACCCGGAAACAGACAAGCAAC |
| | CCAAACTGAACCCCCTCAAAAGCCAAAAAATGGGAGACAATTTCACATGGACTTTGGAAAATATTTTTTTCC |
| | TTTGCATTCATCTCTCAAACTTAGTTTTTATCTTTGACCAACCGAACATGACCAAAAACCAAAAGTGCATTC |
| | AACCTTACCAAAAAAAAAAAAAAAAAAAGAATAAATAAATAACTTTTTAAAAAAGGAAGCTTGGTCCACTTG |
| | CTTGAAGACCCATGCGGGGGTAAGTCCCTTTCTGCCCGTTGGGCTTATGAAACCCCAATGCTGCCCTTTCTG |
| | CTCCTTTCTCCACACCCCCCTTGGGGCCTCCCCTCCACTCCTTCCCAAATCTGTCTCCCCAGAAGACACAGG |
| | AAACAATGTATTGTCTGCCCAGCAATCAAAGGCAATGCTCAAACACCCAAGTGGCCCCCACCCTCAGCCCGC |
| | TCCTGCCCGCCCAGCACCCCCAGGCCCTGGGGGACCTGGGGTTCTCAGACTGCCAAAGAAGCCTTGCCATCT |
| | GGCGCTCCCATGGCTCTTGCAACATCTCCCCTTCGTTTTTGAGGGGGTCATGCCGGGGGAGCCACCAGCCCC |
| | TCACTGGGTTCGGAGGAGAGTCAGGAAGGGCCACGACAAAGCAGAAACATCGGATTTGGGGAACGCGTGTCA |
| | ATCCCTTGTGCCGCAGGGCTGGGCGGGAGAGACTGTTCTGTTCCTTGTGTAACTGTGTTGCTGAAAGACTAC |
| | CTCGTTCTTGTCTTGATGTGTCACCGGGGCAACTGCCTGGGGGCGGGGATGGGGGCAGGGTGGAAGCGGCTC |
| | CCCATTTTATACCAAAGGTGCTACATCTATGTGATGGGTGGGGTGGGGAGGGAATCACTGGTGCTATAGAAA |
| | TTGAGATGCCCCCCCAGGCCAGCAAATGTTCCTTTTTGTTCAAAGTCTATTTTTATTCCTTGATATTTTTCT |
| | TTTTTTTTTTTTTTTTTTGTGGATGGGGACTTGTGAATTTTTCTAAAGGTGCTATTTAACATGGGAGGAGAG |
| | CGTGTGCGGCTCCAGCCCAGCCCGCTGCTCACTTTCCACCCTCTCTCCACCTGCCTCTGGCTTCTCAGGCCT |
| | CTGCTCTCCGACCTCTCTCCTCTGAAACCCTCCTCCACAGCTGCAGCCCATCCTCCCGGCTCCCTCCTAGTC |
| | TGTCCTGCGTCCTCTGTCCCCGGGTTTCAGAGACAACTTCCCAAAGCACAAAGCAGTTTTTCCCCCTAGGGG |
| | TGGGAGGAAGCAAAAGACTCTGTACCTATTTTGTATGTGTATAATAATTTGAGATGTTTTTAATTATTTTGA |
| | TTGCTGGAATAAAGCATGTGGAAATGACCCAAA |
| 8 | GTGCACGGCGTCCCTGAGGGCCCTTCCCAACCTCCCCTGGTCCTGCACTGTCCCGGAGCTCAGGCCCTGGTG |
| | AGGGGCTGGGTCCCGGGTGCCCCCCATGCCCTCCCTGCTGCCAGGCTCCCACTGCCCCTGCACCTGCTTCTC |
| | AGCGCAACAGCTGTGTGTGCCCGTGGTGAGGTTGTGCTGCCTGTGGTGAGGTCCTGTCCTGGCTCCCAGGGT |
| | CCTGGGGGCTGCTGCACTGCCCTCCGCCCTTCCCTGACACTGTCTGCTGCCCCAATCACCGTCACAATAAAA |
| | GAAACTGTGGTCTCTA |
| 9 | GTACATGGAGGCCAACAAGAACATAGGCAAGATCGTCCTGGAACTGCCCCAGTGAAGGAGGATGGGGCAGGA |
| | CAGGACGCGGCCACCCCAGGCCTTTCCAGAGCAAACCTGGAGAAGATTCACAATAGACAGGCCAAGAAACCC |
| | GGTGCTTCCTCCAGAGCCGTTTAAAGCTGATATGAGGAAATAAAGAGTGAACTGGAAAAAAAAAA |
| 10 | GGGAGATGCCCCACAGCACTGCCCTTCCAGAGGCAAGACCAACCAAG |
| 11 | GGGAGATATATATAGGATTGAAGATCTCTCAGTTAAGTCTACATGAAAAGG |
| 12 | GGGACTGCCCTTCCAGAGGCAAGACCAACCAAG |
| 13 | GGGAGTATATTAGTGCTAATTTCCCTCCGTTTGTCCTAGCTTTTCTCTTCTGTCAACCCCACA |
| 14 | GGGAGATAAAAAGCCAGCTCCAGCAGGCGCTGCTCACTCCTCCCCATCCTCTCCCTCTGTCCCTCTGTCCCT |
| | CTGACCCTGCACTGTCCCAGCACC |
| 15 | GGGAGCAATCCTTTCTTTCAGCTGGAGTGCTCCTCAGGAGCCAGCCCCACCCTTAGAAAAG |
| 16 | GGGGTGTCCCATAGTGTTTCCAAACTTGGAAAGGGCGGGGGAGGGCGGGAGGATGCGGAGGGCGGAGGTATG |
| | CAGACAACGAGTCAGAGTTTCCCCTTGAAAGCCTCAAAAGTGTCCACGTCCTCAAAAAGAATGGAACCAATT |
| | TAAGAAGCCAGCCCCGTGGCCACGTCCCTTCCCCCATTCGCTCCCTCCTCTGCGCCCCCGCAGGCTCCTCCC |
| | AGCTGTGGCTGCCCGGGCCCCCAGCCCCAGCCCTCCCATTGGTGGAGGCCCTTTTGGAGGCACCCTAGGGCC |
| | AGGGAAACTTTTGCCGTATAAATAGGGCAGATCCGGGCTTTATTATTTTAGCACCACGGCAGCAGGAGGTTT |
| | CGGCTAAGTTGGAGGTACTGGCCACGACTGCATGCCCGCGCCCGCCAGGTGATACCTCCGCCGGTGACCCAG |
| | GGGCTCTGCGACACAAGGAGTCTGCATGTCTAAGTGCTAGAC |
| 17 | GGGGCTTACTCGGCGCCCGCGCCTCGGGCCGTCGGGAGCGGAGCCTCCTCGGGACCAGGACTTCAGGGCCAC |
| | AGGTGCTGCCAAG |
| 18 | GGGATTCATGAAAATCCACTACTCCAGACAGACGGCTTTGGAATCCACCAGCTACATCCAGCTCCCTGAGGC |
| | AGAGTTGAGA |
| 19 | GGGACAATGACTCCTTTCGGTAAGTGCAGTGGAAGCTGTACACTGCCCAGGCAAAGCGTCCGGGCAGCGTAG |
| | GCGGGCGACTCAGATCCCAGCCAGTGGACTTAGCCCCTGTTTGCTCCTCCGATAACTGGGGTGACCTTGGTT |
| | AATATTCACCAGCAGCCTCCCCCGTTGCCCCTCTGGATCCACTGCTTAAATACGGACGAGGACAGGGCCCTG |
| | TCTCCTCAGCTTCAGGCACCACCACTGACCTGGGACAGTGAATCGACA |
| 20 | GGGAAATAAGAGAGAAAAGAAGAGTAAGAAGAAATATAAGAGCCACC |
| 21 | GGGAGATCAGAGAGAAAAGAAGAGTAAGAAGAAATATAAGAGCCACC |
| 22 | GGAATAAAAGTCTCAACACAACATATACAAAACAAACGAATCTCAAGCAATCAAGCATTCTACTTCTATTGC |
| | AGCAATTTAAATCATTTCTTTTAAAGCAAAAGCAATTTTCTGAAAATTTTCACCATTTACGAACGATAGCAA |
| | C |
| 23 | GGGAGACAAGCTTGGCATTCCGGTACTGTTGGTAAAGCCACC |
| 24 | GGGAGATCAGAGAGAAAAGAAGAGTAAGAAGAAATATAAGAGCCACC |
| 25 | GGAATAAAAGTCTCAACACAACATATACAAAACAAACGAATCTCAAGCAATCAAGCATTCTACTTCTATTGC |
| | AGCAATTTAAATCATTTCTTTTAAAGCAAAAGCAATTTTCTGAAAATTTTCACCATTTACGAACGATAGCAA |
| | C |
| 26 | GGGAGACAAGCTTGGCATTCCGGTACTGTTGGTAAAGCCACC |
| 27 | GGGAATTAACAGAGAAAAGAAGAGTAAGAAGAAATATAAGAGCCACC |
| 28 | GGGAAATTAGACAGAAAAGAAGAGTAAGAAGAAATATAAGAGCCACC |
| 29 | GGGAAATAAGAGAGTAAAGAACAGTAAGAAGAAATATAAGAGCCACC |
| 30 | GGGAAAAAAGAGAGAAAAGAAGACTAAGAAGAAATATAAGAGCCACC |
| 31 | GGGAAATAAGAGAGAAAAGAAGAGTAAGAAGATATATAAGAGCCACC |
| 32 | GGGAAATAAGAGACAAAACAAGAGTAAGAAGAAATATAAGAGCCACC |
| 33 | GGGAAATTAGAGAGTAAAGAACAGTAAGTAGAATTAAAAGAGCCACC |
| 34 | GGGAAATAAGAGAGAATAGAAGAGTAAGAAGAAATATAAGAGCCACC |
| 35 | GGGAAATAAGAGAGAAAAGAAGAGTAAGAAGAAAATTAAGAGCCACC |
| 36 | GGGAAATAAGAGAGAAAAGAAGAGTAAGAAGAAATTTAAGAGCCACC |
| 37 | GGGAAATAAGAGAGAAAAGAAGAGTAAGAAGAAATATAAGAGCCTCC |
| 38 | GGGAAATAAGAGAGAAAAGAAGAGTAAGAAGAAATATATGA |
| 39 | GGGAAATAAGAGAGAAAAGAAGAGTAAGAAGAAATATA |
| 40 | GCGCCTGCCCACCTGCCACCGACTGCTGGAACCCAGCCAGTGGGAGGGCCTGGCCCACCAGAGTCCTGCTCC |
| | CTCACTCCTCGCCCCGCCCCCTGTCCCAGAGTCCCACCTGGGGGCTCTCTCCACCCTTCTCAGAGTTCCAGT |
| | TTCAACCAGAGTTCCAACCAATGGGCTCCATCCTCTGGATTCTGGCCAATGAAATATCTCCCTGGCAGGGTC |
| | CTCTTCTTTTCCCAGAGCTCCACCCCAACCAGGAGCTCTAGTTAATGGAGAGCTCCCAGCACACTCGGAGCT |
| | TGTGCTTTGTCTCCACGCAAAGCGATAAATAAAAGCATTGGTGGCCTTTGGTCTTTGAATAAAGCCTGAGTA |
| | GGAAGTCTAGA |
| 41 | GCCCCTGCCGCTCCCACCCCCACCCATCTGGGCCCCGGGTTCAAGAGAGAGCGGGGTCTGATCTCGTGTAGC |
| | CATATAGAGTTTGCTTCTGAGTGTCTGCTTTGTTTAGTAGAGGTGGGCAGGAGGAGCTGAGGGGCTGGGGCT |
| | GGGGTGTTGAAGTTGGCTTTGCATGCCCAGCGATGCGCCTCCCTGTGGGATGTCATCACCCTGGGAACCGGG |
| | AGTGGCCCTTGGCTCACTGTGTTCTGCATGGTTTGGATCTGAATTAATTGTCCTTTCTTCTAAATCCCAACC |
| | GAACTTCTTCCAACCTCCAAACTGGCTGTAACCCCAAATCCAAGCCATTAACTACACCTGACAGTAGCAATT |
| | GTCTGATTAATCACTGGCCCCTTGAAGACAGCAGAATGTCCCTTTGCAATGAGGAGGAGATCTGGGCTGGGC |
| | GGGCCAGCTGGGGAAGCATTTGACTATCTGGAACTTGTGTGTGCCTCCTCAGGTATGGCAGTGACTCACCTG |
| | GTTTTAATAAAACAACCTGCAACATCTCATGGTCTTTGAATAAAGCCTGAGTAGGAAGTCTAGA |
| 42 | ACACACTCCACCTCCAGCACGCGACTTCTCAGGACGACGAATCTTCTCAATGGGGGGGCGGCTGAGCTCCAG |
| | CCACCCCGCAGTCACTTTCTTTGTAACAACTTCCGTTGCTGCCATCGTAAACTGACACAGTGTTTATAACGT |
| | GTACATACATTAACTTATTACCTCATTTTGTTATTTTTCGAAACAAAGCCCTGTGGAAGAAAATGGAAAACT |
| | TGAAGAAGCATTAAAGTCATTCTGTTAAGCTGCGTAAATGGTCTTTGAATAAAGCCTGAGTAGGAAGTCTAG |
| | A |
| 43 | CATCACATTTAAAAGCATCTCAGCCTACCATGAGAATAAGAGAAAGAAAATGAAGATCAAAAGCTTATTCAT |
| | CTGTTTTTCTTTTTCGTTGGTGTAAAGCCAACACCCTGTCTAAAAAACATAAATTTCTTTAATCATTTTGCC |
| | TCTTTTCTCTGTGCTTCAATTAATAAAAAATGGAAAGAATCTAATAGAGTGGTACAGCACTGTTATTTTTCA |
| | AAGATGTGTTGCTATCCTGAAAATTCTGTAGGTTCTGTGGAAGTTCCAGTGTTCTCTCTTATTCCACTTCGG |
| | TAGAGGATTTCTAGTTTCTTGTGGGCTAATTAAATAAATCATTAATACTCTTCTAATGGTCTTTGAATAAAG |
| | CCTGAGTAGGAAGTCTAGA |
| 44 | GCTGCCTTCTGCGGGGCTTGCCTTCTGGCCATGCCCTTCTTCTCTCCCTTGCACCTGTACCTCTTGGTCTTT |
| | GAATAAAGCCTGAGTAGGAAGGCGGCCGCTCGAGCATGCATCTAGA |
| 45 | GCCAAGCCCTCCCCATCCCATGTATTTATCTCTATTTAATATTTATGTCTATTTAAGCCTCATATTTAAAGA |
| | CAGGGAAGAGCAGAACGGAGCCCCAGGCCTCTGTGTCCTTCCCTGCATTTCTGAGTTTCATTCTCCTGCCTG |
| | TAGCAGTGAGAAAAAGCTCCTGTCCTCCCATCCCCTGGACTGGGAGGTAGATAGGTAAATACCAAGTATTTA |
| | TTACTATGACTGCTCCCCAGCCCTGGCTCTGCAATGGGCACTGGGATGAGCCGCTGTGAGCCCCTGGTCCTG |
| | AGGGTCCCCACCTGGGACCCTTGAGAGTATCAGGTCTCCCACGTGGGAGACAAGAAATCCCTGTTTAATATT |
| | TAAACAGCAGTGTTCCCCATCTGGGTCCTTGCACCCCTCACTCTGGCCTCAGCCGACTGCACAGCGGCCCCT |
| | GCATCCCCTTGGCTGTGAGGCCCCTGGACAAGCAGAGGTGGCCAGAGCTGGGAGGCATGGCCCTGGGGTCCC |
| | ACGAATTTGCTGGGGAATCTCGTTTTTCTTCTTAAGACTTTTGGGACATGGTTTGACTCCCGAACATCACCG |
| | ACGCGTCTCCTGTTTTTCTGGGTGGCCTCGGGACACCTGCCCTGCCCCCACGAGGGTCAGGACTGTGACTCT |
| | TTTTAGGGCCAGGCAGGTGCCTGGACATTTGCCTTGCTGGACGGGGACTGGGGATGTGGGAGGGAGCAGACA |
| | GGAGGAATCATGTCAGGCCTGTGTGTGAAAGGAAGCTCCACTGTCACCCTCCACCTCTTCACCCCCCACTCA |
| | CCAGTGTCCCCTCCACTGTCACATTGTAACTGAACTTCAGGATAATAAAGTGTTTGCCTCCATGGTCTTTGA |
| | ATAAAGCCTGAGTAGGAAGGCGGCCGCTCGAGCATGCATCTAGA |
| 46 | ACTCAATCTAAATTAAAAAAGAAAGAAATTTGAAAAAACTTTCTCTTTGCCATTTCTTCTTCTTCTTTTTTA |
| | ACTGAAAGCTGAATCCTTCCATTTCTTCTGCACATCTACTTGCTTAAATTGTGGGCAAAAGAGAAAAAGAAG |
| | GATTGATCAGAGCATTGTGCAATACAGTTTCATTAACTCCTTCCCCCGCTCCCCCAAAAATTTGAATTTTTT |
| | TTTCAACACTCTTACACCTGTTATGGAAAATGTCAACCTTTGTAAGAAAACCAAAATAAAAATTGAAAAATA |
| | AAAACCATAAACATTTGCACCACTTGTGGCTTTTGAATATCTTCCACAGAGGGAAGTTTAAAACCCAAACTT |
| | CCAAAGGTTTAAACTACCTCAAAACACTTTCCCATGAGTGTGATCCACATTGTTAGGTGCTGACCTAGACAG |
| | AGATGAACTGAGGTCCTTGTTTTGTTTTGTTCATAATACAAAGGTGCTAATTAATAGTATTTCAGATACTTG |
| | AAGAATGTTGATGGTGCTAGAAGAATTTGAGAAGAAATACTCCTGTATTGAGTTGTATCGTGTGGTGTATTT |
| | TTTAAAAAATTTGATTTAGCATTCATATTTTCCATCTTATTCCCAATTAAAAGTATGCAGATTATTTGCCCA |
| | AATCTTCTTCAGATTCAGCATTTGTTCTTTGCCAGTCTCATTTTCATCTTCTTCCATGGTTCCACAGAAGCT |
| | TTGTTTCTTGGGCAAGCAGAAAAATTAAATTGTACCTATTTTGTATATGTGAGATGTTTAAATAAATTGTGA |
| | AAAAAATGAAATAAAGCATGTTTGGTTTTCCAAAAGAACATAT |
| 47 | CGCCGCCGCCCGGGCCCCGCAGTCGAGGGTCGTGAGCCCACCCCGTCCATGGTGCTAAGCGGGCCCGGGTCC |
| | CACACGGCCAGCACCGCTGCTCACTCGGACGACGCCCTGGGCCTGCACCTCTCCAGCTCCTCCCACGGGGTC |
| | CCCGTAGCCCCGGCCCCCGCCCAGCCCCAGGTCTCCCCAGGCCCTCCGCAGGCTGCCCGGCCTCCCTCCCCC |
| | TGCAGCCATCCCAAGGCTCCTGACCTACCTGGCCCCTGAGCTCTGGAGCAAGCCCTGACCCAATAAAGGCTT |
| | TGAACCCAT |
| 48 | GGGGCTAGAGCCCTCTCCGCACAGCGTGGAGACGGGGCAAGGAGGGGGGTTATTAGGATTGGTGGTTTTGTT |
| | TTGCTTTGTTTAAAGCCGTGGGAAAATGGCACAACTTTACCTCTGTGGGAGATGCAACACTGAGAGCCAAGG |
| | GGTGGGAGTTGGGATAATTTTTATATAAAAGAAGTTTTTCCACTTTGAATTGCTAAAAGTGGCATTTTTCCT |
| | ATGTGCAGTCACTCCTCTCATTTCTAAAATAGGGACGTGGCCAGGCACGGTGGCTCATGCCTGTAATCCCAG |
| | CACTTTGGGAGGCCGAGGCAGGCGGCTCACGAGGTCAGGAGATCGAGACTATCCTGGCTAACACGGTAAAAC |
| | CCTGTCTCTACTAAAAGTACAAAAAATTAGCTGGGCGTGGTGGTGGGCACCTGTAGTCCCAGCTACTCGGGA |
| | GGCTGAGGCAGGAGAAAGGCATGAATCCAAGAGGCAGAGCTTGCAGTGAGCTGAGATCACGCCATTGCACTC |
| | CAGCCTGGGCAACAGTGTTAAGACTCTGTCTCAAATATAAATAAATAAATAAATAAATAAATAAATAAATAA |
| | AAATAAAGCGAGATGTTGCCCTCAAA |
| 49 | GGCCCTGCCCCGTCGGACTGCCCCCAGAAAGCCTCCTGCCCCCTGCCAGTGAAGTCCTTCAGTGAGCCCCTC |
| | CCCAGCCAGCCCTTCCCTGGCCCCGCCGGATGTATAAATGTAAAAATGAAGGAATTACATTTTATATGTGAG |
| | CGAGCAAGCCGGCAAGCGAGCACAGTATTATTTCTCCATCCCCTCCCTGCCTGCTCCTTGGCACCCCCATGC |
| | TGCCTTCAGGGAGACAGGCAGGGAGGGCTTGGGGCTGCACCTCCTACCCTCCCACCAGAACGCACCCCACTG |
| | GGAGAGCTGGTGGTGCAGCCTTCCCCTCCCTGTATAAGACACTTTGCCAAGGCTCTCCCCTCTCGCCCCATC |
| | CCTGCTTGCCCGCTCCCACAGCTTCCTGAGGGCTAATTCTGGGAAGGGAGAGTTCTTTGCTGCCCCTGTCTG |
| | GAAGACGTGGCTCTGGGTGAGGTAGGCGGGAAAGGATGGAGTGTTTTAGTTCTTGGGGGAGGCCACCCCAAA |
| | CCCCAGCCCCAACTCCAGGGGCACCTATGAGATGGCCATGCTCAACCCCCCTCCCAGACAGGCCCTCCCTGT |
| | CTCCAGGGCCCCCACCGAGGTTCCCAGGGCTGGAGACTTCCTCTGGTAAACATTCCTCCAGCCTCCCCTCCC |
| | CTGGGGACGCCAAGGAGGTGGGCCACACCCAGGAAGGGAAAGCGGGCAGCCCCGTTTTGGGGACGTGAACGT |
| | TTTAATAATTTTTGCTGAATTCCTTTACAACTAAATAACACAGATATTGTTATAAATAAAATTGT |
| 50 | ATATTAAGGATCAAGCTGTTAGCTAATAATGCCACCTCTGCAGTTTTGGGAACAGGCAAATAAAGTATCAGT |
| | ATACATGGTGATGTACATCTGTAGCAAAGCTCTTGGAGAAAATGAAGACTGAAGAAAGCAAAGCAAAAACTG |
| | TATAGAGAGATTTTTCAAAAGCAGTAATCCCTCAATTTTAAAAAAGGATTGAAAATTCTAAATGTCTTTCTG |
| | TGCATATTTTTTGTGTTAGGAATCAAAAGTATTTTATAAAAGGAGAAAGAACAGCCTCATTTTAGATGTAGT |
| | CCTGTTGGATTTTTTATGCCTCCTCAGTAACCAGAAATGTTTTAAAAAACTAAGTGTTTAGGATTTCAAGAC |
| | AACATTATACATGGCTCTGAAATATCTGACACAATGTAAACATTGCAGGCACCTGCATTTTATGTTTTTTTT |
| | TTCAACAAATGTGACTAATTTGAAACTTTTATGAACTTCTGAGCTGTCCCCTTGCAATTCAACCGCAGTTTG |
| | AATTAATCATATCAAATCAGTTTTAATTTTTTAAATTGTACTTCAGAGTCTATATTTCAAGGGCACATTTTC |
| | TCACTACTATTTTAATACATTAAAGGACTAAATAATCTTTCAGAGATGCTGGAAACAAATCATTTGCTTTAT |
| | ATGTTTCATTAGAATACCAATGAAACATACAACTTGAAAATTAGTAATAGTATTTTTGAAGATCCCATTTCT |
| | AATTGGAGATCTCTTTAATTTCGATCAACTTATAATGTGTAGTACTATATTAAGTGCACTTGAGTGGAATTC |
| | AACATTTGACTAATAAAATGAGTTCATCATGTTGGCAAGTGATGTGGCAATTATCTCTGGTGACAAAAGAGT |
| | AAAATCAAATATTTCTGCCTGTTACAAATATCAAGGAAGACCTGCTACTATGAAATAGATGACATTAATCTG |
| | TCTTCACTGTTTATAATACGGATGGATTTTTTTTCAAATCAGTGTGTGTTTTGAGGTCTTATGTAATTGATG |
| | ACATTTGAGAGAAATGGTGGCTTTTTTTAGCTACCTCTTTGTTCATTTAAGCACCAGTAAAGATCATGTCTT |
| | TTTATAGAAGTGTAGATTTTCTTTGTGACTTTGCTATCGTGCCTAAAGCTCTAAATATAGGTGAATGTGTGA |
| | TGAATACTCAGATTATTTGTCTCTCTATATAATTAGTTTGGTACTAAGTTTCTCAAAAAATTATTAACACAT |
| | GAAAGACAATCTCTAAACCAGAAAAAGAAGTAGTACAAATTTTGTTACTGTAATGCTCGCGTTTAGTGAGTT |
| | TAAAACACACAGTATCTTTTGGTTTTATAATCAGTTTCTATTTTGCTGTGCCTGAGATTAAGATCTGTGTAT |
| | GTGTGTGTGTGTGTGTGTGCGTTTGTGTGTTAAAGCAGAAAAGACTTTTTTAAAAGTTTTAAGTGATAAATG |
| | CAATTTGTTAATTGATCTTAGATCACTAGTAAACTCAGGGCTGAATTATACCATGTATATTCTATTAGAAGA |
| | AAGTAAACACCATCTTTATTCCTGCCCTTTTTCTTCTCTCAAAGTAGTTGTAGTTATATCTAGAAAGAAGCA |
| | ATTTTGATTTCTTGAAAAGGTAGTTCCTGCACTCAGTTTAAACTAAAAATAATCATACTTGGATTTTATTTA |
| | TTTTTGTCATAGTAAAAATTTTAATTTATATATATTTTTATTTAGTATTATCTTATTCTTTGCTATTTGCCA |
| | ATCCTTTGTCATCAATTGTGTTAAATGAATTGAAAATTCATGCCCTGTTCATTTTATTTTACTTTATTGGTT |
| | AGGATATTTAAAGGATTTTTGTATATATAATTTCTTAAATTAATATTCCAAAAGGTTAGTGGACTTAGATTA |
| | TAAATTATGGCAAAAATCTAAAAACAACAAAAATGATTTTTATACATTCTATTTCATTATTCCTCTTTTTCC |
| | AATAAGTCATACAATTGGTAGATATGACTTATTTTATTTTTGTATTATTCACTATATCTTTATGATATTTAA |
| | GTATAAATAATTAAAAAAATTTATTGTACCTTATAGTCTGTCACCAAAAAAAAAAAATTATCTGTAGGTAGT |
| | GAAATGCTAATGTTGATTTGTCTTTAAGGGCTTGTTAACTATCCTTTATTTTCTCATTTGTCTTAAATTAGG |
| | AGTTTGTGTTTAAATTACTCATCTAAGCAAAAAATGTATATAAATCCCATTACTGGGTATATACCCAAAGGA |
| | TTATAAATCATGCTGCTATAAAGACACATGCACACGTATGTTTATTGCAGCACTATTCACAATAGCAAAGAC |
| | TTGGAACCAACCCAAATGTCCATCAATGATAGACTTGATTAAGAAAATGTGCACATATACACCATGGAATAC |
| | TATGCAGCCATAAAAAAGGATGAGTTCATGTCCTTTGTAGGGACATGGATAAAGCTGGAAACCATCATTCTG |
| | AGCAAACTATTGCAAGGACAGAAAACCAAACACTGCATGTTCTCACTCATAGGTGGGAATTGAACAATGAGA |
| | ACACTTGGACACAAGGTGGGGAACACCACACACCAGGGCCTGTCATGGGGTGGGGGGAGTGGGGAGGGATAG |
| | CATTAGGAGATATACCTAATGTAAATGATGAGTTAATGGGTGCAGCACACCAACATGGCACATGTATACATA |
| | TGTAGCAAACCTGCACGTTGTGCACATGTACCCTAGAACTTAAAGTATAATTAAAAAAAAAAAGAAAACAGA |
| | AGCTATTTATAAAGAAGTTATTTGCTGAAATAAATGTGATCTTTCCCATTAAAAAAATAAAGAAATTTTGGG |
| | GTAAAAAAACACAATATATTGTATTCTTGAAAAATTCTAAGAGAGTGGATGTGAAGTGTTCTCACCACAAAA |
| | GTGATAACTAATTGAGGTAATGCACATATTAATTAGAAAGATTTTGTCATTCCACAATGTATATATACTTAA |
| | AAATATGTTATACACAATAAATACATACATTAAAAAATAAGTAAATGTA |
| 51 | CCCACCCTGCACGCCGGCACCAAACCCTGTCCTCCCACCCCTCCCCACTCATCACTAAACAGAGTAAAATGT |
| | GATGCGAATTTTCCCGACCAACCTGATTCGCTAGATTTTTTTTAAGGAAAAGCTTGGAAAGCCAGGACACAA |
| | CGCTGCTGCCTGCTTTGTGCAGGGTCCTCCGGGGCTCAGCCCTGAGTTGGCATCACCTGCGCAGGGCCCTCT |
| | GGGGCTCAGCCCTGAGCTAGTGTCACCTGCACAGGGCCCTCTGAGGCTCAGCCCTGAGCTGGCGTCACCTGT |
| | GCAGGGCCCTCTGGGGCTCAGCCCTGAGCTGGCCTCACCTGGGTTCCCCACCCCGGGCTCTCCTGCCCTGCC |
| | CTCCTGCCCGCCCTCCCTCCTGCCTGCGCAGCTCCTTCCCTAGGCACCTCTGTGCTGCATCCCACCAGCCTG |
| | AGCAAGACGCCCTCTCGGGGCCTGTGCCGCACTAGCCTCCCTCTCCTCTGTCCCCATAGCTGGTTTTTCCCA |
| | CCAATCCTCACCTAACAGTTACTTTACAATTAAACTCAAAGCAAGCTCTTCTCCTCAGCTTGGGGCAGCCAT |
| | TGGCCTCTGTCTCGTTTTGGGAAACCAAGGTCAGGAGGCCGTTGCAGACATAAATCTCGGCGACTCGGCCCC |
| | GTCTCCTGAGGGTCCTGCTGGTGACCGGCCTGGACCTTGGCCCTACAGCCCTGGAGGCCGCTGCTGACCAGC |
| | ACTGACCCCGACCTCAGAGAGTACTCGCAGGGGCGCTGGCTGCACTCAAGACCCTCGAGATTAACGGTGCTA |
| | ACCCCGTCTGCTCCTCCCTCCCGCAGAGACTGGGGCCTGGACTGGACATGAGAGCCCCTTGGTGCCACAGAG |
| | GGCTGTGTCTTACTAGAAACAACGCAAACCTCTCCTTCCTCAGAATAGTGATGTGTTCGACGTTTTATCAAA |
| | GGCCCCCTTTCTATGTTCATGTTAGTTTTGCTCCTTCTGTGTTTTTTTCTGAACCATATCCATGTTGCTGAC |
| | TTTTCCAAATAAAGGTTTTCACTCCTCTC |
| 52 | AGAGGCCTGCCTCCAGGGCTGGACTGAGGCCTGAGCGCTCCTGCCGCAGAGCTGGCCGCGCCAAATAATGTC |
| | TCTGTGAGACTCGAGAACTTTCATTTTTTTCCAGGCTGGTTCGGATTTGGGGTGGATTTTGGTTTTGTTCCC |
| | CTCCTCCACTCTCCCCCACCCCCTCCCCGCCCTTTTTTTTTTTTTTTTTTAAACTGGTATTTTATCTTTGAT |
| | TCTCCTTCAGCCCTCACCCCTGGTTCTCATCTTTCTTGATCAACATCTTTTCTTGCCTCTGTCCCCTTCTCT |
| | CATCTCTTAGCTCCCCTCCAACCTGGGGGGCAGTGGTGTGGAGAAGCCACAGGCCTGAGATTTCATCTGCTC |
| | TCCTTCCTGGAGCCCAGAGGAGGGCAGCAGAAGGGGGTGGTGTCTCCAACCCCCCAGCACTGAGGAAGAACG |
| | GGGCTCTTCTCATTTCACCCCTCCCTTTCTCCCCTGCCCCCAGGACTGGGCCACTTCTGGGTGGGGCAGTGG |
| | GTCCCAGATTGGCTCACACTGAGAATGTAAGAACTACAAACAAAATTTCTATTAAATTAAATTTTGTGTCTC |
| | C |
| 53 | CTCCCTCCATCCCAACCTGGCTCCCTCCCACCCAACCAACTTTCCCCCCAACCCGGAAACAGACAAGCAACC |
| | CAAACTGAACCCCCTCAAAAGCCAAAAAATGGGAGACAATTTCACATGGACTTTGGAAAATATTTTTTTCCT |
| | TTGCATTCATCTCTCAAACTTAGTTTTTATCTTTGACCAACCGAACATGACCAAAAACCAAAAGTGCATTCA |
| | ACCTTACCAAAAAAAAAAAAAAAAAAAGAATAAATAAATAACTTTTTAAAAAAGGAAGCTTGGTCCACTTGC |
| | TTGAAGACCCATGCGGGGGTAAGTCCCTTTCTGCCCGTTGGGCTTATGAAACCCCAATGCTGCCCTTTCTGC |
| | TCCTTTCTCCACACCCCCCTTGGGGCCTCCCCTCCACTCCTTCCCAAATCTGTCTCCCCAGAAGACACAGGA |
| | AACAATGTATTGTCTGCCCAGCAATCAAAGGCAATGCTCAAACACCCAAGTGGCCCCCACCCTCAGCCCGCT |
| | CCTGCCCGCCCAGCACCCCCAGGCCCTGGGGGACCTGGGGTTCTCAGACTGCCAAAGAAGCCTTGCCATCTG |
| | GCGCTCCCATGGCTCTTGCAACATCTCCCCTTCGTTTTTGAGGGGGTCATGCCGGGGGAGCCACCAGCCCCT |
| | CACTGGGTTCGGAGGAGAGTCAGGAAGGGCCACGACAAAGCAGAAACATCGGATTTGGGGAACGCGTGTCAA |
| | TCCCTTGTGCCGCAGGGCTGGGCGGGAGAGACTGTTCTGTTCCTTGTGTAACTGTGTTGCTGAAAGACTACC |
| | TCGTTCTTGTCTTGATGTGTCACCGGGGCAACTGCCTGGGGGCGGGGATGGGGGCAGGGTGGAAGCGGCTCC |
| | CCATTTTATACCAAAGGTGCTACATCTATGTGATGGGTGGGGTGGGGAGGGAATCACTGGTGCTATAGAAAT |
| | TGAGATGCCCCCCCAGGCCAGCAAATGTTCCTTTTTGTTCAAAGTCTATTTTTATTCCTTGATATTTTTCTT |
| | TTTTTTTTTTTTTTTTTGTGGATGGGGACTTGTGAATTTTTCTAAAGGTGCTATTTAACATGGGAGGAGAGC |
| | GTGTGCGGCTCCAGCCCAGCCCGCTGCTCACTTTCCACCCTCTCTCCACCTGCCTCTGGCTTCTCAGGCCTC |
| | TGCTCTCCGACCTCTCTCCTCTGAAACCCTCCTCCACAGCTGCAGCCCATCCTCCCGGCTCCCTCCTAGTCT |
| | GTCCTGCGTCCTCTGTCCCCGGGTTTCAGAGACAACTTCCCAAAGCACAAAGCAGTTTTTCCCCCTAGGGGT |
| | GGGAGGAAGCAAAAGACTCTGTACCTATTTTGTATGTGTATAATAATTTGAGATGTTTTTAATTATTTTGAT |
| | TGCTGGAATAAAGCATGTGGAAATGACCCAAACATAATCCGCAGTGGCCTCCTAATTTCCTTCTTTGGAGTT |
| | GGGGGAGGGGTAGACATGGGGAAGGGGCTTTGGGGTGATGGGCTTGCCTTCCATTCCTGCCCTTTCCCTCCC |
| | CACTATTCTCTTCTAGATCCCTCCATAACCCCACTCCCCTTTCTCTCACCCTTCTTATACCGCAAACCTTTC |
| | TACTTCCTCTTTCATTTTCTATTCTTGCAATTTCCTTGCACCTTTTCCAAATCCTCTTCTCCCCTGCAATAC |
| | CATACAGGCAATCCACGTGCACAACACACACACACACTCTTCACATCTGGGGTTGTCCAAACCTCATACCCA |
| | CTCCCCTTCAAGCCCATCCACTCTCCACCCCCTGGATGCCCTGCACTTGGTGGCGGTGGGATGCTCATGGAT |
| | ACTGGGAGGGTGAGGGGAGTGGAACCCGTGAGGAGGACCTGGGGGCCTCTCCTTGAACTGACATGAAGGGTC |
| | ATCTGGCCTCTGCTCCCTTCTCACCCACGCTGACCTCCTGCCGAAGGAGCAACGCAACAGGAGAGGGGTCTG |
| | CTGAGCCTGGCGAGGGTCTGGGAGGGACCAGGAGGAAGGCGTGCTCCCTGCTCGCTGTCCTGGCCCTGGGGG |
| | AGTGAGGGAGACAGACACCTGGGAGAGCTGTGGGGAAGGCACTCGCACCGTGCTCTTGGGAAGGAAGGAGAC |
| | CTGGCCCTGCTCACCACGGACTGGGTGCCTCGACCTCCTGAATCCCCAGAACACAACCCCCCTGGGCTGGGG |
| | TGGTCTGGGGAACCATCGTGCCCCCGCCTCCCGCCTACTCCTTTTTAAGCTT |
| 54 | TTGGCCAGGCCTGACCCTCTTGGACCTTTCTTCTTTGCCGACAACCACTGCCCAGCAGCCTCTGGGACCTCG |
| | GGGTCCCAGGGAACCCAGTCCAGCCTCCTGGCTGTTGACTTCCCATTGCTCTTGGAGCCACCAATCAAAGAG |
| | ATTCAAAGAGATTCCTGCAGGCCAGAGGCGGAACACACCTTTATGGCTGGGGCTCTCCGTGGTGTTCTGGAC |
| | CCAGCCCCTGGAGACACCATTCACTTTTACTGCTTTGTAGTGACTCGTGCTCTCCAACCTGTCTTCCTGAAA |
| | AACCAAGGCCCCCTTCCCCCACCTCTTCCATGGGGTGAGACTTGAGCAGAACAGGGGCTTCCCCAAGTTGCC |
| | CAGAAAGACTGTCTGGGTGAGAAGCCATGGCCAGAGCTTCTCCCAGGCACAGGTGTTGCACCAGGGACTTCT |
| | GCTTCAAGTTTTGGGGTAAAGACACCTGGATCAGACTCCAAGGGCTGCCCTGAGTCTGGGACTTCTGCCTCC |
| | ATGGCTGGTCATGAGAGCAAACCGTAGTCCCCTGGAGACAGCGACTCCAGAGAACCTCTTGGGAGACAGAAG |
| | AGGCATCTGTGCACAGCTCGATCTTCTACTTGCCTGTGGGGAGGGGAGTGACAGGTCCACACACCACACTGG |
| | GTCACCCTGTCCTGGATGCCTCTGAAGAGAGGGACAGACCGTCAGAAACTGGAGAGTTTCTATTAAAGGTCA |
| | TTTAAACCA |
| 55 | TCCTCCGGGACCCCAGCCCTCAGGATTCCTGATGCTCCAAGGCGACTGATGGGCGCTGGATGAAGTGGCACA |
| | GTCAGCTTCCCTGGGGGCTGGTGTCATGTTGGGCTCCTGGGGCGGGGGCACGGCCTGGCATTTCACGCATTG |
| | CTGCCACCCCAGGTCCACCTGTCTCCACTTTCACAGCCTCCAAGTCTGTGGCTCTTCCCTTCTGTCCTCCGA |
| | GGGGCTTGCCTTCTCTCGTGTCCAGTGAGGTGCTCAGTGATCGGCTTAACTTAGAGAAGCCCGCCCCCTCCC |
| | CTTCTCCGTCTGTCCCAAGAGGGTCTGCTCTGAGCCTGCGTTCCTAGGTGGCTCGGCCTCAGCTGCCTGGGT |
| | TGTGGCCGCCCTAGCATCCTGTATGCCCACAGCTACTGGAATCCCCGCTGCTGCTCCGGGCCAAGCTTCTGG |
| | TTGATTAATGAGGGCATGGGGTGGTCCCTCAAGACCTTCCCCTACCTTTTGTGGAACCAGTGATGCCTCAAA |
| | GACAGTGTCCCCTCCACAGCTGGGTGCCAGGGGCAGGGGATCCTCAGTATAGCCGGTGAACCCTGATACCAG |
| | GAGCCTGGGCCTCCCTGAACCCCTGGCTTCCAGCCATCTCATCGCCAGCCTCCTCCTGGACCTCTTGGCCCC |
| | CAGCCCCTTCCCCACACAGCCCCAGAAGGGTCCCAGAGCTGACCCCACTCCAGGACCTAGGCCCAGCCCCTC |
| | AGCCTCATCTGGAGCCCCTGAAGACCAGTCCCACCCACCTTTCTGGCCTCATCTGACACTGCTCCGCATCCT |
| | GCTGTGTGTCCTGTTCCATGTTCCGGTTCCATCCAAATACACTTTCTGGAACAAA |
| 56 | GCTGGAGCCTCGGTGGCCATGCTTCTTGCCCCTTGGGCCTCCCCCCAGCCCCTCCTCCCCTTCCTGCACCCG |
| | TACCCCCGTGGTCTTTGAATAAAGTCTGAGTGGGCGGC |
| 57 | CGCTCCAGGGGAATCAGGGCATCGCCTCCTTTTCTGGGAGGACACTCCCTTCTGATGGTGAATGGGAACTCC |
| | CTTCCTCCTGCAGCAGCCTGCCTGCAGCTGTCCTGGTAGAACAGTGTGGACATTGCAGAAGCTGTCACTGCC |
| | CCAGAAAGAAAGCACCCCAGAGCC |
| 58 | AATTCTGACATCCGGCGG |
| 59 | GCATATGACT |
| 60 | GAGAATAAACTAGTATTCTTCTGGTCCCCACAGACTCAGAGAGAACCCGCCACC |
| 61 | CTCGAGCTGGTACTGCATGCACGCAATGCTAGCTGCCCCTTTCCCGTCCTGGGTACCCCGAGTCTCCCCCGA |
| | CCTCGGGTCCCAGGTATGCTCCCACCTCCACCTGCCCCACTCACCACCTCTGCTAGTTCCAGACACCTCCCA |
| | AGCACGCAGCAATGCAGCTCAAAACGCTTAGCCTAGCCACACCCCCACGGGAAACAGCAGTGATTAACCTTT |
| | AGCAATAAACGAAAGTTTAACTAAGCTATACTAACCCCAGGGTTGGTCAATTTCGTGCCAGCCACACCCTGG |
| | AGCTAGC |
| 62 | GGGAAATAAGAGAGAAAAGAAGAGTAAGAAGAAATATAAGACCCCGGCGCCGCCACC |
| 63 | GCTGGAGCCTCGGTGGCCTAGCTTCTTGCCCCTTGGGCCTCCCCCCAGCCCCTCCTCCCCTTCCTGCACCCG |
| | TACCCCCGTGGTCTTTGAATAAAGTCTGAGTGGGCGGC |
| 64 | ATGGAGAAAATAGTGCTTCTTCTTGCAATAGTCAGCCTTGTTAAAAGTGATCAGATTTGCATTGGTTACCAT |
| | GCAAACAACTCGACAGAGCAGGTTGACACAATAATGGAAAAGAACGTCACTGTTACACACGCCCAAGACATA |
| | CTGGAAAAGACACACAACGGGAAACTCTGCGATCTAGATGGAGTGAAGCCTCTAATTTTAAGAGATTGTAGT |
| | GTAGCTGGATGGCTCCTCGGGAACCCAATGTGTGACGAATTCCTCAATGTGCCGGAATGGTCTTACATAGTG |
| | GAGAAGATCAATCCAGCCAATGACCTCTGTTACCCAGGGAATTTCAACGACTATGAAGAACTGAAACACCTA |
| | TTGAGCAGAATAAACCATTTTGAGAAAATTCAGATCATCCCCAAAAGTTCTTGGTCAGATCATGAAGCCTCA |
| | GCAGGGGTGAGCTCAGCATGTCCATACCAGGGAAGGTCCTCCTTTTTTAGAAATGTGGTATGGCTTATCAAA |
| | AAGGACAATGCATACCCAACAATAAAGAGAAGTTACAATAATACCAACCAAGAAGATCTTTTGGTATTGTGG |
| | GGGATTCACCATCCAAATGATGCGGCAGAGCAGACAAGGCTCTATCAAAACCCAACTACCTATATTTCCGTT |
| | GGGACATCAACACTAAACCAGAGATTGGTACCAAAAATAGCCACTAGATCTAAGGTAAACGGGCAAAGTGGA |
| | AGGATGGAGTTCTTTTGGACAATTTTAAAACCGAATGATGCAATAAACTTTGAGAGTAATGGAAATTTCATT |
| | GCTCCAGAAAATGCATACAAAATTGTCAAGAAAGGGGACTCAACAATTATGAAAAGTGAGTTGGAATATGGT |
| | AACTGCAACACCAAGTGTCAAACTCCAATAGGGGCGATAAACTCTAGTATGCCATTCCACAACATCCACCCT |
| | CTCACCATCGGGGAATGCCCCAAATATGTGAAATCAAGCAGATTAGTCCTTGCTACTGGGCTCAGAAATAGC |
| | CCTCAACGAGAGACACGAGGACTATTTGGAGCTATAGCAGGTTTTATAGAGGGAGGATGGGGAATGGTAGAT |
| | GGTTGGTATGGGTACCACCATAGCAACGAGCAGGGGAGTGGGTACGCTGCAGACAAAGAATCCACTCAAAAG |
| | GCAATAGATGGAGTCACCAATAAGGTCAACTCGATCATTGACAAAATGAACACTCAGTTTGAGGCTGTTGGA |
| | AGGGAATTTAATAACTTAGAAAGGAGAATAGAAAATTTAAACAAGAAGATGGAAGACGGATTCCTAGATGTC |
| | TGGACTTATAATGCTGAACTTCTGGTTCTCATGGAAAATGAGAGAACTCTAGACTTTCATGACTCAAATGTC |
| | AAGAACCTTTACGACAAGGTCCGACTACAGCTTAGGGATAATGCAAAGGAGCTTGGTAACGGTTGTTTCGAG |
| | TTCTATCACAGATGTGATAATGAATGTATGGAAAGTGTAAGAAACGGAACGTATGACTACCCGCAGTATTCA |
| | GAAGAAGCAAGATTAAAAAGAGAGGAAATAAGTGGAGTAAAATTGGAATCAATAGGAACTTACCAAATACTG |
| | TCAATTTATTCAACAGTGGCGAGCTCCCTAGCACTGGCAATCATGGTGGCTGGTCTATCTTTATGGATGTGC |
| | TCCAATGGATCGTTACAATGCAGAATTTGCATTTAA |
| 65 | ATGAATCCAAATCAGAAGATAATAACCATCGGATCAATCTGTATGGTAATTGGAATAGTTAGCTTAATGTTA |
| | CAAATTGGGAACATGATCTCAATATGGGTCAGTCATTCAATTCAGACAGGGAATCAATGCCAAGCTGAACCA |
| | ATCAGCAATACTAAATTTCTTACTGAGAAAGCTGTGGCTTCAGTAACATTAGCGGGCAATTCATCTCTTTGC |
| | CCCATTAGCGGATGGGCTGTATACAGTAAGGACAACAGTATAAGGATCGGTTCCAGGGGGGATGTGTTTGTT |
| | ATAAGAGAGCCGTTCATCTCATGCTCCCACTTGGAATGCAGAACTTTCTTTTTGACTCAGGGAGCCTTGCTG |
| | AATGACAAGCACTCCAATGGGACTGTCAAAGACAGAAGCCCTCACAGAACATTAATGAGTTGTCCTGTGGGT |
| | GAGGCTCCCTCCCCATATAACTCAAGGTTTGAGTCTGTTGCTTGGTCAGCAAGTGCTTGCCATGATGGCACC |
| | AGTTGGTTGACAATTGGAATTTCTGGTCCAGACAATGGGGCTGTGGCTGTATTGAAATACAATGGCATAATA |
| | ACAGACACCATCAAGAGTTGGAGGAACAACATACTGAGAACTCAAGAGTCTGAATGTGCATGTGTAAATGGC |
| | TCTTGCTTTACTGTAATGACTGATGGACCAAGTAGTGGGCAGGCATCATATAAGATCTTCAAAATGGAAAAA |
| | GGGAAAGTGGTTAAATCAGTCGAATTGGATGCTCCTAATTATCACTATGAGGAGTGCTCCTGTTATCCTGAT |
| | GCCGGCGAAATCACATGTGTGTGCAGGGATAATTGGCATGGCTCAAATAGGCCATGGGTATCTTTCAATCAA |
| | AATTTGGAGTATCAAATAGGATATATATGCAGTGGAGTTTTCGGAGACAATCCACGCCCCAATGATGGAACA |
| | GGTAGTTGTGGTCCGGTGTTCTCTAACGGGGCATATGGGGTAAAAGGGTTTTCATTCAAATACGGCAATGGT |
| | GTTTGGATCGGGAGAACCAAAAGCACTAATTCCAGGAGCGGCTTTGAAATGATTTGGGACCCAAATGGGTGG |
| | ACTGGAACGGACAGTAGCTTTTCGGTGAAGCAAGATATCGTAGCAATAACTGATTGGTCAGGATATAGCGGG |
| | AGTTTTGTCCAGCATCCAGAACTGACAGGATTAGATTGCATAAGACCTTGTTTCTGGGTTGAGTTAATCAGA |
| | GGGCGGCCTAAAGAGAGCACAATTTGGACCAGTGGGAGCAGCATATCTTTTTGTGGTGTAAATAGTGACACT |
| | GTTAGTTGGTCTTGGCCAGACGGTGCTGAGTTGCCATTCACCATTGACAAGTAG |
| 66 | ATGAGTCTTCTAACCGAGGTCGAAACGTACGTACTCTCTATCATCCCGTCAGGCCCCCTCAAAGCCGAGATC |
| | GCACAGAGACTTGAAGATGTCTTTGCAGGGAAGAACACCGATCTTGAGGTTCTCATGGAATGGCTAAAGACA |
| | AGACCAATCCTGTCACCTCTGACTAAGGGGATTTTAGGATTTGTGTTCACGCTCACCGTGCCCAGTGAGCGA |
| | GGACTGCAGCGTAGACGCTTTGTCCAAAATGCCCTTAATGGGAACGGGGATCCAAATAACATGGACAAAGCA |
| | GTTAAACTGTATAGGAAGCTCAAGAGGGAGATAACATTCCATGGGGCCAAAGAAATCTCACTCAGTTATTCT |
| | GCTGGTGCACTTGCCAGTTGTATGGGCCTCATATACAACAGGATGGGGGCTGTGACCACTGAAGTGGCATTT |
| | GGCCTGGTATGTGCAACCTGTGAACAGATTGCTGACTCCCAGCATCGGTCTCATAGGCAAATGGTGACAACA |
| | ACCAATCCACTAATCAGACATGAGAACAGAATGGTTTTAGCCAGCACTACAGCTAAGGCTATGGAGCAAATG |
| | GCTGGATCGAGTGAGCAAGCAGCAGAGGCCATGGAGGTTGCTAGTCAGGCTAGACAAATGGTGCAAGCGATG |
| | AGAACCATTGGGACTCATCCTAGCTCCAGTGCTGGTCTGAAAAATGATCTTCTTGAAAATTTGCAGGCCTAT |
| | CAGAAACGAATGGGGGTGCAGATGCAACGGTTCAAGTGA |
| 67 | ATGAGTCTTCTAACCGAGGTCGAAACGCCTATCAGAAACGAATGGGGGTGCAGATGCAACGGTTCAAGTGAT |
| | CCTCTCACTATTGCCGCAAATATCATTGGGATCTTGCACTTGACATTGTGGATTCTTGATCGTCTTTTTTTC |
| | AAATGCATTTACCGTCGCTTTAAATACGGACTGAAAGGAGGGCCTTCTACGGAAGGAGTGCCAAAGTCTATG |
| | AGGGAAGAATATCGAAAGGAACAGCAGAGTGCTGTGGATGCTGACGATGGTCATTTTGTCAGCATAGAGCTG |
| | GAGTAA |
| 68 | ATGGACTCCAACACCATGTCAAGCTTTCAGGTAGACTGTTTCCTTTGGCATATCCGCAAGCGATTTGCAGAC |
| | AATGGATTGGGTGATGCCCCATTCCTTGATCGGCTCCGCCGAGATCAAAAGTCCTTAAAAGGAAGAGGCAAC |
| | ACCCTTGGCCTCGATATCGAAACAGCCACTCTTGTTGGGAAACAAATCGTGGAATGGATCTTGAAAGAGGAA |
| | TCCAGCGAGACACTTAGAATGACAATTGCATCTGTACCTACTTCGCGCTACCTTTCTGACATGACCCTCGAG |
| | GAAATGTCACGAGACTGGTTCATGCTCATGCCTAGGCAAAAGATAATAGGCCCTCTTTGCGTGCGATTGGAC |
| | CAGGCGATCATGGAAAAGAACATAGTACTGAAAGCGAACTTCAGTGTAATCTTTAACCGATTAGAGACCTTG |
| | ATACTACTAAGGGCTTTCACTGAGGAGGGAGCAATAGTTGGAGAAATTTCACCATTACCTTCTCTTCCAGGA |
| | CATACTTATGAGGATGTCAAAAATGCAGTTGGGGTCCTCATCGGAGGACTTGAATGGAATGGTAACACGGTT |
| | CGAGTCTCTGAAAATATACAGAGATTCGCTTGGAGAAACTGTGATGAGAATGGGAGACCTTCACTACCTCCA |
| | GAGCAGAAATGA |
| 69 | ATGGCGTCTCAAGGCACCAAACGATCATATGAACAAATGGAGACTGGTGGGGAGCGCCAGGATGCCACAGAA |
| | ATCAGAGCATCTGTCGGAAGAATGATTGGTGGAATCGGGAGATTCTACATCCAAATGTGCACTGAACTCAAA |
| | CTCAGTGATTATGATGGACGACTAATCCAGAATAGCATAACAATAGAGAGGATGGTGCTTTCTGCTTTTGAT |
| | GAGAGAAGAAATAAATACCTAGAGGAGCATCCCAGTGCTGGGAAGGACCCTAAGAAAACAGGAGGACCCATA |
| | TATAGAAGAGTAGACGGAAAGTGGATGAGAGAACTCATCCTTTATGACAAAGAAGAAATAAGGAGAGTTTGG |
| | CGCCAAGCAAACAATGGCGAAGATGCAACAGCAGGTCTTACTCATATCATGATTTGGCATTCCAACCTGAAT |
| | GATGCCACATATCAGAGAACAAGAGCGCTTGTTCGCACCGGAATGGATCCCAGAATGTGCTCTCTAATGCAA |
| | GGTTCAACACTTCCCAGAAGGTCTGGTGCCGCAGGTGCTGCGGTGAAAGGAGTTGGAACAATAGCAATGGAG |
| | TTAATCAGAATGATCAAACGTGGAATCAATGACCGAAATTTCTGGAGGGGTGAAAATGGACGAAGGACAAGG |
| | GTTGCTTATGAAAGAATGTGCAATATCCTCAAAGGAAAATTTCAAACAGCTGCCCAGAGGGCAATGATGGAT |
| | CAAGTAAGAGAAAGTCGAAACCCAGGAAACGCTGAGATTGAAGACCTCATTTTCCTGGCACGGTCAGCACTC |
| | ATTCTGAGGGGATCAGTTGCACATAAATCCTGCCTGCCTGCTTGTGTGTATGGGCTTGCAGTAGCAAGTGGG |
| | CATGACTTTGAAAGGGAAGGGTACTCACTGGTCGGGATAGACCCATTCAAATTACTCCAAAACAGCCAAGTG |
| | GTCAGCCTGATGAGACCAAATGAAAACCCAGCTCACAAGAGTCAATTGGTGTGGATGGCATGCCACTCTGCT |
| | GCATTTGAAGATTTAAGAGTATCAAGTTTCATAAGAGGAAAGAAAGTGATTCCAAGAGGAAAGCTTTCCACA |
| | AGAGGGGTCCAGATTGCTTCAAATGAGAATGTGGAAACCATGGACTCCAATACCCTGGAACTGAGAAGCAGA |
| | TACTGGGCCATAAGGACCAGGAGTGGAGGAAATACCAATCAACAAAAGGCATCCGCAGGCCAGATCAGTGTG |
| | CAGCCTACATTCTCAGTGCAGCGGAATCTCCCTTTTGAAAGAGCAACCGTTATGGCAGCATTCAGCGGGAAC |
| | AATGAAGGACGGACATCCGACATGCGAACAGAAGTTATAAGAATGATGGAAAGTGCAAAGCCAGAAGATTTG |
| | TCCTTCCAGGGGGGGGGAGTCTTCGAGCTCTCGGACGAAAAGGCAACGAACCCGATCGTGCCTTCCTTTGAC |
| | ATGAGTAATGAAGGGTCTTATTTCTTCGGAGACAATGCAGAGGAGTATGACAGTTGA |
| 70 | ATGAGTCTTCTAACCGAGGTCGAAACGTACGTTCTTTCTATCATCCCGTCAGGCCCCCTCAAAGCCGAGATC |
| | GCGCAGAGACTGGAAAGTGTCTTTGCAGGAAAGAACACAGATCTTGAGGCTCTCATGGAATGGCTAAAGACA |
| | AGACCAATCTTGTCACCTCTGACTAAGGGAATTTTAGGATTTGTGTTCACGCTCACCGTGCCCAGTGAGCGA |
| | GGACTGCAGCGTAGACGCTTTGTCCAAAATGCCCTAAATGGGAATGGGGACCCGAACAACATGGATAGAGCA |
| | GTTAAACTATACAAGAAGCTCAAAAGAGAAATAACGTTCCATGGGGCCAAGGAGGTGTCACTAAGCTATTCA |
| | ACTGGTGCACTTGCCAGTTGCATGGGCCTCATATACAACAGGATGGGAACAGTGACCACAGAAGCTGCTTTT |
| | GGTCTAGTGTGTGCCACTTGTGAACAGATTGCTGATTCACAGCATCGGTCTCACAGACAGATGGCTACTACC |
| | ACCAATCCACTAATCAGGCATGAAAACAGAATGGTGCTGGCTAGCACTACGGCAAAGGCTATGGAACAGATG |
| | GCTGGATCGAGTGAACAGGCAGCGGAGGCCATGGAGGTTGCTAATCAGACTAGGCAGATGGTACATGCAATG |
| | AGAACTATTGGGACTCATCCTAGCTCCAGTGCTGGTCTGAAAGATGACCTTCTTGAAAATTTGCAGGCCTAC |
| | CAGAAGCGAATGGGAGTGCAGATGCAGCGATTCAAGTGA |
| 71 | ATGAAGACTATCATTGCTTTGAGCTACATTCTATGTCTGGTTTTCACTCAAAAAATTCCTGGAAATGACAAT |
| | AGCACGGCAACGCTGTGCCTTGGGCACCATGCAGTACCAAACGGAACGATAGTGAAAACAATCACAAATGAC |
| | CGAATTGAAGTTACTAATGCTACTGAGTTGGTTCAGAATTCCTCAATAGGTAAAATATGCGACAGTCCTCAT |
| | CAGATCCTTGATGGAGGGAACTGCACACTAATAGATGCTCTATTGGGGGACCCTCAGTGTGACGGCTTTCAA |
| | AATAAGAAATGGGACCTTTTTGTTGAACGAAGCAGAGCCTACAGCAACTGTTACCCTTATGATGTGCCGGAT |
| | TATGCCTCCCTTAGGTCACTAGTTGCCTCATCCGGCACACTGGAGTTTAAAAATGAAAGCTTCAATTGGGCT |
| | GGAGTCACTCAAAACGGAAAAAGTTTTTCTTGCATAAGGGGATCTAGTAGTAGTTTCTTTAGTAGATTAAAT |
| | TGGTTGACCCATTTAAACTACACATATCCAGCACTGAACGTGACTATGCCAAACAAGGAACAATTTGACAAA |
| | TTGTACATTTGGGGGGTTCACCACCCGGGTACGGACAAGGACCAAATCTCCCTGTATGCTCAATCATCAGGA |
| | AGAATCACAGTATCTACCAAAAGAAGCCAACAAGCTGTAATCCCAAATATCGGATCTAGACCCAGAATAAGG |
| | GATATCCCTAGCAGAATAAGCATCTATTGGACAATAGTAAAACCGGGAGACATACTTTTGATTAACAGCACA |
| | GGGAATCTAATTGCTCCTAGGGGTTACTTCAAAATACGAAGTGGGAAAAGCTCAATAATGAGATCAGATGCA |
| | CCCATTGGCAAATGCAAGTCTGAATGCATCACTCCAAATGGAAGCATTCCCAATGACAAACCATTCCAAAAT |
| | GTGAACAGGATCACATACGGGGCCTGTCCCAGATATGTTAAGCAAAGCACTCTGAAATTGGCAACAGGAATG |
| | CGAAATGTACCAGAGAAACAAACTAGAGGCATATTTGGTGCAATAGCGGGTTTCATAGAAAATGGTTGGGAG |
| | GGAATGGTGGATGGTTGGTACGGTTTCAGACATCAAAATTCTGAGGGAAGAGGACAAGCAGCAGATCTCAAA |
| | AGCACTCAAGCAGCAATCGATCAAATCAATGGGAAGCTGAATCGATTGATCGGAAAAACCAACGAGAAATTC |
| | CATCAGATTGAAAAAGAATTCTCAGAAGTAGAAGGAAGAGTTCAAGACCTTGAGAAATATGTTGAGGACACT |
| | AAAATAGATCTCTGGTCATACAACGCGGAGCTTCTTGTTGCCCTGGAGAACCAACATACAATTGATCTAACT |
| | GACTCAGAAATGAACAAACTGTTTGAAAAAACAAAGAAGCAACTGAGGGAAAATGCTGAGGATATGGGAAAT |
| | GGTTGTTTCAAAATATACCACAAATGTGACAATGCCTGCATAGGATCAATAAGAAACGAAACTTATGACCAC |
| | AATGTGTACAGGGATGAAGCATTAAACAACCGGTTCCAGATCAAGGGAGTTGAGCTGAAGTCAGGGTACAAA |
| | GATTGGATCCTATGGATTTCCTTTGCCATATCATGTTTTTTGCTTTGTGTTGCTTTGTTGGGGTTCATCATG |
| | TGGGCCTGCCAAAAGGGCAACATTAGATGCAACATCTGCATTTGA |
| 72 | ATGAATCCAAATCAAAAGATAATAACGATTGGCTCTGTTTCTCTCACAATTTCCACAATATGCTTCTTCATG |
| | CAAATTGCCATCCTGATAACTACTGTAACATTGCATTTCAAGCAATATGAATTCAACTCCCCCCCGAATAAC |
| | CAAGTGATGCTGTGTGAACCAACAATAATAGAAAGAAACATAACAGAGATAGTGTATTTGACCAACACCACC |
| | ATAGAGAAGGAAATATGCCCCAAACCAGCAGAATACAGAAATTGGTCAAAACCGCAATGTGACATTACAGGA |
| | TTTGCACCTTTCTCTAAGGACAATTCGATTAGGCTTTCCGCTGGTGGGGACATCTGGGTGACAAGAGAACCT |
| | TATGTGTCATGCGACCTTGACAAGTGTTATCAATTTGCACTTGGACAGGGAACAACACTAAACAACGTGCAT |
| | TCAAATAACACAGTACGTGATAGGACCCCTTATCGGACTCTATTGATGAATGAGTTGGGTGTTCCTTTCCAT |
| | CTGGGGACCAAGCAAGTGTGCATAGCATGGTCCAGCTCAAGTTGTCACGATGGAAAAGCATGGCTGCATGTT |
| | TGTATAACGGGGGATGATAAAAATGCAACTGCTAGCTTCATTTACAATGGGAGGCTTGTAGATAGTGTTGTT |
| | TCATGGTCCAACGATATTCTCAGGACCCAGGAGTCAGAATGCGTTTGTATCAATGGAACTTGTACAGTAGTA |
| | ATGACTGATGGAAATGCCACAGGAAAAGCTGATACTAAAATACTATTCATTGAGGAGGGGAAAATCGTTCAT |
| | ACTAGCAAATTGTCAGGAAGTGCTCAGCATGTCGAAGAGTGCTCTTGCTATCCTCGATATCCTGGTGTCAGA |
| | TGTGTCTGCAGAGACAACTGGAAAGGATCCAACCGGCCCATCATAGATATAAACATAAAGGATCATAGCATT |
| | GTTTCCAGTTATGTGTGTTCAGGGCTTGTTGGAGACACACCCAGAAAAAGCGACAGCTCCAGCAGTAGCCAT |
| | TGTTTGAATCCTAACAATGAAAAAGGTGGTCATGGAGTGAAAGGCTGGGCCTTTGATGATGGAAATGACGTG |
| | TGGATGGGGAGAACAATCAACGAGACGTCACGCTTAGGGTATGAAACCTTCAAAGTCGTTGAAGGCTGGTCC |
| | AACCCTAAGTCCAAATTGCAGATAAATAGGCAAGTCATAGTGGATAGAGGTGATAGGTCCGGATACAGCGGT |
| | ATTTTCTCTGTTGAAGGCAAAAGCTGCATCAATCGGTGCTTCTATGTGGAGTTGATTAGGGGAAGAAAAGAG |
| | GAAACTGAAGTCTTGTGGACCTCAAACAGTATTGTTGTGTTTTGTGGCACCTCAGGTACATATGGAACAGGC |
| | TCATGGCCTGATGGGGCGGACCTCAATCTCATGCATACATAA |
| 73 | ATGAAGGCAATAATTGTACTACTCATGGTAGTAACATCCAATGCAGATCGAATCTGCACTGGGATAACATCT |
| | TCAAACTCACCTCATGTGGTCAAAACAGCTACTCAAGGGGAGGTCAATGTGACTGGCGTGATACCACTGACA |
| | ACAACACCAACAAAATCTTATTTTGCAAATCTCAAAGGAACAAGGACCAGAGGGAAACTATGCCCGGACTGT |
| | CTCAACTGTACAGATCTGGATGTGGCCTTGGGCAGGCCAATGTGTGTGGGGACCACACCTTCTGCTAAAGCT |
| | TCAATACTCCATGAGGTCAGACCTGTTACATCCGGGTGCTTTCCTATAATGCACGACAGAACAAAAATCAGA |
| | CAACTACCCAATCTTCTCAGAGGATATGAAAAGATCAGGTTATCAACCCAAAACGTTATCGATGCAGAAAAA |
| | GCACCAGGAGGACCCTACAGACTTGGAACCTCAGGATCTTGCCCTAACGCTACCAGTAAAATTGGATTTTTT |
| | TCAACAATGGCTTGGGCTGTTCCAAAGGACAACTACAAAAATGCAACGAACCCACAAACAGTGGAAGTACCA |
| | TACATTTGTACAGAAGGGGAAGACCAAATTACTGTTTGGGGGTTCCATTCGGATAACAAAACCCAAATGAAG |
| | TCCCTCTATGGAGACTCAAATCCTCAAAAGTTCACCTCATCTGCTAATGGAGTGACCACACATTATGTTTCT |
| | CAAATTGGCGACTTCCCAGATCAAACAGAAGACGGAGGACTACCACAAAGCGGCAGAATTGTTGTTGATTAC |
| | ATGGTGCAAAAACCTGGGAAAACAGGAACAATTGTCTATCAAAGGGGTGTTTTGTTGCCTCAAAAGGTGTGG |
| | TGCGCGAGTGGCAGGAGCAAAGTAATAAAAGGGTCATTGCCTTTAATTGGTGAAGCAGATTGCCTTCATGAA |
| | GAATACGGTGGATTAAACAAAAGCAAGCCTTACTACACAGGAAAACATGCAAAAGCCATAGGAAATTGCCCA |
| | ATATGGGTAAAAACACCTTTGAAGCTTGCCAATGGAACCAAATATAGACCTCCTGCAAAACTATTGAAGGAA |
| | AGGGGTTTCTTCGGAGCTATTGCTGGTTTCCTAGAAGGAGGATGGGAAGGAATGATTGCAGGTTGGCACGGA |
| | TACACATCTCACGGAGCACATGGAGTGGCAGTGGCGGCAGACCTTAAGAGTACACAAGAAGCTATAAATAAG |
| | ATAACAAAAAATCTCAATTCTTTGAGTGAACTAGAAGTAAAGAACCTTCAAAGACTAAGTGGTGCCATGGAT |
| | GAACTCCACAACGAAATACTCGAGCTGGATGAAAAAGTGGATGACCTCAGAGCTGACACTATAAGCTCACAA |
| | ATAGAACTTGCAGTCTTGCTTTCCAACGAAGGAATAATAAACAGTGAAGATGAGCATCTATTGGCACTTGAG |
| | AGAAAACTAAAGAAAATGCTGGGTCCCTCTGCTGTAGACATAGGAAACGGATGCTTCGAAACCAAACACAAA |
| | TGCAACCAGACCTGCTTAGACAGGATAGCTGCTGGCACCTTTAATGCAGGAGAATATTCTCTCCCCACTTTT |
| | GACTCATTGAACATTACTGCTGCATCTTTAAATGATGATGGATTGGATAACCATACTATACTGCTCTATTAC |
| | TCAACTGCTGCTTCTAGTTTGGCTGTAACATTAATGCTAGCTATTTTTATTGTTTATATGGTCTCCAGAGAC |
| | AACGTTTCATGCTCCATTTGTCTATAA |
| 74 | ATGCTACCTTCAACTATACAAACGTTAACCTTATTTCTCACATTAGGGGGAGTGCTATTATCACTATATGTG |
| | TCAGCTTCATTGTCATACTTACTATATTCGGATATATTGCTAAAATTTTCACGAACAGAAGTAACTGCACCA |
| | ATAATGCCATTGGATTGTGCAAACGCATCAAATGTCCAGGCTGTGAATCGTTCTGCAACAAAAGGGGTGACA |
| | CCTCTTCTCCCAGAACCGGAGTGGACGTACCCTCGTTTATCTTGCCCGGGCTCAACCTTTCAGAAAGCACTC |
| | CTAATTAGCCCCCATAGATTCGGAGAAACCAAAGGAAACTCAGCTCCCTTAATAATAAGGGAACCTTTTATT |
| | GCTTGTGGACCAAAGGAATGCAAACATTTTGCTCTAACCCATTATGCAGCTCAACCAGGGGGATACTACAAT |
| | GGAACAAGAGAAGACAGAAACAAGCTGAGGCATCTAATTTCAGTCAAATTGGGCAAAATCCCAACAGTAGAA |
| | AACTCTATTTTCCACATGGCAGCTTGGAGTGGGTCCGCATGCCATGATGGTAGAGAATGGACTTACATCGGA |
| | GTTGATGGCCCAGACAGTAATGCATTGCTCAAAATAAAATATGGAGAAGCATATACTGACACATACCATTCC |
| | TATGCAAAAAACATCCTAAGGACACAAGAAAGTGCCTGCAATTGCATCGGGGGAGATTGTTATCTGATGATA |
| | ACTGATGGCCCAGCTTCAGGGATTAGTGAATGCAGATTCCTTAAGATTCGAGAGGGCCGAATAATAAAAGAA |
| | ATATTTCCAACAGGAAGAGTAAAACACACTGAGGAATGCACATGCGGATTTGCCAGCAACAAAACCATAGAA |
| | TGTGCCTGTAGAGATAACAGTTACACAGCAAAAAGACCCTTTGTCAAATTAAATGTGGAGACCGATACAGCG |
| | GAAATAAGATTGATGTGCACAAAGACTTATTTGGACACCCCCAGACCAAATGATGGAAGCATAACAGGGCCT |
| | TGCGAATCTGATGGAAACGAAGGGGGTGGAGGCATCAAGGGAGGATTTGTTCACCAAAGAATGGCATCCAAG |
| | ATTGGAAGGTGGTACTCTCGGACGATGTCTAAAACTCAAAGAATGGGGATGGGACTGTATGTAAAGTATGAT |
| | GGAGACCCATGGACTGACAGTGAAGCCCTTGCTCTTAGTGGAGTAATGGTTTCAATGGGAGAACCTGGTTGG |
| | TATTCCTTTGGCTTCGAAATAAAAGATAAGAAATGTGATGTCCCCTGTATTGGGATAGAAATGGTACATGAT |
| | GGTGGGAAAACGACTTGGCACTCAGCGGCAACAGCCATTTACTGTTTAATGGGCTCAGGACAACTGCTGTGG |
| | GACACTGTCACAGGTGTTAATATGACTCTGTAA |
| 75 | GGGCCCCTATAACTCTCTACGGCTAACCTGAATGGACTACGACATAGTCTAGTCCGCCAAG |
| 76 | ATAGGCGGCGCATGAGAGAAGCCCAGACCAATTACCTACCCAAA |
| 77 | ATACAGCAGCAATTGGCAAGCTGCTTACATAGAACTCGCGGCGATTGGCATGCCGCCTTAAAATTTTTATTT |
| | TATTTTTCTTTTCTTTTCCGAATCGGATTTTGTTTTTAATATTTC |
| 78 | ATTTTGTTTTTAATATTTC |
| 79 | GGCGCGCCCACCCAGCGGCCGCCCGCTACGCCCCAATGATCCGACCAGCAAAACTCGATGTACTTCCGAGGA |
| | ACTGATGTGCATAATGCATCAGGCTGGTACATTAGATCCCCGCTTACCGCGGGCAATATAGCAACACTAAAA |
| | ACTCGATGTACTTCCGAGGAAGCGCAGTGCATAATGCTGCGCAGTGTTGCCACATAACCACTATATTAACCA |
| | TTTATCTAGCGGACGCCAAAAACTCAATGTATTTCTGAGGAAGCGTGGTGCATAATGCCACGCAGCGTCTGC |
| | ATAACTTTTATTATTTCTTTTATTAATCAACAAAATTTTGTTTTTAACATTTC |
| 80 | ATTTTGTTTTTAACATTTC |
| 81 | ATAGGCGGCGCATGAGAGAAGCCCAGACCAATTACCTACCCAAAATGGAGAAAGTTCACGTTGACATCGAGG |
| | AAGACAGCCCATTCCTCAGAGCTTTGCAGCGGAGCTTCCCGCAGTTTGAGGTAGAAGCCAAGCAGGTCACTG |
| | ATAATGACCATGCTAATGCCAGAGCGTTTTCGCATCTGGCTTCAAAACTGATCGAAACGGAGGTGGACCCAT |
| | CCGACACGATCCTTGACATTGGAAGTGCGCCCGCCCGCAGAATGTATTCTAAGCACAAGTATCATTGTATCT |
| | GTCCGATGAGATGTGCGGAAGATCCGGACAGATTGTATAAGTATGCAACTAAGCTGAAGAAAAACTGTAAGG |
| | AAATAACTGATAAGGAATTGGACAAGAAAATGAAGGAGCTCGCCGCCGTCATGAGCGACCCTGACCTGGAAA |
| | CTGAGACTATGTGCCTCCACGACGACGAGTCGTGTCGCTACGAAGGGCAAGTCGCTGTTTACCAGGATGTAT |
| | ACGCGGTTGACGGACCGACAAGTCTCTATCACCAAGCCAATAAGGGAGTTAGAGTCGCCTACTGGATAGGCT |
| | TTGACACCACCCCTTTTATGTTTAAGAACTTGGCTGGAGCATATCCATCATACTCTACCAACTGGGCCGACG |
| | AAACCGTGTTAACGGCTCGTAACATAGGCCTATGCAGCTCTGACGTTATGGAGCGGTCACGTAGAGGGATGT |
| | CCATTCTTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATTACACAT |
| | GTCGGTGTGAGACTATAGTTAGTTGCGACGGGTACGTCGTTAAAAGAATAGCTATCAGTCCAGGCCTGTATG |
| | GGAAGCCTTCAGGCTATGCTGCTACGATGCACCGCGAGGGATTCTTGTGCTGCAAAGTGACAGACACATTGA |
| | ACGGGGAGAGGGTCTCTTTTCCCGTGTGCACGTATGTGCCAGCTACATTGTGTGACCAAATGACTGGCATAC |
| | TGGCAACAGATGTCAGTGCGGACGACGCGCAAAAACTGCTGGTTGGGCTCAACCAGCGTATAGTCGTCAACG |
| | GTCGCACCCAGAGAAACACCAATACCATGAAAAATTACCTTTTGCCCGTAGTGGCCCAGGCATTTGCTAGGT |
| | GGGCAAAGGAATATAAGGAAGATCAAGAAGATGAAAGGCCACTAGGACTACGAGATAGACAGTTAGTCATGG |
| | GGTGTTGTTGGGCTTTTAGAAGGCACAAGATAACATCTATTTATAAGCGCCCGGATACCCAAACCATCATCA |
| | AAGTGAACAGCGATTTCCACTCATTCGTGCTGCCCAGGATAGGCAGTAACACATTGGAGATCGGGCTGAGAA |
| | CAAGAATCAGGAAAATGTTAGAGGAGCACAAGGAGCCGTCACCTCTCATTACCGCCGAGGACGTACAAGAAG |
| | CTAAGTGCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCGGCTCAG |
| | TGGAGACACCTCGTGGCTTGATAAAGGTTACCAGCTACGATGGCGAGGACAAGATCGGCTCTTACGCTGTGC |
| | TTTCTCCGCAGGCTGTACTCAAGAGTGAAAAATTATCTTGCATCCACCCTCTCGCTGAACAAGTCATAGTGA |
| | TAACACACTCTGGCCGAAAAGGGCGTTATGCCGTGGAACCATACCATGGTAAAGTAGTGGTGCCAGAGGGAC |
| | ATGCAATACCCGTCCAGGACTTTCAAGCTCTGAGTGAAAGTGCCACCATTGTGTACAACGAACGTGAGTTCG |
| | TAAACAGGTACCTGCACCATATTGCCACACATGGAGGAGCGCTGAACACTGATGAAGAATATTACAAAACTG |
| | TCAAGCCCAGCGAGCACGACGGCGAATACCTGTACGACATCGACAGGAAACAGTGCGTCAAGAAAGAACTAG |
| | TCACTGGGCTAGGGCTCACAGGCGAGCTGGTGGATCCTCCCTTCCATGAATTCGCCTACGAGAGTCTGAGAA |
| | CACGACCAGCCGCTCCTTACCAAGTACCAACCATAGGGGTGTATGGCGTGCCAGGATCAGGCAAGTCTGGCA |
| | TCATTAAAAGCGCAGTCACCAAAAAAGATCTAGTGGTGAGCGCCAAGAAAGAAAACTGTGCAGAAATTATAA |
| | GGGACGTCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGCTCATAG |
| | CCATTATAAGACCTAAAAAGGCAGTGCTCTGCGGGGATCCCAAACAGTGCGGTTTTTTTAACATGATGTGCC |
| | TGAAAGTGCATTTTAACCACGAGATTTGCACACAAGTCTTCCACAAAAGCATCTCTCGCCGTTGCACTAAAT |
| | CTGTGACTTCGGTCGTCTCAACCTTGTTTTACGACAAAAAAATGAGAACGACGAATCCGAAAGAGACTAAGA |
| | TTGTGATTGACACTACCGGCAGTACCAAACCTAAGCAGGACGATCTCATTCTCACTTGTTTCAGAGGGTGGG |
| | TGAAGCAGTTGCAAATAGATTACAAAGGCAACGAAATAATGACGGCAGCTGCCTCTCAAGGGCTGACCCGTA |
| | AAGGTGTGTATGCCGTTCGGTACAAGGTGAATGAAAATCCTCTGTACGCACCCACCTCAGAACATGTGAACG |
| | TCCTACTGACCCGCACGGAGGACCGCATCGTGTGGAAAACACTAGCCGGCGACCCATGGATAAAAACACTGA |
| | CTGCCAAGTACCCTGGGAATTTCACTGCCACGATAGAGGAGTGGCAAGCAGAGCATGATGCCATCATGAGGC |
| | ACATCTTGGAGAGACCGGACCCTACCGACGTCTTCCAGAATAAGGCAAACGTGTGTTGGGCCAAGGCTTTAG |
| | TGCCGGTGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGGACTCCG |
| | GTCTATTTTCTGCACCCACTGTTCCGTTATCCATTAGGAATAATCACTGGGATAACTCCCCGTCGCCTAACA |
| | TGTACGGGCTGAATAAAGAAGTGGTCCGTCAGCTCTCTCGCAGGTACCCACAACTGCCTCGGGCAGTTGCCA |
| | CTGGAAGAGTCTATGACATGAACACTGGTACACTGCGCAATTATGATCCGCGCATAAACCTAGTACCTGTAA |
| | ACAGAAGACTGCCTCATGCTTTAGTCCTCCACCATAATGAACACCCACAGAGTGACTTTTCTTCATTCGTCA |
| | GCAAATTGAAGGGCAGAACTGTCCTGGTGGTCGGGGAAAAGTTGTCCGTCCCAGGCAAAATGGTTGACTGGT |
| | TGTCAGACCGGCCTGAGGCTACCTTCAGAGCTCGGCTGGATTTAGGCATCCCAGGTGATGTGCCCAAATATG |
| | ACATAATATTTGTTAATGTGAGGACCCCATATAAATACCATCACTATCAGCAGTGTGAAGACCATGCCATTA |
| | AGCTTAGCATGTTGACCAAGAAAGCTTGTCTGCATCTGAATCCCGGCGGAACCTGTGTCAGCATAGGTTATG |
| | GTTACGCTGACAGGGCCAGCGAAAGCATCATTGGTGCTATAGCGCGGCAGTTCAAGTTTTCCCGGGTATGCA |
| | AACCGAAATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCGGATGTG |
| | CACCCTCATATCATGTGGTGCGAGGGGATATTGCCACGGCCACCGAAGGAGTGATTATAAATGCTGCTAACA |
| | GCAAAGGACAACCTGGCGGAGGGGTGTGCGGAGCGCTGTATAAGAAATTCCCGGAAAGCTTCGATTTACAGC |
| | CGATCGAAGTAGGAAAAGCGCGACTGGTCAAAGGTGCAGCTAAACATATCATTCATGCCGTAGGACCAAACT |
| | TCAACAAAGTTTCGGAGGTTGAAGGTGACAAACAGTTGGCAGAGGCTTATGAGTCCATCGCTAAGATTGTCA |
| | ACGATAACAATTACAAGTCAGTAGCGATTCCACTGTTGTCCACCGGCATCTTTTCCGGGAACAAAGATCGAC |
| | TAACCCAATCATTGAACCATTTGCTGACAGCTTTAGACACCACTGATGCAGATGTAGCCATATACTGCAGGG |
| | ACAAGAAATGGGAAATGACTCTCAAGGAAGCAGTGGCTAGGAGAGAAGCAGTGGAGGAGATATGCATATCCG |
| | ACGACTCTTCAGTGACAGAACCTGATGCAGAGCTGGTGAGGGTGCATCCGAAGAGTTCTTTGGCTGGAAGGA |
| | AGGGCTACAGCACAAGCGATGGCAAAACTTTCTCATATTTGGAAGGGACCAAGTTTCACCAGGCGGCCAAGG |
| | ATATAGCAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTAGCACGC |
| | TGCCTTGCTTGTGCATCCATGCCATGACTCCAGAAAGAGTACAGCGCCTAAAAGCCTCACGTCCAGAACAAA |
| | TTACTGTGTGCTCATCCTTTCCATTGCCGAAGTATAGAATCACTGGTGTGCAGAAGATCCAATGCTCCCAGC |
| | CTATATTGTTCTCACCGAAAGTGCCTGCGTATATTCATCCAAGGAAGTATCTCGTGGAAACACCACCGGTAG |
| | ACGAGACTCCGGAGCCATCGGCAGAGAACCAATCCACAGAGGGGACACCTGAACAACCACCACTTATAACCG |
| | AGGATGAGACCAGGACTAGAACGCCTGAGCCGATCATCATCGAAGAGGAAGAAGAGGATAGCATAAGTTTGC |
| | TGTCAGATGGCCCGACCCACCAGGTGCTGCAAGTCGAGGCAGACATTCACGGGCCGCCCTCTGTATCTAGCT |
| | CATCCTGGTCCATTCCTCATGCATCCGACTTTGATGTGGACAGTTTATCCATACTTGACACCCTGGAGGGAG |
| | CTAGCGTGACCAGCGGGGCAACGTCAGCCGAGACTAACTCTTACTTCGCAAAGAGTATGGAGTTTCTGGCGC |
| | GACCGGTGCCTGCGCCTCGAACAGTATTCAGGAACCCTCCACATCCCGCTCCGCGCACAAGAACACCGTCAC |
| | TTGCACCCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGGTCTCCA |
| | ACCCGCCAGGCGTAAATAGGGTGATTACAAGAGAGGAGTTTGAGGCGTTCGTAGCACAACAACAATGACGGT |
| | TTGATGCGGGTGCATACATCTTTTCCTCCGACACCGGTCAAGGGCATTTACAACAAAAATCAGTAAGGCAAA |
| | CGGTGCTATCCGAAGTGGTGTTGGAGAGGACCGAATTGGAGATTTCGTATGCCCCGCGCCTCGACCAAGAAA |
| | AAGAAGAATTACTACGCAAGAAATTACAGTTAAATCCCACACCTGCTAACAGAAGCAGATACCAGTCCAGGA |
| | AGGTGGAGAACATGAAAGCCATAACAGCTAGACGTATTCTGCAAGGCCTAGGGCATTATTTGAAGGCAGAAG |
| | GAAAAGTGGAGTGCTACCGAACCCTGCATCCTGTTCCTTTGTATTCATCTAGTGTGAACCGTGCCTTTTCAA |
| | GCCCCAAGGTCGCAGTGGAAGCCTGTAACGCCATGTTGAAAGAGAACTTTCCGACTGTGGCTTCTTACTGTA |
| | TTATTCCAGAGTACGATGCCTATTTGGACATGGTTGACGGAGCTTCATGCTGCTTAGACACTGCCAGTTTTT |
| | GCCCTGCAAAGCTGCGCAGCTTTCCAAAGAAACACTCCTATTTGGAACCCACAATACGATCGGCAGTGCCTT |
| | CAGCGATCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATAATGAAT |
| | ATTGGGAAACGTTTAAAGAAAACCCCATCAGGCTTACTGAAGAAAACGTGGTAAATTACATTACCAAATTAA |
| | AAGGACCAAAAGCTGCTGCTCTTTTTGCGAAGACACATAATTTGAATATGTTGCAGGACATACCAATGGACA |
| | GGTTTGTAATGGACTTAAAGAGAGACGTGAAAGTGACTCCAGGAACAAAACATACTGAAGAACGGCCCAAGG |
| | TACAGGTGATCCAGGCTGCCGATCCGCTAGCAACAGCGTATCTGTGCGGAATCCACCGAGAGCTGGTTAGGA |
| | GATTAAATGCGGTCCTGCTTCCGAACATTCATACACTGTTTGATATGTCGGCTGAAGACTTTGACGCTATTA |
| | TAGCCGAGCACTTCCAGCCTGGGGATTGTGTTCTGGAAACTGACATCGCGTCGTTTGATAAAAGTGAGGACG |
| | ACGCCATGGCTCTGACCGCGTTAATGATTCTGGAAGACTTAGGTGTGGACGCAGAGCTGTTGACGCTGATTG |
| | AGGCGGCTTTCGGCGAAATTTCATCAATACATTTGCCCACTAAAACTAAATTTAAATTCGGAGCCATGATGA |
| | AATCTGGAATGTTCCTCACACTGTTTGTGAACACAGTCATTAACATTGTAATCGCAAGCAGAGTGTTGAGAG |
| | AACGGCTAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGGGCGAGA |
| | AAGCGCCTTATTTCTGTGGAGGGTTTATTTTGTGTGACTCCGTGACCGGCACAGCGTGCCGTGTGGCAGACC |
| | CCCTAAAAAGGCTGTTTAAGCTTGGCAAACCTCTGGCAGCAGACGATGAACATGATGATGACAGGAGAAGGG |
| | CATTGCATGAAGAGTCAACACGCTGGAACCGAGTGGGTATTCTTTCAGAGCTGTGCAAGGCAGTAGAATCAA |
| | GGTATGAAACCGTAGGAACTTCCATCATAGTTATGGCCATGACTACTCTAGCTAGCAGTGTTAAATCATTCA |
| | GCTACCTGAGAGGGGCCCCTATAACTCTCTACGGCTAACCTGAATGGACTACGACATAGTCTAGTCCGCCAA |
| | GATGGAGAAAATAGTGCTTCTTCTTGCAATAGTCAGCCTTGTTAAAAGTGATCAGATTTGCATTGGTTACCA |
| | TGCAAACAACTCGACAGAGCAGGTTGACACAATAATGGAAAAGAACGTCACTGTTACACACGCCCAAGACAT |
| | ACTGGAAAAGACACACAACGGGAAACTCTGCGATCTAGATGGAGTGAAGCCTCTAATTTTAAGAGATTGTAG |
| | TGTAGCTGGATGGCTCCTCGGGAACCCAATGTGTGACGAATTCCTCAATGTGCCGGAATGGTCTTACATAGT |
| | GGAGAAGATCAATCCAGCCAATGACCTCTGTTACCCAGGGAATTTCAACGACTATGAAGAACTGAAACACCT |
| | ATTGAGCAGAATAAACCATTTTGAGAAAATTCAGATCATCCCCAAAAGTTCTTGGTCAGATCATGAAGCCTC |
| | AGCAGGGGTGAGCTCAGCATGTCCATACCAGGGAAGGTCCTCCTTTTTTAGAAATGTGGTATGGCTTATCAA |
| | AAAGGACAATGCATACCCAACAATAAAGAGAAGTTACAATAATACCAACCAAGAAGATCTTTTGGTATTGTG |
| | GGGGATTCACCATCCAAATGATGCGGCAGAGCAGACAAGGCTCTATCAAAACCCAACTACCTATATTTCCGT |
| | TGGGACATCAACACTAAACCAGAGATTGGTACCAAAAATAGCCACTAGATCTAAGGTAAACGGGCAAAGTGG |
| | AAGGATGGAGTTCTTTTGGACAATTTTAAAACCGAATGATGCAATAAACTTTGAGAGTAATGGAAATTTCAT |
| | TGCTCCAGAAAATGCATACAAAATTGTCAAGAAAGGGGACTCAACAATTATGAAAAGTGAGTTGGAATATGG |
| | TAACTGCAACACCAAGTGTCAAACTCCAATAGGGGCGATAAACTCTAGTATGCCATTCCACAACATCCACCC |
| | TCTCACCATCGGGGAATGCCCCAAATATGTGAAATCAAGCAGATTAGTCCTTGCTACTGGGCTCAGAAATAG |
| | CCCTCAACGAGAGACACGAGGACTATTTGGAGCTATAGCAGGTTTTATAGAGGGAGGATGGGGAATGGTAGA |
| | TGGTTGGTATGGGTACCACCATAGCAACGAGCAGGGGAGTGGGTACGCTGCAGACAAAGAATCCACTCAAAA |
| | GGCAATAGATGGAGTCACCAATAAGGTCAACTCGATCATTGACAAAATGAACACTCAGTTTGAGGCTGTTGG |
| | AAGGGAATTTAATAACTTAGAAAGGAGAATAGAAAATTTAAACAAGAAGATGGAAGACGGATTCCTAGATGT |
| | CTGGACTTATAATGCTGAACTTCTGGTTCTCATGGAAAATGAGAGAACTCTAGACTTTCATGACTCAAATGT |
| | CAAGAACCTTTACGACAAGGTCCGACTACAGCTTAGGGATAATGCAAAGGAGCTTGGTAACGGTTGTTTCGA |
| | GTTCTATCACAGATGTGATAATGAATGTATGGAAAGTGTAAGAAACGGAACGTATGACTACCCGCAGTATTC |
| | AGAAGAAGCAAGATTAAAAAGAGAGGAAATAAGTGGAGTAAAATTGGAATCAATAGGAACTTACCAAATACT |
| | GTCAATTTATTCAACAGTGGCGAGCTCCCTAGCACTGGCAATCATGGTGGCTGGTCTATCTTTATGGATGTG |
| | CTCCAATGGATCGTTACAATGCAGAATTTGCATTTAACTCTCTACGGCTAACCTGAATGGACTACGACATAG |
| | TCTAGTCCGCCAAGATGAATCCAAATCAGAAGATAATAACCATCGGATCAATCTGTATGGTAATTGGAATAG |
| | TTAGCTTAATGTTACAAATTGGGAACATGATCTCAATATGGGTCAGTCATTCAATTCAGACAGGGAATCAAT |
| | GCCAAGCTGAACCAATCAGCAATACTAAATTTCTTACTGAGAAAGCTGTGGCTTCAGTAACATTAGCGGGCA |
| | ATTCATCTCTTTGCCCCATTAGCGGATGGGCTGTATACAGTAAGGACAACAGTATAAGGATCGGTTCCAGGG |
| | GGGATGTGTTTGTTATAAGAGAGCCGTTCATCTCATGCTCCCACTTGGAATGCAGAACTTTCTTTTTGACTC |
| | AGGGAGCCTTGCTGAATGACAAGCACTCCAATGGGACTGTCAAAGACAGAAGCCCTCACAGAACATTAATGA |
| | GTTGTCCTGTGGGTGAGGCTCCCTCCCCATATAACTCAAGGTTTGAGTCTGTTGCTTGGTCAGCAAGTGCTT |
| | GCCATGATGGCACCAGTTGGTTGACAATTGGAATTTCTGGTCCAGACAATGGGGCTGTGGCTGTATTGAAAT |
| | ACAATGGCATAATAACAGACACCATCAAGAGTTGGAGGAACAACATACTGAGAACTCAAGAGTCTGAATGTG |
| | CATGTGTAAATGGCTCTTGCTTTACTGTAATGACTGATGGACCAAGTAGTGGGCAGGCATCATATAAGATCT |
| | TCAAAATGGAAAAAGGGAAAGTGGTTAAATCAGTCGAATTGGATGCTCCTAATTATCACTATGAGGAGTGCT |
| | CCTGTTATCCTGATGCCGGCGAAATCACATGTGTGTGCAGGGATAATTGGCATGGCTCAAATAGGCCATGGG |
| | TATCTTTCAATCAAAATTTGGAGTATCAAATAGGATATATATGCAGTGGAGTTTTCGGAGACAATCCACGCC |
| | CCAATGATGGAACAGGTAGTTGTGGTCCGGTGTTCTCTAACGGGGCATATGGGGTAAAAGGGTTTTCATTCA |
| | AATACGGCAATGGTGTTTGGATCGGGAGAACCAAAAGCACTAATTCCAGGAGCGGCTTTGAAATGATTTGGG |
| | ACCCAAATGGGTGGACTGGAACGGACAGTAGCTTTTCGGTGAAGCAAGATATCGTAGCAATAACTGATTGGT |
| | CAGGATATAGCGGGAGTTTTGTCCAGCATCCAGAACTGACAGGATTAGATTGCATAAGACCTTGTTTCTGGG |
| | TTGAGTTAATCAGAGGGCGGCCTAAAGAGAGCACAATTTGGACCAGTGGGAGCAGCATATCTTTTTGTGGTG |
| | TAAATAGTGACACTGTTAGTTGGTCTTGGCCAGACGGTGCTGAGTTGCCATTCACCATTGACAAGTAGCTCT |
| | CTACGGCTAACCTGAATGGACTACGACATAGTCTAGTCCGCCAAGATGAGTCTTCTAACCGAGGTCGAAACG |
| | TACGTACTCTCTATCATCCCGTCAGGCCCCCTCAAAGCCGAGATCGCACAGAGACTTGAAGATGTCTTTGCA |
| | GGGAAGAACACCGATCTTGAGGTTCTCATGGAATGGCTAAAGACAAGACCAATCCTGTCACCTCTGACTAAG |
| | GGGATTTTAGGATTTGTGTTCACGCTCACCGTGCCCAGTGAGCGAGGACTGCAGCGTAGACGCTTTGTCCAA |
| | AATGCCCTTAATGGGAACGGGGATCCAAATAACATGGACAAAGCAGTTAAACTGTATAGGAAGCTCAAGAGG |
| | GAGATAACATTCCATGGGGCCAAAGAAATCTCACTCAGTTATTCTGCTGGTGCACTTGCCAGTTGTATGGGC |
| | CTCATATACAACAGGATGGGGGCTGTGACCACTGAAGTGGCATTTGGCCTGGTATGTGCAACCTGTGAACAG |
| | ATTGCTGACTCCCAGCATCGGTCTCATAGGCAAATGGTGACAACAACCAATCCACTAATCAGACATGAGAAC |
| | AGAATGGTTTTAGCCAGCACTACAGCTAAGGCTATGGAGCAAATGGCTGGATCGAGTGAGCAAGCAGCAGAG |
| | GCCATGGAGGTTGCTAGTCAGGCTAGACAAATGGTGCAAGCGATGAGAACCATTGGGACTCATCCTAGCTCC |
| | AGTGCTGGTCTGAAAAATGATCTTCTTGAAAATTTGCAGGCCTATCAGAAACGAATGGGGGTGCAGATGCAA |
| | CGGTTCAAGTGAGGCGCGCCCACCCAGCGGCCGCCCGCTACGCCCCAATGATCCGACCAGCAAAACTCGATG |
| | TACTTCCGAGGAACTGATGTGCATAATGCATCAGGCTGGTACATTAGATCCCCGCTTACCGCGGGCAATATA |
| | GCAACACTAAAAACTCGATGTACTTCCGAGGAAGCGCAGTGCATAATGCTGCGCAGTGTTGCCACATAACCA |
| | CTATATTAACCATTTATCTAGCGGACGCCAAAAACTCAATGTATTTCTGAGGAAGCGTGGTGCATAATGCCA |
| | CGCAGCGTCTGCATAACTTTTATTATTTCTTTTATTAATCAACAAAATTTTGTTTTTAACATTTCAAAAAAA |
| | AAAAAAAAAAAAAAAAAAAAAAAAAAAAAAGAAGAGCGTTTAAACACGTGATATCTGGCCTCATGGGCCTTC |
| | CTTTCACTGCCCGCTTTCCAGTCGGGAAACCTGTCGTGCCAGCTGCATTAACATGGTCATAGCTGTTTCCTT |
| | GCGTATTGGGCGCTCTCCGCTTCCTCGCTCACTGACTCGCTGCGCTCGGTCGTTCGGGTAAAGCCTGGGGTG |
| | CCTAATGAGCAAAAGGCCAGCAAAAGGCCAGGAACCGTAAAAAGGCCGCGTTGCTGGCGTTTTTCCATAGGC |
| | TCCGCCCCCCTGACGAGCATCACAAAAATCGACGCTCAAGTCAGAGGTGGCGAAACCCGACAGGACTATAAA |
| | GATACCAGGCGTTTCCCCCTGGAAGCTCCCTCGTGCGCTCTCCTGTTCCGACCCTGCCGCTTACCGGATACC |
| | TGTCCGCCTTTCTCCCTTCGGGAAGCGTGGCGCTTTCTCATAGCTCACGCTGTAGGTATCTCAGTTCGGTGT |
| | AGGTCGTTCGCTCCAAGCTGGGCTGTGTGCACGAACCCCCCGTTCAGCCCGACCGCTGCGCCTTATCCGGTA |
| | ACTATCGTCTTGAGTCCAACCCGGTAAGACACGACTTATCGCCACTGGCAGCAGCCACTGGTAACAGGATTA |
| | GCAGAGCGAGGTATGTAGGCGGTGCTACAGAGTTCTTGAAGTGGTGGCCTAACTACGGCTACACTAGAAGAA |
| | CAGTATTTGGTATCTGCGCTCTGCTGAAGCCAGTTACCTTCGGAAAAAGAGTTGGTAGCTCTTGATCCGGCA |
| | AACAAACCACCGCTGGTAGCGGTGGTTTTTTTGTTTGCAGGCAGCAGATTACGCGCAGAAAAAAAGGATCTC |
| | AAGAAGATCCTTTGATCTTTTCTACGGGGTCTGACGCTCAGTGGAACGAAAACTCACGTTAAGGGATTTTGG |
| | TCATGAATACACGGTGCCTGACTGCGTTAGCAATTTAACTGTGATAAACTACCGCATTAAAGCTTATCGATG |
| | ATAAGCTGTCAAACATGAGAATTCTTAGAAAAACTCATCGAGCATCAAATGAAACTGCAATTTATTCATATC |
| | AGGATTATCAATACCATATTTTTGAAAAAGCCGTTTCTGTAATGAAGGAGAAAACTCACCGAGGCAGTTCCA |
| | TAGGATGGCAAGATCCTGGTATCGGTCTGCGATTCCGACTCGTCCAACATCAATACAACCTATTAATTTCCC |
| | CTCGTCAAAAATAAGGTTATCAAGTGAGAAATCACCATGAGTGACGACTGAATCCGGTGAGAATGGCAAAAG |
| | CTTATGCATTTCTTTCCAGACTTGTTCAACAGGCCAGCCATTACGCTCGTCATCAAAATCACTCGCATCAAC |
| | CAAACCGTTATTCATTCGTGATTGCGCCTGAGCGAGACGAAATACGCGATCGCTGTTAAAAGGACAATTACA |
| | AACAGGAATCGAATGCAACCGGCGCAGGAACACTGCCAGCGCATCAACAATATTTTCACCTGAATCAGGATA |
| | TTCTTCTAATACCTGGAATGCTGTTTTCCCGGGGATCGCAGTGGTGAGTAACCATGCATCATCAGGAGTACG |
| | GATAAAATGCTTGATGGTCGGAAGAGGCATAAATTCCGTCAGCCAGTTTAGTCTGACCATCTCATCTGTAAC |
| | ATCATTGGCAACGCTACCTTTGCCATGTTTCAGAAACAACTCTGGCGCATCGGGCTTCCCATACAATCGATA |
| | GATTGTCGCACCTGATTGCCCGACATTATCGCGAGCCCATTTATACCCATATAAATCAGCATCCATGTTGGA |
| | ATTTAATCGCGGCCTCGAGCAAGACGTTTCCCGTTGAATATGGCTCATAACACCCCTTGTATTACTGTTTAT |
| | GTAAGCAGACAGTTTTATTGTTCATGAGCGGATACATATTTGAATGTATTTAGAAAAATAAACAAATAGGGG |
| | TTCCGCGCACATTTCCCCGAAAAGTGCCACCTAAATTGTAAGCGTTAATATTTTGTTAAAATTCGCGTTAAA |
| | TTTTTGTTAAATCAGCTCATTTTTTAACCAATAGGCCGAAATCGGCAAAATCCCTTATAAATCAAAAGAATA |
| | GACCGAGATAGGGTTGAGTGGCCGCTACAGGGCGCTCCCATTCGCCATTCAGGCTGCGCAACTGTTGGGAAG |
| | GGCGTTTCGGTGCGGGCCTCTTCGCTATTACGCCAGCTGGCGAAAGGGGGATGTGCTGCAAGGCGATTAAGT |
| | TGGGTAACGCCAGGGTTTTCCCAGTCACACGCGTAATACGACTCACTATAG |
| 82 | ATAGGCGGCGCATGAGAGAAGCCCAGACCAATTACCTACCCAAAATGGAGAAAGTTCACGTTGACATCGAGG |
| | AAGACAGCCCATTCCTCAGAGCTTTGCAGCGGAGCTTCCCGCAGTTTGAGGTAGAAGCCAAGCAGGTCACTG |
| | ATAATGACCATGCTAATGCCAGAGCGTTTTCGCATCTGGCTTCAAAACTGATCGAAACGGAGGTGGACCCAT |
| | CCGACACGATCCTTGACATTGGAAGTGCGCCCGCCCGCAGAATGTATTCTAAGCACAAGTATCATTGTATCT |
| | GTCCGATGAGATGTGCGGAAGATCCGGACAGATTGTATAAGTATGCAACTAAGCTGAAGAAAAACTGTAAGG |
| | AAATAACTGATAAGGAATTGGACAAGAAAATGAAGGAGCTCGCCGCCGTCATGAGCGACCCTGACCTGGAAA |
| | CTGAGACTATGTGCCTCCACGACGACGAGTCGTGTCGCTACGAAGGGCAAGTCGCTGTTTACCAGGATGTAT |
| | ACGCGGTTGACGGACCGACAAGTCTCTATCACCAAGCCAATAAGGGAGTTAGAGTCGCCTACTGGATAGGCT |
| | TTGACACCACCCCTTTTATGTTTAAGAACTTGGCTGGAGCATATCCATCATACTCTACCAACTGGGCCGACG |
| | AAACCGTGTTAACGGCTCGTAACATAGGCCTATGCAGCTCTGACGTTATGGAGCGGTCACGTAGAGGGATGT |
| | CCATTCTTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATTACACAT |
| | GTCGGTGTGAGACTATAGTTAGTTGCGACGGGTACGTCGTTAAAAGAATAGCTATCAGTCCAGGCCTGTATG |
| | GGAAGCCTTCAGGCTATGCTGCTACGATGCACCGCGAGGGATTCTTGTGCTGCAAAGTGACAGACACATTGA |
| | ACGGGGAGAGGGTCTCTTTTCCCGTGTGCACGTATGTGCCAGCTACATTGTGTGACCAAATGACTGGCATAC |
| | TGGCAACAGATGTCAGTGCGGACGACGCGCAAAAACTGCTGGTTGGGCTCAACCAGCGTATAGTCGTCAACG |
| | GTCGCACCCAGAGAAACACCAATACCATGAAAAATTACCTTTTGCCCGTAGTGGCCCAGGCATTTGCTAGGT |
| | GGGCAAAGGAATATAAGGAAGATCAAGAAGATGAAAGGCCACTAGGACTACGAGATAGACAGTTAGTCATGG |
| | GGTGTTGTTGGGCTTTTAGAAGGCACAAGATAACATCTATTTATAAGCGCCCGGATACCCAAACCATCATCA |
| | AAGTGAACAGCGATTTCCACTCATTCGTGCTGCCCAGGATAGGCAGTAACACATTGGAGATCGGGCTGAGAA |
| | CAAGAATCAGGAAAATGTTAGAGGAGCACAAGGAGCCGTCACCTCTCATTACCGCCGAGGACGTACAAGAAG |
| | CTAAGTGCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCGGCTCAG |
| | TGGAGACACCTCGTGGCTTGATAAAGGTTACCAGCTACGATGGCGAGGACAAGATCGGCTCTTACGCTGTGC |
| | TTTCTCCGCAGGCTGTACTCAAGAGTGAAAAATTATCTTGCATCCACCCTCTCGCTGAACAAGTCATAGTGA |
| | TAACACACTCTGGCCGAAAAGGGCGTTATGCCGTGGAACCATACCATGGTAAAGTAGTGGTGCCAGAGGGAC |
| | ATGCAATACCCGTCCAGGACTTTCAAGCTCTGAGTGAAAGTGCCACCATTGTGTACAACGAACGTGAGTTCG |
| | TAAACAGGTACCTGCACCATATTGCCACACATGGAGGAGCGCTGAACACTGATGAAGAATATTACAAAACTG |
| | TCAAGCCCAGCGAGCACGACGGCGAATACCTGTACGACATCGACAGGAAACAGTGCGTCAAGAAAGAACTAG |
| | TCACTGGGCTAGGGCTCACAGGCGAGCTGGTGGATCCTCCCTTCCATGAATTCGCCTACGAGAGTCTGAGAA |
| | CACGACCAGCCGCTCCTTACCAAGTACCAACCATAGGGGTGTATGGCGTGCCAGGATCAGGCAAGTCTGGCA |
| | TCATTAAAAGCGCAGTCACCAAAAAAGATCTAGTGGTGAGCGCCAAGAAAGAAAACTGTGCAGAAATTATAA |
| | GGGACGTCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGCTCATAG |
| | CCATTATAAGACCTAAAAAGGCAGTGCTCTGCGGGGATCCCAAACAGTGCGGTTTTTTTAACATGATGTGCC |
| | TGAAAGTGCATTTTAACCACGAGATTTGCACACAAGTCTTCCACAAAAGCATCTCTCGCCGTTGCACTAAAT |
| | CTGTGACTTCGGTCGTCTCAACCTTGTTTTACGACAAAAAAATGAGAACGACGAATCCGAAAGAGACTAAGA |
| | TTGTGATTGACACTACCGGCAGTACCAAACCTAAGCAGGACGATCTCATTCTCACTTGTTTCAGAGGGTGGG |
| | TGAAGCAGTTGCAAATAGATTACAAAGGCAACGAAATAATGACGGCAGCTGCCTCTCAAGGGCTGACCCGTA |
| | AAGGTGTGTATGCCGTTCGGTACAAGGTGAATGAAAATCCTCTGTACGCACCCACCTCAGAACATGTGAACG |
| | TCCTACTGACCCGCACGGAGGACCGCATCGTGTGGAAAACACTAGCCGGCGACCCATGGATAAAAACACTGA |
| | CTGCCAAGTACCCTGGGAATTTCACTGCCACGATAGAGGAGTGGCAAGCAGAGCATGATGCCATCATGAGGC |
| | ACATCTTGGAGAGACCGGACCCTACCGACGTCTTCCAGAATAAGGCAAACGTGTGTTGGGCCAAGGCTTTAG |
| | TGCCGGTGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGGACTCCG |
| | GTCTATTTTCTGCACCCACTGTTCCGTTATCCATTAGGAATAATCACTGGGATAACTCCCCGTCGCCTAACA |
| | TGTACGGGCTGAATAAAGAAGTGGTCCGTCAGCTCTCTCGCAGGTACCCACAACTGCCTCGGGCAGTTGCCA |
| | CTGGAAGAGTCTATGACATGAACACTGGTACACTGCGCAATTATGATCCGCGCATAAACCTAGTACCTGTAA |
| | ACAGAAGACTGCCTCATGCTTTAGTCCTCCACCATAATGAACACCCACAGAGTGACTTTTCTTCATTCGTCA |
| | GCAAATTGAAGGGCAGAACTGTCCTGGTGGTCGGGGAAAAGTTGTCCGTCCCAGGCAAAATGGTTGACTGGT |
| | TGTCAGACCGGCCTGAGGCTACCTTCAGAGCTCGGCTGGATTTAGGCATCCCAGGTGATGTGCCCAAATATG |
| | ACATAATATTTGTTAATGTGAGGACCCCATATAAATACCATCACTATCAGCAGTGTGAAGACCATGCCATTA |
| | AGCTTAGCATGTTGACCAAGAAAGCTTGTCTGCATCTGAATCCCGGCGGAACCTGTGTCAGCATAGGTTATG |
| | GTTACGCTGACAGGGCCAGCGAAAGCATCATTGGTGCTATAGCGCGGCAGTTCAAGTTTTCCCGGGTATGCA |
| | AACCGAAATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCGGATGTG |
| | CACCCTCATATCATGTGGTGCGAGGGGATATTGCCACGGCCACCGAAGGAGTGATTATAAATGCTGCTAACA |
| | GCAAAGGACAACCTGGCGGAGGGGTGTGCGGAGCGCTGTATAAGAAATTCCCGGAAAGCTTCGATTTACAGC |
| | CGATCGAAGTAGGAAAAGCGCGACTGGTCAAAGGTGCAGCTAAACATATCATTCATGCCGTAGGACCAAACT |
| | TCAACAAAGTTTCGGAGGTTGAAGGTGACAAACAGTTGGCAGAGGCTTATGAGTCCATCGCTAAGATTGTCA |
| | ACGATAACAATTACAAGTCAGTAGCGATTCCACTGTTGTCCACCGGCATCTTTTCCGGGAACAAAGATCGAC |
| | TAACCCAATCATTGAACCATTTGCTGACAGCTTTAGACACCACTGATGCAGATGTAGCCATATACTGCAGGG |
| | ACAAGAAATGGGAAATGACTCTCAAGGAAGCAGTGGCTAGGAGAGAAGCAGTGGAGGAGATATGCATATCCG |
| | ACGACTCTTCAGTGACAGAACCTGATGCAGAGCTGGTGAGGGTGCATCCGAAGAGTTCTTTGGCTGGAAGGA |
| | AGGGCTACAGCACAAGCGATGGCAAAACTTTCTCATATTTGGAAGGGACCAAGTTTCACCAGGCGGCCAAGG |
| | ATATAGCAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTAGCACGC |
| | TGCCTTGCTTGTGCATCCATGCCATGACTCCAGAAAGAGTACAGCGCCTAAAAGCCTCACGTCCAGAACAAA |
| | TTACTGTGTGCTCATCCTTTCCATTGCCGAAGTATAGAATCACTGGTGTGCAGAAGATCCAATGCTCCCAGC |
| | CTATATTGTTCTCACCGAAAGTGCCTGCGTATATTCATCCAAGGAAGTATCTCGTGGAAACACCACCGGTAG |
| | ACGAGACTCCGGAGCCATCGGCAGAGAACCAATCCACAGAGGGGACACCTGAACAACCACCACTTATAACCG |
| | AGGATGAGACCAGGACTAGAACGCCTGAGCCGATCATCATCGAAGAGGAAGAAGAGGATAGCATAAGTTTGC |
| | TGTCAGATGGCCCGACCCACCAGGTGCTGCAAGTCGAGGCAGACATTCACGGGCCGCCCTCTGTATCTAGCT |
| | CATCCTGGTCCATTCCTCATGCATCCGACTTTGATGTGGACAGTTTATCCATACTTGACACCCTGGAGGGAG |
| | CTAGCGTGACCAGCGGGGCAACGTCAGCCGAGACTAACTCTTACTTCGCAAAGAGTATGGAGTTTCTGGCGC |
| | GACCGGTGCCTGCGCCTCGAACAGTATTCAGGAACCCTCCACATCCCGCTCCGCGCACAAGAACACCGTCAC |
| | TTGCACCCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGGTCTCCA |
| | ACCCGCCAGGCGTAAATAGGGTGATTACAAGAGAGGAGTTTGAGGCGTTCGTAGCACAACAACAATGACGGT |
| | TTGATGCGGGTGCATACATCTTTTCCTCCGACACCGGTCAAGGGCATTTACAACAAAAATCAGTAAGGCAAA |
| | CGGTGCTATCCGAAGTGGTGTTGGAGAGGACCGAATTGGAGATTTCGTATGCCCCGCGCCTCGACCAAGAAA |
| | AAGAAGAATTACTACGCAAGAAATTACAGTTAAATCCCACACCTGCTAACAGAAGCAGATACCAGTCCAGGA |
| | AGGTGGAGAACATGAAAGCCATAACAGCTAGACGTATTCTGCAAGGCCTAGGGCATTATTTGAAGGCAGAAG |
| | GAAAAGTGGAGTGCTACCGAACCCTGCATCCTGTTCCTTTGTATTCATCTAGTGTGAACCGTGCCTTTTCAA |
| | GCCCCAAGGTCGCAGTGGAAGCCTGTAACGCCATGTTGAAAGAGAACTTTCCGACTGTGGCTTCTTACTGTA |
| | TTATTCCAGAGTACGATGCCTATTTGGACATGGTTGACGGAGCTTCATGCTGCTTAGACACTGCCAGTTTTT |
| | GCCCTGCAAAGCTGCGCAGCTTTCCAAAGAAACACTCCTATTTGGAACCCACAATACGATCGGCAGTGCCTT |
| | CAGCGATCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATAATGAAT |
| | ATTGGGAAACGTTTAAAGAAAACCCCATCAGGCTTACTGAAGAAAACGTGGTAAATTACATTACCAAATTAA |
| | AAGGACCAAAAGCTGCTGCTCTTTTTGCGAAGACACATAATTTGAATATGTTGCAGGACATACCAATGGACA |
| | GGTTTGTAATGGACTTAAAGAGAGACGTGAAAGTGACTCCAGGAACAAAACATACTGAAGAACGGCCCAAGG |
| | TACAGGTGATCCAGGCTGCCGATCCGCTAGCAACAGCGTATCTGTGCGGAATCCACCGAGAGCTGGTTAGGA |
| | GATTAAATGCGGTCCTGCTTCCGAACATTCATACACTGTTTGATATGTCGGCTGAAGACTTTGACGCTATTA |
| | TAGCCGAGCACTTCCAGCCTGGGGATTGTGTTCTGGAAACTGACATCGCGTCGTTTGATAAAAGTGAGGACG |
| | ACGCCATGGCTCTGACCGCGTTAATGATTCTGGAAGACTTAGGTGTGGACGCAGAGCTGTTGACGCTGATTG |
| | AGGCGGCTTTCGGCGAAATTTCATCAATACATTTGCCCACTAAAACTAAATTTAAATTCGGAGCCATGATGA |
| | AATCTGGAATGTTCCTCACACTGTTTGTGAACACAGTCATTAACATTGTAATCGCAAGCAGAGTGTTGAGAG |
| | AACGGCTAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGGGCGAGA |
| | AAGCGCCTTATTTCTGTGGAGGGTTTATTTTGTGTGACTCCGTGACCGGCACAGCGTGCCGTGTGGCAGACC |
| | CCCTAAAAAGGCTGTTTAAGCTTGGCAAACCTCTGGCAGCAGACGATGAACATGATGATGACAGGAGAAGGG |
| | CATTGCATGAAGAGTCAACACGCTGGAACCGAGTGGGTATTCTTTCAGAGCTGTGCAAGGCAGTAGAATCAA |
| | GGTATGAAACCGTAGGAACTTCCATCATAGTTATGGCCATGACTACTCTAGCTAGCAGTGTTAAATCATTCA |
| | GCTACCTGAGAGGGGCCCCTATAACTCTCTACGGCTAACCTGAATGGACTACGACATAGTCTAGTCCGCCAA |
| | GATGGAGAAAATAGTGCTTCTTCTTGCAATAGTCAGCCTTGTTAAAAGTGATCAGATTTGCATTGGTTACCA |
| | TGCAAACAACTCGACAGAGCAGGTTGACACAATAATGGAAAAGAACGTCACTGTTACACACGCCCAAGACAT |
| | ACTGGAAAAGACACACAACGGGAAACTCTGCGATCTAGATGGAGTGAAGCCTCTAATTTTAAGAGATTGTAG |
| | TGTAGCTGGATGGCTCCTCGGGAACCCAATGTGTGACGAATTCCTCAATGTGCCGGAATGGTCTTACATAGT |
| | GGAGAAGATCAATCCAGCCAATGACCTCTGTTACCCAGGGAATTTCAACGACTATGAAGAACTGAAACACCT |
| | ATTGAGCAGAATAAACCATTTTGAGAAAATTCAGATCATCCCCAAAAGTTCTTGGTCAGATCATGAAGCCTC |
| | AGCAGGGGTGAGCTCAGCATGTCCATACCAGGGAAGGTCCTCCTTTTTTAGAAATGTGGTATGGCTTATCAA |
| | AAAGGACAATGCATACCCAACAATAAAGAGAAGTTACAATAATACCAACCAAGAAGATCTTTTGGTATTGTG |
| | GGGGATTCACCATCCAAATGATGCGGCAGAGCAGACAAGGCTCTATCAAAACCCAACTACCTATATTTCCGT |
| | TGGGACATCAACACTAAACCAGAGATTGGTACCAAAAATAGCCACTAGATCTAAGGTAAACGGGCAAAGTGG |
| | AAGGATGGAGTTCTTTTGGACAATTTTAAAACCGAATGATGCAATAAACTTTGAGAGTAATGGAAATTTCAT |
| | TGCTCCAGAAAATGCATACAAAATTGTCAAGAAAGGGGACTCAACAATTATGAAAAGTGAGTTGGAATATGG |
| | TAACTGCAACACCAAGTGTCAAACTCCAATAGGGGCGATAAACTCTAGTATGCCATTCCACAACATCCACCC |
| | TCTCACCATCGGGGAATGCCCCAAATATGTGAAATCAAGCAGATTAGTCCTTGCTACTGGGCTCAGAAATAG |
| | CCCTCAACGAGAGACACGAGGACTATTTGGAGCTATAGCAGGTTTTATAGAGGGAGGATGGGGAATGGTAGA |
| | TGGTTGGTATGGGTACCACCATAGCAACGAGCAGGGGAGTGGGTACGCTGCAGACAAAGAATCCACTCAAAA |
| | GGCAATAGATGGAGTCACCAATAAGGTCAACTCGATCATTGACAAAATGAACACTCAGTTTGAGGCTGTTGG |
| | AAGGGAATTTAATAACTTAGAAAGGAGAATAGAAAATTTAAACAAGAAGATGGAAGACGGATTCCTAGATGT |
| | CTGGACTTATAATGCTGAACTTCTGGTTCTCATGGAAAATGAGAGAACTCTAGACTTTCATGACTCAAATGT |
| | CAAGAACCTTTACGACAAGGTCCGACTACAGCTTAGGGATAATGCAAAGGAGCTTGGTAACGGTTGTTTCGA |
| | GTTCTATCACAGATGTGATAATGAATGTATGGAAAGTGTAAGAAACGGAACGTATGACTACCCGCAGTATTC |
| | AGAAGAAGCAAGATTAAAAAGAGAGGAAATAAGTGGAGTAAAATTGGAATCAATAGGAACTTACCAAATACT |
| | GTCAATTTATTCAACAGTGGCGAGCTCCCTAGCACTGGCAATCATGGTGGCTGGTCTATCTTTATGGATGTG |
| | CTCCAATGGATCGTTACAATGCAGAATTTGCATTTAACTCTCTACGGCTAACCTGAATGGACTACGACATAG |
| | TCTAGTCCGCCAAGATGAATCCAAATCAGAAGATAATAACCATCGGATCAATCTGTATGGTAATTGGAATAG |
| | TTAGCTTAATGTTACAAATTGGGAACATGATCTCAATATGGGTCAGTCATTCAATTCAGACAGGGAATCAAT |
| | GCCAAGCTGAACCAATCAGCAATACTAAATTTCTTACTGAGAAAGCTGTGGCTTCAGTAACATTAGCGGGCA |
| | ATTCATCTCTTTGCCCCATTAGCGGATGGGCTGTATACAGTAAGGACAACAGTATAAGGATCGGTTCCAGGG |
| | GGGATGTGTTTGTTATAAGAGAGCCGTTCATCTCATGCTCCCACTTGGAATGCAGAACTTTCTTTTTGACTC |
| | AGGGAGCCTTGCTGAATGACAAGCACTCCAATGGGACTGTCAAAGACAGAAGCCCTCACAGAACATTAATGA |
| | GTTGTCCTGTGGGTGAGGCTCCCTCCCCATATAACTCAAGGTTTGAGTCTGTTGCTTGGTCAGCAAGTGCTT |
| | GCCATGATGGCACCAGTTGGTTGACAATTGGAATTTCTGGTCCAGACAATGGGGCTGTGGCTGTATTGAAAT |
| | ACAATGGCATAATAACAGACACCATCAAGAGTTGGAGGAACAACATACTGAGAACTCAAGAGTCTGAATGTG |
| | CATGTGTAAATGGCTCTTGCTTTACTGTAATGACTGATGGACCAAGTAGTGGGCAGGCATCATATAAGATCT |
| | TCAAAATGGAAAAAGGGAAAGTGGTTAAATCAGTCGAATTGGATGCTCCTAATTATCACTATGAGGAGTGCT |
| | CCTGTTATCCTGATGCCGGCGAAATCACATGTGTGTGCAGGGATAATTGGCATGGCTCAAATAGGCCATGGG |
| | TATCTTTCAATCAAAATTTGGAGTATCAAATAGGATATATATGCAGTGGAGTTTTCGGAGACAATCCACGCC |
| | CCAATGATGGAACAGGTAGTTGTGGTCCGGTGTTCTCTAACGGGGCATATGGGGTAAAAGGGTTTTCATTCA |
| | AATACGGCAATGGTGTTTGGATCGGGAGAACCAAAAGCACTAATTCCAGGAGCGGCTTTGAAATGATTTGGG |
| | ACCCAAATGGGTGGACTGGAACGGACAGTAGCTTTTCGGTGAAGCAAGATATCGTAGCAATAACTGATTGGT |
| | CAGGATATAGCGGGAGTTTTGTCCAGCATCCAGAACTGACAGGATTAGATTGCATAAGACCTTGTTTCTGGG |
| | TTGAGTTAATCAGAGGGCGGCCTAAAGAGAGCACAATTTGGACCAGTGGGAGCAGCATATCTTTTTGTGGTG |
| | TAAATAGTGACACTGTTAGTTGGTCTTGGCCAGACGGTGCTGAGTTGCCATTCACCATTGACAAGTAGGGGC |
| | CCCTATAACTCTCTACGGCTAACCTGAATGGACTACGACATAGTCTAGTCCGCCAAGATGAGTCTTCTAACC |
| | GAGGTCGAAACGTACGTACTCTCTATCATCCCGTCAGGCCCCCTCAAAGCCGAGATCGCACAGAGACTTGAA |
| | GATGTCTTTGCAGGGAAGAACACCGATCTTGAGGTTCTCATGGAATGGCTAAAGACAAGACCAATCCTGTCA |
| | CCTCTGACTAAGGGGATTTTAGGATTTGTGTTCACGCTCACCGTGCCCAGTGAGCGAGGACTGCAGCGTAGA |
| | CGCTTTGTCCAAAATGCCCTTAATGGGAACGGGGATCCAAATAACATGGACAAAGCAGTTAAACTGTATAGG |
| | AAGCTCAAGAGGGAGATAACATTCCATGGGGCCAAAGAAATCTCACTCAGTTATTCTGCTGGTGCACTTGCC |
| | AGTTGTATGGGCCTCATATACAACAGGATGGGGGCTGTGACCACTGAAGTGGCATTTGGCCTGGTATGTGCA |
| | ACCTGTGAACAGATTGCTGACTCCCAGCATCGGTCTCATAGGCAAATGGTGACAACAACCAATCCACTAATC |
| | AGACATGAGAACAGAATGGTTTTAGCCAGCACTACAGCTAAGGCTATGGAGCAAATGGCTGGATCGAGTGAG |
| | CAAGCAGCAGAGGCCATGGAGGTTGCTAGTCAGGCTAGACAAATGGTGCAAGCGATGAGAACCATTGGGACT |
| | CATCCTAGCTCCAGTGCTGGTCTGAAAAATGATCTTCTTGAAAATTTGCAGGCCTATCAGAAACGAATGGGG |
| | GTGCAGATGCAACGGTTCAAGTGAGGCGCGCCCACCCAGCGGCCGCCCGCTACGCCCCAATGATCCGACCAG |
| | CAAAACTCGATGTACTTCCGAGGAACTGATGTGCATAATGCATCAGGCTGGTACATTAGATCCCCGCTTACC |
| | GCGGGCAATATAGCAACACTAAAAACTCGATGTACTTCCGAGGAAGCGCAGTGCATAATGCTGCGCAGTGTT |
| | GCCACATAACCACTATATTAACCATTTATCTAGCGGACGCCAAAAACTCAATGTATTTCTGAGGAAGCGTGG |
| | TGCATAATGCCACGCAGCGTCTGCATAACTTTTATTATTTCTTTTATTAATCAACAAAATTTTGTTTTTAAC |
| | ATTTCAAAAAAAAAAAAAAAAAAAAAAAAAAAAAAAAAAAAAGAAGAGCGTTTAAACACGTGATATCTGGCC |
| | TCATGGGCCTTCCTTTCACTGCCCGCTTTCCAGTCGGGAAACCTGTCGTGCCAGCTGCATTAACATGGTCAT |
| | AGCTGTTTCCTTGCGTATTGGGCGCTCTCCGCTTCCTCGCTCACTGACTCGCTGCGCTCGGTCGTTCGGGTA |
| | AAGCCTGGGGTGCCTAATGAGCAAAAGGCCAGCAAAAGGCCAGGAACCGTAAAAAGGCCGCGTTGCTGGCGT |
| | TTTTCCATAGGCTCCGCCCCCCTGACGAGCATCACAAAAATCGACGCTCAAGTCAGAGGTGGCGAAACCCGA |
| | CAGGACTATAAAGATACCAGGCGTTTCCCCCTGGAAGCTCCCTCGTGCGCTCTCCTGTTCCGACCCTGCCGC |
| | TTACCGGATACCTGTCCGCCTTTCTCCCTTCGGGAAGCGTGGCGCTTTCTCATAGCTCACGCTGTAGGTATC |
| | TCAGTTCGGTGTAGGTCGTTCGCTCCAAGCTGGGCTGTGTGCACGAACCCCCCGTTCAGCCCGACCGCTGCG |
| | CCTTATCCGGTAACTATCGTCTTGAGTCCAACCCGGTAAGACACGACTTATCGCCACTGGCAGCAGCCACTG |
| | GTAACAGGATTAGCAGAGCGAGGTATGTAGGCGGTGCTACAGAGTTCTTGAAGTGGTGGCCTAACTACGGCT |
| | ACACTAGAAGAACAGTATTTGGTATCTGCGCTCTGCTGAAGCCAGTTACCTTCGGAAAAAGAGTTGGTAGCT |
| | CTTGATCCGGCAAACAAACCACCGCTGGTAGCGGTGGTTTTTTTGTTTGCAGGCAGCAGATTACGCGCAGAA |
| | AAAAAGGATCTCAAGAAGATCCTTTGATCTTTTCTACGGGGTCTGACGCTCAGTGGAACGAAAACTCACGTT |
| | AAGGGATTTTGGTCATGAATACACGGTGCCTGACTGCGTTAGCAATTTAACTGTGATAAACTACCGCATTAA |
| | AGCTTATCGATGATAAGCTGTCAAACATGAGAATTCTTAGAAAAACTCATCGAGCATCAAATGAAACTGCAA |
| | TTTATTCATATCAGGATTATCAATACCATATTTTTGAAAAAGCCGTTTCTGTAATGAAGGAGAAAACTCACC |
| | GAGGCAGTTCCATAGGATGGCAAGATCCTGGTATCGGTCTGCGATTCCGACTCGTCCAACATCAATACAACC |
| | TATTAATTTCCCCTCGTCAAAAATAAGGTTATCAAGTGAGAAATCACCATGAGTGACGACTGAATCCGGTGA |
| | GAATGGCAAAAGCTTATGCATTTCTTTCCAGACTTGTTCAACAGGCCAGCCATTACGCTCGTCATCAAAATC |
| | ACTCGCATCAACCAAACCGTTATTCATTCGTGATTGCGCCTGAGCGAGACGAAATACGCGATCGCTGTTAAA |
| | AGGACAATTACAAACAGGAATCGAATGCAACCGGCGCAGGAACACTGCCAGCGCATCAACAATATTTTCACC |
| | TGAATCAGGATATTCTTCTAATACCTGGAATGCTGTTTTCCCGGGGATCGCAGTGGTGAGTAACCATGCATC |
| | ATCAGGAGTACGGATAAAATGCTTGATGGTCGGAAGAGGCATAAATTCCGTCAGCCAGTTTAGTCTGACCAT |
| | CTCATCTGTAACATCATTGGCAACGCTACCTTTGCCATGTTTCAGAAACAACTCTGGCGCATCGGGCTTCCC |
| | ATACAATCGATAGATTGTCGCACCTGATTGCCCGACATTATCGCGAGCCCATTTATACCCATATAAATCAGC |
| | ATCCATGTTGGAATTTAATCGCGGCCTCGAGCAAGACGTTTCCCGTTGAATATGGCTCATAACACCCCTTGT |
| | ATTACTGTTTATGTAAGCAGACAGTTTTATTGTTCATGAGCGGATACATATTTGAATGTATTTAGAAAAATA |
| | AACAAATAGGGGTTCCGCGCACATTTCCCCGAAAAGTGCCACCTAAATTGTAAGCGTTAATATTTTGTTAAA |
| | ATTCGCGTTAAATTTTTGTTAAATCAGCTCATTTTTTAACCAATAGGCCGAAATCGGCAAAATCCCTTATAA |
| | ATCAAAAGAATAGACCGAGATAGGGTTGAGTGGCCGCTACAGGGCGCTCCCATTCGCCATTCAGGCTGCGCA |
| | ACTGTTGGGAAGGGCGTTTCGGTGCGGGCCTCTTCGCTATTACGCCAGCTGGCGAAAGGGGGATGTGCTGCA |
| | AGGCGATTAAGTTGGGTAACGCCAGGGTTTTCCCAGTCACACGCGTAATACGACTCACTATAG |
| 83 | ATAGGCGGCGCATGAGAGAAGCCCAGACCAATTACCTACCCAAAATGGAGAAAGTTCACGTTGACATCGAGG |
| | AAGACAGCCCATTCCTCAGAGCTTTGCAGCGGAGCTTCCCGCAGTTTGAGGTAGAAGCCAAGCAGGTCACTG |
| | ATAATGACCATGCTAATGCCAGAGCGTTTTCGCATCTGGCTTCAAAACTGATCGAAACGGAGGTGGACCCAT |
| | CCGACACGATCCTTGACATTGGAAGTGCGCCCGCCCGCAGAATGTATTCTAAGCACAAGTATCATTGTATCT |
| | GTCCGATGAGATGTGCGGAAGATCCGGACAGATTGTATAAGTATGCAACTAAGCTGAAGAAAAACTGTAAGG |
| | AAATAACTGATAAGGAATTGGACAAGAAAATGAAGGAGCTCGCCGCCGTCATGAGCGACCCTGACCTGGAAA |
| | CTGAGACTATGTGCCTCCACGACGACGAGTCGTGTCGCTACGAAGGGCAAGTCGCTGTTTACCAGGATGTAT |
| | ACGCGGTTGACGGACCGACAAGTCTCTATCACCAAGCCAATAAGGGAGTTAGAGTCGCCTACTGGATAGGCT |
| | TTGACACCACCCCTTTTATGTTTAAGAACTTGGCTGGAGCATATCCATCATACTCTACCAACTGGGCCGACG |
| | AAACCGTGTTAACGGCTCGTAACATAGGCCTATGCAGCTCTGACGTTATGGAGCGGTCACGTAGAGGGATGT |
| | CCATTCTTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATTACACAT |
| | GTCGGTGTGAGACTATAGTTAGTTGCGACGGGTACGTCGTTAAAAGAATAGCTATCAGTCCAGGCCTGTATG |
| | GGAAGCCTTCAGGCTATGCTGCTACGATGCACCGCGAGGGATTCTTGTGCTGCAAAGTGACAGACACATTGA |
| | ACGGGGAGAGGGTCTCTTTTCCCGTGTGCACGTATGTGCCAGCTACATTGTGTGACCAAATGACTGGCATAC |
| | TGGCAACAGATGTCAGTGCGGACGACGCGCAAAAACTGCTGGTTGGGCTCAACCAGCGTATAGTCGTCAACG |
| | GTCGCACCCAGAGAAACACCAATACCATGAAAAATTACCTTTTGCCCGTAGTGGCCCAGGCATTTGCTAGGT |
| | GGGCAAAGGAATATAAGGAAGATCAAGAAGATGAAAGGCCACTAGGACTACGAGATAGACAGTTAGTCATGG |
| | GGTGTTGTTGGGCTTTTAGAAGGCACAAGATAACATCTATTTATAAGCGCCCGGATACCCAAACCATCATCA |
| | AAGTGAACAGCGATTTCCACTCATTCGTGCTGCCCAGGATAGGCAGTAACACATTGGAGATCGGGCTGAGAA |
| | CAAGAATCAGGAAAATGTTAGAGGAGCACAAGGAGCCGTCACCTCTCATTACCGCCGAGGACGTACAAGAAG |
| | CTAAGTGCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCGGCTCAG |
| | TGGAGACACCTCGTGGCTTGATAAAGGTTACCAGCTACGATGGCGAGGACAAGATCGGCTCTTACGCTGTGC |
| | TTTCTCCGCAGGCTGTACTCAAGAGTGAAAAATTATCTTGCATCCACCCTCTCGCTGAACAAGTCATAGTGA |
| | TAACACACTCTGGCCGAAAAGGGCGTTATGCCGTGGAACCATACCATGGTAAAGTAGTGGTGCCAGAGGGAC |
| | ATGCAATACCCGTCCAGGACTTTCAAGCTCTGAGTGAAAGTGCCACCATTGTGTACAACGAACGTGAGTTCG |
| | TAAACAGGTACCTGCACCATATTGCCACACATGGAGGAGCGCTGAACACTGATGAAGAATATTACAAAACTG |
| | TCAAGCCCAGCGAGCACGACGGCGAATACCTGTACGACATCGACAGGAAACAGTGCGTCAAGAAAGAACTAG |
| | TCACTGGGCTAGGGCTCACAGGCGAGCTGGTGGATCCTCCCTTCCATGAATTCGCCTACGAGAGTCTGAGAA |
| | CACGACCAGCCGCTCCTTACCAAGTACCAACCATAGGGGTGTATGGCGTGCCAGGATCAGGCAAGTCTGGCA |
| | TCATTAAAAGCGCAGTCACCAAAAAAGATCTAGTGGTGAGCGCCAAGAAAGAAAACTGTGCAGAAATTATAA |
| | GGGACGTCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGCTCATAG |
| | CCATTATAAGACCTAAAAAGGCAGTGCTCTGCGGGGATCCCAAACAGTGCGGTTTTTTTAACATGATGTGCC |
| | TGAAAGTGCATTTTAACCACGAGATTTGCACACAAGTCTTCCACAAAAGCATCTCTCGCCGTTGCACTAAAT |
| | CTGTGACTTCGGTCGTCTCAACCTTGTTTTACGACAAAAAAATGAGAACGACGAATCCGAAAGAGACTAAGA |
| | TTGTGATTGACACTACCGGCAGTACCAAACCTAAGCAGGACGATCTCATTCTCACTTGTTTCAGAGGGTGGG |
| | TGAAGCAGTTGCAAATAGATTACAAAGGCAACGAAATAATGACGGCAGCTGCCTCTCAAGGGCTGACCCGTA |
| | AAGGTGTGTATGCCGTTCGGTACAAGGTGAATGAAAATCCTCTGTACGCACCCACCTCAGAACATGTGAACG |
| | TCCTACTGACCCGCACGGAGGACCGCATCGTGTGGAAAACACTAGCCGGCGACCCATGGATAAAAACACTGA |
| | CTGCCAAGTACCCTGGGAATTTCACTGCCACGATAGAGGAGTGGCAAGCAGAGCATGATGCCATCATGAGGC |
| | ACATCTTGGAGAGACCGGACCCTACCGACGTCTTCCAGAATAAGGCAAACGTGTGTTGGGCCAAGGCTTTAG |
| | TGCCGGTGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGGACTCCG |
| | GTCTATTTTCTGCACCCACTGTTCCGTTATCCATTAGGAATAATCACTGGGATAACTCCCCGTCGCCTAACA |
| | TGTACGGGCTGAATAAAGAAGTGGTCCGTCAGCTCTCTCGCAGGTACCCACAACTGCCTCGGGCAGTTGCCA |
| | CTGGAAGAGTCTATGACATGAACACTGGTACACTGCGCAATTATGATCCGCGCATAAACCTAGTACCTGTAA |
| | ACAGAAGACTGCCTCATGCTTTAGTCCTCCACCATAATGAACACCCACAGAGTGACTTTTCTTCATTCGTCA |
| | GCAAATTGAAGGGCAGAACTGTCCTGGTGGTCGGGGAAAAGTTGTCCGTCCCAGGCAAAATGGTTGACTGGT |
| | TGTCAGACCGGCCTGAGGCTACCTTCAGAGCTCGGCTGGATTTAGGCATCCCAGGTGATGTGCCCAAATATG |
| | ACATAATATTTGTTAATGTGAGGACCCCATATAAATACCATCACTATCAGCAGTGTGAAGACCATGCCATTA |
| | AGCTTAGCATGTTGACCAAGAAAGCTTGTCTGCATCTGAATCCCGGCGGAACCTGTGTCAGCATAGGTTATG |
| | GTTACGCTGACAGGGCCAGCGAAAGCATCATTGGTGCTATAGCGCGGCAGTTCAAGTTTTCCCGGGTATGCA |
| | AACCGAAATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCGGATGTG |
| | CACCCTCATATCATGTGGTGCGAGGGGATATTGCCACGGCCACCGAAGGAGTGATTATAAATGCTGCTAACA |
| | GCAAAGGACAACCTGGCGGAGGGGTGTGCGGAGCGCTGTATAAGAAATTCCCGGAAAGCTTCGATTTACAGC |
| | CGATCGAAGTAGGAAAAGCGCGACTGGTCAAAGGTGCAGCTAAACATATCATTCATGCCGTAGGACCAAACT |
| | TCAACAAAGTTTCGGAGGTTGAAGGTGACAAACAGTTGGCAGAGGCTTATGAGTCCATCGCTAAGATTGTCA |
| | ACGATAACAATTACAAGTCAGTAGCGATTCCACTGTTGTCCACCGGCATCTTTTCCGGGAACAAAGATCGAC |
| | TAACCCAATCATTGAACCATTTGCTGACAGCTTTAGACACCACTGATGCAGATGTAGCCATATACTGCAGGG |
| | ACAAGAAATGGGAAATGACTCTCAAGGAAGCAGTGGCTAGGAGAGAAGCAGTGGAGGAGATATGCATATCCG |
| | ACGACTCTTCAGTGACAGAACCTGATGCAGAGCTGGTGAGGGTGCATCCGAAGAGTTCTTTGGCTGGAAGGA |
| | AGGGCTACAGCACAAGCGATGGCAAAACTTTCTCATATTTGGAAGGGACCAAGTTTCACCAGGCGGCCAAGG |
| | ATATAGCAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTAGCACGC |
| | TGCCTTGCTTGTGCATCCATGCCATGACTCCAGAAAGAGTACAGCGCCTAAAAGCCTCACGTCCAGAACAAA |
| | TTACTGTGTGCTCATCCTTTCCATTGCCGAAGTATAGAATCACTGGTGTGCAGAAGATCCAATGCTCCCAGC |
| | CTATATTGTTCTCACCGAAAGTGCCTGCGTATATTCATCCAAGGAAGTATCTCGTGGAAACACCACCGGTAG |
| | ACGAGACTCCGGAGCCATCGGCAGAGAACCAATCCACAGAGGGGACACCTGAACAACCACCACTTATAACCG |
| | AGGATGAGACCAGGACTAGAACGCCTGAGCCGATCATCATCGAAGAGGAAGAAGAGGATAGCATAAGTTTGC |
| | TGTCAGATGGCCCGACCCACCAGGTGCTGCAAGTCGAGGCAGACATTCACGGGCCGCCCTCTGTATCTAGCT |
| | CATCCTGGTCCATTCCTCATGCATCCGACTTTGATGTGGACAGTTTATCCATACTTGACACCCTGGAGGGAG |
| | CTAGCGTGACCAGCGGGGCAACGTCAGCCGAGACTAACTCTTACTTCGCAAAGAGTATGGAGTTTCTGGCGC |
| | GACCGGTGCCTGCGCCTCGAACAGTATTCAGGAACCCTCCACATCCCGCTCCGCGCACAAGAACACCGTCAC |
| | TTGCACCCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGGTCTCCA |
| | ACCCGCCAGGCGTAAATAGGGTGATTACAAGAGAGGAGTTTGAGGCGTTCGTAGCACAACAACAATGACGGT |
| | TTGATGCGGGTGCATACATCTTTTCCTCCGACACCGGTCAAGGGCATTTACAACAAAAATCAGTAAGGCAAA |
| | CGGTGCTATCCGAAGTGGTGTTGGAGAGGACCGAATTGGAGATTTCGTATGCCCCGCGCCTCGACCAAGAAA |
| | AAGAAGAATTACTACGCAAGAAATTACAGTTAAATCCCACACCTGCTAACAGAAGCAGATACCAGTCCAGGA |
| | AGGTGGAGAACATGAAAGCCATAACAGCTAGACGTATTCTGCAAGGCCTAGGGCATTATTTGAAGGCAGAAG |
| | GAAAAGTGGAGTGCTACCGAACCCTGCATCCTGTTCCTTTGTATTCATCTAGTGTGAACCGTGCCTTTTCAA |
| | GCCCCAAGGTCGCAGTGGAAGCCTGTAACGCCATGTTGAAAGAGAACTTTCCGACTGTGGCTTCTTACTGTA |
| | TTATTCCAGAGTACGATGCCTATTTGGACATGGTTGACGGAGCTTCATGCTGCTTAGACACTGCCAGTTTTT |
| | GCCCTGCAAAGCTGCGCAGCTTTCCAAAGAAACACTCCTATTTGGAACCCACAATACGATCGGCAGTGCCTT |
| | CAGCGATCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATAATGAAT |
| | ATTGGGAAACGTTTAAAGAAAACCCCATCAGGCTTACTGAAGAAAACGTGGTAAATTACATTACCAAATTAA |
| | AAGGACCAAAAGCTGCTGCTCTTTTTGCGAAGACACATAATTTGAATATGTTGCAGGACATACCAATGGACA |
| | GGTTTGTAATGGACTTAAAGAGAGACGTGAAAGTGACTCCAGGAACAAAACATACTGAAGAACGGCCCAAGG |
| | TACAGGTGATCCAGGCTGCCGATCCGCTAGCAACAGCGTATCTGTGCGGAATCCACCGAGAGCTGGTTAGGA |
| | GATTAAATGCGGTCCTGCTTCCGAACATTCATACACTGTTTGATATGTCGGCTGAAGACTTTGACGCTATTA |
| | TAGCCGAGCACTTCCAGCCTGGGGATTGTGTTCTGGAAACTGACATCGCGTCGTTTGATAAAAGTGAGGACG |
| | ACGCCATGGCTCTGACCGCGTTAATGATTCTGGAAGACTTAGGTGTGGACGCAGAGCTGTTGACGCTGATTG |
| | AGGCGGCTTTCGGCGAAATTTCATCAATACATTTGCCCACTAAAACTAAATTTAAATTCGGAGCCATGATGA |
| | AATCTGGAATGTTCCTCACACTGTTTGTGAACACAGTCATTAACATTGTAATCGCAAGCAGAGTGTTGAGAG |
| | AACGGCTAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGGGCGAGA |
| | AAGCGCCTTATTTCTGTGGAGGGTTTATTTTGTGTGACTCCGTGACCGGCACAGCGTGCCGTGTGGCAGACC |
| | CCCTAAAAAGGCTGTTTAAGCTTGGCAAACCTCTGGCAGCAGACGATGAACATGATGATGACAGGAGAAGGG |
| | CATTGCATGAAGAGTCAACACGCTGGAACCGAGTGGGTATTCTTTCAGAGCTGTGCAAGGCAGTAGAATCAA |
| | GGTATGAAACCGTAGGAACTTCCATCATAGTTATGGCCATGACTACTCTAGCTAGCAGTGTTAAATCATTCA |
| | GCTACCTGAGAGGGGCCCCTATAACTCTCTACGGCTAACCTGAATGGACTACGACATAGTCTAGTCCGCCAA |
| | GATGGAGAAAATAGTGCTTCTTCTTGCAATAGTCAGCCTTGTTAAAAGTGATCAGATTTGCATTGGTTACCA |
| | TGCAAACAACTCGACAGAGCAGGTTGACACAATAATGGAAAAGAACGTCACTGTTACACACGCCCAAGACAT |
| | ACTGGAAAAGACACACAACGGGAAACTCTGCGATCTAGATGGAGTGAAGCCTCTAATTTTAAGAGATTGTAG |
| | TGTAGCTGGATGGCTCCTCGGGAACCCAATGTGTGACGAATTCCTCAATGTGCCGGAATGGTCTTACATAGT |
| | GGAGAAGATCAATCCAGCCAATGACCTCTGTTACCCAGGGAATTTCAACGACTATGAAGAACTGAAACACCT |
| | ATTGAGCAGAATAAACCATTTTGAGAAAATTCAGATCATCCCCAAAAGTTCTTGGTCAGATCATGAAGCCTC |
| | AGCAGGGGTGAGCTCAGCATGTCCATACCAGGGAAGGTCCTCCTTTTTTAGAAATGTGGTATGGCTTATCAA |
| | AAAGGACAATGCATACCCAACAATAAAGAGAAGTTACAATAATACCAACCAAGAAGATCTTTTGGTATTGTG |
| | GGGGATTCACCATCCAAATGATGCGGCAGAGCAGACAAGGCTCTATCAAAACCCAACTACCTATATTTCCGT |
| | TGGGACATCAACACTAAACCAGAGATTGGTACCAAAAATAGCCACTAGATCTAAGGTAAACGGGCAAAGTGG |
| | AAGGATGGAGTTCTTTTGGACAATTTTAAAACCGAATGATGCAATAAACTTTGAGAGTAATGGAAATTTCAT |
| | TGCTCCAGAAAATGCATACAAAATTGTCAAGAAAGGGGACTCAACAATTATGAAAAGTGAGTTGGAATATGG |
| | TAACTGCAACACCAAGTGTCAAACTCCAATAGGGGCGATAAACTCTAGTATGCCATTCCACAACATCCACCC |
| | TCTCACCATCGGGGAATGCCCCAAATATGTGAAATCAAGCAGATTAGTCCTTGCTACTGGGCTCAGAAATAG |
| | CCCTCAACGAGAGACACGAGGACTATTTGGAGCTATAGCAGGTTTTATAGAGGGAGGATGGGGAATGGTAGA |
| | TGGTTGGTATGGGTACCACCATAGCAACGAGCAGGGGAGTGGGTACGCTGCAGACAAAGAATCCACTCAAAA |
| | GGCAATAGATGGAGTCACCAATAAGGTCAACTCGATCATTGACAAAATGAACACTCAGTTTGAGGCTGTTGG |
| | AAGGGAATTTAATAACTTAGAAAGGAGAATAGAAAATTTAAACAAGAAGATGGAAGACGGATTCCTAGATGT |
| | CTGGACTTATAATGCTGAACTTCTGGTTCTCATGGAAAATGAGAGAACTCTAGACTTTCATGACTCAAATGT |
| | CAAGAACCTTTACGACAAGGTCCGACTACAGCTTAGGGATAATGCAAAGGAGCTTGGTAACGGTTGTTTCGA |
| | GTTCTATCACAGATGTGATAATGAATGTATGGAAAGTGTAAGAAACGGAACGTATGACTACCCGCAGTATTC |
| | AGAAGAAGCAAGATTAAAAAGAGAGGAAATAAGTGGAGTAAAATTGGAATCAATAGGAACTTACCAAATACT |
| | GTCAATTTATTCAACAGTGGCGAGCTCCCTAGCACTGGCAATCATGGTGGCTGGTCTATCTTTATGGATGTG |
| | CTCCAATGGATCGTTACAATGCAGAATTTGCATTTAAGGGCCCCTATAACTCTCTACGGCTAACCTGAATGG |
| | ACTACGACATAGTCTAGTCCGCCAAGATGAATCCAAATCAGAAGATAATAACCATCGGATCAATCTGTATGG |
| | TAATTGGAATAGTTAGCTTAATGTTACAAATTGGGAACATGATCTCAATATGGGTCAGTCATTCAATTCAGA |
| | CAGGGAATCAATGCCAAGCTGAACCAATCAGCAATACTAAATTTCTTACTGAGAAAGCTGTGGCTTCAGTAA |
| | CATTAGCGGGCAATTCATCTCTTTGCCCCATTAGCGGATGGGCTGTATACAGTAAGGACAACAGTATAAGGA |
| | TCGGTTCCAGGGGGGATGTGTTTGTTATAAGAGAGCCGTTCATCTCATGCTCCCACTTGGAATGCAGAACTT |
| | TCTTTTTGACTCAGGGAGCCTTGCTGAATGACAAGCACTCCAATGGGACTGTCAAAGACAGAAGCCCTCACA |
| | GAACATTAATGAGTTGTCCTGTGGGTGAGGCTCCCTCCCCATATAACTCAAGGTTTGAGTCTGTTGCTTGGT |
| | CAGCAAGTGCTTGCCATGATGGCACCAGTTGGTTGACAATTGGAATTTCTGGTCCAGACAATGGGGCTGTGG |
| | CTGTATTGAAATACAATGGCATAATAACAGACACCATCAAGAGTTGGAGGAACAACATACTGAGAACTCAAG |
| | AGTCTGAATGTGCATGTGTAAATGGCTCTTGCTTTACTGTAATGACTGATGGACCAAGTAGTGGGCAGGCAT |
| | CATATAAGATCTTCAAAATGGAAAAAGGGAAAGTGGTTAAATCAGTCGAATTGGATGCTCCTAATTATCACT |
| | ATGAGGAGTGCTCCTGTTATCCTGATGCCGGCGAAATCACATGTGTGTGCAGGGATAATTGGCATGGCTCAA |
| | ATAGGCCATGGGTATCTTTCAATCAAAATTTGGAGTATCAAATAGGATATATATGCAGTGGAGTTTTCGGAG |
| | ACAATCCACGCCCCAATGATGGAACAGGTAGTTGTGGTCCGGTGTTCTCTAACGGGGCATATGGGGTAAAAG |
| | GGTTTTCATTCAAATACGGCAATGGTGTTTGGATCGGGAGAACCAAAAGCACTAATTCCAGGAGCGGCTTTG |
| | AAATGATTTGGGACCCAAATGGGTGGACTGGAACGGACAGTAGCTTTTCGGTGAAGCAAGATATCGTAGCAA |
| | TAACTGATTGGTCAGGATATAGCGGGAGTTTTGTCCAGCATCCAGAACTGACAGGATTAGATTGCATAAGAC |
| | CTTGTTTCTGGGTTGAGTTAATCAGAGGGCGGCCTAAAGAGAGCACAATTTGGACCAGTGGGAGCAGCATAT |
| | CTTTTTGTGGTGTAAATAGTGACACTGTTAGTTGGTCTTGGCCAGACGGTGCTGAGTTGCCATTCACCATTG |
| | ACAAGTAGCTCTCTACGGCTAACCTGAATGGACTACGACATAGTCTAGTCCGCCAAGATGAGTCTTCTAACC |
| | GAGGTCGAAACGTACGTACTCTCTATCATCCCGTCAGGCCCCCTCAAAGCCGAGATCGCACAGAGACTTGAA |
| | GATGTCTTTGCAGGGAAGAACACCGATCTTGAGGTTCTCATGGAATGGCTAAAGACAAGACCAATCCTGTCA |
| | CCTCTGACTAAGGGGATTTTAGGATTTGTGTTCACGCTCACCGTGCCCAGTGAGCGAGGACTGCAGCGTAGA |
| | CGCTTTGTCCAAAATGCCCTTAATGGGAACGGGGATCCAAATAACATGGACAAAGCAGTTAAACTGTATAGG |
| | AAGCTCAAGAGGGAGATAACATTCCATGGGGCCAAAGAAATCTCACTCAGTTATTCTGCTGGTGCACTTGCC |
| | AGTTGTATGGGCCTCATATACAACAGGATGGGGGCTGTGACCACTGAAGTGGCATTTGGCCTGGTATGTGCA |
| | ACCTGTGAACAGATTGCTGACTCCCAGCATCGGTCTCATAGGCAAATGGTGACAACAACCAATCCACTAATC |
| | AGACATGAGAACAGAATGGTTTTAGCCAGCACTACAGCTAAGGCTATGGAGCAAATGGCTGGATCGAGTGAG |
| | CAAGCAGCAGAGGCCATGGAGGTTGCTAGTCAGGCTAGACAAATGGTGCAAGCGATGAGAACCATTGGGACT |
| | CATCCTAGCTCCAGTGCTGGTCTGAAAAATGATCTTCTTGAAAATTTGCAGGCCTATCAGAAACGAATGGGG |
| | GTGCAGATGCAACGGTTCAAGTGAGGCGCGCCCACCCAGCGGCCGCCCGCTACGCCCCAATGATCCGACCAG |
| | CAAAACTCGATGTACTTCCGAGGAACTGATGTGCATAATGCATCAGGCTGGTACATTAGATCCCCGCTTACC |
| | GCGGGCAATATAGCAACACTAAAAACTCGATGTACTTCCGAGGAAGCGCAGTGCATAATGCTGCGCAGTGTT |
| | GCCACATAACCACTATATTAACCATTTATCTAGCGGACGCCAAAAACTCAATGTATTTCTGAGGAAGCGTGG |
| | TGCATAATGCCACGCAGCGTCTGCATAACTTTTATTATTTCTTTTATTAATCAACAAAATTTTGTTTTTAAC |
| | ATTTCAAAAAAAAAAAAAAAAAAAAAAAAAAAAAAAAAAAAAGAAGAGCGTTTAAACACGTGATATCTGGCC |
| | TCATGGGCCTTCCTTTCACTGCCCGCTTTCCAGTCGGGAAACCTGTCGTGCCAGCTGCATTAACATGGTCAT |
| | AGCTGTTTCCTTGCGTATTGGGCGCTCTCCGCTTCCTCGCTCACTGACTCGCTGCGCTCGGTCGTTCGGGTA |
| | AAGCCTGGGGTGCCTAATGAGCAAAAGGCCAGCAAAAGGCCAGGAACCGTAAAAAGGCCGCGTTGCTGGCGT |
| | TTTTCCATAGGCTCCGCCCCCCTGACGAGCATCACAAAAATCGACGCTCAAGTCAGAGGTGGCGAAACCCGA |
| | CAGGACTATAAAGATACCAGGCGTTTCCCCCTGGAAGCTCCCTCGTGCGCTCTCCTGTTCCGACCCTGCCGC |
| | TTACCGGATACCTGTCCGCCTTTCTCCCTTCGGGAAGCGTGGCGCTTTCTCATAGCTCACGCTGTAGGTATC |
| | TCAGTTCGGTGTAGGTCGTTCGCTCCAAGCTGGGCTGTGTGCACGAACCCCCCGTTCAGCCCGACCGCTGCG |
| | CCTTATCCGGTAACTATCGTCTTGAGTCCAACCCGGTAAGACACGACTTATCGCCACTGGCAGCAGCCACTG |
| | GTAACAGGATTAGCAGAGCGAGGTATGTAGGCGGTGCTACAGAGTTCTTGAAGTGGTGGCCTAACTACGGCT |
| | ACACTAGAAGAACAGTATTTGGTATCTGCGCTCTGCTGAAGCCAGTTACCTTCGGAAAAAGAGTTGGTAGCT |
| | CTTGATCCGGCAAACAAACCACCGCTGGTAGCGGTGGTTTTTTTGTTTGCAGGCAGCAGATTACGCGCAGAA |
| | AAAAAGGATCTCAAGAAGATCCTTTGATCTTTTCTACGGGGTCTGACGCTCAGTGGAACGAAAACTCACGTT |
| | AAGGGATTTTGGTCATGAATACACGGTGCCTGACTGCGTTAGCAATTTAACTGTGATAAACTACCGCATTAA |
| | TTTATTCATATCAGGATTATCAATACCATATTTTTGAAAAAGCCGTTTCTGTAATGAAGGAGAAAACTCACC |
| | AGCTTATCGATGATAAGCTGTCAAACATGAGAATTCTTAGAAAAACTCATCGAGCATCAAATGAAACTGCAA |
| | TTTATTCATATCAGGATTATCAATACCATATTTTTGAAAAAGCCGTTTCTGTAATGAAGGAGAAAACTCACC |
| | GAGGCAGTTCCATAGGATGGCAAGATCCTGGTATCGGTCTGCGATTCCGACTCGTCCAACATCAATACAACC |
| | TATTAATTTCCCCTCGTCAAAAATAAGGTTATCAAGTGAGAAATCACCATGAGTGACGACTGAATCCGGTGA |
| | GAATGGCAAAAGCTTATGCATTTCTTTCCAGACTTGTTCAACAGGCCAGCCATTACGCTCGTCATCAAAATC |
| | ACTCGCATCAACCAAACCGTTATTCATTCGTGATTGCGCCTGAGCGAGACGAAATACGCGATCGCTGTTAAA |
| | AGGACAATTACAAACAGGAATCGAATGCAACCGGCGCAGGAACACTGCCAGCGCATCAACAATATTTTCACC |
| | TGAATCAGGATATTCTTCTAATACCTGGAATGCTGTTTTCCCGGGGATCGCAGTGGTGAGTAACCATGCATC |
| | ATCAGGAGTACGGATAAAATGCTTGATGGTCGGAAGAGGCATAAATTCCGTCAGCCAGTTTAGTCTGACCAT |
| | CTCATCTGTAACATCATTGGCAACGCTACCTTTGCCATGTTTCAGAAACAACTCTGGCGCATCGGGCTTCCC |
| | ATACAATCGATAGATTGTCGCACCTGATTGCCCGACATTATCGCGAGCCCATTTATACCCATATAAATCAGC |
| | ATCCATGTTGGAATTTAATCGCGGCCTCGAGCAAGACGTTTCCCGTTGAATATGGCTCATAACACCCCTTGT |
| | ATTACTGTTTATGTAAGCAGACAGTTTTATTGTTCATGAGCGGATACATATTTGAATGTATTTAGAAAAATA |
| | AACAAATAGGGGTTCCGCGCACATTTCCCCGAAAAGTGCCACCTAAATTGTAAGCGTTAATATTTTGTTAAA |
| | ATTCGCGTTAAATTTTTGTTAAATCAGCTCATTTTTTAACCAATAGGCCGAAATCGGCAAAATCCCTTATAA |
| | ATCAAAAGAATAGACCGAGATAGGGTTGAGTGGCCGCTACAGGGCGCTCCCATTCGCCATTCAGGCTGCGCA |
| | ACTGTTGGGAAGGGCGTTTCGGTGCGGGCCTCTTCGCTATTACGCCAGCTGGCGAAAGGGGGATGTGCTGCA |
| | AGGCGATTAAGTTGGGTAACGCCAGGGTTTTCCCAGTCACACGCGTAATACGACTCACTATAG |
| 84 | ATAGGCGGCGCATGAGAGAAGCCCAGACCAATTACCTACCCAAAATGGAGAAAGTTCACGTTGACATCGAGG |
| | AAGACAGCCCATTCCTCAGAGCTTTGCAGCGGAGCTTCCCGCAGTTTGAGGTAGAAGCCAAGCAGGTCACTG |
| | ATAATGACCATGCTAATGCCAGAGCGTTTTCGCATCTGGCTTCAAAACTGATCGAAACGGAGGTGGACCCAT |
| | CCGACACGATCCTTGACATTGGAAGTGCGCCCGCCCGCAGAATGTATTCTAAGCACAAGTATCATTGTATCT |
| | GTCCGATGAGATGTGCGGAAGATCCGGACAGATTGTATAAGTATGCAACTAAGCTGAAGAAAAACTGTAAGG |
| | AAATAACTGATAAGGAATTGGACAAGAAAATGAAGGAGCTCGCCGCCGTCATGAGCGACCCTGACCTGGAAA |
| | CTGAGACTATGTGCCTCCACGACGACGAGTCGTGTCGCTACGAAGGGCAAGTCGCTGTTTACCAGGATGTAT |
| | ACGCGGTTGACGGACCGACAAGTCTCTATCACCAAGCCAATAAGGGAGTTAGAGTCGCCTACTGGATAGGCT |
| | TTGACACCACCCCTTTTATGTTTAAGAACTTGGCTGGAGCATATCCATCATACTCTACCAACTGGGCCGACG |
| | AAACCGTGTTAACGGCTCGTAACATAGGCCTATGCAGCTCTGACGTTATGGAGCGGTCACGTAGAGGGATGT |
| | CCATTCTTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATTACACAT |
| | GTCGGTGTGAGACTATAGTTAGTTGCGACGGGTACGTCGTTAAAAGAATAGCTATCAGTCCAGGCCTGTATG |
| | GGAAGCCTTCAGGCTATGCTGCTACGATGCACCGCGAGGGATTCTTGTGCTGCAAAGTGACAGACACATTGA |
| | ACGGGGAGAGGGTCTCTTTTCCCGTGTGCACGTATGTGCCAGCTACATTGTGTGACCAAATGACTGGCATAC |
| | TGGCAACAGATGTCAGTGCGGACGACGCGCAAAAACTGCTGGTTGGGCTCAACCAGCGTATAGTCGTCAACG |
| | GTCGCACCCAGAGAAACACCAATACCATGAAAAATTACCTTTTGCCCGTAGTGGCCCAGGCATTTGCTAGGT |
| | GGGCAAAGGAATATAAGGAAGATCAAGAAGATGAAAGGCCACTAGGACTACGAGATAGACAGTTAGTCATGG |
| | GGTGTTGTTGGGCTTTTAGAAGGCACAAGATAACATCTATTTATAAGCGCCCGGATACCCAAACCATCATCA |
| | AAGTGAACAGCGATTTCCACTCATTCGTGCTGCCCAGGATAGGCAGTAACACATTGGAGATCGGGCTGAGAA |
| | CAAGAATCAGGAAAATGTTAGAGGAGCACAAGGAGCCGTCACCTCTCATTACCGCCGAGGACGTACAAGAAG |
| | CTAAGTGCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCGGCTCAG |
| | TGGAGACACCTCGTGGCTTGATAAAGGTTACCAGCTACGATGGCGAGGACAAGATCGGCTCTTACGCTGTGC |
| | TTTCTCCGCAGGCTGTACTCAAGAGTGAAAAATTATCTTGCATCCACCCTCTCGCTGAACAAGTCATAGTGA |
| | TAACACACTCTGGCCGAAAAGGGCGTTATGCCGTGGAACCATACCATGGTAAAGTAGTGGTGCCAGAGGGAC |
| | ATGCAATACCCGTCCAGGACTTTCAAGCTCTGAGTGAAAGTGCCACCATTGTGTACAACGAACGTGAGTTCG |
| | TAAACAGGTACCTGCACCATATTGCCACACATGGAGGAGCGCTGAACACTGATGAAGAATATTACAAAACTG |
| | TCAAGCCCAGCGAGCACGACGGCGAATACCTGTACGACATCGACAGGAAACAGTGCGTCAAGAAAGAACTAG |
| | TCACTGGGCTAGGGCTCACAGGCGAGCTGGTGGATCCTCCCTTCCATGAATTCGCCTACGAGAGTCTGAGAA |
| | CACGACCAGCCGCTCCTTACCAAGTACCAACCATAGGGGTGTATGGCGTGCCAGGATCAGGCAAGTCTGGCA |
| | TCATTAAAAGCGCAGTCACCAAAAAAGATCTAGTGGTGAGCGCCAAGAAAGAAAACTGTGCAGAAATTATAA |
| | GGGACGTCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGCTCATAG |
| | CCATTATAAGACCTAAAAAGGCAGTGCTCTGCGGGGATCCCAAACAGTGCGGTTTTTTTAACATGATGTGCC |
| | TGAAAGTGCATTTTAACCACGAGATTTGCACACAAGTCTTCCACAAAAGCATCTCTCGCCGTTGCACTAAAT |
| | CTGTGACTTCGGTCGTCTCAACCTTGTTTTACGACAAAAAAATGAGAACGACGAATCCGAAAGAGACTAAGA |
| | TTGTGATTGACACTACCGGCAGTACCAAACCTAAGCAGGACGATCTCATTCTCACTTGTTTCAGAGGGTGGG |
| | TGAAGCAGTTGCAAATAGATTACAAAGGCAACGAAATAATGACGGCAGCTGCCTCTCAAGGGCTGACCCGTA |
| | AAGGTGTGTATGCCGTTCGGTACAAGGTGAATGAAAATCCTCTGTACGCACCCACCTCAGAACATGTGAACG |
| | TCCTACTGACCCGCACGGAGGACCGCATCGTGTGGAAAACACTAGCCGGCGACCCATGGATAAAAACACTGA |
| | CTGCCAAGTACCCTGGGAATTTCACTGCCACGATAGAGGAGTGGCAAGCAGAGCATGATGCCATCATGAGGC |
| | ACATCTTGGAGAGACCGGACCCTACCGACGTCTTCCAGAATAAGGCAAACGTGTGTTGGGCCAAGGCTTTAG |
| | TGCCGGTGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGGACTCCG |
| | GTCTATTTTCTGCACCCACTGTTCCGTTATCCATTAGGAATAATCACTGGGATAACTCCCCGTCGCCTAACA |
| | TGTACGGGCTGAATAAAGAAGTGGTCCGTCAGCTCTCTCGCAGGTACCCACAACTGCCTCGGGCAGTTGCCA |
| | CTGGAAGAGTCTATGACATGAACACTGGTACACTGCGCAATTATGATCCGCGCATAAACCTAGTACCTGTAA |
| | ACAGAAGACTGCCTCATGCTTTAGTCCTCCACCATAATGAACACCCACAGAGTGACTTTTCTTCATTCGTCA |
| | GCAAATTGAAGGGCAGAACTGTCCTGGTGGTCGGGGAAAAGTTGTCCGTCCCAGGCAAAATGGTTGACTGGT |
| | TGTCAGACCGGCCTGAGGCTACCTTCAGAGCTCGGCTGGATTTAGGCATCCCAGGTGATGTGCCCAAATATG |
| | ACATAATATTTGTTAATGTGAGGACCCCATATAAATACCATCACTATCAGCAGTGTGAAGACCATGCCATTA |
| | AGCTTAGCATGTTGACCAAGAAAGCTTGTCTGCATCTGAATCCCGGCGGAACCTGTGTCAGCATAGGTTATG |
| | GTTACGCTGACAGGGCCAGCGAAAGCATCATTGGTGCTATAGCGCGGCAGTTCAAGTTTTCCCGGGTATGCA |
| | AACCGAAATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCGGATGTG |
| | CACCCTCATATCATGTGGTGCGAGGGGATATTGCCACGGCCACCGAAGGAGTGATTATAAATGCTGCTAACA |
| | GCAAAGGACAACCTGGCGGAGGGGTGTGCGGAGCGCTGTATAAGAAATTCCCGGAAAGCTTCGATTTACAGC |
| | CGATCGAAGTAGGAAAAGCGCGACTGGTCAAAGGTGCAGCTAAACATATCATTCATGCCGTAGGACCAAACT |
| | TCAACAAAGTTTCGGAGGTTGAAGGTGACAAACAGTTGGCAGAGGCTTATGAGTCCATCGCTAAGATTGTCA |
| | ACGATAACAATTACAAGTCAGTAGCGATTCCACTGTTGTCCACCGGCATCTTTTCCGGGAACAAAGATCGAC |
| | TAACCCAATCATTGAACCATTTGCTGACAGCTTTAGACACCACTGATGCAGATGTAGCCATATACTGCAGGG |
| | ACAAGAAATGGGAAATGACTCTCAAGGAAGCAGTGGCTAGGAGAGAAGCAGTGGAGGAGATATGCATATCCG |
| | ACGACTCTTCAGTGACAGAACCTGATGCAGAGCTGGTGAGGGTGCATCCGAAGAGTTCTTTGGCTGGAAGGA |
| | AGGGCTACAGCACAAGCGATGGCAAAACTTTCTCATATTTGGAAGGGACCAAGTTTCACCAGGCGGCCAAGG |
| | ATATAGCAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTAGCACGC |
| | TGCCTTGCTTGTGCATCCATGCCATGACTCCAGAAAGAGTACAGCGCCTAAAAGCCTCACGTCCAGAACAAA |
| | TTACTGTGTGCTCATCCTTTCCATTGCCGAAGTATAGAATCACTGGTGTGCAGAAGATCCAATGCTCCCAGC |
| | CTATATTGTTCTCACCGAAAGTGCCTGCGTATATTCATCCAAGGAAGTATCTCGTGGAAACACCACCGGTAG |
| | ACGAGACTCCGGAGCCATCGGCAGAGAACCAATCCACAGAGGGGACACCTGAACAACCACCACTTATAACCG |
| | AGGATGAGACCAGGACTAGAACGCCTGAGCCGATCATCATCGAAGAGGAAGAAGAGGATAGCATAAGTTTGC |
| | TGTCAGATGGCCCGACCCACCAGGTGCTGCAAGTCGAGGCAGACATTCACGGGCCGCCCTCTGTATCTAGCT |
| | CATCCTGGTCCATTCCTCATGCATCCGACTTTGATGTGGACAGTTTATCCATACTTGACACCCTGGAGGGAG |
| | CTAGCGTGACCAGCGGGGCAACGTCAGCCGAGACTAACTCTTACTTCGCAAAGAGTATGGAGTTTCTGGCGC |
| | GACCGGTGCCTGCGCCTCGAACAGTATTCAGGAACCCTCCACATCCCGCTCCGCGCACAAGAACACCGTCAC |
| | TTGCACCCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGGTCTCCA |
| | ACCCGCCAGGCGTAAATAGGGTGATTACAAGAGAGGAGTTTGAGGCGTTCGTAGCACAACAACAATGACGGT |
| | TTGATGCGGGTGCATACATCTTTTCCTCCGACACCGGTCAAGGGCATTTACAACAAAAATCAGTAAGGCAAA |
| | CGGTGCTATCCGAAGTGGTGTTGGAGAGGACCGAATTGGAGATTTCGTATGCCCCGCGCCTCGACCAAGAAA |
| | AAGAAGAATTACTACGCAAGAAATTACAGTTAAATCCCACACCTGCTAACAGAAGCAGATACCAGTCCAGGA |
| | AGGTGGAGAACATGAAAGCCATAACAGCTAGACGTATTCTGCAAGGCCTAGGGCATTATTTGAAGGCAGAAG |
| | GAAAAGTGGAGTGCTACCGAACCCTGCATCCTGTTCCTTTGTATTCATCTAGTGTGAACCGTGCCTTTTCAA |
| | GCCCCAAGGTCGCAGTGGAAGCCTGTAACGCCATGTTGAAAGAGAACTTTCCGACTGTGGCTTCTTACTGTA |
| | TTATTCCAGAGTACGATGCCTATTTGGACATGGTTGACGGAGCTTCATGCTGCTTAGACACTGCCAGTTTTT |
| | GCCCTGCAAAGCTGCGCAGCTTTCCAAAGAAACACTCCTATTTGGAACCCACAATACGATCGGCAGTGCCTT |
| | CAGCGATCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATAATGAAT |
| | ATTGGGAAACGTTTAAAGAAAACCCCATCAGGCTTACTGAAGAAAACGTGGTAAATTACATTACCAAATTAA |
| | AAGGACCAAAAGCTGCTGCTCTTTTTGCGAAGACACATAATTTGAATATGTTGCAGGACATACCAATGGACA |
| | GGTTTGTAATGGACTTAAAGAGAGACGTGAAAGTGACTCCAGGAACAAAACATACTGAAGAACGGCCCAAGG |
| | TACAGGTGATCCAGGCTGCCGATCCGCTAGCAACAGCGTATCTGTGCGGAATCCACCGAGAGCTGGTTAGGA |
| | GATTAAATGCGGTCCTGCTTCCGAACATTCATACACTGTTTGATATGTCGGCTGAAGACTTTGACGCTATTA |
| | TAGCCGAGCACTTCCAGCCTGGGGATTGTGTTCTGGAAACTGACATCGCGTCGTTTGATAAAAGTGAGGACG |
| | ACGCCATGGCTCTGACCGCGTTAATGATTCTGGAAGACTTAGGTGTGGACGCAGAGCTGTTGACGCTGATTG |
| | AGGCGGCTTTCGGCGAAATTTCATCAATACATTTGCCCACTAAAACTAAATTTAAATTCGGAGCCATGATGA |
| | AATCTGGAATGTTCCTCACACTGTTTGTGAACACAGTCATTAACATTGTAATCGCAAGCAGAGTGTTGAGAG |
| | AACGGCTAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGGGCGAGA |
| | AAGCGCCTTATTTCTGTGGAGGGTTTATTTTGTGTGACTCCGTGACCGGCACAGCGTGCCGTGTGGCAGACC |
| | CCCTAAAAAGGCTGTTTAAGCTTGGCAAACCTCTGGCAGCAGACGATGAACATGATGATGACAGGAGAAGGG |
| | CATTGCATGAAGAGTCAACACGCTGGAACCGAGTGGGTATTCTTTCAGAGCTGTGCAAGGCAGTAGAATCAA |
| | GGTATGAAACCGTAGGAACTTCCATCATAGTTATGGCCATGACTACTCTAGCTAGCAGTGTTAAATCATTCA |
| | GCTACCTGAGAGGGGCCCCTATAACTCTCTACGGCTAACCTGAATGGACTACGACATAGTCTAGTCCGCCAA |
| | GATGGAGAAAATAGTGCTTCTTCTTGCAATAGTCAGCCTTGTTAAAAGTGATCAGATTTGCATTGGTTACCA |
| | TGCAAACAACTCGACAGAGCAGGTTGACACAATAATGGAAAAGAACGTCACTGTTACACACGCCCAAGACAT |
| | ACTGGAAAAGACACACAACGGGAAACTCTGCGATCTAGATGGAGTGAAGCCTCTAATTTTAAGAGATTGTAG |
| | TGTAGCTGGATGGCTCCTCGGGAACCCAATGTGTGACGAATTCCTCAATGTGCCGGAATGGTCTTACATAGT |
| | GGAGAAGATCAATCCAGCCAATGACCTCTGTTACCCAGGGAATTTCAACGACTATGAAGAACTGAAACACCT |
| | ATTGAGCAGAATAAACCATTTTGAGAAAATTCAGATCATCCCCAAAAGTTCTTGGTCAGATCATGAAGCCTC |
| | AGCAGGGGTGAGCTCAGCATGTCCATACCAGGGAAGGTCCTCCTTTTTTAGAAATGTGGTATGGCTTATCAA |
| | AAAGGACAATGCATACCCAACAATAAAGAGAAGTTACAATAATACCAACCAAGAAGATCTTTTGGTATTGTG |
| | GGGGATTCACCATCCAAATGATGCGGCAGAGCAGACAAGGCTCTATCAAAACCCAACTACCTATATTTCCGT |
| | TGGGACATCAACACTAAACCAGAGATTGGTACCAAAAATAGCCACTAGATCTAAGGTAAACGGGCAAAGTGG |
| | AAGGATGGAGTTCTTTTGGACAATTTTAAAACCGAATGATGCAATAAACTTTGAGAGTAATGGAAATTTCAT |
| | TGCTCCAGAAAATGCATACAAAATTGTCAAGAAAGGGGACTCAACAATTATGAAAAGTGAGTTGGAATATGG |
| | TAACTGCAACACCAAGTGTCAAACTCCAATAGGGGCGATAAACTCTAGTATGCCATTCCACAACATCCACCC |
| | TCTCACCATCGGGGAATGCCCCAAATATGTGAAATCAAGCAGATTAGTCCTTGCTACTGGGCTCAGAAATAG |
| | CCCTCAACGAGAGACACGAGGACTATTTGGAGCTATAGCAGGTTTTATAGAGGGAGGATGGGGAATGGTAGA |
| | TGGTTGGTATGGGTACCACCATAGCAACGAGCAGGGGAGTGGGTACGCTGCAGACAAAGAATCCACTCAAAA |
| | GGCAATAGATGGAGTCACCAATAAGGTCAACTCGATCATTGACAAAATGAACACTCAGTTTGAGGCTGTTGG |
| | AAGGGAATTTAATAACTTAGAAAGGAGAATAGAAAATTTAAACAAGAAGATGGAAGACGGATTCCTAGATGT |
| | CTGGACTTATAATGCTGAACTTCTGGTTCTCATGGAAAATGAGAGAACTCTAGACTTTCATGACTCAAATGT |
| | CAAGAACCTTTACGACAAGGTCCGACTACAGCTTAGGGATAATGCAAAGGAGCTTGGTAACGGTTGTTTCGA |
| | GTTCTATCACAGATGTGATAATGAATGTATGGAAAGTGTAAGAAACGGAACGTATGACTACCCGCAGTATTC |
| | AGAAGAAGCAAGATTAAAAAGAGAGGAAATAAGTGGAGTAAAATTGGAATCAATAGGAACTTACCAAATACT |
| | GTCAATTTATTCAACAGTGGCGAGCTCCCTAGCACTGGCAATCATGGTGGCTGGTCTATCTTTATGGATGTG |
| | CTCCAATGGATCGTTACAATGCAGAATTTGCATTTAAGGGCCCCTATAACTCTCTACGGCTAACCTGAATGG |
| | ACTACGACATAGTCTAGTCCGCCAAGATGAATCCAAATCAGAAGATAATAACCATCGGATCAATCTGTATGG |
| | TAATTGGAATAGTTAGCTTAATGTTACAAATTGGGAACATGATCTCAATATGGGTCAGTCATTCAATTCAGA |
| | CAGGGAATCAATGCCAAGCTGAACCAATCAGCAATACTAAATTTCTTACTGAGAAAGCTGTGGCTTCAGTAA |
| | CATTAGCGGGCAATTCATCTCTTTGCCCCATTAGCGGATGGGCTGTATACAGTAAGGACAACAGTATAAGGA |
| | TCGGTTCCAGGGGGGATGTGTTTGTTATAAGAGAGCCGTTCATCTCATGCTCCCACTTGGAATGCAGAACTT |
| | TCTTTTTGACTCAGGGAGCCTTGCTGAATGACAAGCACTCCAATGGGACTGTCAAAGACAGAAGCCCTCACA |
| | GAACATTAATGAGTTGTCCTGTGGGTGAGGCTCCCTCCCCATATAACTCAAGGTTTGAGTCTGTTGCTTGGT |
| | CAGCAAGTGCTTGCCATGATGGCACCAGTTGGTTGACAATTGGAATTTCTGGTCCAGACAATGGGGCTGTGG |
| | CTGTATTGAAATACAATGGCATAATAACAGACACCATCAAGAGTTGGAGGAACAACATACTGAGAACTCAAG |
| | AGTCTGAATGTGCATGTGTAAATGGCTCTTGCTTTACTGTAATGACTGATGGACCAAGTAGTGGGCAGGCAT |
| | CATATAAGATCTTCAAAATGGAAAAAGGGAAAGTGGTTAAATCAGTCGAATTGGATGCTCCTAATTATCACT |
| | ATGAGGAGTGCTCCTGTTATCCTGATGCCGGCGAAATCACATGTGTGTGCAGGGATAATTGGCATGGCTCAA |
| | ATAGGCCATGGGTATCTTTCAATCAAAATTTGGAGTATCAAATAGGATATATATGCAGTGGAGTTTTCGGAG |
| | ACAATCCACGCCCCAATGATGGAACAGGTAGTTGTGGTCCGGTGTTCTCTAACGGGGCATATGGGGTAAAAG |
| | GGTTTTCATTCAAATACGGCAATGGTGTTTGGATCGGGAGAACCAAAAGCACTAATTCCAGGAGCGGCTTTG |
| | AAATGATTTGGGACCCAAATGGGTGGACTGGAACGGACAGTAGCTTTTCGGTGAAGCAAGATATCGTAGCAA |
| | TAACTGATTGGTCAGGATATAGCGGGAGTTTTGTCCAGCATCCAGAACTGACAGGATTAGATTGCATAAGAC |
| | CTTGTTTCTGGGTTGAGTTAATCAGAGGGCGGCCTAAAGAGAGCACAATTTGGACCAGTGGGAGCAGCATAT |
| | CTTTTTGTGGTGTAAATAGTGACACTGTTAGTTGGTCTTGGCCAGACGGTGCTGAGTTGCCATTCACCATTG |
| | ACAAGTAGGGGCCCCTATAACTCTCTACGGCTAACCTGAATGGACTACGACATAGTCTAGTCCGCCAAGATG |
| | AGTCTTCTAACCGAGGTCGAAACGTACGTACTCTCTATCATCCCGTCAGGCCCCCTCAAAGCCGAGATCGCA |
| | CAGAGACTTGAAGATGTCTTTGCAGGGAAGAACACCGATCTTGAGGTTCTCATGGAATGGCTAAAGACAAGA |
| | CCAATCCTGTCACCTCTGACTAAGGGGATTTTAGGATTTGTGTTCACGCTCACCGTGCCCAGTGAGCGAGGA |
| | CTGCAGCGTAGACGCTTTGTCCAAAATGCCCTTAATGGGAACGGGGATCCAAATAACATGGACAAAGCAGTT |
| | AAACTGTATAGGAAGCTCAAGAGGGAGATAACATTCCATGGGGCCAAAGAAATCTCACTCAGTTATTCTGCT |
| | GGTGCACTTGCCAGTTGTATGGGCCTCATATACAACAGGATGGGGGCTGTGACCACTGAAGTGGCATTTGGC |
| | CTGGTATGTGCAACCTGTGAACAGATTGCTGACTCCCAGCATCGGTCTCATAGGCAAATGGTGACAACAACC |
| | AATCCACTAATCAGACATGAGAACAGAATGGTTTTAGCCAGCACTACAGCTAAGGCTATGGAGCAAATGGCT |
| | GGATCGAGTGAGCAAGCAGCAGAGGCCATGGAGGTTGCTAGTCAGGCTAGACAAATGGTGCAAGCGATGAGA |
| | ACCATTGGGACTCATCCTAGCTCCAGTGCTGGTCTGAAAAATGATCTTCTTGAAAATTTGCAGGCCTATCAG |
| | AAACGAATGGGGGTGCAGATGCAACGGTTCAAGTGAGGCGCGCCCACCCAGCGGCCGCCCGCTACGCCCCAA |
| | TGATCCGACCAGCAAAACTCGATGTACTTCCGAGGAACTGATGTGCATAATGCATCAGGCTGGTACATTAGA |
| | TCCCCGCTTACCGCGGGCAATATAGCAACACTAAAAACTCGATGTACTTCCGAGGAAGCGCAGTGCATAATG |
| | CTGCGCAGTGTTGCCACATAACCACTATATTAACCATTTATCTAGCGGACGCCAAAAACTCAATGTATTTCT |
| | GAGGAAGCGTGGTGCATAATGCCACGCAGCGTCTGCATAACTTTTATTATTTCTTTTATTAATCAACAAAAT |
| | TTTGTTTTTAACATTTCAAAAAAAAAAAAAAAAAAAAAAAAAAAAAAAAAAAAAGAAGAGCGTTTAAACACG |
| | TGATATCTGGCCTCATGGGCCTTCCTTTCACTGCCCGCTTTCCAGTCGGGAAACCTGTCGTGCCAGCTGCAT |
| | TAACATGGTCATAGCTGTTTCCTTGCGTATTGGGCGCTCTCCGCTTCCTCGCTCACTGACTCGCTGCGCTCG |
| | GTCGTTCGGGTAAAGCCTGGGGTGCCTAATGAGCAAAAGGCCAGCAAAAGGCCAGGAACCGTAAAAAGGCCG |
| | CGTTGCTGGCGTTTTTCCATAGGCTCCGCCCCCCTGACGAGCATCACAAAAATCGACGCTCAAGTCAGAGGT |
| | GGCGAAACCCGACAGGACTATAAAGATACCAGGCGTTTCCCCCTGGAAGCTCCCTCGTGCGCTCTCCTGTTC |
| | CGACCCTGCCGCTTACCGGATACCTGTCCGCCTTTCTCCCTTCGGGAAGCGTGGCGCTTTCTCATAGCTCAC |
| | GCTGTAGGTATCTCAGTTCGGTGTAGGTCGTTCGCTCCAAGCTGGGCTGTGTGCACGAACCCCCCGTTCAGC |
| | CCGACCGCTGCGCCTTATCCGGTAACTATCGTCTTGAGTCCAACCCGGTAAGACACGACTTATCGCCACTGG |
| | CAGCAGCCACTGGTAACAGGATTAGCAGAGCGAGGTATGTAGGCGGTGCTACAGAGTTCTTGAAGTGGTGGC |
| | CTAACTACGGCTACACTAGAAGAACAGTATTTGGTATCTGCGCTCTGCTGAAGCCAGTTACCTTCGGAAAAA |
| | GAGTTGGTAGCTCTTGATCCGGCAAACAAACCACCGCTGGTAGCGGTGGTTTTTTTGTTTGCAGGCAGCAGA |
| | TTACGCGCAGAAAAAAAGGATCTCAAGAAGATCCTTTGATCTTTTCTACGGGGTCTGACGCTCAGTGGAACG |
| | AAAACTCACGTTAAGGGATTTTGGTCATGAATACACGGTGCCTGACTGCGTTAGCAATTTAACTGTGATAAA |
| | CTACCGCATTAAAGCTTATCGATGATAAGCTGTCAAACATGAGAATTCTTAGAAAAACTCATCGAGCATCAA |
| | ATGAAACTGCAATTTATTCATATCAGGATTATCAATACCATATTTTTGAAAAAGCCGTTTCTGTAATGAAGG |
| | AGAAAACTCACCGAGGCAGTTCCATAGGATGGCAAGATCCTGGTATCGGTCTGCGATTCCGACTCGTCCAAC |
| | ATCAATACAACCTATTAATTTCCCCTCGTCAAAAATAAGGTTATCAAGTGAGAAATCACCATGAGTGACGAC |
| | TGAATCCGGTGAGAATGGCAAAAGCTTATGCATTTCTTTCCAGACTTGTTCAACAGGCCAGCCATTACGCTC |
| | GTCATCAAAATCACTCGCATCAACCAAACCGTTATTCATTCGTGATTGCGCCTGAGCGAGACGAAATACGCG |
| | ATCGCTGTTAAAAGGACAATTACAAACAGGAATCGAATGCAACCGGCGCAGGAACACTGCCAGCGCATCAAC |
| | AATATTTTCACCTGAATCAGGATATTCTTCTAATACCTGGAATGCTGTTTTCCCGGGGATCGCAGTGGTGAG |
| | TAACCATGCATCATCAGGAGTACGGATAAAATGCTTGATGGTCGGAAGAGGCATAAATTCCGTCAGCCAGTT |
| | TAGTCTGACCATCTCATCTGTAACATCATTGGCAACGCTACCTTTGCCATGTTTCAGAAACAACTCTGGCGC |
| | ATCGGGCTTCCCATACAATCGATAGATTGTCGCACCTGATTGCCCGACATTATCGCGAGCCCATTTATACCC |
| | ATATAAATCAGCATCCATGTTGGAATTTAATCGCGGCCTCGAGCAAGACGTTTCCCGTTGAATATGGCTCAT |
| | AACACCCCTTGTATTACTGTTTATGTAAGCAGACAGTTTTATTGTTCATGAGCGGATACATATTTGAATGTA |
| | TTTAGAAAAATAAACAAATAGGGGTTCCGCGCACATTTCCCCGAAAAGTGCCACCTAAATTGTAAGCGTTAA |
| | TATTTTGTTAAAATTCGCGTTAAATTTTTGTTAAATCAGCTCATTTTTTAACCAATAGGCCGAAATCGGCAA |
| | AATCCCTTATAAATCAAAAGAATAGACCGAGATAGGGTTGAGTGGCCGCTACAGGGCGCTCCCATTCGCCAT |
| | TCAGGCTGCGCAACTGTTGGGAAGGGCGTTTCGGTGCGGGCCTCTTCGCTATTACGCCAGCTGGCGAAAGGG |
| | GGATGTGCTGCAAGGCGATTAAGTTGGGTAACGCCAGGGTTTTCCCAGTCACACGCGTAATACGACTCACTA |
| | TAG |
| 85 | ACCATGG |
| 86 | TCATTCAGCTACCTGAGAGGGGCCCCTATAACTCTCTACGGCTAACCTGAATGGACTACGACATAGTCTAGT |
| | CCGCCAAG |
| 87 | GGGCCCCTATAACTCTCTACGGCTAACCTGAATGGACCTGAATGGACTACGACATAGTCTAGTCCGCCAAG |
| 88 | CCCCCCCCCCTAACGTTACTGGCCGAAGCCGCTTGGAATAAGGCCGGTGTGCGTTTGTCTATATGTTATTTT |
| | CCACCATATTGCCGTCTTTTGGCAATGTGAGGGCCCGGAAACCTGGCCCTGTCTTCTTGACGAGCATTCCTA |
| | GGGGTCTTTCCCCTCTCGCCAAAGGAATGCAAGGTCTGTTGAATGTCGTGAAGGAAGCAGTTCCTCTGGAAG |
| | CTTCTTGAAGACAAACAACGTCTGTAGCGACCCTTTGCAGGCAGCGGAACCCCCCACCTGGCGACAGGTGCC |
| | TCTGCGGCCAAAAGCCACGTGTATAAGATACACCTGCAAAGGCGGCACAACCCCAGTGCCACGTTGTGAGTT |
| | GGATAGTTGTGGAAAGAGTCAAATGGCTCTCCTCAAGCGTATTCAACAAGGGGCTGAAGGATGCCCAGAAGG |
| | TACCCCATTGTATGGGATCTGATCTGGGGCCTCGGTGCACATGCTTTACATGTGTTTAGTCGAGGTTAAAAA |
| | ACGTCTAGGCCCCCCGAACCACGGGGACGTGGTTTTCCTTTGAAAAACACGATGATAATATGGCCACAACC |
| 89 | CCCCGGCGCC |
| 90 | CCCCGGC |
| 91 | GCGCCCCGCGGCGCCCCGCG |
| 92 | TAATACGACTCACTATAG |

1. A composition comprising one or more ribonucleic acids (RNAs), each RNA comprising in 5′ to 3′ order:
a) a first nucleotide sequence comprising a 5′-untranslated region (5′-UTR), a fragment and/or a variant thereof,
b) one or more nucleotide sequence(s) encoding a virus-like particle (VLP) forming element;
c) a second nucleotide sequence comprising a 3′-untranslated region (3′-UTR), a fragment and/or a variant thereof, and
wherein the VLP forming element is selected from a hemaglutinin (HA) protein, a neuraminidase (NA) protein, and a matrix-1 (M1) protein of an influenza virus, and
wherein the composition comprises nucleotide sequences encoding each of the HA protein, the NA protein and the M1 protein.

2. The composition of claim 1, wherein the composition comprises a RNA comprising a nucleotide sequence encoding the HA protein, a nucleotide sequence encoding the NA protein, and a nucleotide sequence encoding the M1 protein.

3. The composition of claim 1, wherein the composition comprises a first and a second RNA, wherein the first RNA comprises a nucleotide sequence encoding the HA protein, the NA protein, or the M1 protein; and a second RNA comprising nucleotide sequences encoding a combination of:
d) the HA protein and the M1 protein;
e) the NA protein and the M1 protein; or
f) the HA protein and the NA protein, and wherein the first RNA and second RNA encode different VLP forming elements.

4. The composition of claim 1, wherein the composition comprises a first RNA comprising a nucleotide sequence encoding the HA protein, a second RNA comprising a nucleotide sequence encoding the NA protein, and a third RNA comprising a nucleotide sequence encoding the M1 protein.

5. The composition of any one of claims 1 to 4, wherein:
c) the HA protein is a H1, H2, H3, H4, H5, H6, H7, H8, H9, H10, H11, H12, H13, H14, H15, H16, H17, or H18 protein; and
d) the NA protein is a N1, N2, N3, N4, N5, N6, N7, N8, N9, N10, or N11 protein.

6. The composition of any one of claims 1 to 5, wherein the nucleotide sequence encoding:
d) the HA protein is at least 90% identical to a nucleotide sequence set forth in any one of SEQ ID Nos: 64 or 71;
e) the NA protein is at least 90% identical to a nucleotide sequence set forth in any one of SEQ ID Nos: 65 or 72; and
f) the M1 protein is at least 90% identical to a nucleotide sequence set forth in any one of SEQ ID Nos: 66 or 70.

7. The composition of any one of claims 1 to 6, wherein the nucleotide sequence encoding:
g) the HA protein is selected from a nucleotide sequence set forth in any one of SEQ ID Nos: 64 or 71;
h) the NA protein is selected from a nucleotide sequence set forth in any one of SEQ ID Nos: 65 or 72; and
i) the M1 protein is selected from a nucleotide sequence set forth in any one of SEQ ID Nos: 66 or 70.

8. The composition of any one of claims 1 to 7, wherein the VLP forming elements are from the same influenza virus.

9. The composition of any one of claims 1 to 7, wherein two or more of the VLP forming elements are from different influenza viruses.

10. The composition of claim 1, wherein the one or more RNA(s) comprises one or more additional nucleotide sequence encoding a matrix-2 (M2), nucleoprotein (NP) and/or a non-structural (NS) protein of an influenza virus, wherein the one or more additional nucleotide sequence is located 3′ or 5′ of the one or more nucleotide sequence(s) encoding the VLP forming element.

11. The composition of claim 10, wherein the nucleotide sequence encoding:
c) the NP protein is at least 90% identical to a nucleotide sequence set forth in SEQ ID NO: 69; and
d) the NS protein is at least 90% identical to a nucleotide sequence set forth in SEQ ID NO: 68.

12. The composition of claim 10 or 11, wherein the nucleotide sequence encoding:
c) the NP protein is selected from a nucleotide sequence set forth in SEQ ID NO: 69; and
d) the NS protein is selected from a nucleotide sequence set forth in SEQ ID NO: 68.

13. The composition of any one of claims 10 to 12, wherein the nucleoprotein (NP) and/or the non-structural (NS) are from the same influenza virus.

14. The composition of any one of claims 10 to 12, wherein the nucleoprotein (NP) and/or the non-structural (NS) are from different influenza viruses.

15. The composition of any one of claims 1 to 14, wherein the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 1 collagen (Col1A), alpha 6 collagen (Col6A), alpha-1-antitrypsin (SERPINA1), alpha-1-antichymotrypsin (SERPINA3), arachidonate 5-lipoxygenase (ALOX5), tyrosine hydroxylase (TH gene), tumor protein P53 inducible protein 3 (TP5313), an alphavirus, a fragment and/or a variant thereof.

16. The composition of any one of claims 1 to 15, wherein the first nucleotide sequence comprises the 5′-UTR of a Venezuelan equine encephalitis virus.

17. The composition of any one of claims 1 to 16, wherein the first nucleotide sequence comprises at least one microRNA binding site, an AU rich element (ARE), a GC-rich element, a stem loop, and combinations thereof.

18. The composition of any one of claims 1 to 17, wherein a translation initiation sequence selected from the group consisting of a Kozak consensus sequence, an internal ribosome entry site (IRES), a subgenomic (SG) promoter and combinations thereof is operably linked to the 5′ end of the one or more nucleotide sequence(s) encoding the VLP forming element and/or the one or more additional nucleotide sequence encoding a nucleoprotein (NP) and/or a non-structural (NS) protein.

19. The composition of claim 18, wherein the Kozak consensus sequence comprises or consists of a sequence set forth in SEQ ID NO: 1 (accatgg) or SEQ ID NO: 2 (accatg).

20. The composition of claim 18, wherein the IRES is an IRES from poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

21. The composition of claim 18, wherein the SG promoter is a minimal SG promoter or an extended SG promoter.

22. The composition of claim 21, wherein the SG promoter consists of the sequence set forth in SEQ ID NO: 3.

23. The composition of claim 21, wherein the extended SG promoter comprises the minimal SG promoter extended at the 5′ end with nucleotides occurring in a sequence encoding a non-structural protein of an RNA virus.

24. The composition of claim 21 or 23, wherein the extended SG promoter comprises a sequence set forth in SEQ ID NO: 75 or SEQ ID NO: 86 or SEQ ID NO: 87.

25. The RNA of any one of claims 1 to 24, wherein the second nucleotide sequence comprises the 3′-UTR of creatine kinase, globin, α-actin, albumin, granulocyte colony stimulating factor (G-CSF), collagen, ribophorin I (RPNI), low density lipoprotein receptor-related protein 1 (LRP1), cardiotrophin-like cytokine factor 1 (CLCF1), calreticulin (Calr), procollagen-lysine 2-oxoglutarate5-dioxygenase 1 (Plodl), nucleobindin1 (Nucbl), amino-terminal enhancer of split (AES), human mitochondrial 12S rRNA (mtRNR1), an alphavirus, a fragment and/or a variant thereof.

26. The composition of any one of claims 1 to 25, wherein the second nucleotide sequence comprises the 3′-UTR of a Venezuelan equine encephalitis virus (VEEV) or a Sindbis virus (SIN).

27. The RNA of any one of claims 1 to 26, wherein the second nucleotide sequence comprises at least one microRNA binding site, an AU rich element (ARE), a GC-rich element, a triple helix, a stem loop, one or more stop codons, a 3′CSE of an alphavirus and combinations thereof.

28. The composition of any one of claims 1 to 27, wherein the second nucleotide sequence comprises the 3′-CSE of a Venezuelan equine encephalitis virus (VEEV) or a Sindbis virus (SIN).

29. The composition of any one of claims 1 to 28, wherein the RNA comprises a third nucleotide sequence comprising one or more 3′ tailing sequences located at the 3′end of the second nucleotide sequence.

30. The composition of claim 29, wherein the one or more 3′ tailing sequences are selected from the group consisting of a poly-A sequence, polyadenylation signal, a G-quadruplex, a poly-C sequence, a stem loop and combinations thereof.

31. The composition of any one of claims 1 to 30, wherein the RNA comprises at least one chemically modified nucleotide.

32. The composition of claim 31, wherein the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), 1-methylpseudouridine (m1ψ), and combinations thereof.

33. The composition of any one of claims 1 to 32, wherein the RNA is messenger RNA (mRNA).

34. The composition of claim 33, wherein the mRNA is conventional mRNA (cRNA) or self-amplifying mRNA (sa-mRNA).

35. The composition of claim 34, wherein the sa-mRNA is from an alphavirus selected from the group consisting of Semliki Forest virus (SFV), Sindbis virus (SIN), and Venezuelan equine encephalitis virus (VEEV) and combinations thereof.

36. The composition of any one of claims 1 to 35, wherein the RNA further comprises a 5′ terminal cap structure.

37. The composition of claim 36, wherein the 5′ terminal cap structure is an endogenous cap or analogue thereof.

38. A composition comprising an RNA comprising in 5′ to 3′ order:
a) a nucleotide sequence encoding a hemaglutinin (HA) protein of an influenza virus operably linked to a nucleotide sequence comprising a 5′-untranslated region (5′-UTR), a fragment and/or a variant thereof or a first subgenomic (SG) promoter;
b) a nucleotide sequence encoding a neuraminidase (NA) protein of an influenza virus operably linked to a second subgenomic (SG) promoter; and
c) a nucleotide sequence encoding a matrix-1 (M1) protein of an influenza virus operably linked to a third subgenomic (SG) promoter.

39. The composition of claim 38, wherein the RNA is a sa-mRNA or a cRNA.

40. The composition of claim 38 or 39, wherein the nucleotide sequence encoding a hemaglutinin (HA) protein of an influenza virus is operably linked to a first subgenomic (SG) promoter.

41. The composition of any one of claims 38 to 40, wherein the second subgenomic (SG) promoter comprises the sequence set forth in SEQ ID NO: 3 or SEQ ID NO: 75.

42. The composition of any one of claims 38 to 41, wherein the third subgenomic (SG) promoter comprises the sequence set forth in SEQ ID NO: 3 or SEQ ID NO: 75.

43. The composition of any one of claims 38 to 42, wherein the second subgenomic (SG) promoter comprises the sequence set forth in SEQ ID NO: 75 and the third subgenomic (SG) promoter comprises the sequence set forth in SEQ ID NO: 3.

44. The composition of any one of claims 1 to 43 wherein the RNA are formulated in a lipid nanoparticle (LNP).

45. The composition of claim 44, wherein each RNA is formulated together in the LNP.

46. The composition of claim 44, wherein each RNA is formulated separately in the LNP.

47. The composition of any one of claims 1 to 46, wherein the composition is an immunogenic composition.

48. A pharmaceutical composition comprising an immunogenic composition of claim 47 and a pharmaceutically acceptable carrier.

49. The immunogenic composition of claim 47 or the pharmaceutical composition of claim 48 for use as a vaccine.

50. The immunogenic composition of claim 47, or the pharmaceutical composition of claim 48 for use in the treatment or prevention or delaying progression of influenza or influenza virus infection.

51. A method of treating or preventing or delaying progression of influenza in a subject, the method comprising administering the immunogenic composition of claim 47, or the pharmaceutical composition of claim 48 to a subject in need thereof.

52. Use of the composition of any one of claims 1 to 46, the immunogenic composition of claim 47, or the pharmaceutical composition of claim 48 in the manufacture of a medicament for treating or preventing or delaying progression of influenza in a subject in need thereof.

53. The immunogenic composition of claim 47, or the pharmaceutical composition of claim 48 for use in inducing an immune response in a subject in need thereof.

54. A method of inducing an immune response in a subject, the method comprising administering the composition of any one of claims 1 to 46, the immunogenic composition of claim 47, or the pharmaceutical composition of claim 48 to a subject in need thereof.

55. Use of the composition of any one of claims 1 to 46, or the immunogenic composition of claim 47, or the pharmaceutical composition of claim 48 in the manufacture of a medicament for inducing an immune response in a subject in need thereof.

56. The composition of claim 53, the method of claim 54 or the use of claim 55, wherein the immune response is a humoral and/or a cell-mediated immune response.

57. A method of expressing a virus-like particle (VLP) in a subject comprising administering the composition of any one of claims 1 to 46, the immunogenic composition of claim 47, or the pharmaceutical composition of claim 48 to the subject.

58. Use of the composition of any one of claims 1 to 46, or the immunogenic composition of claim 47, or the pharmaceutical composition of claim 48 in the manufacture of a medicament for expressing a VLP in a subject in need thereof.

59. The immunogenic composition of claim 47, or the pharmaceutical composition of claim 48 for use in a method of expressing a VLP in a subject in need thereof.

60. A method of inducing in a subject an immune response to influenza virus, the method comprising administering to the subject a composition of any one of claims 1 to 46, the immunogenic composition of claim 47, or the pharmaceutical composition of claim 48.

61. Use of the composition of any one of claims 1 to 46, or the immunogenic composition of claim 47, or the pharmaceutical composition of claim 48 in the manufacture of a medicament for inducing an immune response to influenza virus in a subject in need thereof.

62. The composition of any one of claims 1 to 46, the immunogenic composition of claim 47, or the pharmaceutical composition of claim 48 for use in inducing an immune response to influenza virus in a subject in need thereof.
